SEQUENCE LISTING (134 sequences)

SEQ ID NO: 1 - RNA, 3 nt, Artificial Sequence
  source: /note=“Description of Artificial Sequence: Synthetic oligonucleotide”
aug

SEQ ID NO: 2 - DNA, 717 bp, Artificial Sequence
  source: /note=“Description of Artificial Sequence: Synthetic polynucleotide”
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 3 - DNA, 57 bp, Artificial Sequence
  source: /note=“Description of Artificial Sequence: Synthetic oligonucleotide”
gctactaacttcagcctgctgaagcaggctggcgacgtggaggagaaccctggacct

SEQ ID NO: 4 - DNA, 221 bp, Artificial Sequence
  source: /note=“Description of Artificial Sequence: Synthetic polynucleotide”
gtaaaaagaggtgaaacctattatgtgtgagcagggcacagacgttgaaactggagccaggagaagtattggcaggctttaggttattaggtggttactctgtcttaaaaatgttctggctttcttcctgcatccactggcatactcatggtctgtttttaaatattttaattcccatttacaaagtgatttacccacaagcccaacctgtctgtcttcag

SEQ ID NO: 5 - DNA, 552 bp, Artificial Sequence
  source: /note=“Description of Artificial Sequence: Synthetic polynucleotide”
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataata

SEQ ID NO: 6 - DNA, 6926 bp, Artificial Sequence
  source: /note=“Description of Artificial Sequence: Synthetic polynucleotide”
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattccattgagaaatgactgagttccggtgctctcaagtcattgatctttgtcgacttttatttggtctctgtaataacgacttcaaaaacattaaattctgttgcgaagccagtaagctacaaaaagaaaaaacaagagagaatgctatagtcgtatagtatagtttcccgactatctgatacccattacttatctagggggaatgcgaacccaaaattttatcagttttctcggatatcgatagatattggggaataaatttaaataaataaattttgggcgggtttagggcgtggcaaaaagttttttggcaaatcgctagaaatttacaagacttataaaattatgaaaaaatacaacaaaattttaaacacgtgggcgtgacagttttgggcggttttagggcgttagagtaggcgaggacagggttacatcgactaggctttgatcctgatcaagaatatatatactttataccgcttccttctacatgttacctatttttcaacgaatctagtatacctttttactgtacgatttatgggtataataataagctaaatcgagactaagttttattgttatatatattttttttattttatgcagaaattaattaaaccggtcctgcaggtgatcaggcgcgccggttaccggccggccccgcggagcgtaagtattcaaaattccaaaattttttactagaaatattcgattttttaataggcagtttctatactattgtatactattgtagattcgttgaaaagtatgtaacaggaagaataaagcatttccgaccatgtaaagtatatatattcttaataaggatcaatagccgagtcgatctcgccatgtccgtctgtcttattgttttattaccgccgagacatcaggaactataaaagctagaaggatgagttttagcatacagattctagagacaaggacgcagagcaagtttgttgatccatgctgccacgctttaactttctcaaattgcccaaaactgccatgcccacatttttgaactattttcgaaattttttcataattgtattactcgtgtaaatttccatcaatttgccaaaaaactttttgtcacgcgttaacgccctaaagccgccaatttggtcacgcccacactattgagcaattatcaaattttttctcattttattccccaatatctatcgatatccccgattatgaaattattaaatttcgcgttcgcattcacactagctgagtaacgagtatctgatagttggggaaatcgacttattttttatatacaatgaaaatgaatttaatcatatgaatatcgattatagctttttatttaatatgaatatttatttgggcttaaggtgtaacctcctcgacataagactcacatggcgcaggcacattgaagacaaaaatactcattgtcgggtctcgcaccctccagcagcacctaaaattatgtcttcaattattgccaacattggagacacaattagtctgtggcacctcaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - skipped

SEQ ID NO: 8 - DNA, 708 bp, Artificial Sequence
  source: /note=“Description of Artificial Sequence: Synthetic polynucleotide”
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 9 - RNA, 100 nt, Artificial Sequence
  source: /note=“Description of Artificial Sequence: Synthetic polynucleotide”
cugagaugcagguacauccagcugaugagucccaaauaggacgaaagccauaccagccgaaaggcccuuggcaggguuccuggauuccacugcuauccac

SEQ ID NO: 10 - DNA, 516 bp, Artificial Sequence
  source: /note=“Description of Artificial Sequence: Synthetic polynucleotide”
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 11 - DNA, 264 bp, Artificial Sequence
  source: /note=“Description of Artificial Sequence: Synthetic polynucleotide”
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcct

SEQ ID NO: 12 - DNA, 273 bp, Artificial Sequence
  source: /note=“Description of Artificial Sequence: Synthetic polynucleotide”
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgctgatagtaact

SEQ ID NO: 13 - DNA, 330 bp, Artificial Sequence
  source: /note=“Description of Artificial Sequence: Synthetic polynucleotide”
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggaagcggagctactaacttcagcctgctgaagcaggctggagacgtggaggagaaccctggacctct

SEQ ID NO: 14 - DNA, 20 bp, Artificial Sequence
  source: /note=“Description of Artificial Sequence: Synthetic oligonucleotide”
ggtggctcccaggcgcagtt

SEQ ID NO: 15 - DNA, 20 bp, Artificial Sequence
  source: /note=“Description of Artificial Sequence: Synthetic oligonucleotide”
ggtggctcccagttactatc

SEQ ID NO: 16 - DNA, 20 bp, Artificial Sequence
  source: /note=“Description of Artificial Sequence: Synthetic oligonucleotide”
ggtggctcccagaggtccag

SEQ ID NO: 17 - DNA, 873 bp, Artificial Sequence
  source: /note=“Description of Artificial Sequence: Synthetic polynucleotide”
gggagccaccatggactacaaggacgacgacgacaagatcatcaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggctccggcgagggcaggggaagtcttctaacatgcggggacgtggaggaaaatcccggcccagactataaggacgacgacgacaaaatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctct

SEQ ID NO: 18 - DNA, 762 bp, Artificial Sequence
  source: /note=“Description of Artificial Sequence: Synthetic polynucleotide”
gggagccaccatggactacaaggacgacgacgacaagatcatcaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgctgatagtaagactataaggacgacgacgacaaaatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgatagtaact

SEQ ID NO: 19 - DNA, 330 bp, Artificial Sequence; identical to SEQ ID NO: 13
  source: /note=“Description of Artificial Sequence: Synthetic polynucleotide”

SEQ ID NO: 20 - DNA, 264 bp, Artificial Sequence; identical to SEQ ID NO: 11
  source: /note=“Description of Artificial Sequence: Synthetic polynucleotide”

SEQ ID NO: 21 - DNA, 273 bp, Artificial Sequence; identical to SEQ ID NO: 12
  source: /note=“Description of Artificial Sequence: Synthetic polynucleotide”

SEQ ID NO: 22 - DNA, 954 bp, Artificial Sequence
  source: /note=“Description of Artificial Sequence: Synthetic polynucleotide”
gggacctaacgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttcaagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatgggctccggcgagggcaggggaagtcttctaacatgcggggacgtggaggaaaatcccggcccagactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggaagcggagctactaacttcagcctgctgaagcaggctggagacgtggaggagaaccctggacctct

SEQ ID NO: 23 - DNA, 1314 bp, Artificial Sequence
  source: /note=“Description of Artificial Sequence: Synthetic polynucleotide”
gggacctaacgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttcaagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatgggctccggcgagggcaggggaagtcttctaacatgcggggacgtggaggaaaatcccggcccagactacaaggacgacgacgacaagatcatcgactataaagacgacgacgataaaggtggcgactataaggacgacgacgacaaagccattgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggaccttgatagtaact

SEQ ID NO: 24 - DNA, 1305 bp, Artificial Sequence
  source: /note=“Description of Artificial Sequence: Synthetic polynucleotide”
gggacctaacgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttcaagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatgggctccggcgagggcaggggaagtcttctaacatgcggggacgtggaggaaaatcccggcccagactacaaggacgacgacgacaagatcatcgactataaagacgacgacgataaaggtggcgactataaggacgacgacgacaaagccattgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctct

SEQ ID NO: 25 - DNA, 882 bp, Artificial Sequence
  source: /note=“Description of Artificial Sequence: Synthetic polynucleotide”
gggagccaccatggactacaaggacgacgacgacaagatcatcaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggctccggcgagggcaggggaagtcttctaacatgcggggacgtggaggaaaatcccggcccagactataaggacgacgacgacaaaatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggaccttgatagtaact

SEQ ID NO: 26 - DNA, 179 bp, Artificial Sequence
  source: /note=“Description of Artificial Sequence: Synthetic polynucleotide”
aaaaaacaaaaaacaaaacggctattatgcgttaccggcgagacgctacggacttgggaaaatccgttgaccttaaacggtcgtgtgggttcaagtccctccacccccacgccggaaacgcaatagccgaaaaacaaaaaacaaaaaaaacaaaaaaaaaaccaaaaaaacaaaacaca

SEQ ID NO: 27 - DNA, 43 bp, Artificial Sequence
  source: /note=“Description of Artificial Sequence: Synthetic primer”
cgcggatcctaatacgactcactatagggagacccaagctggc

SEQ ID NO: 28 - DNA, 79 bp, Artificial Sequence
  source: /note=“Description of Artificial Sequence: Synthetic primer”
aatagccgttttgttttttggattaccagtgtgccatagtgcaggatcacatcgtcgtggtattcactccagagcgatg

SEQ ID NO: 29 - DNA, 76 bp, Artificial Sequence
  source: /note=“Description of Artificial Sequence: Synthetic primer”
aatagccgttttgttttttggattaccagtgtgccatagtgcaggatcacacgggggaggggcaaacaacagatgg

SEQ ID NO: 30 - DNA, 84 bp, Artificial Sequence
  source: /note=“Description of Artificial Sequence: Synthetic primer”
aatagccgttttgttttttggattaccagtgtgccatagtgcaggatcacgctttttgcaaaagcctaggcctccaaaaaagcc

SEQ ID NO: 31 - DNA, 77 bp, Artificial Sequence
  source: /note=“Description of Artificial Sequence: Synthetic primer”
aatagccgttttgttttttggattaccagtgtgccatagtgcaggatcactagcaccgcctacatacctcgctctgc

SEQ ID NO: 32 - DNA, 80 bp, Artificial Sequence
  source: /note=“Description of Artificial Sequence: Synthetic primer”
aatagccgttttgttttttggattaccagtgtgccatagtgcaggatcacctatgtggcgcggtattatcccgtattgac

SEQ ID NO: 33 - DNA, 84 bp, Artificial Sequence
  source: /note=“Description of Artificial Sequence: Synthetic primer”
aatagccgttttgttttttggattaccagtgtgccatagtgcaggatcacatttcgataagccagtaagcagtgggttctctag

SEQ ID NO: 34 - DNA, 20 bp, Artificial Sequence
  source: /note=“Description of Artificial Sequence: Synthetic primer”
attcttgcccgcctgatgaa

SEQ ID NO: 35 - DNA, 20 bp, Artificial Sequence
  source: /note=“Description of Artificial Sequence: Synthetic primer”
ttgctcatggaaaacggtgt

SEQ ID NO: 36 - DNA, 20 bp, Artificial Sequence
  source: /note=“Description of Artificial Sequence: Synthetic primer”
tgatcctgcactatggcaca

SEQ ID NO: 37 - DNA, 20 bp, Artificial Sequence
  source: /note=“Description of Artificial Sequence: Synthetic primer”
ctggactagtggatccgagc

SEQ ID NO: 38 - DNA, 23 bp, Artificial Sequence
  source: /note=“Description of Artificial Sequence: Synthetic primer”
gacgaggcccagagcaagagagg

SEQ ID NO: 39 - DNA, 22 bp, Artificial Sequence
  source: /note=“Description of Artificial Sequence: Synthetic primer”
ggtgttgaaggtctcaaacatg

SEQ ID NO: 40 - DNA, 20 bp, Artificial Sequence
  source: /note=“Description of Artificial Sequence: Synthetic primer”
tgtgggcaatgtcatcaaaa

SEQ ID NO: 41 - skipped

SEQ ID NO: 42 - DNA, 21 bp, Artificial Sequence
  source: /note=“Description of Artificial Sequence: Synthetic primer”
gaagcacttgctacctcttgc

SEQ ID NO: 43 - DNA, 20 bp, Artificial Sequence
  source: /note=“Description of Artificial Sequence: Synthetic primer”
atttggtaaggcctgagctg

SEQ ID NO: 44 - DNA, 20 bp, Artificial Sequence
  source: /note=“Description of Artificial Sequence: Synthetic primer”
tcgctggtatcactcgtctg

SEQ ID NO: 45 - DNA, 20 bp, Artificial Sequence
  source: /note=“Description of Artificial Sequence: Synthetic primer”
gattctgaagaccgccagag

SEQ ID NO: 46 - DNA, 20 bp, Artificial Sequence
  source: /note=“Description of Artificial Sequence: Synthetic primer”
ctctcctgttgtgcttctcc

SEQ ID NO: 47 - DNA, 22 bp, Artificial Sequence
  source: /note=“Description of Artificial Sequence: Synthetic primer”
gtcaaagttcatcctgtccttg

SEQ ID NO: 48 - DNA, 2270 bp, Artificial Sequence
  source: /note=“Description of Artificial Sequence: Synthetic polynucleotide”
gggaatagccgaaaaacaaaaaacaaaaaaaacaaaaaaaaaaccaaaaaaacaaaacacaacgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgtagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatgggctccggcgagggcaggggaagtcttctaacatgcggggacgtggaggaaaatcccggcccagactacaaggacgacgacgacaagatcatcgactataaagacgacgacgataaaggtggcgactataaggacgacgacgacaaagccattgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagtgaaacagactttgaattttgaccttctcaagttggcgggagacgtggagtccaaccctggacctgactacaaggacgacgacgacaagatcatcgactataaagacgacgacgataaaggtggcgactataaggacgacgacgacaaagccattatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggaccttaaaaaaaacaaaaaacaaaacggctatt

SEQ ID NO: 49 - DNA, 2279 bp, Artificial Sequence
  source: /note=“Description of Artificial Sequence: Synthetic polynucleotide”
gggaatagccgaaaaacaaaaaacaaaaaaaacaaaaaaaaaaccaaaaaaacaaaacacaacgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgtagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatgggctccggcgagggcaggggaagtcttctaacatgcggggacgtggaggaaaatcccggcccagactacaaggacgacgacgacaagatcatcgactataaagacgacgacgataaaggtggcgactataaggacgacgacgacaaagccattgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagtgaaacagactttgaattttgaccttctcaagttggcgggagacgtggagtccaaccctggaccttgatagtaagactacaaggacgacgacgacaagatcatcgactataaagacgacgacgataaaggtggcgactataaggacgacgacgacaaagccattatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggaccttaaaaaaaacaaaaaacaaaacggctatt

SEQ ID NO: 50 - RNA, 51 nt, Artificial Sequence
  source: /note=“Description of Artificial Sequence: Synthetic oligonucleotide”
ucauaaucaauuuauuauuuucuuuuauuuuauucacauaauuuuguuuuu

SEQ ID NO: 51 - RNA, 19 nt, Artificial Sequence
  source: /note=“Description of Artificial Sequence: Synthetic oligonucleotide”
auuuuguuuuuaacauuuc

SEQ ID NO: 52 - RNA, 70 nt, Artificial Sequence
  source: /note=“Description of Artificial Sequence: Synthetic oligonucleotide”
ucauaaucaauuuauuauuuucuuuuauuuuauucacauaauuuuguuuuuauuuuguuuuuaacauuuc

SEQ ID NO: 53 - RNA, 1545 nt, Artificial Sequence
  source: /note=“Description of Artificial Sequence: Synthetic polynucleotide”
aaaauccguugaccuuaaacggucguguggguucaagucccuccacccccacgccggaaacgcaauagccgaaaaacaaaaaacaaaaaaaacaaaaaaaaaaccaaaaaaacaaaacacauuaaaacagccuguggguugaucccacccacaggcccauugggcgcuagcacucugguaucacgguaccuuugugcgccuguuuuauacccccucccccaacuguaacuuagaaguaacacacaccgaucaacagucagcguggcacaccagccacguuuugaucaagcacuucuguuaccccggacugaguaucaauagacugcucacgcgguugaaggagaaagcguucguuauccggccaacuacuucgaaaaaccuaguaacaccguggaaguugcagaguguuucgcucagcacuaccccaguguagaucaggucgaugagucaccgcauuccccacgggcgaccguggcgguggcugcguuggcggccugcccauggggaaacccaugggacgcucuaauacagacauggugcgaagagucuauugagcuaguugguaguccuccggccccugaaugcggcuaauccuaacugcggagcacacacccucaagccagagggcagugugucguaacgggcaacucugcagcggaaccgacuacuuuggguguccguguuucauuuuauuccuauacuggcugcuuauggugacaauugagagaucguuaccauauagcuauuggauuggccauccggugacuaauagagcuauuauauaucccuuuguuggguuuauaccacuuagcuugaaagagguuaaaacauuacaauucauuguuaaguugaauacagcaaaaugggagucaaaguucuguuugcccugaucugcaucgcuguggccgaggccaagcccaccgagaacaacgaagacuucaacaucguggccguggccagcaacuucgcgaccacggaucucgaugcugaccgcgggaaguugcccggcaagaagcugccgcuggaggugcucaaagagauggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccauggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuaaucauaaucaauuuauuauuuucuuuuauuuuauucacauaauuuuguuuuuauuuuguuuuuaacauuucaaaaaacaaaaaacaaaacggcuauuaugcguuaccggcgagacgcuacggacuu

SEQ ID NO: 54 - RNA, 1545 nt, Artificial Sequence; identical to SEQ ID NO: 53
  source: /note=“Description of Artificial Sequence: Synthetic polynucleotide”

SEQ ID NO: 55 - DNA, 20 bp, Artificial Sequence
  source: /note=“Description of Artificial Sequence: Synthetic primer”
caccgctcaggacaatcctt

SEQ ID NO: 56 - DNA, 741 bp, Artificial Sequence
  source: /note=“Description of Artificial Sequence: Synthetic polynucleotide”
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 57 - DNA, 558 bp, Artificial Sequence
  source: /note=“Description of Artificial Sequence: Synthetic polynucleotide”
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 58 - DNA, 566 bp, Artificial Sequence
  source: /note=“Description of Artificial Sequence: Synthetic polynucleotide”
acgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttcaagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatg

SEQ ID NO: 59 - RNA, 121 nt, Artificial Sequence
  source: /note=“Description of Artificial Sequence: Synthetic polynucleotide”
aaaauccguugaccuuaaacggucguguggguucaagucccuccacccccacgccggaaacgcaauagccgaaaaacaaaaaacaaaaaaaacaaaaaaaaaaccaaaaaaacaaaacaca

SEQ ID NO: 60 - DNA, 254 bp, Artificial Sequence
  source: /note=“Description of Artificial Sequence: Synthetic polynucleotide”
ataccagccgaaaggcccttggcagagaggtctgaaaagacctctgctgactatgtgatcttattaaaattaggttaaatttcgaggttaaaaatagttttaatattgctatagtcttagaggtcttgtatatttatacttaccacacaagatggaccggagcagccctccaatatctagtgtaccctcgtgctcgctcaaacattaagtggtgttgtgcgaaaagaatctcacttcaagaaaaagaaactagt

SEQ ID NO: 61 - protein, 8 aa, Artificial Sequence
  source: /note=“Description of Artificial Sequence: Synthetic peptide”
  VARIANT (2)..(2): /replace=“Ile”
  MOD_RES (4)..(4): Any amino acid
  SITE (1)..(8): /note=“Variant residues given in the sequence have no preference with respect to those in the annotations for variant positions”
DVEXNPGP

SEQ ID NO: 62 - RNA, 96 nt, Hepatitis delta virus
ggcucaucucgacaagaggcggcaguccucaguacucuuacucuuuucuguaaagaggagacugcuggacucgccgcccaaguucgagcaugagcc

SEQ ID NO: 63 - RNA, 74 nt, Hepatitis delta virus
ggcuagaggcggcaguccucaguacucuuacucuuuucuguaaagaggagacugcuggacucgccgcccgagcc

SEQ ID NO: 64 - protein, 15 aa, Artificial Sequence
  source: /note=“Description of Artificial Sequence: Synthetic peptide”
GLNDIFEAQKIEWHE

SEQ ID NO: 65 - protein, 26 aa, Artificial Sequence
  source: /note=“Description of Artificial Sequence: Synthetic peptide”
KRRWKKNFIAVSAANRFKKISSSGAL

SEQ ID NO: 66 - protein, 6 aa, Artificial Sequence
  source: /note=“Description of Artificial Sequence: Synthetic peptide”
EEEEEE

SEQ ID NO: 67 - protein, 13 aa, Artificial Sequence
  source: /note=“Description of Artificial Sequence: Synthetic peptide”
GAPVPYPDPLEPR

SEQ ID NO: 68 - protein, 8 aa, Artificial Sequence
  source: /note=“Description of Artificial Sequence: Synthetic peptide”
DYKDDDDK

SEQ ID NO: 69 - protein, 9 aa, Artificial Sequence
  source: /note=“Description of Artificial Sequence: Synthetic peptide”
YPYDVPDYA

SEQ ID NO: 70 - protein, 6 aa, Artificial Sequence
  source: /note=“Description of Artificial Sequence: Synthetic 6xHis tag”
HHHHHH

SEQ ID NO: 71 - protein, 10 aa, Artificial Sequence
  source: /note=“Description of Artificial Sequence: Synthetic peptide”
EQKLISEEDL

SEQ ID NO: 72 - protein, 18 aa, Artificial Sequence
  source: /note=“Description of Artificial Sequence: Synthetic peptide”
TKENPRSNQEESYDDNES

SEQ ID NO: 73 - protein, 15 aa, Artificial Sequence
  source: /note=“Description of Artificial Sequence: Synthetic peptide”
KETAAAKFERQHMDS

SEQ ID NO: 74 - protein, 38 aa, Artificial Sequence
  source: /note=“Description of Artificial Sequence: Synthetic polypeptide”
MDEKTTGWRGGHVVEGLAGELEQLRARLEHHPQGQREP

SEQ ID NO: 75 - protein, 13 aa, Artificial Sequence
  source: /note=“Description of Artificial Sequence: Synthetic peptide”
SLAELLNAGLGGS

SEQ ID NO: 76 - protein, 8 aa, Artificial Sequence
  source: /note=“Description of Artificial Sequence: Synthetic peptide”
TQDPSRVG

SEQ ID NO: 77 - protein, 12 aa, Artificial Sequence
  source: /note=“Description of Artificial Sequence: Synthetic peptide”
PDRVRAVSHWSS

SEQ ID NO: 78 - protein, 8 aa, Artificial Sequence
  source: /note=“Description of Artificial Sequence: Synthetic peptide”
WSHPQFEK

SEQ ID NO: 79 - protein, 6 aa, Artificial Sequence
  source: /note=“Description of Artificial Sequence: Synthetic peptide”
CCPGCC

SEQ ID NO: 80 - protein, 10 aa, Artificial Sequence
  source: /note=“Description of Artificial Sequence: Synthetic peptide”
EVHTNQDPLD

SEQ ID NO: 81 - protein, 14 aa, Artificial Sequence
  source: /note=“Description of Artificial Sequence: Synthetic peptide”
GKPIPNPLLGLDST

SEQ ID NO: 82 - protein, 11 aa, Artificial Sequence
  source: /note=“Description of Artificial Sequence: Synthetic peptide”
YTDIEMNRLGK

SEQ ID NO: 83 - protein, 8 aa, Artificial Sequence
  source: /note=“Description of Artificial Sequence: Synthetic peptide”
DLYDDDDK

SEQ ID NO: 84 - protein, 9 aa, Artificial Sequence
  source: /note=“Description of Artificial Sequence: Synthetic peptide”
  VARIANT (1)..(1): /replace=“His” or “ ”
  VARIANT (2)..(2): /replace=“Gly” or “ ”
  VARIANT (3)..(3): /replace=“Val” or “Ile” or “Ser” or “Met”
  MOD_RES (5)..(5): Any amino acid
  SITE (1)..(9): /note=“Variant residues given in the sequence have no preference with respect to those in the annotations for variant positions”
GDDEXNPGP

SEQ ID NO: 85 - protein, 9 aa, Artificial Sequence
  source: /note=“Description of Artificial Sequence: Synthetic peptide”
GDVESNPGP

SEQ ID NO: 86 - protein, 9 aa, Artificial Sequence
  source: /note=“Description of Artificial Sequence: Synthetic peptide”
GDIEENPGP

SEQ ID NO: 87 - protein, 7 aa, Artificial Sequence
  source: /note=“Description of Artificial Sequence: Synthetic peptide”
VEPNPGP

SEQ ID NO: 88 - protein, 7 aa, Artificial Sequence
  source: /note=“Description of Artificial Sequence: Synthetic peptide”
IETNPGP

SEQ ID NO: 89 - protein, 9 aa, Artificial Sequence
  source: /note=“Description of Artificial Sequence: Synthetic peptide”
GDIESNPGP

SEQ ID NO: 90 - protein, 9 aa, Artificial Sequence
  source: /note=“Description of Artificial Sequence: Synthetic peptide”
GDVELNPGP

SEQ ID NO: 91 - protein, 9 aa, Artificial Sequence
  source: /note=“Description of Artificial Sequence: Synthetic peptide”
GDIETNPGP

SEQ ID NO: 92 - protein, 8 aa, Artificial Sequence
  source: /note=“Description of Artificial Sequence: Synthetic peptide”
GDVENPGP

SEQ ID NO: 93 - protein, 9 aa, Artificial Sequence
  source: /note=“Description of Artificial Sequence: Synthetic peptide”
GDVEENPGP

SEQ ID NO: 94 - protein, 9 aa, Artificial Sequence
  source: /note=“Description of Artificial Sequence: Synthetic peptide”
GDVEQNPGP

SEQ ID NO: 95 - protein, 7 aa, Artificial Sequence
  source: /note=“Description of Artificial Sequence: Synthetic peptide”
IESNPGP

SEQ ID NO: 96 - protein, 9 aa, Artificial Sequence
  source: /note=“Description of Artificial Sequence: Synthetic peptide”
GDIELNPGP

SEQ ID NO: 97 - protein, 9 aa, Artificial Sequence
  source: /note=“Description of Artificial Sequence: Synthetic peptide”
HDIETNPGP

SEQ ID NO: 98 - protein, 9 aa, Artificial Sequence
  source: /note=“Description of Artificial Sequence: Synthetic peptide”
HDVETNPGP

SEQ ID NO: 99 - protein, 9 aa, Artificial Sequence
  source: /note=“Description of Artificial Sequence: Synthetic peptide”
HDVEMNPGP

SEQ ID NO: 100 - protein, 9 aa, Artificial Sequence
  source: /note=“Description of Artificial Sequence: Synthetic peptide”
GDMESNPGP

SEQ ID NO: 101 - protein, 9 aa, Artificial Sequence
  source: /note=“Description of Artificial Sequence: Synthetic peptide”
GDVETNPGP

SEQ ID NO: 102 - protein, 9 aa, Artificial Sequence
  source: /note=“Description of Artificial Sequence: Synthetic peptide”
GDIEQNPGP

SEQ ID NO: 103 - protein, 8 aa, Artificial Sequence
  source: /note=“Description of Artificial Sequence: Synthetic peptide”
DSEFNPGP

SEQ ID NO: 104 - RNA, 17 nt, Artificial Sequence
  source: /note=“Description of Artificial Sequence: Synthetic oligonucleotide”
uagagaaacucgcggag

SEQ ID NO: 105 - DNA, 29 bp, Artificial Sequence
  source: /note=“Description of Artificial Sequence: Synthetic oligonucleotide”
tttttcggctattcccaatagccgttttg

SEQ ID NO: 106 - DNA, 20 bp, Artificial Sequence
  source: /note=“Description of Artificial Sequence: Synthetic oligonucleotide”
ggctattcccaatagccgtt

SEQ ID NO: 107 - DNA, 20 bp, Artificial Sequence
  source: /note=“Description of Artificial Sequence: Synthetic primer”
acgacggtgtgtgcatgtat

SEQ ID NO: 108 - DNA, 20 bp, Artificial Sequence
  source: /note=“Description of Artificial Sequence: Synthetic primer”
ttcccaccacttcaggtctc

SEQ ID NO: 109 - DNA, 20 bp, Artificial Sequence
  source: /note=“Description of Artificial Sequence: Synthetic primer”
tacgcctgcaactgtgttgt

SEQ ID NO: 110 - DNA, 20 bp, Artificial Sequence
  source: /note=“Description of Artificial Sequence: Synthetic primer”
tcgatgatcttgtcgtcgtc

SEQ ID NO: 111 - DNA, 21 bp, Artificial Sequence
  source: /note=“Description of Artificial Sequence: Synthetic primer”
agggctgcttttaactctggt

SEQ ID NO: 112 - DNA, 21 bp, Artificial Sequence
  source: /note=“Description of Artificial Sequence: Synthetic primer”
ccccacttgattttggaggga

SEQ ID NO: 113 - DNA, 19 bp, Artificial Sequence
  source: /note=“Description of Artificial Sequence: Synthetic primer”
ggcaccatgggaagtgatt

SEQ ID NO: 114 - RNA, 17 nt, Artificial Sequence
  source: /note=“Description of Artificial Sequence: Synthetic oligonucleotide”
gaggugcucaaagagau

SEQ ID NO: 115 - DNA, 35 bp, Artificial Sequence
  source: /note=“Description of Artificial Sequence: Synthetic oligonucleotide”
ccgttgtggtctcccagataaacagtattttgtcc

SEQ ID NO: 116 - DNA, 74 bp, Artificial Sequence
  source: /note=“Description of Artificial Sequence: Synthetic primer”
ataccagccgaaaggcccttggcagagaggtctgaaaagacctctgctgactatgtgatcttattaaaattagg

SEQ ID NO: 117 - DNA, 74 bp, Artificial Sequence
  source: /note=“Description of Artificial Sequence: Synthetic primer”
gaaattaatacgactcactatagggagaccacaacggtttccctcctctataccagccgaaaggcccttggcag

SEQ ID NO: 118 - DNA, 79 bp, Artificial Sequence
  source: /note=“Description of Artificial Sequence: Synthetic primer”
acataccagatttcgatctggagaggtgaagaatacgaccacctagaggtctgaaaagacctctgctgactatgtgatc

SEQ ID NO: 119 - DNA, 70 bp, Artificial Sequence
  source: /note=“Description of Artificial Sequence: Synthetic primer”
gaaattaatacgactcactatagggagaccacaacggtttccctcctctaaaacataccagatttcgatc

SEQ ID NO: 120 - DNA, 1027 bp, Artificial Sequence
  source: /note=“Description of Artificial Sequence: Synthetic polynucleotide”
gacacgcggccttccaagcagttagggaaaccgacttctttgaagaagaaagctgactatgtgatcttattaaaattaggttaaatttcgaggttaaaaatagttttaatattgctatagtcttagaggtcttgtatatttatacttaccacacaagatggaccggagcagccctccaatatctagtgtaccctcgtgctcgctcaaacattaagtggtgttgtgcgaaaagaatctcacttcaagaaaaagaaactagtatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaactcgagctcggtacctgtccgcggtcgcgacgtacgcgggcggccgccataaattggatccatatatagggcccgggttataattacctcaggtcgacgtcccatggttttgtatagaatttacggctagcgccggatgcgacgccggtcgcgtcttatccggccttcctatatcaggcggtgtttaagacgccgccgcttcgcccaaatccttatgccggttcgacgactggacaaaatactgtttatct

SEQ ID NO: 121 - DNA, 1231 bp, Artificial Sequence
  source: /note=“Description of Artificial Sequence: Synthetic polynucleotide”
gacacgcggccttccaagcagttagggaaaccgacttctttgaagaagaaagctgactatgtgatcttattaaaattaggttaaatttcgaggttaaaaatagttttaatattgctatagtcttagaggtcttgtatatttatacttaccacacaagatggaccggagcagccctccaatatctagtgtaccctcgtgctcgctcaaacattaagtggtgttgtgcgaaaagaatctcacttcaagaaaaagaaactagtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcgagctcggtacctgtccgcggtcgcgacgtacgcgggcggccgccataaattggatccatatatagggcccgggttataattacctcaggtcgacgtcccatggttttgtatagaatttacggctagcgccggatgcgacgccggtcgcgtcttatccggccttcctatatcaggcggtgtttaagacgccgccgcttcgcccaaatccttatgccggttcgacgactggacaaaatactgtttatct

SEQ ID NO: 122 - DNA, 56 bp, Artificial Sequence
  source: /note=“Description of Artificial Sequence: Synthetic primer”
gaaattaatacgactcactatagggagaccacaacggtttccctgactatgtgatc

SEQ ID NO: 123 - DNA, 31 bp, Artificial Sequence
  source: /note=“Description of Artificial Sequence: Synthetic primer”
agataaacagtattttgtccagtcgtcgaac

SEQ ID NO: 124 - DNA, 35 bp, Artificial Sequence
  source: /note=“Description of Artificial Sequence: Synthetic oligonucleotide”
ggacaaaatactgtttatctgggagaccacaacgg

SEQ ID NO: 125 - DNA, 20 bp, Artificial Sequence
  source: /note=“Description of Artificial Sequence: Synthetic primer”
agatttcgttggggactggc

SEQ ID NO: 126 - DNA, 20 bp, Artificial Sequence
  source: /note=“Description of Artificial Sequence: Synthetic primer”
ctggagacgtggaggagaac

SEQ ID NO: 127 - DNA, 20 bp, Artificial Sequence
  source: /note=“Description of Artificial Sequence: Synthetic primer”
ccaaaagacggcaatatggt

SEQ ID NO: 128 - DNA, 32 bp, Artificial Sequence
  source: /note=“Description of Artificial Sequence: Synthetic oligonucleotide”
gtcaacggattttcccaagtccgtagcgtctc

SEQ ID NO: 129 - RNA, 558 nt, Artificial Sequence
  source: /note=“Description of Artificial Sequence: Synthetic polynucleotide”
augggagucaaaguucuguuugcccugaucugcaucgcuguggccgaggccaagcccaccgagaacaacgaagacuucaacaucguggccguggccagcaacuucgcgaccacggaucucgaugcugaccgcgggaaguugcccggcaagaagcugccgcuggaggugcucaaagagauggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccauggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuaa

SEQ ID NO: 130 - RNA, 552 nt, Artificial Sequence
  source: /note=“Description of Artificial Sequence: Synthetic polynucleotide”
acguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaaua

SEQ ID NO: 131 - DNA, 54 bp, Artificial Sequence
  source: /note=“Description of Artificial Sequence: Synthetic oligonucleotide”
gagggcaggggaagtctactaacatgcggggacgtggaggaaaatcccggccca

SEQ ID NO: 132 - DNA, 60 bp, Artificial Sequence
  source: /note=“Description of Artificial Sequence: Synthetic oligonucleotide”
cagtgtactaattatgctctcttgaaattggctggagatgttgagagcaacccaggtccc

SEQ ID NO: 133 - DNA, 796 bp, Artificial Sequence
  source: /note=“Description of Artificial Sequence: Synthetic polynucleotide”
gtaagaagcaaggtttcatttaggggaagggaaatgattcaggacgagagtctttgtgctgctgagtgcctgtgatgaagaagcatgttagtcctgggcaacgtagcgagaccccatctctacaaaaaatagaaaaattagccaggtatagtggcgcacacctgtgattccagctacgcaggaggctgaggtgggaggattgcttgagcccaggaggttgaggctgcagtgagctgtaatcatgccactactccaacctgggcaacacagcaaggaccctgtctcaaaagctacttacagaaaagaattaggctcggcacggtagctcacacctgtaatcccagcactttgggaggctgaggcgggcagatcacttgaggtcaggagtttgagaccagcctggccaacatggtgaaaccttgtctctactaaaaatatgaaaattagccaggcatggtggcacattcctgtaatcccagctactcgggaggctgaggcaggagaatcacttgaacccaggaggtggaggttgcagtaagccgagatcgtaccactgtgctctagccttggtgacagagcgagactgtcttaaaaaaaaaaaaaaaaaaaaaagaattaattaaaaatttaaaaaaaaatgaaaaaaagctgcatgcttgttttttgtttttagttattctacattgttgtcattattaccaaatattggggaaaatacaacttacagaccaatctcaggagttaaatgttactacgaaggcaaatgaactatgcgtaatgaacctggtaggcatta

SEQ ID NO: 134 - RNA, 55 nt, Artificial Sequence
  source: /note=“Description of Artificial Sequence: Synthetic oligonucleotide”
aaaaaacaaaaaacaaaacggcuauuaugcguuaccggcgagacgcuacggacuu

(19) United States
(12) Patent Application Publication
(10) Pub. No.: US 2022/0143062 A1
(43) Pub. Date: May 12, 2022

(54) CIRCULAR POLYRIBONUCLEOTIDES AND PHARMACEUTICAL COMPOSITIONS THEREOF

(71) Applicant: Flagship Pioneering Innovations VI, LLC, Cambridge, MA (US)

(72) Inventors: Avak KAHVEJIAN, Lexington, MA (US); Nicholas McCartney PLUGIS, Duxbury, MA (US); Alexandra Sophie DE BOER, Somerville, MA (US); Catherine CIFUENTES-ROJAS, Brookline, MA (US); Ki Young PAEK, Brighton, MA (US); Michael Donato MELFI, Boston, MA (US)

(21) Appl. No.: 17/433,639

(22) PCT Filed: Mar. 4, 2020
(86) PCT No.: PCT/US2020/021037

§ 371 Date: Aug. 25, 2021

Related U.S. Application Data
(60) Provisional application No. 62/967,545, filed on Jan. 29, 2020.
(60) Provisional application No. 62/840,174, filed on Apr. 29, 2019.
(60) Provisional application No. 62/825,683, filed on Mar. 28, 2019.
(60) Provisional application No. 62/813,666, filed on Mar. 4, 2019.

(51) Int. Cl. A61K 31/7088; C12N 15/10; C12N 15/113

(52) U.S. Cl. CPC A61K 31/7088; C12N 2310/532; C12N 15/113; C12N 15/1003

(57) ABSTRACT

This invention relates generally to pharmaceutical compositions and preparations of circular polyribonucleotides and uses thereof.

### CROSS REFERENCE

This application claims the benefit of U.S. Provisional Application No. 62/813,666, filed Mar. 4, 2019, U.S. Provisional Application No. 62/825,683, filed Mar. 28, 2019, U.S. Provisional Application No. 62/840,174, filed Apr. 29, 2019, and U.S. Provisional Application No. 62/967,545, filed Jan. 29, 2020, the entire contents of which are incorporated by reference.

### BACKGROUND

Certain circular polyribonucleotides are ubiquitously present in human tissues and cells, including tissues and cells of healthy individuals.

### SUMMARY

The present disclosure provides pharmaceutical compositions or preparations of circular polyribonucleotide molecules having specified or reduced amounts of linear polyribonucleotide molecules, and methods related thereto. The inventors have found that linear polyribonucleotide molecules in circular polyribonucleotide pharmaceutical compositions or preparations should be detected, monitored and/or controlled, e.g., reduced or purified from the circular polyribonucleotide pharmaceutical compositions or preparations.

### Pharmaceutical Preparations

In one aspect, a pharmaceutical preparation of circular polyribonucleotide molecules comprises a level of linear polyribonucleotide molecules that is below a predetermined threshold when measured by a specified method, e.g., the preparation comprises a level of linear polyribonucleotide molecules meeting a pharmaceutical release specification, e.g., the preparation comprises a level of linear polyribonucleotide molecules meeting a specification described herein below (e.g., a w/v specification or w/w specification). In some cases, the specification may be a level below a detection limit when measured by a specified method.

In another aspect, a pharmaceutical preparation of circular polyribonucleotide molecules comprises no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.

In another aspect, a pharmaceutical preparation of circular polyribonucleotide molecules comprises at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) circular polyribonucleotide molecules relative to the total ribonucleotide molecules in the pharmaceutical preparation. In some embodiments, at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) of total ribonucleotide molecules in the pharmaceutical preparation are circular polyribonucleotide molecules. In some embodiments, at least 91% (w/w), 92% (w/w), 93% (w/w), 94% (w/w), 95% (w/w), 96% (w/w), 97% (w/w), 98% (w/w) or 99% (w/w) of total ribonucleotide molecules in the pharmaceutical preparation are circular polyribonucleotide molecules.

In another aspect, a pharmaceutical preparation of circular polyribonucleotide molecules has a level of linear polyribonucleotide molecules that is reduced by at least 30% (w/w), at least 40% (w/w), at least 50% (w/w), at least 60% (w/w), at least 70% (w/w), at least 80% (w/w), at least 90% (w/w), or at least 95% (w/w) after a purification step (e.g., after one or a plurality of purification steps) compared to the level of linear polyribonucleotide molecules in the preparation prior to the purification step(s).

In another aspect, a pharmaceutical preparation of circular polyribonucleotide molecules comprises circular polyribonucleotide molecules and no more than 5% (w/w) nicked polyribonucleotide molecules of the total ribonucleotide molecules in the pharmaceutical preparation. In some embodiments, the pharmaceutical composition comprises no more than 9% (w/w), 8% (w/w), 7% (w/w), 6% (w/w), 5% (w/w), 4% (w/w), 3% (w/w), 2% (w/w), 1% (w/w), or 0.5% (w/w) nicked polyribonucleotide molecules of the total ribonucleotide molecules in the pharmaceutical preparation. In some embodiments, the pharmaceutical composition comprises no more than 2% (w/w) nicked polyribonucleotide molecules of the total ribonucleotide molecules in the pharmaceutical preparation.

In another aspect, a pharmaceutical preparation of circular polyribonucleotide molecules comprises circular polyribonucleotide molecules and no more than 0.5% (w/w), 1% (w/w), 2% (w/w), 3% (w/w), 4% (w/w), 5% (w/w), 6% (w/w), 7% (w/w), 8% (w/w), 9% (w/w), or 10% (w/w) linear polyribonucleotide molecules of the total ribonucleotide molecules in the preparation.

In some embodiments of each aspect recited above, the pharmaceutical preparation of circular polyribonucleotide molecules comprises circular polyribonucleotide molecules and no more than 0.5% (w/w), 1% (w/w), 2% (w/w), or 5% (w/w) linear polyribonucleotide molecules of the total ribonucleotide molecules in the preparation.

In some embodiments of each aspect recited above, the circular polyribonucleotide molecules include a sequence, or plurality of sequences, encoding expression product(s), e.g., therapeutic expression products, e.g., encoding a therapeutic protein or nucleic acid. In some embodiments of each aspect recited above, the circular polyribonucleotide molecules include a sequence, or plurality of sequences, comprising a scaffold (e.g., an aptamer sequence).

In some embodiments of each aspect recited above, the level of linear polyribonucleotide molecules in a pharmaceutical preparation of circular polyribonucleotide molecules may be measured by any suitable method, including microscopy, spectrophotometry, fluorometry, denaturing urea polyacrylamide gel electrophoresis imaging, UV-Vis spectrophotometry, RNA electrophoresis, RNAse H analysis, UV spectroscopic or fluorescence detectors, light scattering techniques, surface plasmon resonance (SPR) with or without the use of methods of separation including HPLC, by HPLC, chip or gel based electrophoresis with or without using either pre- or post-separation derivatization methodologies, using methods of detection that use silver or dye stains or radioactive decay for detection of linear polyribonucleotide molecules, or methods that utilize microscopy, visual methods or a spectrophotometer, or any combination thereof.

In some embodiments of each aspect recited above, a pharmaceutical preparation of circular polyribonucleotide molecules also produces a reduced level of one or more marker(s) of an immune or inflammatory response after administration to a subject when the pharmaceutical preparation has undergone an enrichment or purification step (or a plurality of purification steps) to reduce linear polyribonucleotides, compared to prior to the purification step(s). In some embodiments, the one or more marker(s) of an immune or inflammatory response is expression of a cytokine or an immunogenic related gene. In some embodiments, the one or more marker(s) of an immune or inflammatory response is expression of a gene selected from the group consisting of RIG-I, MDA5, PKR, IFN-beta, OAS, and OASL.

In some embodiments of each aspect recited above, a pharmaceutical preparation of circular polyribonucleotide molecules is further substantially free of an impurity, e.g., a process-related impurity or a product-related substance. In some embodiments, the process-related impurity comprises a protein (e.g., a cell protein such as a host cell protein), a deoxyribonucleic acid (e.g., a cell deoxyribonucleic acid such as a host cell deoxyribonucleic acid), monodeoxyribonucleotide or dideoxyribonucleotide molecules, an enzyme (e.g., a nuclease or ligase), a reagent component, a gel component, or a chromatographic material. In some embodiments, the impurity is selected from: a buffer reagent, a ligase, a nuclease (e.g., exonuclease or endonuclease), RNase inhibitor, RNase R, deoxyribonucleotide molecules, acrylamide gel debris, and monodeoxyribonucleotide molecules. In some embodiments, the pharmaceutical preparation comprises protein contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of protein contamination per milligram (mg) of the circular polyribonucleotide molecules.

In some embodiments of each aspect recited above, the pharmaceutical preparation is further substantially free of a pharmaceutical impurity or contaminant, e.g., the pharmaceutical preparation comprises less than 10 EU/kg of, or lacks, endotoxin as measured by a Limulus amebocyte lysate test. In some embodiments, the pharmaceutical preparation comprises a bioburden of less than 100 CFU/100 ml or less than 10 CFU/100 ml before sterilization. In some embodiments, the pharmaceutical preparation is a sterile pharmaceutical preparation. In some embodiments, the sterile pharmaceutical preparation supports growth of fewer than 100 viable microorganisms as tested under aseptic conditions. In some embodiments, the pharmaceutical preparation meeting the standard of the U.S. Pharmacopeia chapter 71 (USP <71>) published as of the filing date of the instant application. In some embodiments, the pharmaceutical preparation meets the standard of U.S. Pharmacopeia chapter 85 (USP <85>) published as of the filing date of the instant application.

In some embodiments of each aspect recited above, a linear polyribonucleotide molecule of the preparation comprises a linear polyribonucleotide molecule counterpart of the circular polyribonucleotide molecules or a fragment of the linear polyribonucleotide molecule counterpart of the circular polyribonucleotide molecules. In some embodiments of each aspect recited above, a linear polyribonucleotide molecule of the preparation comprises a linear polyribonucleotide molecule counterpart (e.g., a pre-circularized version) of the circular polyribonucleotide molecules. In some embodiments, the linear polyribonucleotide molecules comprise a linear polyribonucleotide molecule counterpart of a circular polyribonucleotide molecule or a fragment thereof, a linear polyribonucleotide molecule non-counterpart of the circular polyribonucleotide molecule or a fragment thereof, or a combination thereof. In some embodiments, the linear polyribonucleotide molecules comprise a linear polyribonucleotide molecule counterpart of a circular polyribonucleotide molecule (e.g., a pre-circularized version), a linear polyribonucleotide molecule non-counterpart of the circular polyribonucleotide molecule, or a combination thereof. In some embodiments of each aspect recited above, a linear polyribonucleotide molecule fragment is a fragment that is at least 1, 2, 3, 4, 5, 6, 7, 8, 9, 10, 20, 30, 40, 50, 60, 70, 80, 90, 100, 200, 300, 400, 500, 1000, 2000, 3000, 4000, 5000, 6000, 7000, 8000, 9000, 10000, 11000, 12000, or more nucleotides in length, or any nucleotide number therebetween.

In some embodiments of each aspect recited above, the circular polyribonucleotide molecules comprise a quasi-helical structure. In some embodiments, the circular polyribonucleotide molecules comprise a quasi-double stranded secondary structure. In some embodiments of each aspect recited above, the circular polyribonucleotide molecules comprise one or more expression sequences and a stagger element at a 3′ end of at least one expression sequence. In some embodiments of each aspect recited above, the circular polyribonucleotide molecules comprise one or more aptamer sequences. In some embodiments of each aspect recited above, the circular polyribonucleotide molecules have a sequence encoding an endogenous or naturally occurring circular polyribonucleotide sequence.

In some embodiments of each aspect recited above, the pharmaceutical preparation is an intermediate pharmaceutical preparation of a final circular polyribonucleotide drug product. In some embodiments, the pharmaceutical preparation is a drug substance or active pharmaceutical ingredient (API). In some embodiments, the pharmaceutical preparation is a drug product for administration to a subject.

In some embodiments of each aspect recited above, the pharmaceutical preparation comprises a concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 4 mg/mL, 5 mg/mL, 10 mg/mL, 100 mg/mL, 200 mg/mL, or 500 mg/mL circular polyribonucleotide molecules.

In some embodiments of each aspect recited above, the pharmaceutical preparation comprises zero DNA, is substantially free of DNA, or no more than 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, or 1 μg/mL of DNA. In some embodiments, the DNA comprises monodeoxyribonucleotide, dideoxyribonucleotide molecules, polydeoxyribonucleotide molecules, or any combination thereof. In some embodiments, the pharmaceutical preparation has an A260/A280 absorbance ratio of from about 1.6 to 2.3 as measured by a spectrophotometer. In some embodiments, DNA concentration of the pharmaceutical preparation is measured after a total DNA digestion by enzymes that digest nucleosides by quantitative liquid chromatography-mass spectrometry (LC-MS), in which the content of DNA is back calculated from a standard curve of each base (i.e., A, C, G, T) as measured by LC-MS.

In some embodiments of each aspect recited above, the amount of linear polyribonucleotide molecules as compared to circular polyribonucleotide molecules is determined using the method of Example 2 or Example 3. In some embodiments, the amount of linear polyribonucleotide molecules in the pharmaceutical preparation is determined using the method of Example 2. In some embodiments, the amount of circular polyribonucleotide molecules in the pharmaceutical preparation is determined using the method of Example 3.

### Methods of Making Pharmaceutical Preparations of Polyribonucleotides

In another aspect, a method of making a pharmaceutical composition comprises: a) providing a preparation of circular polyribonucleotide molecules, b) processing the preparation to reduce the amount of linear polyribonucleotide molecules, c) optionally evaluating the amount of linear polyribonucleotide molecules in the preparation before, during, and/or after the processing step, and d) further processing the preparation to produce a pharmaceutical composition for pharmaceutical use. In some embodiments, the further processing of step d) comprises one or more of: i) processing the preparation to substantially remove DNA and/or protein (e.g., a cell protein such as a host cell protein) and/or endotoxin; ii) evaluating the amount of DNA and/or protein (e.g., a cell protein such as a host cell protein) and/or endotoxin in the preparation; iii) formulating the preparation with a pharmaceutical excipient; and iv) optionally, concentrating the preparation.

In another aspect, a method of making a pharmaceutical drug substance comprises: a) providing a preparation of circular polyribonucleotide molecules, b) evaluating the amount of linear polyribonucleotide molecules in the preparation, and c) processing the preparation of circular polyribonucleotide molecules as a pharmaceutical drug substance if the preparation meets a reference criterion (e.g., a pharmaceutical release criterion, e.g., a pharmaceutical release criterion or reference criterion described herein) for an amount of linear polyribonucleotide molecules present in the preparation.

In another aspect, a method of making a pharmaceutical drug substance, comprises: a) providing a plurality of linear polyribonucleotide molecules; b) circularizing the plurality of linear polyribonucleotide molecules to provide a preparation of circular polyribonucleotide molecules; c) evaluating the amount of linear polyribonucleotide molecules remaining in the preparation; and d) processing the preparation of circular polyribonucleotide molecules as a pharmaceutical drug substance if the preparation meets a reference criterion for an amount of linear polyribonucleotide molecules present in the preparation.

In another aspect, a method of making a a pharmaceutical drug substance, comprises a) providing a plurality of linear polyribonucleotide molecules; b) circularizing the plurality of linear polyribonucleotide molecules to provide a preparation of circular polyribonucleotide molecules; c) evaluating the amount of linear and/or nicked polyribonucleotide molecules remaining in the preparation; and d) processing the preparation of circular polyribonucleotide molecules as a pharmaceutical drug substance if the preparation meets a reference criterion for an amount of linear and/or nicked polyribonucleotide molecules present in the preparation.

In another aspect, a method of making a pharmaceutical drug product comprises: a) providing a plurality of linear polyribonucleotide molecules; b) circularizing the plurality of linear polyribonucleotide molecules to provide a preparation of circular polyribonucleotide molecules; c) measuring the amount of linear and/or nicked polyribonucleotide molecules in the preparation; d) formulating the preparation of circular polyribonucleotide molecules as a pharmaceutical drug product if the preparation meets a reference criterion for an amount of linear and/or nicked polyribonucleotide molecules present in the preparation; and e) labelling and shipping the pharmaceutical drug product if it meets a reference criterion for the amount of linear polyribonucleotide molecules present in the pharmaceutical drug product.

In another aspect, a method of making a pharmaceutical drug product comprises: a) providing a preparation of circular polyribonucleotide molecules, b) formulating the preparation of circular polyribonucleotide molecules as a pharmaceutical drug product if it meets a reference criterion for an amount of linear polyribonucleotide molecules present in the preparation, c) measuring the amount of linear polyribonucleotide molecules in a sample of a pharmaceutical drug product, and d) formulating, labelling and/or shipping the pharmaceutical drug product if it meets a reference criterion for the amount of linear polyribonucleotide molecules present in the pharmaceutical drug product.

In another aspect, a method of making a pharmaceutical drug product comprises: a) providing a plurality of linear polyribonucleotide molecules; b) circularizing the plurality of linear polyribonucleotide molecules to provide a preparation of circular polyribonucleotide molecules; c) measuring the amount of linear polyribonucleotide molecules in the preparation; d) formulating the preparation of circular polyribonucleotide molecules as a pharmaceutical drug product if the preparation meets a reference criterion for an amount of linear polyribonucleotide molecules present in the preparation; and e) labelling and shipping the pharmaceutical drug product if it meets a reference criterion for the amount of linear polyribonucleotide molecules present in the pharmaceutical drug product.

In another aspect, a method of making a pharmaceutical composition comprises: a) providing a plurality of linear polyribonucleotide molecules; b) circularizing the linear polyribonucleotide molecules to provide a preparation of circular polyribonucleotide molecules; c) processing the preparation to substantially remove linear polyribonucleotide molecules remaining in the preparation; d) optionally evaluating the amount of linear polyribonucleotide molecules in the preparation remaining after the processing step; and e) further processing the preparation to produce the pharmaceutical composition for pharmaceutical use. In some embodiments, the method further comprises one or more of f) processing the preparation to substantially remove deoxyribonucleotide molecules; g) evaluating the amount of deoxyribonucleotide molecules in the preparation; h) formulating the preparation with a pharmaceutical excipient; i) concentrating the preparation; and j) documenting the amount of deoxyribonucleotide molecules in the preparation in a print or digital media. In some embodiments, the method further comprises: f) processing the preparation to substantially remove protein contamination; g) evaluating the amount of protein contamination in the preparation; h) formulating the preparation with a pharmaceutical excipient; and i) concentrating the preparation. In some embodiments, the further processing of step d) comprises one or more of: f) processing the preparation to substantially remove endotoxin; g) evaluating the amount of endotoxin in the preparation; h) formulating the preparation with a pharmaceutical excipient; and i) concentrating the preparation.

In some embodiments of each of the above aspects, the circularizing step is performed by splint ligation. In some embodiments of each of the above aspects, the formulating the preparation of circular polyribonucleotide molecules comprising combining the preparation of circular polyribonucleotide molecules with a pharmaceutical excipient.

In some embodiments of each of the the above aspects, the method further comprises documenting the amount of polyribonucleotide molecules (e.g., linear polyribonucleotide molecules and/or circular polyribonucleotide molecules) in the preparation in a print or digital media, e.g., in a certificate of analysis for the preparation.

In some embodiments of each of the the above aspects, the formulating step comprises combining the preparation of circular polyribonucleotide molecules with a pharmaceutical excipient.

In some embodiments of each of the the above aspects, the reference criterion is a pharmaceutical release specification for a preparation of circular polyribonucleotide molecules. For example, the reference criterion may be one or more of: (a) the amount of linear polyribonucleotide molecules present in the pharmaceutical preparation is no more than a certain amount, e.g., 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 5 μg/ml, 10 μg/ml, 50 μg/ml, 100 μg/ml, 200 ug/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/ml, 10 mg/ml, 50 mg/ml, 100 mg/ml, 200 mg/ml, 300 mg/ml, 400 mg/ml, 500 mg/ml, 600 mg/ml, 700 mg/ml or 750 mg/ml of linear polyribonucleotide molecules; (b) the pharmaceutical drug product or pharmaceutical drug substance comprises a concentration of at least a certain amount, e.g., 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 5 mg/mL, 10 mg/mL, 100 mg/mL, 200 mg/mL, 500 mg/mL, 600 mg/ml, 700 mg/ml, or 750 mg/ml circular polyribonucleotide molecules; or (c) the pharmaceutical drug product or pharmaceutical drug substance comprises at least a certain amount, e.g., at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) circular polyribonucleotide molecules relative to total ribonucleotide molecules in the pharmaceutical preparation.

In some embodiments of each of the above aspects, a reference criterion for the amount of linear and/or nicked polyribonucleotide molecules present in the preparation is select from: a) no more than 20%, 15%, 10%, 5%, 2%, 1%, or 0.5% (w/w) linear polyribonucleotide molecules relative to the total ribonucleotide molecules in the preparation; b) no more than 5%, 4%, 3%, 2%, 1%, 0.5%, or 0.1% (w/w) nicked polyribonucleotide molecules relative to the total ribonucleotide molecules in the preparation; or c) no more than 20%, 15%, 10%, 5%, 2%, 1%, or 0.5% (w/w) combined linear and nicked polyribonucleotide molecules relative to the total ribonucleotide molecules in the preparation.

In some embodiments of each of the above aspects, at least 80% (w/w) of total ribonucleotide molecules in the pharmaceutical preparation are circular polyribonucleotide molecules. In some embodiments of each of the above aspects, the pharmaceutical composition comprises no more than 20% (w/w) linear polyribonucleotide molecules of the total ribonucleotide molecules in the preparation. In some embodiments of each of the above aspects, the pharmaceutical composition comprises no more than 10% (w/w) linear polyribonucleotide molecules of the total ribonucleotide molecules in the preparation.

In some embodiments of each of the the above aspects, circular polyribonucleotide molecules (e.g., relative to total ribonucleotide molecules) in the pharmaceutical preparation is measured by microscopy, by spectrophotometry, by fluorometry, by denaturing urea polyacrylamide gel electrophoresis imaging, by UV-Vis spectrophotometry, by RNA electrophoresis, by RNAse H analysis, by UV spectroscopic or fluorescence detectors, by light scattering techniques, by surface plasmon resonance (SPR) with or without the use of methods of separation including HPLC, by HPLC, by chip or gel based electrophoresis with or without using either pre or post separation derivatization methodologies, by using methods of detection that use silver or dye stains or radioactive decay for detection of linear polyribonucleotide molecules, or by methods that utilize microscopy, visual methods or a spectrophotometer. For example, the amount of circular polyribonucleotide relative to total ribonucleotide molecules may determined using the method of Example 2 or Example 3.

In some embodiments of each of the the above aspects, the pharmaceutical drug product or pharmaceutical drug substance further: (a) comprises less than 10 EU/kg or lacks endotoxin as measured by the Limulus amebocyte lysate test; (b) comprises a bioburden of less than 100 CFU/100 ml or less than 10 CFU/100 ml before sterilization; (c) is a sterile drug product or sterile drug substance; (d) supports growth of fewer than 100 viable microorganisms as tested under aseptic conditions; and/or (e) meets the standard of USP <71> or USP <85>.

In some embodiments of each of the the above aspects, the circular polyribonucleotide molecules comprise one or more expression sequences and a stagger element at a 3′ end of at least one expression sequence.

In some embodiments of each of the the above aspects, the preparation further meets a reference criterion for the amount of DNA (e.g., cell DNA such as host cell DNA) present in the preparation. In some embodiments, the reference criterion for the amount of DNA molecules present in the preparation is the presence of no more than a certain amount, e.g., zero DNA molecules, substantially free of DNA molecules, or no more than 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml, 1 μg/mL, 5 μg/mL, 10 μg/mL, or 100 μg/mL of DNA molecules.

In some embodiments of each of the above aspects, the preparation further meets a reference criterion for the amount of protein contamination (e.g., cell protein such host cell protein or process related protein impurity, e.g., an enzyme) present in the preparation. In some embodiments, the reference criterion for the amount of protein contamination present in the preparation is less than a certain amount, e.g., less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of protein contamination per milligram (mg) of circular polyribonucleotide molecules. In some embodiments of each of the above aspects, a protein contamination comprises an enzyme.

In some embodiments of each of the above aspects, the pharmaceutical drug product or pharmaceutical drug substance comprises an A260/A280 absorbance ratio of from about 1.6 to 2.3 as measured by a spectrophotometer.

In some embodiments of each of the above aspects, the linear polyribonucleotide molecules comprise a linear polyribonucleotide molecule counterpart of the circular polyribonucleotide molecules or a fragment of the linear polyribonucleotide molecule counterpart of the circular polyribonucleotide molecules. In some embodiments of each of the above aspects, the linear polyribonucleotide molecules comprise a linear polyribonucleotide molecule counterpart of the circular polyribonucleotide molecules (e.g., a pre-circularized version). In some embodiments of each of the above aspects, the linear polyribonucleotide molecules comprise a linear polyribonucleotide molecule counterpart of the circular polyribonucleotide molecules or a fragment thereof, a linear polyribonucleotide molecule non-counterpart of the circular polyribonucleotide molecules or a fragment thereof, or a combination thereof. In some embodiments of each of the above aspects, the linear polyribonucleotide molecules comprise a linear polyribonucleotide molecule counterpart of the circular polyribonucleotide molecules (e.g., a pre-circularized version), a linear polyribonucleotide molecule non-counterpart of the circular polyribonucleotide molecules, or a combination thereof.

In some embodiments of each aspect recited above, the circular polyribonucleotide molecules include a sequence, or plurality of sequences, encoding expression product(s), e.g., therapeutic expression products, e.g., encoding a therapeutic protein or nucleic acid. In some embodiments of each aspect recited above, the circular polyribonucleotide molecules have a sequence comprising a scaffold (e.g., an aptamer sequence). In some embodiments of each of the above aspects, the circular polyribonucleotide molecules have a sequence encoding an endogenous or naturally occurring circular polyribonucleotide sequence. In such embodiments, the pharmaceutical preparation may further meet a reference criterion for circular polyribonucleotide molecules having a sequence, e.g., a sequence having at least 80% (e.g., 85%, 90%, 95%, 97%, 99%, 100%, or any percentage therebetween) sequence identity to a reference sequence encoding the expression product.

### Methods of Use

In another aspect, a method of delivering a circular polyribonucleotide molecule to a cell or tissue of a subject, or to subject, comprises administering a pharmaceutical preparation as described herein, a pharmaceutical composition as described herein, a pharmaceutical drug substance as described herein, or a pharmaceutical drug product as described herein to the cell or tissue of the subject, or to the subject, wherein the circular polyribonucleotide molecule is detected in the cell, tissue, or subject, e.g., at least 3 days (e.g., at least 4, 5, 6, 7, 10, 12, 15, 20, 24 days or more, or any day therebetween) after the administering step.

In another aspect, a method of delivering a circular polyribonucleotide molecule to a cell or tissue of a subject, or to subject, comprises administering a pharmaceutical preparation as described herein, a pharmaceutical composition as described herein, a pharmaceutical drug substance as described herein, or a pharmaceutical drug product as described herein to the cell or tissue of the subject, or to the subject, wherein the circular polyribonucleotide or a product translated from the circular polyribonucleotide is detected in the cell, tissue, or subject at least 3 days after the administering step.

In another aspect, a method of delivering a therapeutic product to a cell or tissue of a subject, or to a subject in need thereof, comprises administering a pharmaceutical preparation as described herein, a pharmaceutical composition as described herein, a pharmaceutical drug substance as described herein, or a pharmaceutical drug product as described herein to the cell or tissue of the subject, or to the subject. In some embodiments of each above aspect, the circular polyribonucleotide molecules of the composition or preparation comprise circular polyribonucleotide molecules having a sequence comprising the therapeutic product and the therapeutic product transcribed or translated from the circular polyribonucleotide molecules is detected in the cell, tissue, or subject, e.g., at least 3 days (e.g., at least 4, 5, 6, 7, 10, 12, 15, 20, 24 days or more, or any day therebetween) after the administering step. In some embodiments of this aspect, the circular polyribonucleotide molecules of the composition or preparation comprise circular polyribonucleotide molecules having a sequence comprising an aptamer and the circular polyribonucleotide molecule is detected in the cell, tissue, or subject at least 3 days (e.g., at least 4, 5, 6, 7, 10, 12, 15, 20, 24 days or more, or any day therebetween) after the administering step. In some embodiments of this aspect, the circular polyribonucleotides of the composition or preparation comprise circular polyribonucleotide molecules having a endogenous or naturally occurring circular polyribonucleotide molecule sequence and the endogenous or naturally occurring circular polyribonucleotide molecule is detected in the cell, tissue, or subject at least 3 days (e.g., at least 4, 5, 6, 7, 10, 12, 15, 20, 24 days or more, or any day therebetween) after the administering step.

In another aspect, a parenteral nucleic acid delivery system comprises (i) a pharmaceutical preparation as described herein, a pharmaceutical composition as described herein, a pharmaceutical drug substance as described herein, or a pharmaceutical drug product as described herein, and (ii) a parenterally acceptable diluent. In some embodiments of this aspect, the pharmaceutical preparation, the pharmaceutical composition, the pharmaceutical drug substance, or the pharmaceutical drug product is free of any carrier.

In another aspect, a method of delivering a circular polyribonucleotide comprises parenterally administering to a subject in need thereof, a pharmaceutical preparation as described herein, a pharmaceutical composition as described herein, a pharmaceutical drug substance as described herein, or a pharmaceutical drug product as described herein. In some embodiments of this aspect, the circular polyribonucleotide is in an amount effective to elicit or induce a biological response in the subject. In some embodiments of this aspect, the circular polyribonucleotide is in an amount effective to have a biological effect on a cell or tissue in the subject. In some embodiments of this aspect, parenteral administration is intravenously, intramuscularly, ophthalmically or topically.

In another aspect, a method of delivering a circular polyribonucleotide to a cell or tissue of a subject comprises parenterally administering to the cell or tissue, a pharmaceutical preparation as described herein, a pharmaceutical composition as described herein, a pharmaceutical drug substance as described herein, or a pharmaceutical drug product as described herein. In some embodiments of this aspect, parenteral administration is intravenously, intramuscularly, ophthalmically or topically.

In some embodiments of each above aspect, the method further comprises evaluating the presence of the circular polyribonucleotide molecules or product translated from the circular polyribonucleotide molecules in the cell, tissue or subject before the administering step. In some embodiments of each above aspect, the method further comprises evaluating the presence of the circular polyribonucleotide molecules or a product translated from the circular polyribonucleotide molecules in the cell, tissue or subject after the administering step (e.g., 24 hours, 48 hours, 72 hours, 4 days, 7 days, 14 days or longer, or any day therebetween, after the administering step). In some embodiments of each of the above aspects, the pharmaceutical preparation, the pharmaceutical composition, the pharmaceutical drug substance, or the pharmaceutical drug product comprises a diluent (e.g., parenterally acceptable diluent) and is free of any carrier.

### Definitions

The present invention will be described with respect to particular embodiments and with reference to certain figures but the invention is not limited thereto but only by the claims. Terms as set forth hereinafter are generally to be understood in their common sense unless indicated otherwise.

The terms “obtainable by”, “producible by” or the like are used to indicate that a claim or embodiment refers to compound, composition, product, etc. per se, i. e. that the compound, composition, product, etc. can be obtained or produced by a method which is described for manufacture of the compound, composition, product, etc., but that the compound, composition, product, etc. may be obtained or produced by other methods than the described one as well. The terms “obtained by”, “produced by” or the like indicate that the compound, composition, product, is obtained or produced by a recited specific method. It is to be understood that the terms “obtainable by”, “producible by” and the like also disclose the terms “obtained by”, “produced by” and the like as a preferred embodiment of “obtainable by”, “producible by” and the like.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as a preferred embodiment, such compound, composition, product, etc. is for use in treating, modulating, etc.

The wording “compound, composition, product, etc. for use in . . . ” or “use of a compound, composition, product, etc in the manufacture of a medicament, pharmaceutical composition, veterinary composition, diagnostic composition, etc. for . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc.

The term “pharmaceutical composition” is intended to also disclose that the circular polyribonucleotide comprised within a pharmaceutical composition can be used for the treatment of the human or animal body by therapy. It is thus meant to be equivalent to the “a circular polyribonucleotide for use in therapy”.

The circular polyribonucleotide molecules, compositions comprising such circular polyribonucleotide molecules, methods of making and using such circular polyribonucleotides, etc. as described herein are based in part on the examples which illustrate the effect of linear RNA molecules in circular RNA preparations (e.g., Examples 1-12), and (e.g., Examples 13 et seq) the making and using of circular polyribonucleotide effectors comprising different elements, for example a replication element, an expression sequence, a stagger element and an encryptogen (see, e.g., Example 13) or for example an expression sequence, a stagger element and a regulatory element (see, e.g., Examples 34 and 44), and their technical effects (e.g., increased translation efficiency than a linear counterpart in Examples 43 and 44 and increased half-life over a linear counterpart in Example 33 and Example 60). It is on the basis of inter alia these examples that the description hereinafter contemplates various variations of the specific findings and combinations considered in the examples.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules

As used herein, the terms “circRNA” or “circular polyribonucleotide” or “circular RNA” or “circular polyribonucleotide molecule” are used interchangeably and mean a polyribonucleotide molecule that has a structure having no free ends (i.e., no free 3′ and/or 5′ ends), for example a polyribonucleotide molecule that forms a circular or end-less structure through covalent or non-covalent bonds.

As used herein, the term “fragment” means any portion of a nucleotide molecule that is at least one nucleotide shorter than the nucleotide molecule. For example, a nucleotide molecule can be a linear polyribonucleotide molecule and a fragment thereof can be a monoribonucleotide or any number of contiguous polyribonucleotides that are a portion of the linear polyribonucleotide molecule. As another example, a nucleotide molecule can be a circular polyribonucleotide molecule and a fragment thereof can be a polyribonucleotide or any number of contiguous polyribonucleotides that are a portion of the circular polyribonucleotide molecule.

As used herein, the term “encryptogen” is a nucleic acid sequence or structure of the circular polyribonucleotide that aids in reducing, evading, and/or avoiding detection by an immune cell and/or reduces induction of an immune response against the circular polyribonucleotide.

As used herein, the term “expression sequence” is a nucleic acid sequence that encodes a product, e.g., a peptide or polypeptide, or a regulatory nucleic acid. An exemplary expression sequence that codes for a peptide or polypeptide can comprise a plurality of nucleotide triads, each of which can code for an amino acid and is termed as a “codon”.

As used herein, the term “immunoprotein binding site” is a nucleotide sequence that binds to an immunoprotein. In some embodiments, the immunoprotein binding site aids in masking the circular polyribonucleotide as exogenous, for example, the immunoprotein binding site can be bound by a protein (e.g., a competitive inhibitor) that prevents the circular polyribonucleotide from being recognized and bound by an immunoprotein, thereby reducing or avoiding an immune response against the circular polyribonucleotide. As used herein, the term “immunoprotein” is any protein or peptide that is associated with an immune response, e.g., such as against an immunogen, e.g., the circular polyribonucleotide. Non-limiting examples of immunoprotein include T cell receptors (TCRs), antibodies (immunoglobulins), major histocompatibility complex (MHC) proteins, complement proteins, and RNA binding proteins.

As used herein, the terms “linear RNA” or “linear polyribonucleotide” or “linear polyribonucleotide molecule” are used interchangeably and mean polyribonucleotide molecule having a 5′ and 3′ end. One or both of the 5′ and 3′ ends may be free ends or joined to another moiety. As used herein, a linear RNA has not undergone circularization (e.g., is pre-circularized) and can be used as a starting material for circularization through, for example, splint ligation, or chemical, enzymatic, ribozyme- or splicing-catalyzed circularization methods.

As used herein, the terms “nicked RNA” or “nicked linear polyribonucleotide” or “nicked linear polyribonucleotide molecule” are used interchangeably and mean a polyribonucleotide molecule having a 5′ and 3′ end that results from nicking or degradation of a circular RNA.

As used herein, the term “non-circular RNA” means total nicked RNA and linear RNA.

As used herein, the term “modified ribonucleotide” is a nucleotide with at least one modification to the sugar, the nucleobase, or the internucleoside linkage.

As used herein, the phrase “quasi-helical structure” is a higher order structure of the circular polyribonucleotide, wherein at least a portion of the circular polyribonucleotide folds into a helical structure.

As used herein, the phrase “quasi-double-stranded secondary structure” is a higher order structure of the circular polyribonucleotide, wherein at least a portion of the circular polyribonucleotide creates an internal double strand.

As used herein, the term “regulatory element” is a moiety, such as a nucleic acid sequence, that modifies expression of an expression sequence within the circular polyribonucleotide.

As used herein, the term “repetitive nucleotide sequence” is a repetitive nucleic acid sequence within a stretch of DNA or RNA or throughout a genome. In some embodiments, the repetitive nucleotide sequence includes poly CA or poly TG (UG) sequences. In some embodiments, the repetitive nucleotide sequence includes repeated sequences in the Alu family of introns.

As used herein, the term “replication element” is a sequence and/or motifs useful for replication or that initiate transcription of the circular polyribonucleotide.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPG P, where x=any amino acid. In some embodiments, the stagger element may include a chemical moiety, such as glycerol, a non-nucleic acid linking moiety, a chemical modification, a modified nucleic acid, or any combination thereof.

As used herein, the term “substantially resistant” is one that has at least 50%, 55%, 60%, 65%, 70%, 75%, 80%, 85%, 90%, 95%, 96%, 97%, 98% or 99% resistance to an effector as compared to a reference.

As used herein, the term “stoichiometric translation” is a substantially equivalent production of expression products translated from the circular polyribonucleotide. For example, for a circular polyribonucleotide having two expression sequences, stoichiometric translation of the circular polyribonucleotide means that the expression products of the two expression sequences have substantially equivalent amounts, e.g., amount difference between the two expression sequences (e.g., molar difference) can be about 0, or less than 1%, 2%, 3%, 4%, 5%, 6%, 7%, 8%, 9%, 10%, 15%, or 20%, or any percentage therebetween.

As used herein, the term “translation initiation sequence” is a nucleic acid sequence that initiates translation of an expression sequence in the circular polyribonucleotide.

As used herein, the term “termination element” is a moiety, such as a nucleic acid sequence, that terminates translation of the expression sequence in the circular polyribonucleotide.

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “circularization efficiency” is a measurement of resultant circular polyribonucleotide versus its non-circular starting material.

As used herein, the term “immunogenic” is a potential to induce an immune response to a substance. In some embodiments, an immune response may be induced when an immune system of an organism or a certain type of immune cells is exposed to an immunogenic substance. The term “non-immunogenic” is a lack of or absence of an immune response above a detectable threshold to a substance. In some embodiments, no immune response is detected when an immune system of an organism or a certain type of immune cells is exposed to a non-immunogenic substance. In some embodiments, a non-immunogenic circular polyribonucleotide as provided herein, does not induce an immune response above a pre-determined threshold when measured by an immunogenicity assay. For example, when an immunogenicity assay is used to measure antibodies raised against a circular polyribonucleotide or inflammatory markers, a non-immunogenic polyribonucleotide as provided herein can lead to production of antibodies or markers at a level lower than a predetermined threshold. The predetermined threshold can be, for instance, at most 1.5 times, 2 times, 3 times, 4 times, or 5 times the level of antibodies or markers raised by a control reference. As another example, when an immunogenicity assay is used to measure the innate immune response against a circular polyribonucleotide (such as measuring an inflammatory marker), a non-immunogenic polyribonucleotide as provided herein can lead to production of an innate immune response at a level lower than a predetermined threshold. The predetermined threshold can be, for instance, at most 1.5 times, 2 times, 3 times, 4 times, or 5 times the level of a marker produced by an innate response for a control reference.

As used herein, the term “impurity” is an undesired substance present in the a composition, e.g., a pharmaceutical composition as described herein. In some embodiments, an impurity is a process-related impurity. In some embodiments, an impurity is a product-related substance other than the desired product in the final composition, e.g., other than the active drug ingredient, e.g., circular polyribonucleotide, as described herein. As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance are deoxyribonucleotide fragments. In some embodiments, the product-related substance are deoxyribonucleotide monomers. In some embodiments, the product-related substance is one or more of: derivatives or fragments of polyribonucleotides described herein, e.g., fragments of 10, 9, 8, 7, 6, 5, or 4 ribonucleic acids, monoribonucleic acids, diribonucleic acids, or triribonucleic acids.

As used herein, the term “substantially free” is the level of a component in a composition, preparation, or product, or any intermediate thereof that is lower than the level required to induce a biological, chemical, physical, and/or pharmacological effect. In some embodiments, a composition, preparation, or product is substantially free of a component if the level of the component is detectable only in trace amounts or the level is less than the level detectable by a relevant detection technique (e.g., chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre or post separation derivatization methodologies using detection techniques based on mass spectrometry, UV-visible, fluorescence, light scattering, refractive index, or that use silver or dye stains or radioactive decay for detection. Alternatively, whether a composition, preparation, or product is substantially free of a component may be determined without the use of separation technologies by mass spectrometry, by microscopy, by circular dichroism (CD) spectroscopy, by UV or UV-vis spectrophotometry, by fluorometry (e.g., Qubit), by RNAse H analysis, by surface plasmon resonance (SPR), or by methods that utilize silver or dye stains or radioactive decay for detection).

As used herein, the term “linear counterpart” is a polyribonucleotide molecule (and its fragments) having the same or similar nucleotide sequence (e.g., 100%, 95%, 90%, 85%, 80%, 75%, or any percentage therebetween sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

As used herein, the term “aptamer sequence” is a non-naturally occurring, or synthetic oligonucleotide that specifically binds to a target molecule. Typically an aptamer is from 20 to 500 nucleotides. Typically an aptamer binds to its target through secondary structure rather than sequence homology. In some embodiments, the synthetic oligonucleotide can have the same sequence as a naturally occurring oligonucleotide that specifically binds to a target molecule.

As used herein, the term “carrier” means a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier.

The term “diluent” means a vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the term “parenterally acceptable diluent” is a diluent used for parenteral administration of a composition (e.g., a composition comprising a circular polyribonucleotide).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 shows the binding of probes to circular and linear RNA and subsequent degradation of the RNA by RNase H. Circular RNA is detected as a a single cleaved linear band compared to linear and concatemeric RNA, which is detected as multiple bands. Degradation was detected by running samples on a denaturing polyacrylamide gel and comparing degradation bands with or without addition of RNase H.

FIG. 2 shows linear RNA content quantified on a denaturing polyacrylamide gel by comparing linear RNA band intensity to a linear RNA standard.

FIG. 3 shows the quantification of RNA extracted from different bands of a denaturing polyacrylamide gel.

FIG. 4 shows the persistence of purified circular RNA preparations over time in BJ fibroblast cells as compared to unpurified circular RNA preparations.

FIG. 5 shows the expression levels of purified circular RNA preparations over time in BJ fibroblast cells as compared to circular RNA preparations containing different amounts of linear RNA.

FIG. 6 shows that purified circular RNA has higher expression following injection into mice as well as longer expression as measured in the liver ex vivo 14 days following administration.

FIG. 7 shows a graph of the ratio of circular RNA to linear RNA before and after purification as calculated by measuring the band intensities in the 6% Urea PAGE gel. Quantification of the bands were as follows: before purification, 42.6% of the RNA product was circular RNA and 57.4% of the RNA product was linear RNA (unpurified RNA); after purification, 50.4% of the RNA product was circular RNA and 49.6% of the RNA product was linear RNA (purified circRNA).

FIG. 8 shows Gaussia Luciferase activity in cells at 6, 24, 48, 72, 96 and 120 hours post-transfection in experiments using circular RNA of 84% purity, circular RNA of 71% purity, and vehicle only.

FIG. 9 shows cells transfected with the gel purified circular RNA preparation alone compared to cells transfected with both the combined circular RNA and linear counterpart RNA. circRNA only showed increased stability compared to combined circular RNA and linear counterpart RNA preparations in a dose dependent manner.

FIG. 10 shows cells transfected with the gel purified circular RNA preparation only showed minimal expression of innate immune genes such as RIG-I, MDA-5, OAS, and IFN-B compared to cells transfected with both the combined circular RNA and linear counterpart RNA, which exhibited upregulation of these innate immune genes in a dose dependent manner.

FIG. 11 shows a schematic of a control circular RNA that has an intron and expresses GFP.

FIG. 12 shows a schematic of an exemplary circular RNA that has a synthetic riboswitch (in red) regulating the expression of the GFP from the circular RNA in the presence or absence of ligands to the riboswitch.

FIG. 13 is a schematic demonstrating in vivo protein expression in mouse model from an exemplary circular RNA that harbors an encryptogen (intron).

FIG. 14 shows a schematic of an exemplary circular RNA that has one double-stranded RNA segment, which can be subject to dot blot analysis for its structural information.

FIG. 15 shows a schematic of an exemplary circular RNA that has a quasi-helical structure (HDVmin), which can be subject to SHAPE analysis for its structural information.

FIG. 16 shows a schematic of an exemplary circular RNA that has a functional quasi-helical structure (HDVmin), which demonstrates HDAg binding activity.

FIG. 17 is a schematic demonstrating transcription, self-cleavage, and ligation of an exemplary self-replicable circular RNA.

FIG. 18 shows a schematic of an exemplary circular RNA that is preserved during mitosis and persists in daughter cells. BrdU pulse as shown is used for labeling the divided cells.

FIG. 19 is a denaturing PAGE gel image demonstrating in vitro production of different exemplary circular RNAs.

FIG. 20 is a graph summarizing circularization efficiencies of different exemplary circular RNAs.

FIG. 21 is a denaturing PAGE gel image demonstrating decreased degradation susceptibility of an exemplary circular RNA as compared to its linear counterpart.

FIG. 22 is a denaturing PAGE gel image demonstrating exemplary circular RNAs after an exemplary purification process.

FIG. 23 is a Western blot image demonstrating expression of Flag protein (˜15 kDa) by an exemplary circular RNA that lacks TRES, cap, 5′ and 3′ UTRs.

FIG. 24 is Western blot image demonstrating rolling-circle translation of an exemplary circular RNA.

FIG. 25 shows Western blot images demonstrating production of discrete proteins or continuous long peptides from different exemplary circular RNAs with or without an exemplary stagger element.

FIG. 26 is a Western blot image showing the comparison of protein expression between different exemplary circular RNAs with an exemplary stagger element or a termination element (stop codon).

FIG. 27 is a graph summarizing the signal intensity from Western blot analysis of the protein products translated from the two exemplary circular RNAs.

FIG. 28 is a graph summarizing the luciferase activity of translation products of an exemplary circular RNA and its linear counterpart, in comparison with a vehicle control.

FIG. 29 is a graph summarizing RNA quantities at different collection time points in a time course experiment testing half-life of an exemplary circular RNA compared to a linear RNA.

FIG. 30A is a graph showing qRT-PCR analysis of linear and circular RNA levels 24 hours after delivery to cells using primers that captured both linear and circular RNA.

FIG. 30B is a graph showing qRT-PCR analysis of linear and circular RNA levels using a primer specific for the circular RNA.

FIG. 31 is an image showing a blot of cell lysates from circular RNA and linear RNA probed for EGF protein and a beta-tubulin loading control.

FIG. 32 is a graph showing qRT-PCR analysis of immune related genes from 293T cells transfected with circular RNA or linear RNA.

FIG. 33 is a graph showing luciferase activity of protein expressed from circular RNA via rolling circle translation.

FIG. 34 is a graph showing luciferase activity of protein expressed from circular RNA or linear RNA.

FIG. 35 is a graph showing luciferase activity of protein expressed from linear RNA or circular RNA via rolling circle translation.

FIG. 36 is a graph showing luciferase activity of protein expressed from circular RNA via IRES translation initiation.

FIG. 37 is a graph showing luciferase activity of protein expressed from circular RNA via IRES initiation and rolling circle translation.

FIG. 38 is an image showing a protein blot of expression products from circular RNA or linear RNA.

FIG. 39 is an image showing a protein blot of expression products from circular RNA or linear RNA with a stagger element.

FIG. 40 shows predicted structure with a quasi-double stranded structure of an exemplary circular RNA.

FIG. 41 shows predicted structure with a quasi-helical structure of an exemplary circular RNA.

FIG. 42 shows predicted structure with a quasi-helical structure linked with a repetitive sequence of an exemplary circular RNA.

FIG. 43 demonstrates experimental data that degradation by RNAse H of an exemplary circular RNA produced nucleic acid degradation products consistent with a circular and not a concatemeric RNA.

FIG. 44 shows an electrophoresis image of the different lengths of DNA that were generated for the creation of a wide variety of RNA lengths.

FIG. 45 shows experimental data that confirmed the circularization of RNAs using RNAse R treatment and qPCR analysis against circular junctions of a wide variety of lengths.

FIG. 46 shows generation of exemplary circular RNA with a miRNA binding site.

FIG. 47 shows generation of exemplary circular RNA by self-splicing.

FIG. 48 shows generation of exemplary circular RNA with a protein binding site.

FIG. 49 shows experimental data demonstrating the higher stability of circular RNA in a dividing cell as compared to linear controls.

FIG. 50 shows experimental data demonstrating the protein expression from exemplary circular RNAs with a plurality of expression sequences and the rolling circle translation of exemplary circular RNAs with multiple expression sequences.

FIG. 51 shows experimental data demonstrating the reduced toxicity to transfected cells of an exemplary circular RNA as compared to linear control.

FIG. 52 shows that exemplary circular RNA was translated at a higher level as compared to linear RNA under stress condition.

FIG. 53 shows generation of circular RNAs with a riboswitch.

FIG. 54A, FIG. 54B, and FIG. 54C show that the modified circular RNAs were translated in cells.

FIG. 55A, FIG. 55B, and FIG. 55C show that modified circular RNAs have reduced immunogenicity as compared to unmodified circular RNAs to cells as assessed by MDA5, OAS and IFN-beta expression in the transfected cells.

FIG. 56 shows that after injection into mice, circular RNA was detected at higher levels than linear RNA in livers of mice at 3, 4, and 7 days post-injection

FIG. 57A and FIG. 57B show that after injection of circular RNA or linear RNA expressing Gaussia Luciferase into mice, Gaussia Luciferase activity was detected in plasma at 1, 2, 7, 11, 16, and 23 days post-dosing of circular RNA, while its activity was only detected in plasma at 1, and 2 days post-dosing of modified linear RNA.

FIG. 58 shows that after injection of RNA, circular RNA but not linear RNA, was detected in the liver and spleen at 16 days post-administration of RNA.

FIG. 59 shows that after injection of RNA, linear RNA but not circular RNA, showed immunogenicity as assessed by RIG-I, MDA-5, IFN-B and OAS.

### DETAILED DESCRIPTION

This invention relates generally to pharmaceutical compositions and preparations of circular polyribonucleotides and uses thereof.

In some aspects, the invention described herein comprises circular RNA compositions, preparations and methods of using and making circular RNA compositions and preparations, particularly pharmaceutical circular RNA compositions and preparations, having reduced, controlled, or specified levels of linear RNA. As described herein, e.g., in Examples 1-12, the presence of linear RNA in circular RNA preparations can affect, for example, expression levels, persistence, half life, and/or stability of the circular RNA; and/or immune response to the preparations.

Table 1 is intended to provide a brief outline of the contents of the Detailed Description, which is by no means exclusive or limiting. Certain aspects of the Detailed Description may not be reflected in the Table 1 Detailed Description Outline.

TABLE 1
Detailed Description Outline

| Starting | | |
|---|---|---|
| Paragraph | | |
| No. | Main Heading | Subheading |
| | Circular Polyribonucleotides | |
| | Methods of Making Circular | |
| | RNA | |
| | Detection of Linear and | |
| | Circular RNA | |
| | Purification of Circular RNA | |
| | Pharmaceutical Compositions | |
| | Methods of Manufacturing | |
| | Pharmaceutical Circular RNA | |
| | Preparations | |
| | Circularization | |
| | Extracellular Circularization | Splicing Element |
| | | Other Circularizations |
| | | Methods |
| | Expression Sequences | Peptides or Polypeptides |
| | | Regulatory Elements |
| | | Translation Initiation |
| | | Sequence |
| | | IRES |
| | | Termination Element |
| | | Stagger Element |
| | | Regulatory Nucleic Acids |
| | Translation Efficiency | Rolling Circle Translation |
| | Untranslated Regions | PolyA Sequence |
| | | RNA-Binding |
| | | Protein-Binding |
| | | Encrytogen |
| | | Riboswitches |
| | | Aptazyme |
| | Replication Element | |
| | Scaffold Sequences | |
| | Other Sequences | Nucleotide Spacer Sequences |
| | | Non-Nucleic Acid Linkers |
| | Stability/Half-life | |
| | Modifications | |
| | Structure | |
| | Delivery | |
| | Methods of Delivery | Cell and Vesicle-Based |
| | | Carriers |
| | Methods of Expression | |

### Circular Polyribonucleotides

In some embodiments, the circular RNAs have a sequence, or plurality of sequences, encoding an expression product(s), e.g., therapeutic expression product(s), e.g., the circular RNAs encode a therapeutic protein or nucleic acid. In some embodiments, the circular RNAs have a sequence, or plurality of sequences, comprising an aptamer. In some embodiments, the circular RNAs have a sequence encoding a sequence having at least 80% (e.g., 85%, 90%, 95%, 97%, 99%, 100% or any percentage therebetween) sequence identity to a an endogenous or naturally occurring circular polyribonucleotide sequence. In some embodiments, the circular RNAs and preparations do not elicit an unwanted immune response in a mammal, e.g., a human.

In some embodiments, the circular polyribonucleotide has a half-life of at least that of its linear counterpart, e.g., linear expression sequence, or a linear polyribonucleotide. In some embodiments, the circular polyribonucleotide has a half-life that is greater than that of its linear counterpart. In some embodiments, the half-life is greater by about 5%, 10%, 15%, 20%, 25%, 30%, 35%, 40%, 45%, 50%, or greater, or any percentage therebetween. In some embodiments, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween. In some embodiments, the circular polyribonucleotide has a half-life or persistence in a cell while the cell is dividing. In some embodiments, the circular polyribonucleotide has a half-life or persistence in a cell post division. In certain embodiments, the circular polyribonucleotide has a half-life or persistence in a dividing cell for greater than about 10 minutes to about 30 days, or at least about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the circular polyribonucleotide modulates a cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the circular polyribonucleotide may be of a sufficient size to accommodate a binding site for a ribosome. One of skill in the art can appreciate that the maximum size of a circular polyribonucleotide can be as large as is within the technical constraints of producing a circular polyribonucleotide, and/or using the circular polyribonucleotide. While not being bound by theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide is limited by the ability of packaging and delivering the RNA to a target. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides may be useful.

In some embodiments, the circular polyribonucleotide comprises one or more elements described elsewhere herein. In some embodiments, the elements may be separated from one another by a spacer sequence or linker. In some embodiments, the elements may be separated from one another by 1 ribonucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides, any amount of nucleotides therebetween. In some embodiments, one or more elements are contiguous with one another, e.g., lacking a spacer element. In some embodiments, one or more elements in the circular polyribonucleotide is conformationally flexible. In some embodiments, the conformational flexibility is due to the sequence being substantially free of a secondary structure. In some embodiments, the circular polyribonucleotide comprises a secondary or tertiary structure that accommodates one or more desired functions or characteristics described herein, e.g., accommodate a binding site for a ribosome, e.g., translation, e.g., rolling circle translation.

In some embodiments, the circular polyribonucleotide comprises particular sequence characteristics. For example, the circular polyribonucleotide may comprise a particular nucleotide composition. In some such embodiments, the circular polyribonucleotide may include one or more purine rich regions (adenine or guanosine). In some such embodiments, the circular polyribonucleotide may include one or more purine rich regions (adenine or guanosine). In some embodiments, the circular polyribonucleotide may include one or more AU rich regions or elements (AREs). In some embodiments, the circular polyribonucleotide may include one or more adenine rich regions.

In some embodiments, the circular polyribonucleotide may include one or more repetitive elements described elsewhere herein.

In some embodiments, the circular polyribonucleotide comprises one or more modifications described elsewhere herein.

In some embodiments, the circular polyribonucleotide comprises one or more expression sequences and is configured for persistent expression in a cell of a subject in vivo. In some embodiments, the circular polyribonucleotide is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

In some embodiments, the circular polyribonucleotide is capable of replicating or replicates in a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, cultured cells, primary cells or cell lines, stem cells, progenitor cells, differentiated cells, germ cells, cancer cells (e.g., tumorigenic, metastic), non-tumorigenic cells (normal cells), fetal cells, embryonic cells, adult cells, mitotic cells, non-mitotic cells, or any combination thereof. In some embodiments, the invention includes a cell comprising the circular polyribonucleotide described herein, wherein the cell is a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, a cultured cell, a primary cell or a cell line, a stem cell, a progenitor cell, a differentiated cell, a germ cell, a cancer cell (e.g., tumorigenic, metastic), a non-tumorigenic cell (normal cells), a fetal cell, an embryonic cell, an adult cell, a mitotic cell, a non-mitotic cell, or any combination thereof. In some embodiments, the cell is modified to comprise the circular polyribonucleotide.

### Methods of Making Circular RNA

In some embodiments, the making of a circular polyribonucleotide includes making a deoxyribonucleic acid sequence that is non-naturally occurring and can be produced using recombinant technology (methods described below; e.g., derived in vitro using a DNA plasmid) or chemical synthesis. In some embodiments, the circularizing of a linear polyribonucleotide is performed by splint ligation.

It is within the scope of the invention that a DNA molecule used to produce an RNA circle can comprise a DNA sequence of a naturally-occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The circular polyribonucleotide may be prepared according to any available technique including, but not limited to chemical synthesis and enzymatic synthesis. In some embodiments, a linear primary construct or linear mRNA may be cyclized, or concatemerized to create a circular polyribonucleotide described herein. The mechanism of cyclization or concatemerization may occur through methods such as, but not limited to, chemical, enzymatic, splint ligation, or ribozyme catalyzed methods. The newly formed 5′-/3′-linkage may be an intramolecular linkage or an intermolecular linkage.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813; International Publication No. WO2016197121; International Publication No. WO2010084371; the contents of each of which are herein incorporated by reference in their entireties).

For example, Examples 13 et seq. herein describe methods of making and characterizing pharmaceutical circular RNA preparations.

### Detection of Linear and Circular RNA

The inventors have found that the presence of linear RNA in pharmaceutical circular RNA preparations can have unexpected and sometimes undesirable effects. Thus, the invention features, inter alia, pharmaceutical compositions and preparations wherein circular RNAs are enriched, separated, and/or purified relative to linear RNA; methods (e.g., methods of manufacturing circular RNA preparations) whereby linear RNAs can be monitored, evaluated and/or controlled; and methods of using such pharmaceutical compositions and preparations, e.g., to deliver an effector, such as a therapeutic effector or scaffold (e.g., an aptamer sequence), to a cell, tissue or subject. In some embodiments, a circular RNA preparation has no more than a threshold level of linear RNA, e.g., a circular RNA preparation is enriched over linear RNA or purified to reduce linear RNA.

Generally, detection and quantitation of an element in a pharmaceutical preparation includes the use of a reference standard that is either the component of interest (e.g., circular RNA, linear RNA, fragment, impurity, etc.) or is a similar material (e.g., using a linear RNA structure of the same sequence as a circular RNA structure as a standard for circular RNA), or includes the use of an internal standard or signal from a test sample. In some embodiments, the standard is used to establish the response from a detector for a known or relative amount of material (response factor). In some embodiments, the response factor is determined from a standard at one or multiple concentrations (e.g., using linear regression analysis). In some embodiments, the response factor is then used to determine the amount of the material of interest from the signal due to that component. In some embodiments, the response factor is a value of one or is assumed to have a value of one.

In some embodiments, detection and quantification of linear versus circular RNA in the pharmaceutical composition is determined using a comparison to a linear version of the circular polyribonucleotides. In some embodiments, the mass of total ribonucleotide in the pharmaceutical composition is determined using a standard curve generated using a linear version of the circular polyribonucleotide and assuming a response factor of one. In some embodiments, a w/w percentage of circular polyribonucleotide in the pharmaceutical preparation is determined by a comparison of a standard curve generated by band intensities of multiple known amounts of a linear version of the circular polyribonucleotide to a band intensity of a the circular polyribonucleotide in the pharmaceutical preparation. In some embodiments, the bands are produced during gel-base electrophoresis, and the band intensities are measured by a gel imager (e.g., an E-gel Imager). For example, the amount of linear polyribonucleotide as compared to circular polyribonucleotide can be determined using the methods of Example 2 and/or Example 3. In some embodiments, a circular polyribonucleotide preparation comprises less than a threshold amount (e.g., where the threshold amount is a reference criterion, e.g., a pharmaceutical release specification for the circular polyribonucleotide preparation) of linear polyribonucleotide molecules when evaluated as described herein.

In some embodiments, detection and quantification of nicked versus total RNA in the pharmaceutical composition is determined by sequencing after gel extraction of the preparation comprising the circular RNA. In some embodiments, detection and quantification of nicked versus linear RNA in the pharmaceutical composition is determined by sequencing after gel extraction of the preparation comprising the circular RNA. For example, the amount of nicked polyribonucleotide as compared to total RNA can be determined using the methods of Example 5. For example, the amount of nicked polyribonucleotide as compared to linear RNA can be determined using the methods of Example 5. In some embodiments, a circular polyribonucleotide preparation comprises less than a threshold amount (e.g., where the threshold amount is a reference criterion, e.g., a pharmaceutical release specification for the circular polyribonucleotide preparation) of nicked RNA, linear RNA, or combined linear and nicked RNA when evaluated as described herein. For example, the reference criterion for the amount of linear polyribonucleotide molecules present in the preparation is no more than 30%, 20%, 15%, 10%, 1%, 0.5%, or 0.1% linear polyribonucleotide molecules, or any percentage therebetween, relative to total ribonucleotide molecules in the preparation. In some embodiments, the reference criterion for the amount of nicked polyribonucleotide molecules present in the preparation is no more than 30%, 20%, 15%, 10%, 1%, 0.5%, or 0.1%, or any percentage therebetween, nicked polyribonucleotide molecules relative to total ribonucleotide molecules in the preparation. In some embodiments, the reference criterion for the amount of linear and nicked polyribonucleotide molecules present in the preparation is no more than 40%, 30%, 20%, 15%, 10%, 1%, 0.5%, or 0.1%, or any percentage therebetween, combined linear polyribonucleotide and nicked polyribonucleotide molecules relative to total ribonucleotide molecules in the preparation.

In some embodiments, the standard is run under the same conditions as the sample. For example, the standard is run with the same type of gel, same buffer, and same exposure as the sample. In further embodiments, the standard is run in parallel with the sample. In some embodiments, a quantification of an element is repeated (e.g., twice or in triplicate) in a plurality of samples from the subject preparation to obtain a mean result. In some embodiments, quantitation of a linear RNA is measured using parallel capillary electrophoresis (e.g., using a Fragment Analyzer or analytical HPLC with UV detection).

### Purification of Circular RNA

Circular polyribonucleotides may be separated, enriched, or purified from unwanted substances (such as unwanted (e.g., linear) RNA, enzymes, DNA). In some embodiments, the unwanted substances are present in, or originating from, a process of making and/or manufacturing the circular polyribonucleotides. Circular polyribonucleotides described herein may be enriched and/or purified prior to formulation in a pharmaceutical preparation, pharmaceutical composition, pharmaceutical drug substance, or pharmaceutical drug product. Circular polyribonucleotides described herein may be enriched and/or purified during or after formulation in a pharmaceutical preparation, pharmaceutical composition, pharmaceutical drug substance, or pharmaceutical drug product.

In some embodiments, the circular RNAs may be purified during or after production to remove undesirable elements, e.g., linear RNA, or nicked RNA, as well as recognized impurities, e.g., free ribonucleic acids (e.g., monoribonucleic acids, diribonucleic acids, or triribonucleic acids), DNA (e.g., cell DNA, such as host cell DNA), cell or process-related protein impurities (e.g., cell or process-related impurities), etc. In some embodiments, an impurity is a process-related impurity. In some embodiments, the process-related impurity is a protein (e.g., a cell protein), a nucleic acid (e.g., a cell nucleic acid), a buffer or buffer reagent, an enzyme, a media/reagent component (e.g., media, media additive, transition metal, or vitamin), a preparatory or analytical gel component (e.g., acrylamide debris), DNA, or a chromatographic material. A buffer reagent can be MgCl₂, DTT, ATP, SDS, Na, glycogen, Tris-HCL, or EtOH. A buffer reagent can include, but is not limited to, acetate, Tris, bicarbonate, phosphate, citric acid, lactate, or TEA. An enzyme can be a ligase. A ligase can be T4 RNA ligase 2. In some embodiments, an impurity is a buffer reagent, a media/reagent component, a salt, a ligase, a nuclease, an RNase inhibitor, RNase R, linear polyribonucleotide molecules, deoxyribonucleotide molecules, acrylamide debris, or mononucleotide molecules.

In some embodiments, the circular polyribonucleotides may be enriched or purified by any known method commonly used in the art. Examples of non-limiting purification methods include column chromatography, gel excision, size exclusion, etc.

In some embodiments, a circular polyribonucleotide is purified by gel purification e.g., UREA gel separation, e.g., as described in Example 3. For example, a circular RNA may be resolved on a denaturing PAGE and bands corresponding to the circular RNAs may be excised and the circular RNA may be eluted from the band using known methods. The eluted circular RNA may then be analyzed.

In some embodiments, a circular polyribonucleotide is purified by chromatography, e.g., hydrophobic interaction chromatography (HIC), mixed-mode chromatography, liquid chromatography, e.g., reverse-phase ion-pair chromatography (IP-RP), ion-exchange chromatography (IE), affinity chromatography (AC), and size-exclusion chromatography (SEC), and any combinations thereof.

In some embodiments, a circular polyribonucleotide is purified by utilizing a structural feature of the circular polyribonucleotide to separate it from a linear RNA or an impurity. In some embodiments, the circular polyribonucleotide is purified by utilizing a structural feature (e.g., a lack of free ends) such as described in Example 9. For example, circular RNA is enriched from a preparation comprising a mixed pool of circular RNA and linear RNA counterpart containing the same nucleotide sequences using polyadenylation of the linear RNA counterpart or fragments thereof. The 3′ end of the linear RNA counterpart or fragments thereof can be polyadenylated using poly(A) polymerase, resulting in the addition of a 3′ polyadenine tail. In some embodiments, the 3′ polyadenine tail enables a pulldown of the linear RNA and fragments thereof using a column, such as an affinity column, to enrich for the circular RNA. Poly(A) polymerase can also incorporate modified adenines such as the biotinylated N6-ATP analog. This addition biotinylated N6-ATP analog into the 3′ polyadenine tail of enables a pulldown of the linear RNA and fragments thereof in a system such as a biotin-streptavidin binding system. In contrast, circularized RNA does not have a 3′ end, and therefore is not polyadenylated by the poly(A) polymerase, does not have a polyadenylated tail for conjugation, and is not captured in the pulldown. Therefore, the circular RNA is enriched in the preparation after the pulldown.

In some embodiments, the circular polyribonucleotide is purified by utilizing a structural feature of the linear RNA (e.g., presence of free ends). For example, circular RNA is enriched from a preparation comprising a mixed pool of circular RNA and linear RNA counterpart containing the same nucleotide sequences using polyadenylation of the linear RNA counterpart. Exonucleases can be added to the mixed pool to hydrolyze the linear RNA. In some embodiments, an exonuclease can be 3′ exonuclease or a 5′ exonuclease. In some embodiments, a 3′ exonuclease and a 5′ exonuclease can be used.

In an embodiment, a circular polyribonucleotide preparation (e.g., a circular polyribonucleotide pharmaceutical preparation or composition or an intermediate in the production of the circular polyribonucleotide preparation) is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), or 100% (w/w) pure on a mass basis. Purity may be measured by any one of a number of analytical techniques known to one skilled in the art, such as, but not limited to, the use of separation technologies such as chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre- or post-separation derivatization methodologies using detection techniques based on mass spectrometry, UV-visible, fluorescence, light scattering, refractive index, or that use silver or dye stains or radioactive decay for detection. Alternatively, purity may be determined without the use of a separation technology by mass spectrometry, by microscopy, by circular dichroism (CD) spectroscopy, by UV or UV-vis spectrophotometry, by fluorometry (e.g., Qubit), by RNAse H analysis, by surface plasmon resonance (SPR), or by methods that utilize silver or dye stains or radioactive decay for detection.

In some embodiments, purity can be measured by biological test methodologies (e.g., cell-based or receptor-based tests). In some embodiments, at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w) or 100% (w/w) of the total of mass ribonucleotide in the a preparation described herein is contained in circular polyribonucleotide molecules. The percent may be measured by any one of a number of analytical techniques known to one skilled in the art such as, but not limited to, the use of a separation technology such as chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre- or post-separation derivatization methodologies using detection techniques based on mass spectrometry, UV-visible, fluorescence, light scattering, refractive index, or that use silver or dye stains or radioactive decay for detection. Alternatively, purity may be determined without the use of separation technologies by mass spectrometry, by microscopy, by circular dichroism (CD) spectroscopy, by UV or UV-vis spectrophotometry, by fluorometry (e.g., Qubit), by RNAse H analysis, by surface plasmon resonance (SPR), or by methods that utilize silver or dye stains or radioactive decay for detection.

In an embodiment, a circular polyribonucleotide preparation (e.g., a circular polyribonucleotide pharmaceutical preparation or composition or an intermediate in the production of the circular polyribonucleotide preparation) has a circular polyribonucleotide concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1000 μg/mL, 5000 μg/mL, 10,000 μg/mL, 100,000 μg/mL, 200 mg/mL, 300 mg/mL, 400 mg/mL, 500 mg/mL, 600 mg/mL, 650 mg/mL, 700 mg/mL, or 750 mg/mL. In an embodiment, a circular polyribonucleotide preparation (e.g., a circular polyribonucleotide pharmaceutical preparation or composition or an intermediate in the production of the circular polyribonucleotide preparation) is substantially free of mononucleotide or has a mononucleotide content of no more than 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL. In an embodiment, a circular polyribonucleotide preparation (e.g., a circular polyribonucleotide pharmaceutical preparation or composition or an intermediate in the production of the circular polyribonucleotide preparation) has a mononucleotide content from the limit of detection up to 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL.

In an embodiment, a circular polyribonucleotide preparation (e.g., a circular polyribonucleotide pharmaceutical preparation or composition or an intermediate in the production of the circular polyribonucleotide preparation) has mononucleotide content no more than 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), or any percentage therebetween of total nucleotides on a mass basis, wherein total nucleotide content is the total mass of deoxyribonucleotide molecules and ribonucleotide molecules.

In an embodiment, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 6 Ong/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/mL, 10 mg/mL, 50 mg/mL, 100 mg/mL, 200 mg/mL, 300 mg/mL, 400 mg/mL, 500 mg/mL, 600 mg/mL, 650 mg/mL, 700 mg/mL, or 750 mg/mL. In an embodiment, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, from the limit of detection of up to 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/ml, 10 mg/ml, 50 mg/ml, 100 mg/ml, 200 mg/ml, 300 mg/ml, 400 mg/ml, 500 mg/ml, 600 mg/ml, 650 mg/ml, 700 mg/ml, or 750 mg/ml.

In an embodiment, a circular polyribonucleotide preparation (e.g., a circular polyribonucleotide pharmaceutical preparation or composition or an intermediate in the production of the circular polyribonucleotide preparation) has a nicked RNA content of no more than 10% (w/w), 9.9% (w/w), 9.8% (w/w), 9.7% (w/w), 9.6% (w/w), 9.5% (w/w), 9.4% (w/w), 9.3% (w/w), 9.2% (w/w), 9.1% (w/w), 9% (w/w), 8% (w/w), 7% (w/w), 6% (w/w), 5% (w/w), 4% (w/w), 3% (w/w), 2% (w/w), 1% (w/w), 0.5% (w/w), or 0.1% (w/w), or percentage therebetween. In an embodiment, a circular polyribonucleotide preparation (e.g., a circular polyribonucleotide pharmaceutical preparation or composition or an intermediate in the production of the circular polyribonucleotide preparation) has a nicked RNA content that as low as zero or is substantially free of nicked RNA.

In an embodiment, a circular polyribonucleotide preparation (e.g., a circular polyribonucleotide pharmaceutical preparation or composition or an intermediate in the production of the circular polyribonucleotide preparation) has a combined linear RNA and nicked RNA content of no more than 30% (w/w), 25% (w/w), 20% (w/w), 15% (w/w), 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or 0.1% (w/w), or percentage therebetween. In an embodiment, a circular polyribonucleotide preparation (e.g., a circular polyribonucleotide pharmaceutical preparation or composition or an intermediate in the production of the circular polyribonucleotide preparation) has a combined nicked RNA and linear RNA content that is as low as zero or is substantially free of nicked and linear RNA.

In some embodiments, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, of no more than the detection limit of analytical methodologies, such as methods utilizing mass spectrometry, UV spectroscopic or fluorescence detectors, light scattering techniques, surface plasmon resonance (SPR) with or without the use of methods of separation including HPLC, by HPLC, chip or gel based electrophoresis with or without using either pre or post separation derivatization methodologies, methods of detection that use silver or dye stains or radioactive decay, or microscopy, visual methods or a spectrophotometer.

In an embodiment, a circular polyribonucleotide preparation (e.g., a circular polyribonucleotide pharmaceutical preparation or composition or an intermediate in the production of the circular polyribonucleotide preparation) has no more than 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of linear RNA, e.g., as measured by the methods in Example 2.

In some embodiments, the linear polyribonucleotide molecules of the circular polyribonucleotide preparation comprise the linear counterpart or a fragment thereof of the circular polyribonucleotide molecule. In some embodiments, the linear polyribonucleotide molecules of the circular polyribonucleotide preparation comprise the linear counterpart (e.g., a pre-circularized version). In some embodiments, the linear polyribonucleotide molecules of the circular polyribonucleotide preparation comprise a non-counterpart or fragment thereof to the circular polyribonucleotide. In some embodiments, the linear polyribonucleotide molecules of the circular polyribonucleotide preparation comprise a non-counterpart to the circular polyribonucleotide. In some embodiments, the linear polyribonucleotide molecules comprises a combination of the counterpart of the circular polyribonucleotide and a non-counterpart or fragment thereof of the circular polyribonucleotide. In some embodiments, the linear polyribonucleotide molecules comprises a combination of the counterpart of the circular polyribonucleotide and a non-counterpart of the circular polyribonucleotide. In some embodiments, a linear polyribonucleotide molecule fragment is a fragment that is at least 1, 2, 3, 4, 5, 6, 7, 8, 9, 10, 20, 30, 40, 50, 60, 70, 80, 90, 100, 200, 300, 400, 500, 1000, 2000, 3000, 4000, 5000, 6000, 7000, 8000, 9000, 10000, 11000, 12000, or more nucleotides in length, or any nucleotide number therebetween.

In some embodiments, a circular polyribonucleotide preparation (e.g., a circular polyribonucleotide pharmaceutical preparation or composition or an intermediate in the production of the circular polyribonucleotide preparation) has an A260/A280 absorbance ratio from about 1.6 to about 2.3, e.g., as measured by spectrophotometer. In some embodiments, the A260/A280 absorbance ratio is about 1.4, 1.5, 1.6, 1.7, 1.8, 1.9, 2.0, 2.1, 2.2, 2.3, 2.4, 2.5, or any number therebetween. In some embodiments, a circular polyribonucleotide (e.g., a circular polyribonucleotide pharmaceutical preparation or composition or an intermediate in the production of the circular polyribonucleotide) has an A260/A280 absorbance ratio greater than about 1.8, e.g., as measured by spectrophotometer. In some embodiments, the A260/A280 absorbance ratio is about 1.6, 1.7, 1.8, 1.9, 2.0, 2.1, 2.2, 2.3, 2.4, 2.5, 2.6, 2.7, 2.8, 2.9, or greater.

In some embodiments, a circular polyribonucleotide preparation (e.g., a circular polyribonucleotide pharmaceutical preparation or composition or an intermediate in the production of the circular polyribonucleotide preparation) is substantially free of an impurity. In various embodiments, the level of at least one impurity in a composition comprising the circular polyribonucleotide is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity. In some embodiments, the level of at least one process-related impurity is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity. In some embodiments, the level of at least one product-related substance is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the a composition prior to purification or treatment to remove the impurity. In some embodiments, a circular polyribonucleotide preparation (e.g., a circular polyribonucleotide pharmaceutical preparation or composition or an intermediate in the production of the circular polyribonucleotide preparation) is further substantially free of a process-related impurity. In some embodiments, the process-related impurity comprises a protein (e.g., a cell protein, such as a host cell protein), a deoxyribonucleic acid (e.g., a cell deoxyribonucleic acid, such as a host cell deoxyribonucleic acid), monodeoxyribonucleotide or dideoxyribonucleotide molecules, an enzyme (e.g., a nuclease, such as an endonuclease or exonuclease, or ligase), a reagent component, a gel component, or a chromatographic material. In some embodiments, the impurity is selected from: a buffer reagent, a ligase, a nuclease, RNase inhibitor, RNase R, deoxyribonucleotide molecules, acrylamide gel debris, and monodeoxyribonucleotide molecules. In some embodiments, the pharmaceutical preparation comprises protein (e.g., cell protein, such as a host cell protein) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of protein contamination per milligram (mg) of the circular polyribonucleotide molecules.

In an embodiment, a circular polyribonucleotide preparation (e.g., a circular polyribonucleotide pharmaceutical preparation or composition or an intermediate in the production of the circular polyribonucleotide preparation) is substantially free of DNA content e.g., template DNA or cell DNA (e.g., host cell DNA), has a DNA content, as low as zero, or has a DNA content of no more than 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL.

In an embodiment, a circular polyribonucleotide preparation (e.g., a circular polyribonucleotide pharmaceutical preparation or composition or an intermediate in the production of the circular polyribonucleotide preparation) is substantially free of DNA content, has a DNA content as low as zero, or has DNA content no more than 0.001% (w/w), 0.01% (w/w), 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of total nucleotides on a mass basis, wherein total nucleotide molecules is the total mass of deoxyribonucleotide content and ribonucleotide molecules. In an embodiment, a circular polyribonucleotide preparation (e.g., a circular polyribonucleotide pharmaceutical preparation or composition or an intermediate in the production of the circular polyribonucleotide preparation) is substantially free of DNA content, has DNA content as low as zero, or has DNA content no more than 0.001% (w/w), 0.01% (w/w), 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of total nucleotides on a mass basis as measured after a total DNA digestion by enzymes that digest nucleosides by quantitative liquid chromatography-mass spectrometry (LC-MS), in which the content of DNA is back calculated from a standard curve of each base (i.e., A, C, G, T) as measured by LC-MS.

In an embodiment, a circular polyribonucleotide preparation (e.g., a circular polyribonucleotide pharmaceutical preparation or composition or an intermediate in the production of the circular polyribonucleotide preparation) has a protein (e.g., cell protein (CP), e.g., enzyme, a production-related protein, e.g., carrier protein) contamination of no more than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml. In an embodiment, a circular polyribonucleotide (e.g., a circular polyribonucleotide pharmaceutical preparation or composition or an intermediate in the production of the circular polyribonucleotide) has a protein (e.g., production-related protein such as a cell protein (CP), e.g., enzyme) contamination from the limit of detection of up to 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, a circular polyribonucleotide preparation (e.g., a circular polyribonucleotide pharmaceutical preparation or composition or an intermediate in the production of the circular polyribonucleotide preparation) has a protein (e.g., production-related protein such as a cell protein (CP), e.g., enzyme) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circular polyribonucleotide. In an embodiment, a circular polyribonucleotide (e.g., a circular polyribonucleotide pharmaceutical preparation or composition or an intermediate in the production of the circular polyribonucleotide) has a protein (e.g., production-related protein such as a cell protein (CP), e.g., enzyme) contamination from the level of detection up to 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circular polyribonucleotide.

In an embodiment, a circular polyribonucleotide preparation (e.g., a circular polyribonucleotide pharmaceutical preparation or composition or an intermediate in the production of the circular polyribonucleotide preparation) has low levels or is substantially absent of endotoxins, e.g., as measured by the Limulus amebocyte lysate (LAL) test. In some embodiments, the pharmaceutical preparation or compositions or an intermediate in the production of the circular polyribonucleotides comprises less than 20 EU/kg (weight), 10 EU/kg, 5 EU/kg, 1 EU/kg, or lacks endotoxin as measured by the Limulus amebocyte lysate test. In an embodiment, a circular polyribonucleotide composition has low levels or absence of a nuclease or a ligase.

In some embodiments, a circular polyribonucleotide preparation (e.g., a circular polyribonucleotide pharmaceutical preparation or composition or an intermediate in the production of the circular polyribonucleotide preparation) comprises no greater than about 50% (w/w), 45% (w/w), 40% (w/w), 35% (w/w), 30% (w/w), 25% (w/w), 20% (w/w), 19% (w/w), 18% (w/w), 17% (w/w), 16% (w/w), 15% (w/w), 14% (w/w), 13% (w/w), 12% (w/w), 11% (w/w), 10% (w/w), 9% (w/w), 8% (w/w), 7% (w/w), 6% (w/w), 5% (w/w), 4% (w/w), 3% (w/w), 2% (w/w), 1% (w/w) of at least one enzyme, e.g., polymerase, e.g., RNA polymerase.

In an embodiment, a circular polyribonucleotide preparation (e.g., a circular polyribonucleotide pharmaceutical preparation or composition or an intermediate in the production of the circular polyribonucleotide preparation) is sterile or substantially free of microorganisms, e.g., the composition or preparation supports the growth of fewer than 100 viable microorganisms as tested under aseptic conditions, the composition or preparation meets the standard of USP <71>, and/or the composition or preparation meets the standard of USP <85>. In some embodiments, the pharmaceutical preparation comprises a bioburden of less than 100 CFU/100 ml, 50 CFU/100 ml, 40 CFU/100 ml, 30 CFU/100 ml, 200 CFU/100 ml, 10 CFU/100 ml, or 10 CFU/100 ml before sterilization.

In some embodiments, the circular polyribonucleotide preparation can be further purified using known techniques in the art for removing impurities, such as column chromatography or pH/vial inactivation.

In some embodiments, the circular polyribonucleotide preparation produces a reduced level of one more markers of an immune or inflammatory response after administration to a subject when the circular polyribonucleotide preparation has undergone a purification step (or a plurality of purification steps) compared to prior to the purification step(s). The purification can be performed as described herein, e.g., as in Examples 1-8. In some embodiments, the one or more markers of an immune or inflammatory response is a cytokine or immune response related gene. In some embodiments, the one or more markers of an immune or inflammatory response is expression of a gene, such as RIG-I, MDA5, PKR, IFN-beta, OAS, and OASL.

In an embodiment, a circular polyribonucleotide preparation (e.g., a circular polyribonucleotide pharmaceutical preparation or composition or an intermediate in the production of the circular polyribonucleotide preparation) expresses an expression product, e.g., protein, e.g., in-vitro translation activity, e.g., as measured by an assay described in Example 3.

### Pharmaceutical Compositions

The present invention includes compositions in combination with one or more pharmaceutically acceptable excipients. Pharmaceutical compositions may optionally comprise one or more additional active substances, e.g. therapeutically and/or prophylactically active substances. Pharmaceutical compositions may optionally comprise an inactive substance that serves as a vehicle or medium for the compositions described herein (e.g., compositions comprising circular polyribonucleotides, such as any one of the inactive ingredients approved by the United States Food and Drug Administration (FDA) and listed in the Inactive Ingredient Database). Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference). Non-limiting examples of an inactive substance include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

### Methods of Manufacturing Pharmaceutical Circular RNA Preparations

A method for manufacturing a pharmaceutical composition, a pharmaceutical drug substance, or a pharmaceutical drug product as disclosed herein can comprise processing a preparation of circular polyribonucleotides to reduce linear RNA and/or nicked RNA, evaluating the amount of remaining linear RNA and/or nicked RNA, and further processing the preparation to produce a pharmaceutical composition, drug substance, or drug product for pharmaceutical use.

A method for manufacturing a pharmaceutical composition, a pharmaceutical drug substance, or a pharmaceutical drug product as disclosed herein can comprise providing a preparation of circular polyribonucleotides, assessing the preparation for the amount of linear RNA and/or nicked RNA, and processing the preparation to produce a pharmaceutical composition, drug substance, or drug product for pharmaceutical use, if the assessment meets a pre-determined reference criterion for linear RNA and/or nicked, such as a pharmaceutical release specification.

A method for testing a pharmaceutical composition, a pharmaceutical drug substance, or a pharmaceutical drug product as disclosed herein can comprise providing a preparation of circular polyribonucleotides, assessing the preparation for the amount of linear RNA, and determining if the assessment meets a pre-determined reference criterion for linear RNA, such as a pharmaceutical release specification.

A method for testing a pharmaceutical composition, a pharmaceutical drug substance, or a pharmaceutical drug product as disclosed herein can comprise providing a preparation of circular polyribonucleotides, assessing the preparation for the amount of nicked RNA, and determining if the assessment meets a pre-determined reference criterion for nicked RNA, such as a pharmaceutical release specification.

For example, the reference criterion for the amount of linear polyribonucleotide molecules present in the preparation is the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.

For example, the reference criterion for the amount of circular polyribonucleotide molecules present in the preparation is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) molecules of the total ribonucleotide molecules in the pharmaceutical preparation.

For example, the reference criterion for the amount of linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) linear polyribonucleotide molecules of the total ribonucleotide molecules in the pharmaceutical preparation.

For example, the reference criterion for the amount of nicked polyribonucleotide molecules present in the preparation is no more than 0.5% (w/w), 1% (w/w), 2% (w/w), 5% (w/w), 10% (w/w), or 15% (w/w) nicked polyribonucleotide molecules of the total ribonucleotide molecules in the pharmaceutical preparation.

For example, the reference criterion for the amount of combined nicked and linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) combined nicked and linear polyribonucleotide molecules of the total ribonucleotide molecules in the pharmaceutical preparation. In some embodiments, a pharmaceutical preparation is an intermediate pharmaceutical preparation of a final circular polyribonucleotide drug product. In some embodiments, a pharmaceutical preparation is a drug substance or active pharmaceutical ingredient (API). In some embodiments, a pharmaceutical preparation is a drug product for administration to a subject.

In some embodiments, a preparation of circular polyribonucleotides is (before, during or after the reduction of linear RNA) further processed to substantially remove DNA, protein contamination (e.g., cell protein such as a host cell protein or protein process impurities), endotoxin, mononucleotide molecules, and/or a process-related impurity.

In some embodiments, the preparation of circular polyribonucleotides is subsequently combined with a pharmaceutical excipient, e.g., if it meets a specification for linear RNA levels. In some embodiments, the pharmaceutical excipient comprises an inorganic or organic buffer to control pH, a sugar, an amino acid or any other material for circular polyribonucleotide stability, sodium chloride or any other material for adjusting tonicity, or a surfactant such as a non-ionic surfactant. In some embodiments the pharmaceutical excipient comprises a monosaccharide, a disaccharide (e.g., sucrose, lactose, or trehalose), a trisaccharide, a polysaccharide, an amino sugar (e.g., meglumine), a polyalcohol, a salt (e.g., sodium bicarbonate, sodium phosphate, or sodium chloride), magnesium stearate, an amino acid (e.g., histidine or arginine), a surfactant (e.g., glycerol or polysorbate 80), a chelating agent (e.g., EDTA), camphorsulfonic acid, or a lyoprotectant (e.g., clyclodextrin). In some embodiments, the pharmaceutical excipient comprises citrate buffer. In some embodiments, the pharmaceutical excipient comprises a donor methyl group S-adenosylmethionine (SAM). In some embodiments, the pharmaceutical excipient comprises Alpha-Terpineol; Alpha-Tocopherol; Alpha-Tocopherol Acetate; Alpha-Tocopherol; 1,2,6-Hexanetriol; 1,2-Dimyristoyl-Sn-Glycero-3-(Phospho-S—); 1-Glycerol; 1,2-Dimyristoyl-Sn-Glycero-3-; Phosphocholine, 1,2-Dioleoyl-Sn-Glycero-3-Phosphocholine; 1,2-Dipalmitoyl-Sn-Glycero-3-(Phospho-); Rac-(1-Glycerol); 1,2-Distearoyl-Sn-Glycero-3-(Phospho-Rac-); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adipic Acid; Aerotex Resin 3730; Alanine (Infusion); Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl Alpha-Ionone; Almond Oil; Aluminum Acetate; Aluminum Chlorhydroxy Allantoinate; Aluminum Hydroxide; Aluminum Hydroxide-Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear; Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic; Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl); Pentaerythritol (Crosslinked); Carbomer Homopolymer Type C (Allyl); Pentaerythritol (Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax 1; Cetyl Palmitate; Cetylpyridinium Chloride; Chlorobutanol; Chlorobutanol Hemihydrate I; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa; Coco-Glycerides; Coconut Oil; Coconut Oil, Hydrogenated; Coconut Oil/Palm Kernel Oil Glycerides; Cocoyl Caprylocaprate; Cola Nitida Seed Extract; Collagen; Coloring Suspension; Corn Oil; Cottonseed Oil; Cream Base; Creatine; Creatinine; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl; Methacrylate-Methyl Methacrylate, Copolymer; Dimethyldioctadecylammonium Bentonite; Dimethylsiloxane/Methylvinylsiloxane, Copolymer; Dinoseb Ammonium Salt; Dipalmitoylphosphatidylglycerol; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Egg Phospholipids; Entsufon; Entsufon; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer; Ethylene-Vinyl Acetate Copolymer; Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10; Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor Fig 827118; Flavor Raspberry Pfc-8407; Flavor Rhodia Pharmaceutical No. Rf 451; Fluorochlorohydrocarbons; Formaldehyde; Formaldehyde; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1 c; Fragrance H-6540; Fragrance Herbal 10396; Fragrance Nj-1085; Fragrance P O Fl-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl; Diethylamine; Glyceryl Trioleate; Glycine; Glycine Hydrochloride; Glycol Distearate; Glycol Stearate; Guanidine Hydrochloride; Guar Gum; Hair Conditioner (18nl95-lm); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6; Esters; Hydrogenated Polybutene 635-690; Hydroxide Ion; Hydroxyethyl Cellulose; Hydroxyethylpiperazine Ethane Sulfonic; Hydroxymethyl Cellulose; Hydroxyoctacosanyl Hydroxystearate; Hydroxypropyl Cellulose; Hydroxypropyl Methylcellulose 2906; Hydroxypropyl-Bcyclodextrin; Hypromellose 2208 (15000 Mpa·S); Hypromellose 2910 (15000 Mpa·S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate-Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid; Lactic Acid; Lactobionic Acid; Lactose; Lactose Monohydrate; Lactose, Hydrous; Laneth; Lanolin; Lanolin Alcohol—Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4 T; Laurie Diethanolamide; Laurie Myristic Diethanolamide; Lauroyl Sarcosine; Lauryl Lactate; Lauryl Sulfate; Lavandula Angustifolia Flowering; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methane Sulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystalline Wax; Mineral Oil; Mono and Diglyceride; Monostearyl Citrate; Monothioglycerol; Multisterol Extract; Myristyl Alcohol; Myristyl Lactate; Myristyl-gamma-Picolinium Chloride; N-(Carbamoyl-Methoxy Peg-40)-1,2-; Distearoyl-Cephalin Sodium; N,N-Dimethylacetamide; Niacinamide; Nioxime; Nitric Acid; Peg-2 Stearate; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus sylvestris); Piperazine Hexahydrate; Plastibase-50w; Polacrilin Iontophoresis; Polidronium Chloride; Poloxamer 124; Poloxamer 181; Poloxamer 182; Poloxamer 188; Poloxamer 237; Poloxamer 407; Poly(Bis(P-Carboxyphenoxy)Propane; Anhydride, Sebacic Acid; Poly(Dimethylsiloxane/Methylvinylsiloxane/Methylhydrogensiloxane) Dimethylvinyl, Dimethylhydroxy, or Trimethyl Endblocked; Poly(Dl-Lactic-Co-Glycolic Acid); Poly(Dl-Lactic-Co-Glycolic Acid); Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric; Oxide Black (<1%); Polyethylene Low Density; Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7; Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate, Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel Iontophoresis; Povidone K17; Povidone K25; Povidone K29/32; Povidone K30; Povidone K90; Povidone K90f, Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl; Urea/Methylparaben/Propylparben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Saf flower Oil; Sd Alcohol 3 a; Sd Alcohol 40; Sd Alcohol 40-2; Sd Alcohol 40b; Sepineo P 600; Serine; Sesame Oil; Shea Butter; Silastic Medical Adhesive; Silicone Type A; Silica; Silicon; Silicon Dioxide; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic or Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic, or Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Efhyihexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene, Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal; Collagen; Stearyl AlcoholSterile Water; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether Beta-Cyclodextrin Intramuscular; Sulfur Dioxide; Sulfuric Acid; Sulfurous Acid; Surfactol Qs; Tagatose, D-; Talc; Tall Oil; Tallow Glycerides; Tartaric Acid; Tartaric Acid; Tenox; Tenox-2; Tert-Butyl Alcohol; Tert-Butyl Hydroperoxide; Tert-Butylhydroquinone; Tetrakis (2-Methoxyisobutylisocyanide)Copper(I); Tetrafluoroborate; Tetrapropyl Orthosilicate; Tetrofosmin; Theophylline; Thimerosal; Threonine; Thymol; Tin; Titanium Dioxide; Tocopherol; Tocophersolan; Triacetin; Tricaprylin; Trichloromonofluoromethane; Trideceth-10; Triethanolamine Lauryl Sulfate; Trifluoroacetic Acid; Triglycerides, Medium Chain; Trihydroxy stearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine; Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying, Wecobee Fs; White Ceresin Wax; White Wax; Xanthan Gum; Zinc; Zinc Acetate; Zinc Carbonate; Zinc Chloride; or Zinc Oxide. In some embodiments, the preparation of circular polyribonucleotides is combined with a lipid nanoparticle (LNP).

In some embodiments, the preparation of circular polyribonucleotides is subsequently combined with a pharmaceutical excipient comprising a disaccharide, such as sucrose, lactose, or trehalose. In some embodiments, the preparation of circular polyribonucleotides is subsequently combined with a pharmaceutical excipient comprising sucrose. In some embodiments, the preparation of circular polyribonucleotides is subsequently combined with a pharmaceutical excipient comprising a polysaccharide. In some embodiments, the preparation of circular polyribonucleotides is subsequently combined with a pharmaceutical excipient comprising a surfactant, such as glycerol or polysorbate 80. In some embodiments, the preparation of circular polyribonucleotides is subsequently combined with a pharmaceutical excipient comprising alpha-tocopherol. In some embodiments, the preparation of circular polyribonucleotides is subsequently combined with a pharmaceutical excipient comprising phosphocholine. In some embodiments, the preparation of circular polyribonucleotides is subsequently combined with a pharmaceutical excipient comprising an alcohol. In some embodiments, the preparation of circular polyribonucleotides is subsequently combined with a pharmaceutical excipient comprising isopropyl alcohol. In some embodiments, the preparation of circular polyribonucleotides is subsequently combined with a pharmaceutical excipient comprising lanolin alcohol. In some embodiments, the preparation of circular polyribonucleotides is subsequently combined with a pharmaceutical excipient comprising human albumin. In some embodiments, the preparation of circular polyribonucleotides is subsequently combined with a pharmaceutical excipient comprising aluminum hydroxide gel F 500. In some embodiments, the preparation of circular polyribonucleotides is subsequently combined with a pharmaceutical excipient comprising aspartic acid. In some embodiments, the preparation of circular polyribonucleotides is subsequently combined with a pharmaceutical excipient comprising barium sulfate. In some embodiments, the preparation of circular polyribonucleotides is subsequently combined with a pharmaceutical excipient comprising benzoic acid. In some embodiments, the preparation of circular polyribonucleotides is subsequently combined with a pharmaceutical excipient comprising calcium. In some embodiments, the preparation of circular polyribonucleotides is subsequently combined with a pharmaceutical excipient comprising calcium chloride. In some embodiments, the preparation of circular polyribonucleotides is subsequently combined with a pharmaceutical excipient comprising carboxymethylcellulose. In some embodiments, the preparation of circular polyribonucleotides is subsequently combined with a pharmaceutical excipient comprising citric acid. In some embodiments, the preparation of circular polyribonucleotides is subsequently combined with a pharmaceutical excipient comprising ethylene glycol. In some embodiments, the preparation of circular polyribonucleotides is subsequently combined with a pharmaceutical excipient comprising ferric chloride. In some embodiments, the preparation of circular polyribonucleotides is subsequently combined with a pharmaceutical excipient comprising hydrocarbon gel. In some embodiments, the preparation of circular polyribonucleotides is subsequently combined with a pharmaceutical excipient comprising magnesium chloride. In some embodiments, the preparation of circular polyribonucleotides is subsequently combined with a pharmaceutical excipient comprising niacinamide. In some embodiments, the preparation of circular polyribonucleotides is subsequently combined with a pharmaceutical excipient comprising polyethylene glycol. In some embodiments, the preparation of circular polyribonucleotides is subsequently combined with a pharmaceutical excipient comprising potassium chloride. In some embodiments, the preparation of circular polyribonucleotides is subsequently combined with a pharmaceutical excipient comprising propylene glycol. In some embodiments, the preparation of circular polyribonucleotides is subsequently combined with a pharmaceutical excipient comprising sodium carbonate. In some embodiments, the preparation of circular polyribonucleotides is subsequently combined with a pharmaceutical excipient comprising sodium chloride. In some embodiments, the preparation of circular polyribonucleotides is subsequently combined with a pharmaceutical excipient comprising sodium lactate. In some embodiments, the preparation of circular polyribonucleotides is subsequently combined with a pharmaceutical excipient comprising zinc acetate.

In some embodiments, the amount of an impurity (e.g., a cell protein, a cell nucleic acid, an enzyme, a reagent component, a gel component, or a chromatographic material, protein contamination, or endotoxin contamination) is measured to determine if the pharmaceutical composition, pharmaceutical drug substance or pharmaceutical drug product meets a reference criterion.

For example, the reference criterion for the amount of DNA present in the preparation is the presence of zero DNA molecules, substantially free of DNA molecules, or no more than 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml, 1000 μg/mL, 5000 μg/mL, 10,000 μg/mL, or 100,000 μg/mL of DNA.

For example, the reference criterion for the amount of protein contamination present in the preparation is the presence of a protein contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of the protein contamination per milligram (mg) of the circular polyribonucleotide molecules.

In some embodiments, the amount of endotoxin present in the pharmaceutical composition, pharmaceutical drug substance or pharmaceutical drug product is less than 20 EU/kg (weight), 10 EU/kg, 5 EU/kg, 1 EU/kg, or is below a predetermined threshold, e.g., the preparation comprises a level of endotoxin below a limit of detection by a specified method. In some embodiments, the reference criterion is a pharmaceutical release specification.

In some embodiments, the pharmaceutical composition, pharmaceutical drug substance or pharmaceutical drug product is a sterile drug product or or substantially free of microorganisms (e.g., supports growth of fewer than 100 viable microorganisms as tested under aseptic conditions). In some embodiments the pharmaceutical composition, pharmaceutical drug substance or pharmaceutical drug product meets the standard of USP <71> and/or the standard of USP <85>. In some embodiments, the pharmaceutical composition, pharmaceutical drug substance or pharmaceutical drug product is further labelled and shipped for pharmaceutical use. In some embodiments, the pharmaceutical composition, pharmaceutical drug substance or pharmaceutical drug product comprises a bioburden of less than 100 CFU/100 ml, 50 CFU/100 ml, 40 CFU/100 ml, 30 CFU/100 ml, 200 CFU/100 ml, 10 CFU/100 ml, or 10 CFU/100 ml before sterilization.

In some embodiments, the pharmaceutical composition, pharmaceutical drug substance or pharmaceutical drug product comprises a concentration of at least 0.1 ng/mL, 0.5 ng/mL, 1 ng/mL, 5 ng/mL, 10 ng/mL, 50 ng/mL, 0.1 μg/mL, 0.5 μg/mL, 1 pig/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 5 mg/mL, 10 mg/mL, 100 mg/mL, or 500 mg/mL circular polyribonucleotide molecules.

In some embodiments, the pharmaceutical compositions, pharmaceutical drug substance, or pharmaceutical drug product can be further purified using known techniques in the art for removing impurities, such as column chromatography or pH/vial inactivation.

### Circularization

In one embodiment, a linear circular polyribonucleotide may be cyclized, or concatemerized. In some embodiments, the linear circular polyribonucleotide may be cyclized in vitro prior to formulation and/or delivery. In some embodiments, the linear circular polyribonucleotide may be cyclized within a cell.

Extracellular Circularization

In some embodiments, the linear circular polyribonucleotide is cyclized, or concatemerized using a chemical method to form a circular polyribonucleotide. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In one embodiment, a DNA or RNA ligase may be used to enzymatically link a 5′-phosphorylated nucleic acid molecule (e.g., a linear circular polyribonucleotide) to the 3′-hydroxyl group of a nucleic acid (e.g., a linear nucleic acid) forming a new phosphodiester linkage. In an example reaction, a linear circular polyribonucleotide is incubated at 37° C. for 1 hour with 1 to 10 units of T4 RNA ligase (New England Biolabs, Ipswich, Mass.) according to the manufacturer's instructions. The ligation reaction may occur in the presence of a linear nucleic acid capable of base-pairing with both the 5′- and 3′-region in juxtaposition to assist the enzymatic ligation reaction. In one embodiment, the ligation is splint ligation. For example, a splint ligase, like RNA ligase 2, can be used for splint ligation. For splint ligation, a single stranded polynucleotide (splint), like a single stranded DNA, can be designed to hybridize with both termini of a linear polyribonucleotide, so that the two termini can be juxtaposed upon hybridization with the single-stranded splint. RNA ligase 2 can thus catalyze the ligation of the juxtaposed two termini of the linear polyribonucleotide, generating a covalently linked circular polyribonucleotide.

In one embodiment, a DNA or RNA ligase may be used in the synthesis of the circular polynucleotides. As a non-limiting example, the ligase may be a circ ligase or circular ligase.

In one embodiment, either the 5′- or 3′-end of the linear circular polyribonucleotide can encode a ligase ribozyme sequence such that during in vitro transcription, the resultant linear circular polyribonucleotide includes an active ribozyme sequence capable of ligating the 5′-end of the linear circular polyribonucleotide to the 3′-end of the linear circular polyribonucleotide. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In one embodiment, a linear circular polyribonucleotide may be cyclized or concatermerized by using at least one non-nucleic acid moiety. In one aspect, the at least one non-nucleic acid moiety may react with regions or features near the 5′ terminus and/or near the 3′ terminus of the linear circular polyribonucleotide in order to cyclize or concatermerize the linear circular polyribonucleotide. In another aspect, the at least one non-nucleic acid moiety may be located in or linked to or near the 5′ terminus and/or the 3′ terminus of the linear circular polyribonucleotide. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In one embodiment, a linear circular polyribonucleotide may be cyclized or concatermerized due to a non-nucleic acid moiety that causes an attraction between atoms, molecular surfaces at, near or linked to the 5′ and 3′ ends of the linear circular polyribonucleotide. As a non-limiting example, one or more linear circular polyribonucleotides may be cyclized or concatermized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In one embodiment, the linear circular polyribonucleotide may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′terminus may associate with each other causing a linear circular polyribonucleotide to cyclize or concatemerize. In another aspect, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in US patent application No. US20030082768, the contents of which is here in incorporated by reference in its entirety.

In some embodiments, the linear circular polyribonucleotide may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear circular polyribonucleotide into a 5′ monophosphate may occur by a two-step reaction comprising: (a) contacting the 5′ nucleotide of the linear circular polyribonucleotide with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

Splicing Element

In some embodiment, the circular polyribonucleotide includes at least one splicing element. In a circular polyribonucleotide as provided herein, a splicing element can be a complete splicing element that can mediate splicing of the circular polyribonucleotide. Alternatively, the splicing element can also be a residual splicing element from a completed splicing event. For instance, in some cases, a splicing element of a linear polyribonucleotide can mediate a splicing event that results in circularization of the linear polyribonucleotide, thereby the resultant circular polyribonucleotide comprises a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the circular polyribonucleotide includes a splicing element adjacent each expression sequence. In some embodiments, the splicing element is on one or both sides of each expression sequence, leading to separation of the expression products, e.g., peptide(s) and or polypeptide(s).

In some embodiments, the circular polyribonucleotide includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the circular polyribonucleotide includes at least one repetitive nucleotide sequence described elsewhere herein as an internal splicing element. In such embodiments, the repetitive nucleotide sequence may include repeated sequences from the Alu family of introns. In some embodiments, a splicing-related ribosome binding protein can regulate circular polyribonucleotide biogenesis (e.g. the Muscleblind and Quaking (QKI) splicing factors).

In some embodiments, the circular polyribonucleotide may include canonical splice sites that flank head-to-tail junctions of the circular polyribonucleotide.

In some embodiments, the circular polyribonucleotide may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of the 5′-OH group onto the 2′, 3′-cyclic phosphate of the same molecule forming a 3′, 5′-phosphodiester bridge.

In some embodiments, the circular polyribonucleotide may include a multimeric repeating RNA sequence that harbors a HPR element. The HPR comprises a 2′,3′-cyclic phosphate and a 5′-OH termini. The HPR element self-processes the 5′- and 3′-ends of the linear circular polyribonucleotide, thereby ligating the ends together.

In some embodiments, the circular polyribonucleotide may include a sequence that mediates self-ligation. In one embodiment, the circular polyribonucleotide may include a HDV sequence (e.g., HDV replication domain conserved sequence, GGCUCAUCUCGACAAGAGGCGGCAGUCCUCAGUACUCUUACUCUUUUCUGUAAAG AGGAGACUGCUGGACUCGCCGCCCAAGUUCGAGCAUGAGCC or GGCUAGAGGCGGCAGUCCUCAGUACUCUUACUCUUUUCUGUAAAGAGGAGACUG CUGGACUCGCCGCCCGAGCC) to self-ligate. In one embodiment, the circular polyribonucleotide may include loop E sequence (e.g., in PSTVd) to self-ligate. In another embodiment, the circular polyribonucleotide may include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene td, and the intervening sequence (IVS) rRNA of Tetrahymena.

Other Circularization Methods

In some embodiments, linear circular polyribonucleotides may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

### Expression Sequences

Peptides or Polypeptides

In some embodiments, the circular polyribonucleotide comprises at least one expression sequence that encodes a peptide or polypeptide. Such peptide may include, but is not limited to, small peptide, peptidomimetic (e.g., peptoid), amino acids, and amino acid analogs. The peptide may be linear or branched. Such peptide may have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such peptide may include, but is not limited to, a neurotransmitter, a hormone, a drug, a toxin, a viral or microbial particle, a synthetic molecule, and agonists or antagonists thereof.

The polypeptide may be linear or branched. The polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

Non-limiting examples of a peptide or polypeptide expressed by an expression sequence in the subject circular polyribonucleotide include those described in [0149], [0150], and [0152] of International Patent Publication No. WO2019118919A1, which is incorporated herein by reference in its entirety.

In some embodiments, the circular polyribonucleotide includes an expression sequence encoding a protein e.g., a therapeutic protein. In some embodiments, therapeutic proteins that can be expressed from the circular polyribonucleotide disclosed herein have antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity. Some examples of therapeutic proteins may include, but are not limited to, an enzyme replacement protein, a protein for supplementation, a protein vaccination, antigens (e.g., tumor antigens, viral, bacterial), hormones, cytokines, antibodies, immunotherapy (e.g., cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, and a CRISPR system or component thereof.

In some embodiments, exemplary proteins that can be expressed from the circular polyribonucleotide disclosed herein include an intracellular protein or cytosolic protein. In some embodiments, the circular polyribonucleotide expresses a reporter molecule, e.g., a NanoLuc® luciferase (nLuc). In some embodiments, exemplary proteins that can be expressed from the circular polyribonucleotide disclosed herein include a secretary protein, for instance, a secretary enzyme. In some cases, the circular polyribonucleotide expresses a secretary protein that can have a short half-life therapeutic in the blood, or can be a protein with a subcellular localization signal, or protein with secretory signal peptide. In some embodiments, the circular polyribonucleotide expresses a Gaussia Luciferase (GLuc). In some cases, the circular polyribonucleotide expresses a non-human protein, for instance, a fluorescent protein, an energy-transfer acceptor, or a protein-tag like Flag, Myc, or His. In some embodiments, exemplary proteins that can be expressed from the circular polyribonucleotide include a GFP. In some embodiments, the circular polyribonucleotide expresses tagged proteins, e.g., fusion proteins or engineered proteins containing a protein tag, e.g., chitin binding protein (CBP), maltose binding protein (MBP), Fc tag, glutathione-S-transferase (GST), SNAP-tag, tandem protein A (ZZ) tag, Halo-tag, AviTag (GLNDIFEAQKIEWHE), Calmodulin-tag (KRRWKKNFIAVSAANRFKKISSSGAL); polyglutamate tag (EEEEEE); E-tag (GAPVPYPDPLEPR); FLAG-tag (DYKDDDDK), HA-tag (YPYDVPDYA); His-tag (e.g., HHHHHH); Myc-tag (EQKLISEEDL); NE-tag (TKENPRSNQEESYDDNES); S-tag (KETAAAKFERQHMDS); SBP-tag (MDEKTTGWRGGHVVEGLAGELEQLRARLEHHPQGQREP); Softag 1 (SLAELLNAGLGGS); Softag 3 (TQDPSRVG); Spot-tag (PDRVRAVSHWSS); Strep-tag (Strep-tag II: WSHPQFEK); TC tag (CCPGCC); Ty tag (EVHTNQDPLD); V5 tag (GKPIPNPLLGLDST); VSV-tag (YTDIEMNRLGK); or Xpress tag (DLYDDDDK).

In some embodiments, the circular polyribonucleotide expresses an antigen binding protein, e.g., an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can comprise more than one expression sequence, each of which expresses a portion of an antibody, and the sum of which can constitute the antibody. In some cases, the circular polyribonucleotide comprises one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

Regulatory Elements

In some embodiments, the circular polyribonucleotide comprises a regulatory element, e.g., a sequence that modifies expression of an expression sequence within the circular polyribonucleotide.

A regulatory element may include a sequence that is located adjacent to an expression sequence that encodes an expression product. A regulatory element may be linked operatively to the adjacent sequence. A regulatory element may increase an amount of product expressed as compared to an amount of the expressed product when no regulatory element exists. In addition, one regulatory element can increase an amount of products expressed for multiple expression sequences attached in tandem. Hence, one regulatory element can enhance the expression of one or more expression sequences. Multiple regulatory element are well-known to persons of ordinary skill in the art.

A regulatory element as provided herein can include a selective translation sequence. As used herein, the term “selective translation sequence” can refer to a nucleic acid sequence that selectively initiates or activates translation of an expression sequence in the circular polyribonucleotide, for instance, certain riboswitch aptazymes. In some embodiments, the regulatory element is a translation modulator. A translation modulator can modulate translation of the expression sequence in the circular polyribonucleotide. A translation modulator can be a translation enhancer or suppressor. In some embodiments, a translation initiation sequence can function as a regulatory element. Nucleotides flanking a codon that initiates translation, such as, but not limited to, a start codon or an alternative start codon, are known to affect the translation efficiency, the length and/or the structure of the circular polyribonucleotide. (See e.g., Matsuda and Mauro PLoS ONE, 2010 5: 11; the contents of which are herein incorporated by reference in its entirety). Masking any of the nucleotides flanking a codon that initiates translation may be used to alter the position of translation initiation, translation efficiency, length and/or structure of the circular polyribonucleotide.

In one embodiment, a masking agent may be used near the start codon or alternative start codon in order to mask or hide the codon to reduce the probability of translation initiation at the masked start codon or alternative start codon. In another embodiment, a masking agent may be used to mask a start codon of the circular polyribonucleotide in order to increase the likelihood that translation will initiate at an alternative start codon.

A regulatory element as provided herein can include any of the regulatory elements that are described in [0156]-[0161] of International Patent Publication No. WO2019118919A1, which is incorporated herein by reference in its entirety.

Translation Initiation Sequence

In some embodiments, the circular polyribonucleotide encodes a polypeptide and may comprise a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide.

The circular polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, the circular polyribonucleotide may initiate at a codon which is not the first start codon, e.g., AUG. Translation of the circular polyribonucleotide may initiate at an alternative translation initiation sequence, such as those described in [0164] of International Patent Publication No. WO2019118919A1, which is incorporated herein by reference in its entirety.

In some embodiments, translation is initiated by eukaryotic initiation factor 4A (eIF4A) treatment with Rocaglates (translation is repressed by blocking 43S scanning, leading to premature, upstream translation initiation and reduced protein expression from transcripts bearing the RocA-eIF4A target sequence, see for example, www.nature.com/articles/nature17978).

IRES

In some embodiments, the circular polyribonucleotide described herein comprises an internal ribosome entry site (IRES) element. A suitable IRES element to include in a circular polyribonucleotide comprises an RNA sequence capable of engaging an eukaryotic ribosome, such as those described in [0166]-[0167] of International Patent Publication No. WO2019118919A1, which is incorporated herein by reference in its entirety.

In some embodiments, the circular polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the circular polyribonucleotide includes one or more IRES sequences on one or both sides of each expression sequence, leading to separation of the resulting peptide(s) and or polypeptide(s).

Termination Element

In some embodiments, the circular polyribonucleotide includes one or more expression sequences and each expression sequence may or may not have a termination element. In some embodiments, the circular polyribonucleotide includes one or more expression sequences and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide comprises a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences comprises two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell.

Stagger Element

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

In some embodiments, the circular polyribonucleotide includes a stagger element. To avoid production of a continuous expression product, e.g., peptide or polypeptide, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular polyribonucleotide. The stagger element may include, but is not limited to a 2A-like, or CHYSEL (cis-acting hydrolase element) sequence. In some embodiments, the stagger element encodes a sequence with a C-terminal consensus sequence that is X₁X₂X₃EX₅NPGP, where X₁ is absent or G or H, X₂ is absent or D or G, X₃ is D or V or I or S or M, and X₅ is any amino acid. In some embodiments, this sequence comprises a non-conserved sequence of amino-acids with a strong alpha-helical propensity followed by the consensus sequence −D(V/I)ExNPG P, where x=any amino acid. Some non-limiting examples of stagger elements includes GDVESNPGP, GDIEENPGP, VEPNPGP, IETNPGP, GDIESNPGP, GDVELNPGP, GDIETNPGP, GDVENPGP, GDVEENPGP, GDVEQNPGP, IESNPGP, GDIELNPGP, HDIETNPGP, HDVETNPGP, HDVEMNPGP, GDMESNPGP, GDVETNPGP, GDIEQNPGP, and DSEFNPGP.

In some embodiments, the stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element is present on one or both sides of each expression sequence, leading to translation of individual peptide(s) and or polypeptide(s) from each expression sequence.

In some embodiments, a stagger element comprises one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g. to a linking phosphate/to a phosphodiester linkage/to the phosphodiester backbone), and any combination thereof that can induce ribosomal pausing during translation. Some of the exemplary modifications provided herein are described elsewhere herein.

In some embodiments, the stagger element is present in the circular polyribonucleotide in other forms. For example, in some exemplary circular polyribonucleotides, a stagger element comprises a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence. In some embodiments, the first stagger element comprises a termination element and separates an expression product of the first expression sequence from an expression product of its succeeding expression sequences, thereby creating discrete expression products. In some cases, the circular polyribonucleotide comprising the first stagger element upstream of the first translation initiation sequence of the succeeding sequence in the circular polyribonucleotide is continuously translated, while a corresponding circular polyribonucleotide comprising a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element comprises a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some cases, the circular polyribonucleotide comprising the first stagger element upstream of the first translation initiation sequence of the first expression sequence in the circular polyribonucleotide is continuously translated, while a corresponding circular polyribonucleotide comprising a stagger element upstream of a second translation initiation sequence of a second expression sequence in the corresponding circular polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular polyribonucleotide than a distance between the first stagger element and the first translation initiation in the circular polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular polyribonucleotide comprises more than one expression sequence.

Regulatory Nucleic Acids

In some embodiments, the circular polyribonucleotide comprises one or more expression sequences that encode regulatory nucleic acid, e.g., that modifies expression of an endogenous gene and/or an exogenous gene. In some embodiments, the expression sequence of a circular polyribonucleotide as provided herein can comprise a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

In one embodiment, the regulatory nucleic acid targets a gene such as a host gene. The regulatory nucleic acids may include any of the regulatory nucleic acids described in [0177] and [0181]-[0189] of International Patent Publication No. WO2019118919A1, which is incorporated herein by reference in its entirety.

In some embodiments, the circular polyribonucleotide comprises a guide RNA (gRNA). In some embodiments, the circular polyribonucleotide comprises a guide RNA or encodes the guide RNA. A gRNA short synthetic RNA composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The gRNA may recognize specific DNA sequences (e.g., sequences adjacent to or within a promoter, enhancer, silencer, or repressor of a gene).

In one embodiment, the gRNA is used as part of a CRISPR system for gene editing. For the purposes of gene editing, the circular polyribonucleotide may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur; for Cpf1 at least about 16 nucleotides of gRNA sequence is needed to achieve detectable DNA cleavage.

The circular polyribonucleotide may modulate expression of RNA encoded by a gene. Because multiple genes can share some degree of sequence homology with each other, in some embodiments, the circular polyribonucleotide can be designed to target a class of genes with sufficient sequence homology. In some embodiments, the circular polyribonucleotide can contain a sequence that has complementarity to sequences that are shared amongst different gene targets or are unique for a specific gene target. In some embodiments, the circular polyribonucleotide can be designed to target conserved regions of an RNA sequence having homology between several genes thereby targeting several genes in a gene family (e.g., different gene isoforms, splice variants, mutant genes, etc.). In some embodiments, the circular polyribonucleotide can be designed to target a sequence that is unique to a specific RNA sequence of a single gene.

In some embodiments, the expression sequence has a length less than 5000 bps (e.g., less than about 5000 bps, 4000 bps, 3000 bps, 2000 bps, 1000 bps, 900 bps, 800 bps, 700 bps, 600 bps, 500 bps, 400 bps, 300 bps, 200 bps, 100 bps, 50 bps, 40 bps, 30 bps, 20 bps, 10 bps, or less). In some embodiments, the expression sequence has, independently or in addition to, a length greater than 10 bps (e.g., at least about 10 bps, 20 bps, 30 bps, 40 bps, 50 bps, 60 bps, 70 bps, 80 bps, 90 bps, 100 bps, 200 bps, 300 bps, 400 bps, 500 bps, 600 bps, 700 bps, 800 bps, 900 bps, 1000 kb, 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, 10 kb, 20 kb or greater).

In some embodiments, the expression sequence comprises one or more of the features described herein, e.g., a sequence encoding one or more peptides or proteins, one or more regulatory element, one or more regulatory nucleic acids, e.g., one or more non-coding RNAs, other expression sequences, and any combination thereof.

### Translation Efficiency

In some embodiments, the translation efficiency of a circular polyribonucleotide as provided herein is greater than a reference, e.g., a linear counterpart, a linear expression sequence, or a linear circular polyribonucleotide. In some embodiments, a circular polyribonucleotide as provided herein has the translation efficiency that is at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 600%, 70%, 800%, 900%, 1000%, 2000%, 5000%, 10000%, 100000%, or more greater than that of a reference. In some embodiments, a circular polyribonucleotide has a translation efficiency 10% greater than that of a linear counterpart. In some embodiments, a circular polyribonucleotide has a translation efficiency 300% greater than that of a linear counterpart.

In some embodiments, the circular polyribonucleotide produces stoichiometric ratios of expression products. Rolling circle translation continuously produces expression products at substantially equivalent ratios. In some embodiments, the circular polyribonucleotide has a stoichiometric translation efficiency, such that expression products are produced at substantially equivalent ratios. In some embodiments, the circular polyribonucleotide has a stoichiometric translation efficiency of multiple expression products, e.g., products from 2, 3, 4, 5, 6, 7, 8, 9, 10, 11, 12, or more expression sequences.

Rolling Circle Translation

In some embodiments, once translation of the circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 10⁵ rounds, or at least 10⁶ rounds of translation of the circular polyribonucleotide.

In some embodiments, the rolling circle translation of the circular polyribonucleotide leads to generation of polypeptide product that is translated from more than one round of translation of the circular polyribonucleotide (“continuous” expression product). In some embodiments, the circular polyribonucleotide comprises a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the amount ratio of the discrete products over the total polypeptides is tested in an in vitro translation system. In some embodiments, the in vitro translation system used for the test of amount ratio comprises rabbit reticulocyte lysate. In some embodiments, the amount ratio is tested in an in vivo translation system, such as a eukaryotic cell or a prokaryotic cell, a cultured cell or a cell in an organism.

### Untranslated Regions

In some embodiments, the circular polyribonucleotide comprises untranslated regions (UTRs). UTRs of a genomic region comprising a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full length human intron, e.g., ZKSCAN1.

In some embodiments, the circular polyribonucleotide comprises a UTR with one or more stretches of Adenosines and Uridines embedded within. These AU rich signatures are may increase turnover rates of the expression product.

Introduction, removal or modification of UTR AU rich elements (AREs) may be useful to modulate the stability, or immunogenicity (e.g., the level of one or more marker of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide. Exemplary UTRs that can be used in a circular polyribonucleotide provided herein include those described in [0200]-[0201] of International Patent Publication No. WO2019118919A1, which is incorporated herein by reference in its entirety.

PolyA Sequence

In some embodiments, the circular polyribonucleotide may include a poly-A sequence. In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019118919A1, which is incorporated herein by reference in its entirety.

In some embodiments, the circular polyribonucleotide comprises a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, expression efficiency, etc.

RNA-Binding

In some embodiments, the circular polyribonucleotide comprises one or more RNA binding sites. microRNAs (or miRNA) are short noncoding RNAs that bind to the 3′UTR of nucleic acid molecules and down-regulate gene expression either by reducing nucleic acid molecule stability or by inhibiting translation. The circular polyribonucleotide may comprise one or more microRNA target sequences, microRNA sequences, or microRNA seeds. Such sequences may correspond to any known microRNA, such as those taught in US Publication US2005/0261218, US Publication US2005/0059005, and [0207]-[0215] of International Patent Publication No. WO2019118919A1, the contents of which are incorporated herein by reference in their entirety.

Protein-Binding

In some embodiments, the circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host's immune system, have modulated degradation, or modulated translation, by masking the circular polyribonucleotide from components of the host's immune system.

In some embodiments, the circular polyribonucleotide comprises at least one immunoprotein binding site, for example to evade immune responses, e.g., CTL (cytotoxic T lymphocyte) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present invention, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences comprising a ribosome binding site, e.g., an initiation codon.

Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

In some embodiments, the circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

Encryptogen

As described herein, the circular polyribonucleotide comprises an encryptogen to reduce, evade or avoid the innate immune response of a cell. In one aspect, provided herein are circular polyribonucleotide which when delivered to cells, results in a reduced immune response from the host as compared to the response triggered by a reference compound, e.g. a linear polynucleotide corresponding to the described circular polyribonucleotide or a circular polyribonucleotide lacking an encryptogen. In some embodiments, the circular polyribonucleotide has less immunogenicity (e.g., a lower level of one or more marker of an immune or inflammatory response) than a counterpart lacking an encryptogen.

In some embodiments, an encryptogen enhances stability. There is growing body of evidence about the regulatory roles played by the UTRs in terms of stability of a nucleic acid molecule and translation. The regulatory features of a UTR may be included in the encryptogen to enhance the stability of the circular polyribonucleotide.

In some embodiments, 5′ or 3′UTRs can constitute encryptogens in a circular polyribonucleotide. For example, removal or modification of UTR AU rich elements (AREs) may be useful to modulate the stability or immunogenicity (e.g., the modulate the level of one or more marker of an immune or inflammatory response) of the circular polyribonucleotide.

In some embodiments, removal of modification of AU rich elements (AREs) in expression sequence, e.g., translatable regions, can be useful to modulate the stability or immunogenicity (e.g., modulate the level of one or more marker of an immune or inflammatory response) of the circular polyribonucleotide

In some embodiments, an encryptogen comprises miRNA binding site or binding site to any other non-coding RNAs. For example, incorporation of miR-142 sites into the circular polyribonucleotide described herein may not only modulate expression in hematopoietic cells, but also reduce or abolish immune responses to a protein encoded in the circular polyribonucleotide.

In some embodiments, an encryptogen comprises one or more protein binding sites that enable a protein, e.g., an immunoprotein, to bind to the RNA sequence. By engineering protein binding sites into the circular polyribonucleotide, the circular polyribonucleotide may evade or have reduced detection by the host's immune system, have modulated degradation, or modulated translation, by masking the circular polyribonucleotide from components of the host's immune system. In some embodiments, the circular polyribonucleotide comprises at least one immunoprotein binding site, for example to evade immune responses, e.g., CTL responses. In some embodiments, the immunoprotein binding site is a nucleotide sequence that binds to an immunoprotein and aids in masking the circular polyribonucleotide as exogenous.

In some embodiments, an encryptogen comprises one or more modified nucleotides. Exemplary modifications can include any modification to the sugar, the nucleobase, the internucleoside linkage (e.g. to a linking phosphate/to a phosphodiester linkage/to the phosphodiester backbone), and any combination thereof that can prevent or reduce immune response against the circular polyribonucleotide. Some of the exemplary modifications provided herein are described in details below.

In some embodiments, the circular polyribonucleotide includes one or more modifications as described elsewhere herein to reduce an immune response from the host as compared to the response triggered by a reference compound, e.g. a circular polyribonucleotide lacking the modifications. In particular, the addition of one or more inosine has been shown to discriminate RNA as endogenous versus viral. See for example, Yu, Z. et al. (2015) RNA editing by ADAR1 marks dsRNA as “self”. Cell Res. 25, 1283-1284, which is incorporated by reference in its entirety.

In some embodiments, the circular polyribonucleotide includes one or more expression sequences for shRNA or an RNA sequence that can be processed into siRNA, and the shRNA or siRNA targets RIG-I and reduces expression of RIG-I. RIG-I can sense foreign circular RNA and leads to degradation of foreign circular RNA. Therefore, a circular polynucleotide harboring sequences for RIG-1-targeting shRNA, siRNA or any other regulatory nucleic acids can reduce immunity, e.g., host cell immunity, against the circular polyribonucleotide.

In some embodiments, the circular polyribonucleotide lacks a sequence, element, or structure, that aids the circular polyribonucleotide in reducing, evading, or avoiding an innate immune response of a cell. In some such embodiments, the circular polyribonucleotide may lack a polyA sequence, a 5′ end, a 3′ end, phosphate group, hydroxyl group, or any combination thereof.

Riboswitches

In some embodiments, the circular polyribonucleotide comprises one or more riboswitches.

A riboswitch is typically considered a part of the circular polyribonucleotide that can directly bind a small target molecule, and whose binding of the target affects RNA translation, the expression product stability and activity (Tucker B J, Breaker R R (2005), Curr Opin Struct Biol 15 (3): 342-8). Thus, the circular polyribonucleotide that includes a riboswitch is directly involved in regulating its own activity, depending on the presence or absence of its target molecule. In some embodiments, a riboswitch has a region of aptamer-like affinity for a separate molecule. Thus, in the broader context of the instant invention, any aptamer included within a non-coding nucleic acid could be used for sequestration of molecules from bulk volumes. Downstream reporting of the event via “(ribo)switch” activity may be especially advantageous.

In some embodiments, the riboswitch may have an effect on gene expression including, but not limited to, transcriptional termination, inhibition of translation initiation, mRNA self-cleavage, and in eukaryotes, alteration of splicing pathways. The riboswitch may function to control gene expression through the binding or removal of a trigger molecule. Thus, subjecting a circular polyribonucleotide that includes the riboswitch to conditions that activate, deactivate or block the riboswitch to alter expression. Expression can be altered as a result of, for example, termination of transcription or blocking of ribosome binding to the RNA. Binding of a trigger molecule or an analog thereof can, depending on the nature of the riboswitch, reduce or prevent expression of the RNA molecule or promote or increase expression of the RNA molecule. Some examples of riboswitches are described herein.

In some embodiments, the riboswitch is a cyclic di-GMP riboswitches, a FMN riboswitch (also RFN-element), a glmS riboswitch, a Glutamine riboswitches, a Glycine riboswitch, a Lysine riboswitch (also L-box), a PreQ1 riboswitch (e.g., PreQ1-l riboswitches and PreQ1-ll riboswitches), a Purine riboswitch, a SAH riboswitch, a SAM riboswitch, a SAM-SAH riboswitch, a Tetrahydrofolate riboswitch, a theophylline binding riboswitch, a thymine pyrophosphate binding riboswitch, a T. tengcongensis glmS catalytic riboswitch, a TPP riboswitch (also THI-box), a Moco riboswitch, or a Adenine sensing add-A riboswitch, each of which is described in [0235]-[0252] of International Patent Publication No. WO2019118919A1, which is incorporated herein by reference in its entirety.

Aptazyme

In some embodiments, the circular polyribonucleotide comprises an aptazyme. Aptazyme is a switch for conditional expression in which an aptamer region is used as an allosteric control element and coupled to a region of catalytic RNA (a “ribozyme” as described below). In some embodiments, the aptazyme is active in cell type specific translation. In some embodiments, the aptazyme is active under cell state specific translation, e.g., virally infected cells or in the presence of viral nucleic acids or viral proteins.

A ribozyme (from ribonucleic acid enzyme, also called RNA enzyme or catalytic RNA) is a RNA molecule that catalyzes a chemical reaction. Some non-limiting examples of ribozymes include hammerhead ribozyme, VL ribozyme, leadzyme, hairpin ribozyme, and other ribozymes described in [0254]-[0259] of International Patent Publication No. WO2019118919A1, which is incorporated herein by reference in its entirety.

### Replication Element

The circular polyribonucleotide may encode a sequence and/or motifs useful for replication. Replication of a circular polyribonucleotide may occur by generating a complement circular polyribonucleotide. In some embodiments, the circular polyribonucleotide includes a motif to initiate transcription, where transcription is driven by either endogenous cellular machinery (DNA-dependent RNA polymerase) or an RNA-depended RNA polymerase encoded by the circular polyribonucleotide. The product of rolling-circle transcriptional event may be cut by a ribozyme to generate either complementary or propagated circular polyribonucleotide at unit length. The ribozymes may be encoded by the circular polyribonucleotide, its complement, or by an RNA sequence in trans. In some embodiments, the encoded ribozymes may include a sequence or motif that regulates (inhibits or promotes) activity of the ribozyme to control circular RNA propagation. In some embodiments, unit-length sequences may be ligated into a circular form by a cellular RNA ligase. In some embodiments, the circular polyribonucleotide includes a replication element that aids in self amplification. Examples of such replication elements include those described in [0280]-[0282] of International Patent Publication No. WO2019118919A1, which is incorporated herein by reference in its entirety.

In some embodiments, the circular polyribonucleotide is substantially resistant to degradation, e.g., by exonucleases.

In some embodiments, the circular polyribonucleotide replicates within a cell. In some embodiments, the circular polyribonucleotide replicates within in a cell at a rate of between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween. In some embodiments, the circular polyribonucleotide is replicated within a cell and is passed to daughter cells. In some embodiments, a cell passes at least one circular polyribonucleotide to daughter cells with an efficiency of at least 25%, 50%, 60%, 70%, 80%, 85%, 90%, 95%, or 99%. In some embodiments, cell undergoing meiosis passes the circular polyribonucleotide to daughter cells with an efficiency of at least 25%, 50%, 60%, 70%, 80%, 85%, 90%, 95%, or 99%. In some embodiments, a cell undergoing mitosis passes the circular polyribonucleotide to daughter cells with an efficiency of at least 25%, 50%, 60%, 70%, 80%, 85%, 90%, 95%, or 99%.

In some embodiments, the circular polyribonucleotide replicates within the host cell. In one embodiment, the circular polyribonucleotide is capable of replicating in a mammalian cell, e.g., human cell.

While in some embodiments the circular polyribonucleotide replicates in the host cell, the circular polyribonucleotide does not integrate into the genome of the host, e.g., with the host's chromosomes. In some embodiments, the circular polyribonucleotide has a negligible recombination frequency, e.g., with the host's chromosomes. In some embodiments, the circular polyribonucleotide has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### Scaffold Sequences

In some embodiments, the circular polyribonucleotide molecules comprise one or more scaffold sequences. A scaffold sequence can be an aptamer sequence. In some embodiments of each aspect recited above, the circular polyribonucleotide molecules have a sequence encoding an endogenous or naturally occurring circular polyribonucleotide sequence.

In some embodiments, circRNA binds one or more targets. In some embodiments, a circRNA is a circular aptamer. In one embodiment, a circRNA comprises one or more binding sites that bind to one or more targets. In one embodiment, the circ RNA comprises an aptamer sequence. In one embodiment, circRNA binds both a DNA target and a protein target and e.g., mediates transcription. In another embodiment, circRNA brings together a protein complex and e.g., mediates post-translational modifications or signal transduction. In another embodiment, circRNA binds two or more different targets, such as proteins, and e.g., shuttles these proteins to the cytoplasm, or mediates degradation of one or more of the targets.

In some embodiments, circRNA binds at least one of DNA, RNA, and proteins and thereby regulates cellular processes (e.g., alter protein expression, modulate gene expression, modulate cell signaling, etc.). In some embodiments, synthetic circRNA includes binding sites for interaction with a target or at least one moiety, e.g., a binding moiety, of DNA, RNA or proteins of choice to thereby compete in binding with the endogenous counterpart.

In some embodiments, the circular RNA forms a complex that regulates the cellular process (e.g., alter protein expression, modulate gene expression, modulate cell signaling, etc.). In some embodiments, the circular RNA sensitizes a cell to a cytotoxic agent (e.g., a chemotherapeutic agent) by binding to a target (e.g., a transcription factor), which results in reduce cell viability. For example, sensitizing the cell to the cytoxic agent results in decreased cell viability after the delivery of the cytotoxic agent and the circular RNA. In some embodiments, the decreased cell viability is decreased by at least 10%, 20%, 30%, 40%, 50%, 60%, 70%, 80%, or 90%, or any percentage therein.

In some embodiments, the complex is detectable for at least 5 days after delivery of the circular RNA to cell. In some embodiments, the complex is detectable for at 6 days, 7 days, 8 days, 9 days, 10 days, 11 days, 12 days, 13 days, 14 days, 15 days, or 16 days after delivery of the circular RNA to the cell.

In one embodiment, synthetic circRNA binds and/or sequesters miRNAs. In another embodiment, synthetic circRNA binds and/or sequesters proteins. In another embodiment, synthetic circRNA binds and/or sequesters mRNA. In another embodiment, synthetic circRNA binds and/or sequesters ribosomes. In another embodiment, synthetic circRNA binds and/or sequesters circRNA. In another embodiment, synthetic circRNA binds and/or sequesters long-noncoding RNA (lncRNA) or any other non-coding RNA, e.g., miRNA, tRNA, rRNA, snoRNA, ncRNA, siRNA, long-noncoding RNA, shRNA. Besides binding and/or sequestration sites, the circRNA may include a degradation element, which will result in degradation of the bound and/or sequestered RNA and/or protein.

In one embodiment, a circRNA comprises a lncRNA or a sequence of a lncRNA, e.g., a circRNA comprises a sequence of a naturally occurring, non-circular lncRNA or a fragment thereof. In one embodiment, a lncRNA or a sequence of a lncRNA is circularized, with or without a spacer sequence, to form a synthetic circRNA.

In one embodiment, a circRNA has ribozyme activity. In one embodiment, a circRNA can be used to act as a ribozyme and cleave pathogenic or endogenous RNA, DNA, small molecules or protein. In one embodiment, a circRNA has enzymatic activity. In one embodiment, synthetic circRNA is able to specifically recognize and cleave RNA (e.g., viral RNA). In another embodiment circRNA is able to specifically recognize and cleave proteins. In another embodiment circRNA is able to specifically recognize and degrade small molecules.

In one embodiment, a circRNA is an immolating or self-cleaving or cleavable circRNA. In one embodiment, a circRNA can be used to deliver RNA, e.g., miRNA, tRNA, rRNA, snoRNA, ncRNA, siRNA, long-noncoding RNA, shRNA. In one embodiment, synthetic circRNA is made up of microRNAs separated by (1) self-cleavable elements (e.g., hammerhead, splicing element), (2) cleavage recruitment sites (e.g., ADAR), (3) a degradable linker (e.g., glycerol), (4) a chemical linker, and/or (5) a spacer sequence. In another embodiment, synthetic circRNA is made up of siRNAs separated by (1) self-cleavable elements (e.g., hammerhead, splicing element), (2) cleavage recruitment sites (e.g., ADAR), (3) a degradable linker (e.g., glycerol), (4), chemical linker, and/or (5) a spacer sequence.

In one embodiment, a circRNA is a transcriptionally/replication competent circRNA. This circRNA can encode any type of RNA. In one embodiment, a synthetic circRNA has an anti-sense miRNA and a transcriptional element. In one embodiment, after transcription, linear functional miRNAs are generated from a circRNA. In one embodiment, a circRNA is a translation incompetent circular polyribonucleotide.

In one embodiment, a circRNA has one or more of the above attributes in combination with a translating element.

### Other Sequences

In some embodiments, the circular polyribonucleotide further includes another nucleic acid sequence. In some embodiments, the circular polyribonucleotide may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the circular polyribonucleotide includes an siRNA to target a different loci of the same gene expression product as the circular polyribonucleotide. In one embodiment, the circular polyribonucleotide includes an siRNA to target a different gene expression product as the circular polyribonucleotide.

In some embodiments, the circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent that is comparable to or similar to in the absence of exonuclease. In some embodiments, the circular polyribonucleotide lacks degradation by exonucleases. In some embodiments, the circular polyribonucleotide has reduced degradation when exposed to exonuclease. In some embodiments, the circular polyribonucleotide lacks binding to a cap-binding protein In some embodiments, the circular polyribonucleotide lacks a 5′ cap.

In some embodiments, the circular polyribonucleotide lacks a 5′-UTR and is competent for protein express from its one or more expression sequences. In some embodiments, the circular polyribonucleotide lacks a 3′-UTR and is competent for protein express from its one or more expression sequences. In some embodiments, the circular polyribonucleotide lacks a poly-A sequence and is competent for protein express from its one or more expression sequences. In some embodiments, the circular polyribonucleotide lacks a termination element and is competent for protein express from its one or more expression sequences. In some embodiments, the circular polyribonucleotide lacks an internal ribosomal entry site and is competent for protein express from its one or more expression sequences. In some embodiments, the circular polyribonucleotide lacks a cap and is competent for protein express from its one or more expression sequences. In some embodiments, the circular polyribonucleotide lacks a 5′-UTR, a 3′-UTR, and an IRES, and is competent for protein express from its one or more expression sequences. In some embodiments, the circular polyribonucleotide comprises one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (siRNA, lncRNAs, shRNA), and a sequence that encodes a therapeutic mRNA or protein.

The other sequence may have a length from about 2 to about 10000 nts, about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

As a result of its circularization, the circular polyribonucleotide may include certain characteristics that distinguish it from linear RNA. For example, the circular polyribonucleotide is less susceptible to degradation by exonuclease as compared to linear RNA. As such, the circular polyribonucleotide is more stable than a linear RNA, especially when incubated in the presence of an exonuclease. The increased stability of the circular polyribonucleotide compared with linear RNA makes circular polyribonucleotide more useful as a cell transforming reagent to produce polypeptides and can be stored more easily and for longer than linear RNA. The stability of the circular polyribonucleotide treated with exonuclease can be tested using methods standard in art which determine whether RNA degradation has occurred (e.g., by gel electrophoresis).

Moreover, unlike linear RNA, the circular polyribonucleotide is less susceptible to dephosphorylation when the circular polyribonucleotide is incubated with phosphatase, such as calf intestine phosphatase.

Nucleotide Spacer Sequences

In some embodiments, the circular polyribonucleotide comprises a spacer sequence.

In some embodiments, the circular polyribonucleotide comprises at least one spacer sequence. In some embodiments, the circular polyribonucleotide comprises 1, 2, 3, 4, 5, 6, 7 or more spacer sequences.

In some embodiments, the circular polyribonucleotide comprises one or more spacer sequence configured according to descriptions in [0295]-[0302] of International Patent Publication No. WO2019118919A1, which is incorporated herein by reference in its entirety.

Non-Nucleic Acid Linkers

The circular polyribonucleotide described herein may also comprise a non-nucleic acid linker. In some embodiments, the circular polyribonucleotide described herein has a non-nucleic acid linker between one or more of the sequences or elements described herein. In one embodiment, one or more sequences or elements described herein are linked with the linker. The non-nucleic acid linker may be a chemical bond, e.g., one or more covalent bonds or non-covalent bonds. In some embodiments, the non-nucleic acid linker is a peptide or protein linker. Such a linker may be between 2-30 amino acids, or longer. The linker includes flexible, rigid or cleavable linkers, such as those described in [0304]-[0307] of International Patent Publication No. WO2019118919A1, which is incorporated herein by reference in its entirety.

### Stability/Half-Life

In some embodiments, a circular polyribonucleotide preparation provided herein has an increased half-life over a reference, e.g., a linear polyribonucleotide having the same nucleotide sequence but is not circularized (e.g., linear counterpart). In some embodiments, the circular polyribonucleotide is resistant to degradation, e.g., exonuclease. In some embodiments, the circular polyribonucleotide is resistant to self-degradation. In some embodiments, the circular polyribonucleotide lacks an enzymatic cleavage site, e.g., a dicer cleavage site. In some embodiments, the circular polyribonucleotide has a half-life at least about 5%, at least about 10%, at least about 20%, at least about 30%, at least about 40%, at least about 50%, at least about 60%, at least about 70%, at least about 80%, at least about 90%, at least about 100%, at least about 120%, at least about 140%, at least about 150%, at least about 160%, at least about 180%, at least about 200%, at least about 300%, at least about 400%, at least about 500%, at least about 600%, at least about 700% at least about 800%, at least about 900%, at least about 1000% or at least about 10000%, longer than a reference, e.g., a linear counterpart.

In some embodiments, the circular polyribonucleotide persists in a cell during cell division. In some embodiments, the circular polyribonucleotide persists in daughter cells after mitosis. In some embodiments, the circular polyribonucleotide is replicated within a cell and is passed to daughter cells. In some embodiments, the circular polyribonucleotide comprises a replication element that mediates self-replication of the circular polyribonucleotide. In some embodiments, the replication element mediates transcription of the circular polyribonucleotide into a linear polyribonucleotide that is complementary to the circular polyribonucleotide (linear complementary). In some embodiments, the linear complementary polyribonucleotide can be circularized in vivo in cells into a complementary circular polyribonucleotide. In some embodiments, the complementary polyribonucleotide can further self-replicate into another circular polyribonucleotide, which has the same or similar nucleotide sequence as the starting circular polyribonucleotide. One exemplary self-replication element includes HDV replication domain (as described by Beeharry et al, Virol, 2014, 450-451:165-173). In some embodiments, a cell passes at least one circular polyribonucleotide to daughter cells with an efficiency of at least 25%, 50%, 60%, 70%, 80%, 85%, 90%, 95%, or 99%. In some embodiments, cell undergoing meiosis passes the circular polyribonucleotide to daughter cells with an efficiency of at least 25%, 50%, 60%, 70%, 80%, 85%, 90%, 95%, or 99%. In some embodiments, a cell undergoing mitosis passes the circular polyribonucleotide to daughter cells with an efficiency of at least 25%, 50%, 60%, 70%, 80%, 85%, 90%, 95%, or 99%.

### Modifications

The circular polyribonucleotide may include one or more substitutions, insertions and/or additions, deletions, and covalent modifications with respect to reference sequences, in particular, the parent polyribonucleotide, are included within the scope of this invention.

In some embodiments, the circular polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197). In some embodiments, the first isolated nucleic acid comprises messenger RNA (mRNA). In some embodiments, the mRNA comprises at least one nucleoside selected from the group such as those described in [0311] of International Patent Publication No. WO2019118919A1, which is incorporated herein by reference in its entirety.

The circular polyribonucleotide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the circular polyribonucleotide includes at least one N(6)methyladenosine (m6A) modification to increase translation efficiency. In some embodiments, the N(6)methyladenosine (m6A) modification can reduce immunogenicity (e.g., reduce the level of one or more marker of an immune or inflammatory response) of the circular polyribonucleotide.

In some embodiments, the modification may include a chemical or cellular induced modification. For example, some non-limiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the ribonucleotides of the circular polyribonucleotide may enhance immune evasion. The circular polyribonucleotide may be synthesized and/or modified by methods well established in the art, such as those described in “Current protocols in nucleic acid chemistry,” Beaucage, S. L. et al. (Eds.), John Wiley & Sons, Inc., New York, N.Y., USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the circular polyribonucleotide. In some embodiments, the modification includes a bi-orthogonal nucleotides, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more ribonucleotides of the circular polyribonucleotide may, as well as backbone modifications, include modification or replacement of the phosphodiester linkages. Specific examples of circular polyribonucleotide include, but are not limited to circular polyribonucleotide including modified backbones or no natural internucleoside linkages such as internucleoside modifications, including modification or replacement of the phosphodiester linkages. Circular polyribonucleotides having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the circular polyribonucleotide will include ribonucleotides with a phosphorus atom in its internucleoside backbone.

Modified circular polyribonucleotide backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the circular polyribonucleotide may be negatively or positively charged.

The modified nucleotides, which may be incorporated into the circular polyribonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the circular polyribonucleotide is expected to reduce the innate immune response through weaker binding/activation of cellular innate immune molecules.

In specific embodiments, a modified nucleoside includes an alpha-thio-nucleoside (e.g., 5′-0-(l-thiophosphate)-adenosine, 5′-0-(l-thiophosphate)-cytidine (a-thio-cytidine), 5′-0-(l-thiophosphate)-guanosine, 5′-0-(l-thiophosphate)-uridine, or 5′-0-(1-thiophosphate)-pseudouridine).

Other internucleoside linkages that may be employed according to the present invention, including internucleoside linkages which do not contain a phosphorous atom, are described herein.

In some embodiments, the circular polyribonucleotide may include one or more cytotoxic nucleosides. For example, cytotoxic nucleosides may be incorporated into circular polyribonucleotide, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, l-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-l-(tetrahydrofuran-2-yl)pyrimidine-2,4(lH,3H)-dione), troxacitabine, tezacitabine, 2′-deoxy-2′-methylidenecytidine (DMDC), and 6-mercaptopurine. Additional examples include fludarabine phosphate, N4-behenoyl-l-beta-D-arabinofuranosylcytosine, N4-octadecyl-1-beta-D-arabinofuranosylcytosine, N4-palmitoyl-l-(2-C-cyano-2-deoxy-beta-D-arabino-pentofuranosyl) cytosine, and P-4055 (cytarabine 5′-elaidic acid ester).

The circular polyribonucleotide may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the circular polyribonucleotide, or in a given predetermined sequence region thereof. In some embodiments, the circular polyribonucleotide includes a pseudouridine. In some embodiments, the circular polyribonucleotide includes an inosine, which may aid in the immune system characterizing the circular polyribonucleotide as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, all nucleotides in the circular polyribonucleotide (or in a given sequence region thereof) are modified. In some embodiments, the modification may include an m6A, which may augment expression; an inosine, which may attenuate an immune response; pseudouridine, which may increase RNA stability, or translational readthrough (stagger element), an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in the circular polyribonucleotide. One of ordinary skill in the art will appreciate that the nucleotide analogs or other modification(s) may be located at any position(s) of the circular polyribonucleotide, such that the function of the circular polyribonucleotide is not substantially decreased. A modification may also be a non-coding region modification. The circular polyribonucleotide may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

### Structure

In some embodiments, the circular polyribonucleotide comprises a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure. In some embodiments, a segment having a quasi-double-stranded secondary structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides. In some embodiments, the circular polyribonucleotide has one or more segments (e.g., 2, 3, 4, 5, 6, or more) having a quasi-double-stranded secondary structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides.

In some embodiments, one or more sequences of the circular polyribonucleotide include substantially single stranded vs double stranded regions. In some embodiments, the ratio of single stranded to double stranded may influence the functionality of the circular polyribonucleotide.

In some embodiments, one or more sequences of the circular polyribonucleotide that are substantially single stranded. In some embodiments, one or more sequences of the circular polyribonucleotide that are substantially single stranded may include a protein- or RNA-binding site. In some embodiments, the circular polyribonucleotide sequences that are substantially single stranded may be conformationally flexible to allow for increased interactions. In some embodiments, the sequence of the circular polyribonucleotide is purposefully engineered to include such secondary structures to bind or increase protein or nucleic acid binding.

In some embodiments, the circular polyribonucleotide sequences that are substantially double stranded. In some embodiments, one or more sequences of the circular polyribonucleotide that are substantially double stranded may include a conformational recognition site, e.g., a riboswitch or aptazyme. In some embodiments, the circular polyribonucleotide sequences that are substantially double stranded may be conformationally rigid. In some such instances, the conformationally rigid sequence may sterically hinder the circular polyribonucleotide from binding a protein or a nucleic acid. In some embodiments, the sequence of the circular polyribonucleotide is purposefully engineered to include such secondary structures to avoid or reduce protein or nucleic acid binding.

There are 16 possible base-pairings, however of these, six (AU, GU, GC, UA, UG, CG) may form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, a segment having a quasi-helical structure has at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the circular polyribonucleotide has one or more segments (e.g., 2, 3, 4, 5, 6, or more) having a quasi-helical structure. In some embodiments, the segments are separated by 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides. In some embodiments, the circular polyribonucleotide includes at least one of a U-rich or A-rich sequence or a combination thereof. In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure. In some embodiments, the circular polyribonucleotide has an intramolecular triple quasi-helix structure that aids in stabilization.

In some embodiments, the circular polyribonucleotide has at least one binding site, e.g., at least one protein binding site, at least one miRNA binding site, at least one lncRNA binding site, at least one tRNA binding site, at least one rRNA binding site, at least one snRNA binding site, at least one siRNA binding site, at least one piRNA binding site, at least one snoRNA binding site, at least one snRNA binding site, at least one exRNA binding site, at least one scaRNA binding site, at least one Y RNA binding site, at least one hnRNA binding site, and/or at least one tRNA motif.

In some embodiments, the circular polyribonucleotide is configured to comprise a higher order structure, such as those described in International Patent Publication No. WO2019118919A1, which is incorporated herein by reference in its entirety.

### Delivery

The circular polyribonucleotide described herein may also be included in pharmaceutical compositions with a carrier or without a carrier.

Pharmaceutical compositions described herein may be formulated for example including a carrier, such as a pharmaceutical carrier and/or a polymeric carrier, e.g., a liposome, and delivered by known methods to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof. Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

In some embodiments, the circular polyribonucleotides may be delivered in a naked delivery formulation. A naked delivery formulation delivers a circular polyribonucleotide to a cell without the aid of a carrier and without covalent modification of the circular polyribonucleotide or partial or complete encapsulation of the circular polyribonucleotide.

A naked delivery formulation is a formulation that is free from a carrier and wherein the circular polyribonucleotide is without a covalent modification that binds a moiety that aids in delivery to a cell and the circular polyribonucleotide is not partially or completely encapsulated. In some embodiments, an circular polyribonucleotide without covalent modification that binds to a moiety that aids in delivery to a cell may be a polyribonucleotide that is not covalently bound to a moiety, such as a protein, small molecule, a particle, a polymer, or a biopolymer that aids in delivery to a cell. A polyribonucleotide without covalent modification that binds to a moiety that aids in delivery to a cell may not contain a modified phosphate group. For example, a polyribonucleotide without covalent modification that binds to a moiety that aids in delivery to a cell may not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation may be free of any or all of: transfection reagents, cationic carriers, carbohydrate carriers, nanoparticle carriers, or protein carriers. For example, a naked delivery formulation may be free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, l,2-Dioleoyl-3-Trimethylammonium-Propane (DOTAP), N-[1-(2,3-dioleoyloxy)propyl]-N,N,N-trimethylammonium chloride (DOTMA), l-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-l-propanaminium trifluoroacetate (DOSPA), 3B—[N—(N\N′-Dimethylaminoethane)-carbamoyl]Cholesterol Hydrochloride (DC-Cholesterol HC1), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

A naked delivery formulation may comprise a non-carrier excipient. In some embodiments, a non-carrier excipient may comprise an inactive ingredient that does not exhibit an active cell-penetrating effect. In some embodiments, a non-carrier excipient may comprise a buffer, for example PBS. In some embodiments, a non-carrier excipient may be a solvent, a non-aqueous solvent, a diluent, a suspension aid, a surface active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation may comprise a diluent, such as a parenterally acceptable diluent. A diluent (e.g., a parenterally acceptable diluent) may be a liquid diluent or a solid diluent. In some embodiments, a diluent (e.g., a parenterally acceptable diluent) may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

In some embodiments, the pharmaceutical preparation as disclosed herein, the pharmaceutical composition as disclosed herein, the pharmaceutical drug substance of as disclosed, or the pharmaceutical drug product as disclosed herein is in parenteral nucleic acid delivery system. The parental nucleic acid delivery system can comprise the pharmaceutical preparation as disclosed herein, the pharmaceutical composition as disclosed herein, the pharmaceutical drug substance of as disclosed, or the pharmaceutical drug product as disclosed herein, and a parenterally acceptable diluent. In some embodiments, the pharmaceutical preparation as disclosed herein, the pharmaceutical composition as disclosed herein, the pharmaceutical drug substance of as disclosed, or the pharmaceutical drug product as disclosed herein in the parenteral nucleic acid delivery system is free of any carrier.

The invention is further directed to a host or host cell comprising the circular polyribonucleotide described herein. In some embodiments, the host or host cell is a vertebrate, mammal (e.g., human), or other organism or cell.

In some embodiments, the circular polyribonucleotide has a decreased, or fails to produce a, undesired response by the host's immune system as compared to the response triggered by a reference compound, e.g., a linear polynucleotide corresponding to the described circular polyribonucleotide or a circular polyribonucleotide lacking an encryptogen. In embodiments, the circular polyribonucleotide is non-immunogenic in the host. Some immune responses include, but are not limited to, humoral immune responses (e.g. production of antigen-specific antibodies) and cell-mediated immune responses (e.g., lymphocyte proliferation).

In some embodiments, a host or a host cell is contacted with (e.g., delivered to or administered to) the circular polyribonucleotide. In some embodiments, the host is a mammal, such as a human. The amount of the circular polyribonucleotide, expression product, or both in the host can be measured at any time after administration. In certain embodiments, a time course of host growth in a culture is determined. If the growth is increased or reduced in the presence of the circular polyribonucleotide, the circular polyribonucleotide or expression product or both is identified as being effective in increasing or reducing the growth of the host.

### Methods of Delivery

A method of delivering a circular polyribonucleotide molecule as described herein to a cell, tissue or subject, comprises administering the pharmaceutical composition, pharmaceutical drug substance or pharmaceutical drug product as described herein to the cell, tissue, or subject.

In some embodiments, the method of delivering is an in vivo method. For example, a method of delivering of a circular polyribonucleotide as described herein comprises parenterally administering to a subject in need thereof, the pharmaceutical composition, pharmaceutical drug substance or pharmaceutical drug product as described herein to the subject in need thereof. As another example, a method of delivering a circular polyribonucleotide to a cell or tissue of a subject, comprises administering parenterally to the cell or tissue the pharmaceutical composition, pharmaceutical drug substance or pharmaceutical drug product as described herein. In some embodiments, the circular polyribonucleotide is in an amount effective to elicit a biological response in the subject. In some embodiments, the circular polyribonucleotide is an amount effective to have a biological effect on the cell or tissue in the subject. In some embodiments, the the pharmaceutical composition, pharmaceutical drug substance or pharmaceutical drug product as described herein comprises a carrier. In some embodiments the pharmaceutical composition, pharmaceutical drug substance or pharmaceutical drug product as described herein comprises a diluent and is free of any carrier. In some embodiments, parenteral administration is intravenously, intramuscularly, ophthalmically, or topically.

In some embodiments, the pharmaceutical composition, pharmaceutical drug substance or pharmaceutical drug product is administered orally. In some embodiments the pharmaceutical composition, pharmaceutical drug substance or pharmaceutical drug product is administered nasally. In some embodiments, the pharmaceutical composition, pharmaceutical drug substance or pharmaceutical drug product is administered by inhalation. In some embodiments the pharmaceutical composition, pharmaceutical drug substance or pharmaceutical drug product is administered topically. In some embodiments the pharmaceutical composition, pharmaceutical drug substance or pharmaceutical drug product is administered ophthalmically. In some embodiments the pharmaceutical composition, pharmaceutical drug substance or pharmaceutical drug product is administered rectally. In some embodiments the pharmaceutical composition, pharmaceutical drug substance or pharmaceutical drug product is administered by injection. The administration can be systemic administration or local administration. In some embodiments the pharmaceutical composition, the pharmaceutical drug substance, or the pharmaceutical drug product is administered parenterally. In some embodiments the pharmaceutical composition, the pharmaceutical drug substance, or the pharmaceutical drug product is administered intravenously, intraarterially, intraperitoneally, intradermally, intracranially, intrathecally, intralymphaticly, subcutaneously, or intramuscularly. In some embodiments, the pharmaceutical composition, the pharmaceutical drug substance, or the pharmaceutical drug product is administered via intraocular administration, intracochlear (inner ear) administration, or intratracheal administration. In some embodiments, any of the methods of delivery as described herein are performed with a carrier. In some embodiments, any methods of delivery as described herein are performed without the aid of a carrier or cell penetrating agent.

In some embodiments, the circular polyribonucleotide or a product translated from the circular polyribonucleotide is detected in the cell, tissue, or subject at least 1 day, at least 2 days, at least 3 days, at least 4 days, or at least 5 days after the administering step. In some embodiments, the presence of the circular polyribonucleotide or a product translated from the circular polyribonucleotide is evaluated in the cell, tissue, or subject before the administering step. In some embodiments, the presence of the circular polyribonucleotide or a product translated from the circular polyribonucleotide is evaluated in the cell, tissue, or subject after the administering step.

Cell and Vesicle-Based Carriers

A circular RNA composition or preparation described herein can be administered to a cell in a vesicle or other membrane-based carrier.

In embodiments, a circular RNA composition or preparation described herein is administered in or via a cell, vesicle or other membrane-based carrier. In one embodiment, the circular RNA composition or preparation can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the circular RNA composition or preparation described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide), or cationic carriers (e.g., a cationic lipopolymer or transfection reagent). Non-limiting examples of carbohydrate carriers include phytoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropyleneimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, l,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-l-propanaminium trifluoroacetate (DOSPA), 3B—[N—(N\N′-Dimethylaminoethane)-carbamoyl]Cholesterol Hydrochloride (DC-Cholesterol HC1), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a circular RNA composition or preparation described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a circular RNA composition or preparation described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the circular RNA composition or preparation described herein.

Virosomes and virus-like particles (VLPs) can also be used as carriers to deliver a circular RNA composition or preparation described herein to targeted cells.

Plant nanovesicles and plant messenger packs (PMPs), e.g., as described in WO2011097480, WO2013070324, WO2017004526, or WO2020041784 can also be used as carriers to deliver the circular RNA composition or preparation described herein.

### Methods of Expression

The present invention includes a method for protein expression, comprising translating at least a region of the circular polyribonucleotide provided herein.

In some embodiments, the methods for protein expression comprises translation of at least 10%, at least 20%, at least 30%, at least 40%, at least 50%, at least 60%, at least 70%, at least 80%, at least 90%, or at least 95% of the total length of the circular polyribonucleotide into polypeptides. In some embodiments, the methods for protein expression comprises translation of the circular polyribonucleotide into polypeptides of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids. In some embodiments, the methods for protein expression comprises translation of the circular polyribonucleotide into polypeptides of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, or about 1000 amino acids. In some embodiments, the methods comprise translation of the circular polyribonucleotide into continuous polypeptides as provided herein, discrete polypeptides as provided herein, or both.

In some embodiments, the translation of at least a region of the circular polyribonucleotide takes place in vitro, such as rabbit reticulocyte lysate. In some embodiments, the translation of the at least a region of the circular polyribonucleotide takes place in vivo, for instance, after transfection of a eukaryotic cell, or transformation of a prokaryotic cell such as a bacteria.

In some aspects, the present disclosure provides methods of in vivo expression of one or more expression sequences in a subject, comprising: administering a circular polyribonucleotide to a cell of the subject wherein the circular polyribonucleotide comprises the one or more expression sequences; and expressing the one or more expression sequences from the circular polyribonucleotide in the cell. In some embodiments, the circular polyribonucleotide is configured such that expression of the one or more expression sequences in the cell at a later time point is equal to or higher than an earlier time point. In some embodiments, the circular polyribonucleotide is configured such that expression of the one or more expression sequences in the cell over a time period of at least 7, 8, 9, 10, 12, 14, 16, 18, 20, 22, 23, or more days does not decrease by greater than about 40%. In some embodiments, the circular polyribonucleotide is configured such that expression of the one or more expression sequences in the cell is maintained at a level that does not vary by more than about 40% for at least 7, 8, 9, 10, 12, 14, 16, 18, 20, 22, 23, or more days. In some embodiments, the administration of the circular polyribonucleotide is conducted using any delivery method described herein. In some embodiments, the circular polyribonucleotide is administered to the subject via intravenous injection. In some embodiments, the administration of the circular polyribonucleotide includes, but is not limited to, prenatal administration, neonatal administration, postnatal administration, oral, by injection (e.g., intravenous, intra-arterial, intraperitoneal, intradermal, subcutaneous and intramuscular), by ophthalmic administration and by intranasal administration.

In some embodiments, the methods for protein expression comprise modification, folding, or other post-translation modification of the translation product. In some embodiments, the methods for protein expression comprise post-translation modification in vivo, e.g., via cellular machinery.

All references and publications cited herein are hereby incorporated by reference.

The above described embodiments can be combined to achieve the afore-mentioned functional characteristics. This is also illustrated by the below examples which set forth exemplary combinations and functional characteristics achieved.

### Numbered Embodiments Set #1

[1] A pharmaceutical preparation of circular polyribonucleotide molecules, the pharmaceutical preparation comprising no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.
[2] A pharmaceutical preparation of circular polyribonucleotide molecules, the pharmaceutical preparation comprising no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) linear polyribonucleotide molecules of the total ribonucleotide molecules in the pharmaceutical preparation.
[3] A pharmaceutical preparation of circular polyribonucleotide molecules, wherein at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) of total ribonucleotide molecules in the pharmaceutical preparation are circular polyribonucleotide molecules.
[4] A pharmaceutical preparation of circular polyribonucleotide molecules, the pharmaceutical preparation having a level of linear RNA reduced by at least 30% (w/w), at least 40% (w/w), at least 50% (w/w), at least 60% (w/w), at least 70% (w/w), at least 80% (w/w), at least 90% (w/w), or at least 95% (w/w) after one or a plurality of purification steps compared to a level of the RNA prior to the one or a plurality of purification steps.
[5] A pharmaceutical preparation of paragraph 1, 2, 3, or 4 having a reduced level of one or more markers of an immune or inflammatory response after purification compared to prior to purification.
[6] The pharmaceutical preparation of paragraph 5, wherein the one or more markers of an immune or inflammatory response is a cytokine or an immunogenic related gene.
[7] The pharmaceutical preparation of any one of paragraphs 5 or 6, wherein the one or more markers of an immune or inflammatory response is expression of a gene selected from the group consisting of RIG-I, MDA5, PKR, IFN-beta, OAS, and OASL.
[8] A pharmaceutical preparation of circular polyribonucleotide molecules comprising no more than 30% (w/w) linear polyribonucleotide molecules of the total ribonucleotide molecules in the pharmaceutical preparation and substantially free of a process-related impurity selected from a host cell protein, a host cell deoxyribonucleic acid, an enzyme, a reagent component, a gel component, or a chromatographic material.
[9] The pharmaceutical preparation of any one prior paragraph, wherein the linear polyribonucleotide molecules comprise a linear polyribonucleotide molecule counterpart of the circular polyribonucleotide molecules.
[10] The pharmaceutical preparation of any one of paragraphs [1]-[2], or [9], wherein the linear polyribonucleotide molecules comprises a linear polyribonucleotide molecule counterpart of the circular polyribonucleotide molecules, a linear polyribonucleotide molecule non-counterpart of the circular polyribonucleotide molecules, or a combination thereof.
[11] The pharmaceutical preparation of any one of paragraphs [1]-[10], the pharmaceutical preparation comprises less than 10 EU/kg or lacks endotoxin as measured by a Limulus amebocyte lysate test.
[12] The pharmaceutical preparation of any one of paragraphs 1-[11], wherein the pharmaceutical preparation comprises a bioburden of less than 100 CFU/100 ml or less than 10 CFU/100 ml before sterilization.
[13] The pharmaceutical preparation of any one of paragraphs [1]-[11], wherein the pharmaceutical preparation is a sterile pharmaceutical preparation.
[14] The pharmaceutical preparation of paragraph [12], wherein the sterile pharmaceutical preparation supports growth of fewer than 100 viable microorganisms as tested under aseptic conditions.
[15] The pharmaceutical preparation of any one of paragraphs [1]-[14], the pharmaceutical preparation meeting the standard of USP <71>.
[16] The pharmaceutical preparation of any one of paragraphs [1]-[15], the pharmaceutical preparation meeting the standard of USP <85>.
[17] The pharmaceutical preparation of any one of paragraphs 1-[16], wherein the circular polyribonucleotide molecules comprises a quasi-helical structure.
[18] The pharmaceutical preparation of any one of paragraphs 1-[17], wherein the circular polyribonucleotide molecules comprises a quasi-double stranded secondary structure.
[19] The pharmaceutical preparation of any one of paragraphs [1]-[18], wherein the circular polyribonucleotide molecules comprise one or more expression sequences and a stagger element at a 3′ end of at least one expression sequence.
[20] The pharmaceutical preparation of any one of paragraphs [1]-[19], wherein the pharmaceutical preparation is an intermediate pharmaceutical preparation of a final drug product.
[21] The pharmaceutical preparation of any one of paragraphs [1]-[19], wherein the pharmaceutical preparation is a final drug product for administration to a subject.
[22] The pharmaceutical preparation of any one of paragraphs [1]-[21], the pharmaceutical preparation comprising a concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 5 mg/mL, 10 mg/mL, 100 mg/mL, 200 mg/mL, or 500 mg/mL, 600 mg/mL, 650 mg/mL, 700 mg/mL, or 750 mg/mL circular polyribonucleotide molecules.
[23] The pharmaceutical preparation of any one of paragraphs [1]-[22], the preparation comprising no more than 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL of deoxyribonucleotide molecules.
[24] The pharmaceutical preparation of any one of paragraphs [1]-[23], the pharmaceutical preparation comprising an A260/A280 absorbance ratio of from about 1.6 to 2.3 as measured by a spectrophotometer.
[25] The pharmaceutical preparation of any one of paragraphs [1]-[24], the pharmaceutical preparation comprising a protein contamination of less than 1 pg, 10 pg, 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of the protein contamination per milligram (mg) of the circular polyribonucleotide molecules.
[26] The pharmaceutical preparation of paragraph [23], wherein the protein contamination comprises an enzyme.
[27] The pharmaceutical preparation of paragraph [24], wherein the enzyme is a nuclease or a ligase.
[28] The pharmaceutical preparation of any one of paragraphs [1]-[27], wherein an amount of linear polyribonucleotide molecules as compared to circular polyribonucleotide molecules is determined using the method of Example 2 or Example 3.
[29] The pharmaceutical preparation of any one of paragraphs [1], [2], or [9]-[28], wherein an amount of linear polyribonucleotide molecules in the pharmaceutical preparation is determined using the method of Example 2.
[30] The pharmaceutical preparation of any one of paragraphs [3], [9], or [11]-[29], wherein an amount of circular polyribonucleotide molecules in the pharmaceutical preparation is determined using the method of Example 3.
[31] A method of making a pharmaceutical composition, comprising:
   a) providing a preparation of circular polyribonucleotide molecules;
   b) processing the preparation to substantially remove linear polyribonucleotide molecules remaining in the preparation;
   c) optionally evaluating the amount of linear polyribonucleotide molecules remaining in the preparation after the processing step; and
   d) further processing the preparation to produce the pharmaceutical composition for pharmaceutical use.
[32] The method of paragraph [31], wherein further processing of step d) comprises one or more of:
   e) processing the preparation to substantially remove deoxyribonucleotide molecules;
   f) evaluating the amount of deoxyribonucleotide molecules in the preparation;
   g) formulating the preparation with a pharmaceutical excipient;
   h) concentrating the preparation; and
   i) documenting the amount of deoxyribonucleotide molecules in the preparation in a print or digital media.
[33] The method of any one of paragraphs [31]-[32], wherein further processing of step d) comprises one or more of:
   e) processing the preparation to substantially remove protein contamination;
   f) evaluating the amount of protein contamination in the preparation;
   g) formulating the preparation with a pharmaceutical excipient; and
   h) concentrating the preparation.
[34] The method of any one of paragraphs [31]-[33], wherein further processing of step d) comprises one or more of:
   e) processing the preparation to substantially remove endotoxin;
   f) evaluating the amount of endotoxin in the preparation;
   g) formulating the preparation with a pharmaceutical excipient; and
   h) concentrating the preparation.
[35] The method of any one of paragraphs [31]-[34], wherein the linear polyribonucleotide molecules comprises a linear polyribonucleotide molecule counterpart of the circular polyribonucleotide molecules.
[36] The method of any one of paragraphs [31]-[35], wherein the linear polyribonucleotide molecules comprises a linear polyribonucleotide molecule counterpart of the circular polyribonucleotide molecules, a linear polyribonucleotide molecule non-counterpart of the circular polyribonucleotide molecules, or a combination thereof.
[37] The method of any one of paragraphs [31]-[36], wherein the protein contamination comprises an enzyme.
[38] A method of making a pharmaceutical drug substance, comprising:
   a) providing a preparation of circular polyribonucleotide molecules;
   b) evaluating the amount of linear polyribonucleotide molecules remaining in the preparation; and
   c) processing the preparation of circular polyribonucleotide molecules as a pharmaceutical drug substance if the preparation meets a reference criterion for an amount of linear polyribonucleotide molecules present in the preparation.
[39] A method of making a pharmaceutical drug product, comprising:
   a) providing a preparation of circular polyribonucleotide molecules;
   b) measuring the amount of linear polyribonucleotide molecules in the preparation;
   c) formulating the preparation of circular polyribonucleotide molecules as the pharmaceutical drug product if it meets a reference criterion for an amount of linear polyribonucleotide molecules present in the preparation; and
   d) labelling and shipping the pharmaceutical drug product if it meets a reference criterion for the amount of linear polyribonucleotide molecules present in the pharmaceutical drug product.
[40] The method of any one of paragraphs [38]-[39], wherein the formulating the preparation of circular polyribonucleotide molecules comprises combining the preparation of circular polyribonucleotide molecules with a pharmaceutical excipient.
[41] The method of any one of paragraphs [38]-[40], wherein the reference criterion for the amount of linear polyribonucleotide molecules present in the preparation is a pharmaceutical release specification.
[42] The method of paragraph [41], wherein the pharmaceutical preparation further meets a reference criterion for the sequence of the circular polyribonucleotide molecules, e.g., a sequence having at least 80% (e.g., 85%, 90%, 95%, 97%, 99%, 100%) sequence identity to a reference circular polyribonucleotide sequence.
[43] The method of any one of paragraphs [38]-[42], wherein the reference criterion for the amount of linear polyribonucleotide molecules present in the preparation is the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.
[44] The method of any one of paragraphs [38]-[42], wherein the reference criterion for the amount of linear polyribonucleotide molecules present in the preparation is the presence of no more than a specified amount of (e.g., an undetectable level or level below a detection limit when measured) of linear polyribonucleotide molecules when measured by microscopy, by spectrophotometry, by fluorometry, by denaturing urea polyacrylamide gel electrophoresis imaging, by UV-Vis spectrophotometry, by RNA electrophoresis, or by RNAse H analysis.
[45] The method of any one of paragraphs [38]-[44], wherein the linear polyribonucleotide molecules comprises a linear polyribonucleotide molecule counterpart of the circular polyribonucleotide molecules.
[46] The method of any one of paragraphs [38]-[45], wherein the linear polyribonucleotide molecules comprises a linear polyribonucleotide molecule counterpart of the circular polyribonucleotide molecules, a linear polyribonucleotide molecule non-counterpart of the circular polyribonucleotide molecules, or a combination thereof.
[47] The method of any one of paragraphs [38]-[46], wherein the pharmaceutical drug product or pharmaceutical drug substance comprises a concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 5 mg/mL, 10 mg/mL, 100 mg/mL, 200 mg/mL, or 500 mg/mL circular polyribonucleotide molecules.
[48] The method of any one of paragraphs [38]-[47], wherein the pharmaceutical drug product or pharmaceutical drug substance comprises at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to total ribonucleotide molecules in the pharmaceutical preparation.
[49] The method of paragraph [48], wherein the at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to total ribonucleotide molecules in the pharmaceutical preparation is measured by microscopy, by spectrophotometry, by fluorometry, by denaturing urea polyacrylamide gel electrophoresis imaging, by UV-Vis spectrophotometry, by RNA electrophoresis, by RNAse H analysis, by UV spectroscopic or fluorescence detectors, by light scattering techniques, by surface plasmon resonance (SPR) with or without the use of methods of separation including HPLC, by HPLC, by chip or gel based electrophoresis with or without using either pre or post separation derivatization methodologies, by using methods of detection that use silver or dye stains or radioactive decay for detection of linear polyribonucleotide molecules, or by methods that utilize microscopy, visual methods or a spectrophotometer.
[50] The method of paragraph [48] or [49], wherein the amount of circular polyribonucleotide relative to total ribonucleotide molecules is determined using the method of Example 3.
[51] The method of any one of paragraphs [38]-[50], wherein the pharmaceutical drug product or pharmaceutical drug substance comprises less than 10 EU/kg or lacks endotoxin as measured by the Limulus amebocyte lysate test.
[52] The method of any one of paragraphs [38]-[51], wherein the pharmaceutical drug product or pharmaceutical drug substance comprises a bioburden of less than 100 CFU/100 ml or less than 10 CFU/100 ml before sterilization.
[53] The method of any one of paragraphs [38]-[52], wherein the pharmaceutical drug product or pharmaceutical drug substance is a sterile drug product or sterile drug substance.
[54] The method of paragraph [53], wherein the sterile drug product or sterile drug substance supports growth of fewer than 100 viable microorganisms as tested under aseptic conditions.
[55] The method of any one of paragraphs [38]-[54], wherein the pharmaceutical drug product or pharmaceutical drug substance meets the standard of USP <71>.
[56] The method of any one of paragraphs [38]-[55], wherein the pharmaceutical drug product or pharmaceutical drug substance meets the standard of USP <85>.
[57] The method of any one of paragraphs [38]-[56], wherein the circular polyribonucleotide molecules comprise one or more expression sequences and a stagger element at a 3′ end of at least one expression sequence.
[58] The method of any one of paragraphs [38]-[57], wherein the preparation further meets a reference criterion for the amount of deoxyribonucleotide molecules present in the preparation.
[59] The method of paragraph [58], wherein the reference criterion for the amount of deoxyribonucleotide molecules present in the preparation is the presence of no more than 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml, 1000 μg/mL, 5000 μg/mL, 10,000 μg/mL, or 100,000 μg/mL of deoxyribonucleotide molecules.
[60] The method of any one of paragraphs [38]-[59], wherein the preparation further meets a reference criterion for the amount of protein contamination present in the preparation.
[61] The method of paragraph [60], wherein the reference criterion for the amount of protein contamination present in the preparation is the presence of a protein contamination of less than 1 pg, 10 pg, 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of the protein contamination per milligram (mg) of the circular polyribonucleotide molecules.
[62] The method of any one of paragraphs [38]-[61], wherein the pharmaceutical drug product or pharmaceutical drug substance comprises an A260/A280 absorbance ratio of from about 1.6 to 2.3 as measured by a spectrophotometer.
[63] A method of delivering a circular polyribonucleotide to a subject or to a cell or tissue of a subject, comprising administering the pharmaceutical preparation of any one of paragraphs [1]-[27], the pharmaceutical composition of any one of paragraphs [31]-[37], the pharmaceutical drug substance of any one of paragraphs [38]-[62], or the pharmaceutical drug product of any one of paragraphs [39]-[62] to the cell or tissue of the subject, wherein the circular polyribonucleotide or a product translated from the circular polyribonucleotide is detected in the cell, tissue, or subject at least 3 days after the administering step.
[64] The method of paragraph [63], further comprising evaluating the presence the circular polyribonucleotide or a product translated from the circular polyribonucleotide in the cell, tissue or subject before the administering step.
[65] The method of any one of paragraphs [63]-[64], further comprising evaluating the presence the circular polyribonucleotide or a product translated from the circular polyribonucleotide in the cell, tissue or subject after the administering step.
[66] A parenteral nucleic acid delivery system comprising (i) the pharmaceutical preparation of any one of paragraphs [1]-[27], the pharmaceutical composition of any one of paragraphs [31]-[37], the pharmaceutical drug substance of any one of paragraphs [38]-[62], or the pharmaceutical drug product of any one of paragraphs [39]-[62], and (ii) a parenterally acceptable diluent.
[67] The parenteral nucleic acid delivery system of paragraph [66], wherein the pharmaceutical preparation of any one of paragraphs [1]-[27], the pharmaceutical composition of any one of paragraphs [31]-[37], the pharmaceutical drug substance of any one of paragraphs [38]-[62], or the pharmaceutical drug product of any one of paragraphs [39]-[62] is free of any carrier.
[68] A method of delivering a circular polyribonucleotide to a subject comprising parenterally administering to a subject in need thereof the pharmaceutical preparation of any one of paragraphs [1]-[27], the pharmaceutical composition of any one of paragraphs [31]-[37], the pharmaceutical drug substance of any one of paragraphs [38]-[62], or the pharmaceutical drug product of any one of paragraphs [39]-[62] to a subject in need thereof.
[69] The method of paragraph [68], wherein the circular polyribonucleotide is in an amount effective to elicit a biological response in the subject
[70] The method of paragraph [68], wherein the circular polyribonucleotide is in an amount effective to have a biological effect on a cell or tissue in the subject.
[71] A method delivering a circular polyribonucleotide to a cell or tissue of a subject, comprising administering parenterally to the cell or tissue the pharmaceutical preparation of any one of paragraphs [1]-[27], the pharmaceutical composition of any one of paragraphs [31]-[37], the pharmaceutical drug substance of any one of paragraphs [38]-[62], or the pharmaceutical drug product of any one of paragraphs [39]-[62].
[72] The method of any one of paragraphs [68]-[71], wherein the the pharmaceutical preparation of any one of paragraphs [1]-[27], the pharmaceutical composition of any one of paragraphs [31]-[37], the pharmaceutical drug substance of any one of paragraphs [38]-[62], or the pharmaceutical drug product of any one of paragraphs [39]-[62] comprises a carrier.
[73] The method of any one of paragraphs [68]-[71], wherein the the pharmaceutical preparation of any one of paragraphs [1]-[27], the pharmaceutical composition of any one of paragraphs [31]-[37], the pharmaceutical drug substance of any one of paragraphs [38]-[62], or the pharmaceutical drug product of any one of paragraphs [39]-[62] comprises a diluent and is free of any carrier.
[74] The method of any one of paragraphs [68]-[73], wherein parenteral administration is intravenously, intramuscularly, ophthalmically or topically.

### Numbered Embodiments Set #2

[1] A method of making a pharmaceutical composition, comprising:
   a) providing a plurality of linear polyribonucleotide molecules;
   b) circularizing the linear polyribonucleotides to generate a preparation of circular polyribonucleotides;
   c) processing the preparation of circular polyribonucleotides to substantially remove remaining linear polyribonucleotide molecules;
   d) optionally evaluating an amount of linear polyribonucleotide molecules in the preparation after the processing step; and
   e) further processing the preparation to produce the pharmaceutical composition for pharmaceutical use.
[2] The method of paragraph [1], wherein further processing of step e) comprises one or more of:
   f) processing the preparation to substantially remove deoxyribonucleotide molecules;
   g) evaluating the amount of deoxyribonucleotide molecules in the preparation;
   h) formulating the preparation with a pharmaceutical excipient;
   i) concentrating the preparation; and
   j) documenting the amount of deoxyribonucleotide molecules in the preparation in a print or digital media.
[3] The method of any one of paragraphs [1]-[2], wherein further processing of step e) comprises one or more of:
   f) processing the preparation to substantially remove protein contamination;
   g) evaluating the amount of protein contamination in the preparation;
   h) formulating the preparation with a pharmaceutical excipient; and
   i) concentrating the preparation.
[4] The method of any one of paragraphs [1]-[3], wherein further processing of step e) comprises one or more of:
   f) processing the preparation to substantially remove endotoxin;
   g) evaluating the amount of endotoxin in the preparation;
   h) formulating the preparation with a pharmaceutical excipient; and
   i) concentrating the preparation.
[5] The method of any one of paragraphs [1]-[4], wherein the linear polyribonucleotide molecules comprises a linear polyribonucleotide molecule counterpart of the circular polyribonucleotide molecules or a fragment of the linear polyribonucleotide molecule counterpart of the circular polyribonucleotide molecules.
[6] The method of any one of paragraphs [1]-[5], wherein the linear polyribonucleotide molecules comprises a linear polyribonucleotide molecule counterpart of the circular polyribonucleotide molecules or a fragment thereof, a linear polyribonucleotide molecule non-counterpart of the circular polyribonucleotide molecules or a fragment thereof, or a combination thereof.
[7] The method of any one of paragraphs [1]-[6], wherein the pharmaceutical composition comprises no more than 20%, 15%, 10%, 5%, 2%, 1%, or 0.5% (w/w) combined linear and nicked polyribonucleotide molecules relative to the total ribonucleotide molecules in the preparation.
[8] The method of any one of paragraphs [1]-[7], wherein the circularizing comprises splint ligation.
[9] The method of any one of paragraphs [1]-[7], wherein the protein contamination comprises an enzyme.
[10] The method of any one of paragraphs [1]-[9], wherein the amount of linear polyribonucleotide molecules as compared to circular polyribonucleotide molecules is determined using the method of Example 2 or Example 3.
[11] The method of any one of paragraphs [1]-[10], wherein the amount of linear polyribonucleotide molecules in the preparation is determined using the method of Example 2.
[12] The method of any one of paragraphs [1]-[11], wherein the amount of circular polyribonucleotide molecules in the preparation is determined using the method of Example 3.
[13] The method of any one of paragraphs [1]-[12], wherein the pharmaceutical composition comprises no more than 30%, 20%, 15%, 10%, 5%, 2%, 1%, or 0.5% (w/w), linear polyribonucleotide molecules of the total ribonucleotide molecules in the preparation.
[14] A pharmaceutical preparation of circular polyribonucleotide molecules, the pharmaceutical preparation comprising circular polyribonucleotide molecules and no more than 5%, 4%, 3%, 2%, 1%, 0.5%, or 0.1% (w/w) nicked polyribonucleotide molecules of the total ribonucleotide molecules in the pharmaceutical preparation
[15] The pharmaceutical preparation of paragraph [14], having a reduced level of one or more markers of an immune or inflammatory response after purification compared to prior to purification.
[16] The pharmaceutical preparation of paragraph [15], wherein the one or more markers of an immune or inflammatory response is a cytokine or an immunogenic related gene.
[17] The pharmaceutical preparation of any one of paragraphs [15] or [16], wherein the one or more markers of an immune or inflammatory response is expression of a gene selected from the group consisting of RIG-I, MDA5, PKR, IFN-beta, OAS, and OASL.
[18] The pharmaceutical preparation of any one of paragraphs [14]-[17] that is substantially free of a process-related impurity selected from a cell protein, a cell deoxyribonucleic acid, an enzyme, a reagent component, a gel component, or a chromatographic material.
[19] The pharmaceutical preparation of any one of paragraphs [14]-[18], wherein the nicked linear polyribonucleotide molecules are post-circularized nicked linear polyribonucleotides.
[20] The pharmaceutical preparation of any one of paragraphs [14]-[19], the pharmaceutical preparation comprises less than 10 EU/kg or lacks endotoxin as measured by a Limulus amebocyte lysate test.
[21] The pharmaceutical preparation of any one of paragraphs [14]-[20], wherein the pharmaceutical preparation comprises a bioburden of less than 100 CFU/100 ml or less than 10 CFU/100 ml before sterilization.
[22] The pharmaceutical preparation of any one of paragraphs [14]-[21], wherein the pharmaceutical preparation is a sterile pharmaceutical preparation.
[23] The pharmaceutical preparation of paragraph [22], wherein the sterile pharmaceutical preparation supports growth of fewer than 100 viable microorganisms as tested under aseptic conditions.
[24] The pharmaceutical preparation of any one of paragraphs [14]-[23], the pharmaceutical preparation meeting the standard of USP <71>.
[25] The pharmaceutical preparation of any one of paragraphs [14]-[24], the pharmaceutical preparation meeting the standard of USP <85>.
[26] The pharmaceutical preparation of any one of paragraphs [14]-[25], wherein the circular polyribonucleotide molecules comprise one or more expression sequences and a stagger element at a 3′ end of at least one expression sequence.
[27] The pharmaceutical preparation of any one of paragraphs [14]-[26], wherein the pharmaceutical preparation is an intermediate pharmaceutical preparation of a final drug product.
[28] The pharmaceutical preparation of any one of paragraphs [14]-[27], wherein the pharmaceutical preparation is a final drug product for administration to a subject.
[29] The pharmaceutical preparation of any one of paragraphs [14]-[28], wherein the pharmaceutical preparation comprises a concentration of at least 0.1 ng/mL of the circular polyribonucleotide molecules.
[30] The pharmaceutical preparation of any one of paragraphs [14]-[29], wherein the pharmaceutical preparation comprises no more than about 9% (w/w), 8% (w/w), 7% (w/w), 6% (w/w), 5% (w/w), 4% (w/w), 3% (w/w), 2% (w/w), 1% (w/w), or 0.5% (w/w) nicked polyribonucleotide molecules.
[31] The pharmaceutical preparation of any one paragraphs [14]-[30], at least 80% (w/w) of total ribonucleotide molecules in the pharmaceutical preparation are circular polyribonucleotide molecules.
[32] The pharmaceutical preparation of any one of paragraphs [14]-[31], wherein the pharmaceutical preparation comprises no more than 20% (w/w) linear polyribonucleotide molecules of the total ribonucleotide molecules in the preparation.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used. Examples 1-3, 6, 9, 10, and 16, and their corresponding Figures as described in [0356]-[0375], [0393]-[0405], and [0433]-[0436] of International Patent Publication No. WO2019118919A1, are incorporated herein by reference in their entirety.

Table 2 below is intended to provide a brief summary of the content of each example described below, which by no means is exclusive. Certain aspects of the examples may not be reflected in the Descriptions in Table 2.

TABLE 2
Brief Summary of the Examples

| Example | Descriptions |
|---|---|
| 1 | Concatemerized RNA was detected in circular RNA preparations after |
| | purification |
| 2 | Linear RNA was detected in circular RNA preparations after purification and |
| | content was quantified with the aid of a standard curve |
| 3 | Circular RNA was purified using gel excision and extraction method and content |
| | of linear RNA was quantified |
| 4 | Production of 91% (w/w) pure circular RNA molecules relative to the total |
| | ribonucleotide molecules in the preparation |
| 5 | Quantification of nicked circular RNA relative to total RNA molecules in the |
| | preparation |
| 6 | Protein expression from cells transfected with the gel purified circular RNA |
| | preparation was detected at higher levels and for longer periods of time than cells |
| | transfected with unpurified RNA |
| 7 | Linear RNA content of circular RNA preparations negatively affected expression |
| | in a dose dependent manner in cells |
| 8 | Linear RNA in circular RNA preparations negatively affected expression levels |
| | and time |
| 9 | Enrichment of circular RNA from a preparation via polyadenylation of linear |
| | RNA using biotin-labelled adenoside and subsequent streptavidin-mediated |
| | capture |
| 10 | Reduction of linear RNA present in circular RNA preparations increased |
| | expression of the encoded protein |
| 11 | Linear RNA content of circular RNA preparations affected circular RNA stability |
| | in a dose dependent manner in cells |
| 12 | Linear RNA content of circular RNA preparations affected innate |
| | immunogenicity in a dose dependent manner in cells |
| 13 | In vivo assessment of immunogenicity of the circular RNA after transfection into |
| | cells |
| 14 | Control of protein expression from riboswitch on the circular RNA |
| 15 | In vivo assessment of immunogenicity of the circular RNA after delivery in mice |
| 16 | Circular RNA with a double stranded RNA segment |
| 17 | Circular RNA with an quasi-double stranded RNA segment |
| 18 | Circular RNA with a functional quasi-helical structure |
| 19 | Self transcription/replication of circular RNA including HDV replication |
| | domain(s), antigenomic replication competent ribozyme and a nuclear localization |
| | signal |
| 20 | Preservation of a circular RNA through cell division into daughter cells |
| 21 | In vivo production of circular RNA using splint ligation |
| 22 | Assessment of circularization efficiency of RNA splint ligation for producing |
| | circular RNA |
| 23 | Circular RNA exhibited lower susceptibility to degradation by RNase R compared |
| | to linear RNA |
| 24 | Purification of circular RNA via electrophoresis |
| 25 | Protein was expressed in vitro from a circular RNA |
| 26 | Protein was expressed in vitro from a circular RNA lacking IRES |
| 27 | Protein was expressed in vitro from a circular RNA in the absence of a cap |
| 28 | Protein was expressed in vitro from a circular RNA in the absence of 5′ UTR |
| 29 | Protein was expressed in vitro from a circular RNA in the absence of 3′ UTR |
| 30 | Protein was expressed in vitro from a circular RNA in the absence of a |
| | termination element (stop codon) |
| 31 | Discrete proteins were expressed in vitro from a circular RNA in the absence of |
| | termination element (stop codons) |
| 32 | Proteins were expressed via rolling circle translation from a circular RNA in the |
| | absence of termination element (stop codons) |
| 33 | Biologically active protein (luciferase) was expressed from a circular RNA in |
| | vitro |
| 34 | Circular RNA had a prolonged half-life in vitro as compared to a linear RNA. |
| 35 | Circular RNA had a prolonged half-life in vivo after transfected into cells as |
| | compared to a linear RNA. |
| 36 | Circular RNA was translated via rolling circle translation in cells |
| 37 | Circular RNA had reduced immunogenic gene expression in cells |
| 38 | Circular RNA was translated via rolling circle translation in cells |
| 39 | Circular RNA had higher levels of luciferase activity or increased protein |
| | produced as compared to its linear RNA counterpart. |
| 40 | Circular RNA without a termination element (stop codon) produced higher levels |
| | of protein product having functional luciferase activity than its linear RNA |
| | counterpart. |
| 41 | Circular RNA initiated protein expression with an IRES and produced higher |
| | levels of protein product having functional luciferase activity than circular RNA |
| | with Kozak initiated protein expression. |
| 42 | Rolling circle translation of circular RNA that lacks a termination element |
| | initiated greater protein production with an IRES and produced more protein |
| | product having functional luciferase activity as compared to a circular RNA with |
| | a termination element Kozak translation initiation |
| 43 | Circular RNA produced higher levels of protein having the correct molecular |
| | weight as compared to its linear RNA counterpart. |
| 44 | Discrete protein products were translated via rolling circle translation from |
| | circular RNA lacking a termination element (stop codon) and having a stagger |
| | element in cells; and the circular RNA with a stagger element expressed more |
| | protein product having the correct molecular weight than its linear counterpart. |
| 45 | Circular RNA possessing both quasi-double stranded and helical structure was |
| | generated |
| 46 | Circular RNA possessing a quasi-helical structure linked with a repetitive |
| | sequence was generated |
| 47 | Circular RNA was demonstrated to be circular and not concatemeric by RNase H |
| | degradation test |
| 48 | Generation of large circular RNAs |
| 49 | Generation of a circular RNA with a protein binding site |
| 50 | Generation of circular RNA with regulatory nucleic acid sites |
| 51 | Generation of circular RNA by self-splicing |
| 52 | Generation of circular RNA with splicing element and encryptogen |
| 53 | Circular RNA during was detected to persist during cell division |
| 54 | Circular RNA expressed multiple proteins from a single construct via rolling |
| | circle translation |
| 55 | Transfection of circular RNA in cells demonstrated reduced toxicity compared to |
| | linear RNA |
| 56 | Circular RNA expressed better under stress conditions than linear RNA |
| 57 | Control of protein expression from circular RNA via riboswitch on the circular |
| | RNA |
| 58 | Modified circular RNAs were generated and shown to express proteins in |
| | transfected cells and induce reduced immunogenic related gene expression as |
| | compared to unmodified circular RNA |
| 59 | Circular RNA had a longer half-life/increased stability in vivo after delivery into |
| | mice as compared to its linear counterpart |
| 60 | Circular RNA expressed protein in vivo for prolonged periods of time, with levels |
| | of protein activity in the plasma at multiple days post injection |

### Example 1: Characterization of a Circular RNA Preparation by Assessing RNAse H-Produced Nucleic Acid Degradation Products

This Example demonstrates that assessment of a circular RNA preparation for RNAse H-produced nucleic acid degradation products can detect linear and concatemerized versus circular products.

RNA, when incubated with a ligase, can either not react or form an intra- or intermolecular bond, generating a circular (no free ends) or a concatemeric RNA (linear), respectively. Treatment of each type of RNA with a complementary DNA primer and RNAse H, a nonspecific endonuclease that recognizes DNA/RNA duplexes, is expected to produce a unique number of degradation products of specific sizes depending on the starting RNA material.

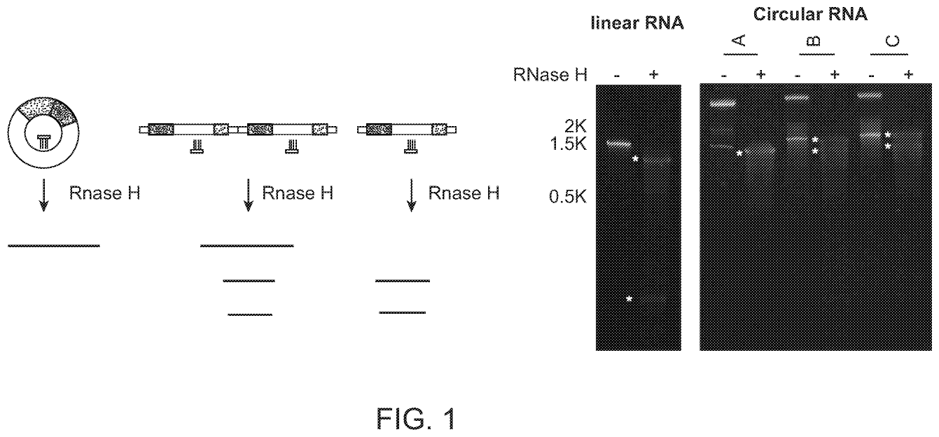

A ligated RNA may be shown to be circular RNA without concatemeric RNA contamination or circular RNA with concatemeric RNA contamination, based on the number and size of RNAs produced by RNAse H degradation. When the primer and RNase H are added to circular RNA, a single primer duplexes with the circular RNA and RNase H degrades the DNA/RNA duplex region to result in a single linear RNA product. When a primer and RNase H are added to a concatemer, at least two primers duplex with the concatemeric RNA and RNase H degrades the DNA/RNA duplexes to result in three products; one product is the RNA from the 5′ end to the first primer binding region, one product is the RNA between the first primer binding region and the next primer binding region which may include multiple RNAs depending on the number of concatemers ligated together, and a final product is the RNA from the last primer binding region to the 3′ end. When a primer and RNase H are added to linear RNA, a single primer duplexes with the linear RNA to result in one product for RNA from the 5′ end to the primer binding region and another product for the primer binding region to the 3′ end. The left side cartoon of FIG. 1 illustrates this strategy.

In this Example, circular RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Nanoluciferase (Nluc) and two spacer elements flanking the IRES-ORF.

To test circularization status of the RNA, 0.05 pmole/μl of linear or circular RNA preparation was incubated with 0.25 U/μl of RNAse H, an endoribonuclease that digests DNA/RNA duplexes, and 0.3 pmole/μl oligomer complementary to Nluc RNA (CACCGCTCAGGACAATCCTT) at 37° C. for 30 min. After incubation, the reaction mixture was analyzed by 6% denaturing PAGE. The gel was stained with SYBR-green and visualized by E-gel Imager. The band intensity on the visualized gel was measured and analyzed by ImageJ.

The right side of FIG. 1 shows the actual cleavage products in this experiment. The number of bands in the linear RNA lane incubated with RNAse H endonuclease produced two bands as expected, whereas a single band was detected in the circular RNA lane in the case of lane A, indicating that the circular RNA was in fact circular and not concatemeric. In the case of lane B & lane C, bands from linear and concatemer contamination were visible after RNase H treatment due to the presence of multiple smaller fragment bands appearing in the RNAse H lanes.

### Example 2: Linear RNA in Circular RNA Preparations (Standard Curve)

This Example demonstrates calculation of linear RNA in a circular RNA preparation.

In this Example, the circular RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Nanoluciferase (Nluc) and two spacer elements flanking the IRES-ORF.

Splint ligated circular RNA was generated by treatment of the transcribed linear RNA and a DNA splint with T4 DNA ligase 2 (New England Biolabs, Inc., M0239).

To purify the circular RNAs, ligation mixtures were resolved on 4% denaturing PAGE and RNA bands corresponding to each of the circular RNAs were excised. Excised RNA gel fragments were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol. Eluted circular RNA was analyzed by 6% denaturing PAGE. The gel was stained with SYBR-green and visualized by E-gel Imager.

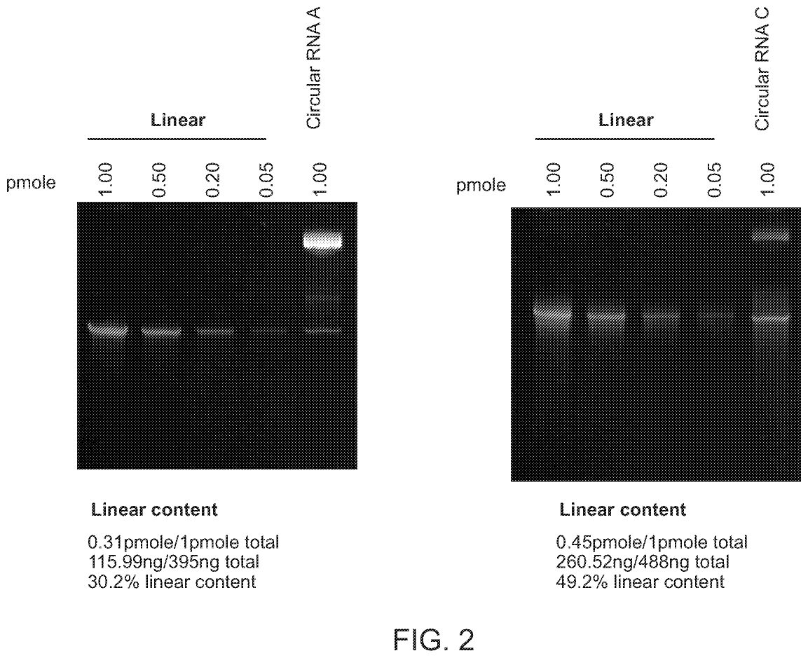

The amount of RNA on the gel was determined by comparing the band intensity of known amount and same size of RNA (standard RNA). A standard curve was generated to determine the amount of unknown sample on the gel (FIG. 2). To generate a standard curve 1, 0.5, 0.2 and 0.05 pmoles of linear counterpart of the circular RNA were loaded parallel with a circular RNA preparation on a 6% denaturing PAGE. The denaturing gel was stained with SYBR-green and visualized by E-gel Imager. Then each band intensity on the gel was measured and analyzed by ImageJ. The standard curve for linear RNA was generated through analysis of band intensity of the RNA loaded in each of the different lanes (R²>0.98 in all cases), and the amount of linear RNA in the circular RNA preparation was determined based on the linear RNA standard curve.

The amount of linear RNA in the different samples was as follows for circular RNA preparation A: linear RNA was calculated to be approximately 0.31 mole/mole, or 115.99 ng/395 ng, or 30.2%. For circular RNA preparation C: linear RNA was calculated to be approximately 0.45 mole/mole, or 260.52 ng/488 ng, or 49.2%.

### Example 3: Linear RNA in Circular RNA Preparations (Gel Excision and Extraction)

This Example demonstrates purification and quantification of circular RNA in a preparation.

In this Example, the circular RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (New England Biolabs, Inc., M0356) following the manufacturer's instructions, and purified again with the RNA purification system. Circular RNAs were designed to include an IRES with an ORF encoding Nanoluciferase (Nluc) and two spacer elements flanking the IRES-ORF.

Splint ligated circular RNA was generated by treatment of the transcribed linear RNA and a DNA splint with T4 DNA ligase 2 (New England Biolabs, Inc., M0239).

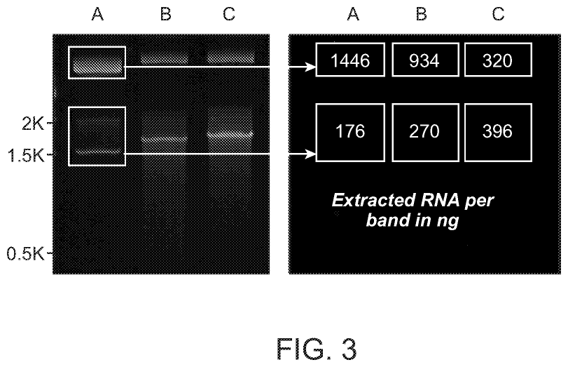

To purify the circular RNAs, ligation mixtures were resolved on 6% denaturing PAGE and RNA bands corresponding to each of the circular RNAs were excised. Excised RNA gel fragments were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol in the presence of 0.3M sodium acetate. Eluted circular RNA was analyzed by 6% denaturing PAGE. The gel was stained with SYBR-green and visualized by E-gel Imager (FIG. 3). Visible bands were again excised and crushed using by gel breaker. To extract RNA, gel elution buffer (0.5M NaOAc, 1 mM EDTA, 0.1% SDS) was added to the crushed gel and incubated for 1 hr at 37° C. Supernatant was harvested, and RNA was eluted once again by adding gel elution buffer to the crushed gel and incubated for an hour. Gel debris was removed by spin X column and extracted RNA was ethanol precipitated. Extracted RNA from individual band was measured by Qubit3 fluorometer.

The extracted RNA from the different samples were quantified as follows: (Preparation A) approximately 1446 ng circular RNA and 176 ng linear RNA (89.1% circular RNA); (Preparation B) approximately 934 ng circular RNA and 270 ng linear RNA (77.5% circular RNA); and (Preparation C) approximately 320 ng circular RNA and 396 ng linear RNA (44.6% circular RNA).

### Example 4: Linear RNA in Circular RNA Preparations

This Example demonstrates production of 91% (w/w) pure circular RNA molecules relative to the total ribonucleotide molecules in the preparation and subsequent dosing in mice to generate a biological effect.

In this Example, circular RNAs included an IRES, an ORF encoding Gaussia Luciferase (GLuc), and two spacer elements flanking the IRES-ORF.

In this example, the circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA 5′-TTTTTCGGCTATTCCCAATAGCCGTTTTG-3′ and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNA storage solution (ThermoFisher Scientific, cat #AM7000).

Linear polyribonucleotides remained in the final circular RNA product preparation. The purity of circular RNA and percentage of remaining linear RNA in the final product preparation was quantified for each batch by running final product preparations on 6% TBE-urea gels and analyzing bands using ImageJ. Purity was assessed by calculating the intensity of circular RNA compared to the total RNA intensity and noted as a percentage. Here, circular RNA was of 91% (w/w) purity relative to the total RNA in the preparation.

Prior to administration, PBS and 10% TransIT carrier were added to achieve the desired final circular RNA concentration of 0.25 pmole in a 100 uL final volume. Mice received a single intravenous tail-vein injection of 0.25 pmole of the circular RNA encoding the Gaussia Luciferase ORF (100 uL).

Blood samples (˜25 uL) was collected from each mouse by submolar drawing. Blood was collected into EDTA tubes, at 0, 6 hours, 1, 2, 3, 7, 14, 21, 28 and 35 days post-dosing. Plasma was isolated by centrifugation for 30 minutes at 1300 g at 4□C and the activity of Gaussia Luciferase, a secreted enzyme, was tested using a Gaussia Luciferase activity assay (Thermo Scientific Pierce). Briefly, 50 uL of 1× GLuc substrate was added to 5 uL of plasma to carry out the GLuc luciferase activity assay. Plates were read immediately after mixing in a luminometer instrument (Promega).

Gaussia Luciferase activity was detected in plasma at 6 hours and 1, 2, 3, 7, 14, and 21 days post-dosing of circular RNA. Highest expression of circular RNA was observed approximately 2 days post-injection and high levels of expression were maintained for prolonged periods of time and was still detectable at 21 days. At all timepoints, these levels of activity were greater than those observed for the negative control (PBS vehicle only).

This Example demonstrates that circular RNA of 91% (w/w) purity relative to the total RNA in the preparation was successfully produced, successfully delivered via intravenous injection and was able to express protein detectable in blood for prolonged periods of time.

### Example 5: Quantification of Nicked Circular RNA in Gel-Purified Circular RNA

This Example demonstrates that circular RNA by purified by gel extraction contains no more than 1.1% (w/w) nicked RNA relative to the total RNA molecules in the preparation.

In this Example, RNAs included an IRES, an ORF encoding Gaussia Luciferase (GLuc), and two spacer elements flanking the IRES-ORF.

In this example, the circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH-treated linear RNA was circularized using a splint DNA (5′-TTTTTCGGCTATTCCCAATAGCCGTTTTG-3′) and T4 RNA ligase 2 (New England Biolabs, M0239). This results in circularization of the RNA at the ligation junction to generate circular RNA. Circular RNA was Urea-PAGE purified on a 4% PAGE gel, eluted in a buffer (0.5 M Sodium Acetate, 0.1% SDS, 1 mM EDTA), ethanol precipitated and resuspended in RNA storage solution (ThermoFisher Scientific, cat #AM7000). In this example, purified circular RNA was evaluated to have a purity of 80% (w/w) relative to the total RNA in the preparation.

In this example, the sequence of linear RNAs was assessed by next generation sequencing. The purified circular RNA preparation (80% purity) was prepared for the NGS pipeline using the library preparation method described in TruSeq Small RNA Workflow (Illumina, RS-200-0012). This method preserved 3′ end identity with high fidelity. Briefly, adapters were ligated to any RNA molecules with available 3′ or 5′ ends in the solution. Intact circular RNA will not undergo this ligation—as a result, this step selected for non-circular RNA only. These products are then reverse transcribed and amplified to generate cDNA libraries which were subsequently purified, quality-controlled and multiplexed. Libraries then underwent sequencing on an Illumina Miniseq machine.

In a similar manner to that described above, linear RNA product from in vitro transcription after RppH-treatment was processed for sequencing.

Sequencing results of both the linear RNA product of IVT and non-circular RNA in the gel-purified circular RNA preparation were compared by mapping reads back to the template sequence used to generate the circular RNA and evaluating the number of reads that map over the ligation junction.

In this analysis, the non-circular RNA that remains is assumed to be a mixture of nicked RNA and residual linear RNA product from IVT. In this example, if the non-circular RNA is assumed to comprise only nicked RNA, the percentage of fragments that map over the ligation junction is expected to be 50%. If the non-circular RNA is assumed to comprise only residual linear RNA product from IVT, the percentage of fragments that maps over the ligation junction is expected to be 0%. Using these statistical assumptions and control experiments, a standard curve was generated that enabled quantification of nicked RNA. This yielded a calculation of 5.4% of non-circular RNA as nicked RNA.

This Example demonstrates that 5.4% (w/w) of the non-circular RNA fraction of a purified circular RNA preparation (equivalent to 1.1% (w/w) of the total RNA) was nicked RNA.

### Example 6: Linear RNA Present in Circular RNA Preparations Affected Expression Levels and Persistence In Vitro

This Example demonstrates that the presence of linear RNA in a circular RNA preparation affects levels of protein expression and persistence of circular RNA in cells.

In this Example, the circular RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Gaussia luciferase (gluc) and two spacer elements flanking the IRES-ORF.

Splint ligated circular RNA was generated by treatment of the transcribed linear RNA and a DNA splint with T4 DNA ligase 2 (New England Biolabs, Inc., M0239).

Ligation mixtures were gel purified to remove template DNA, proteins, and linear (non-circularized) RNA. The RNA preparations were resolved on 4% denaturing PAGE and RNA bands corresponding to each of the circular RNAs were excised. Excised RNA gel fragments were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

Persistence of gel purified circular and unpurified RNA preparations during cell division was monitored in BJ fibroblast cells. Cells in a 96-well plate were suspension (reverse) transfected with equal amounts of either gel purified circular RNA or unpurified RNA preparations using a lipid-based transfection reagent (ThermoFisher Scientific (LMRNA003).

Gaussia Luciferase enzyme activity was monitored at 6 hrs and 1-5 days post-administration as protein expression measurements using a luciferase enzyme assay (Thermo Scientific Pierce, 16158) following manufacturer's instructions. In brief, 1× coelenterazine substrate was added to cell supernatants from the transfected wells. Plates were read immediately after substrate addition on a luminometer (Promega).

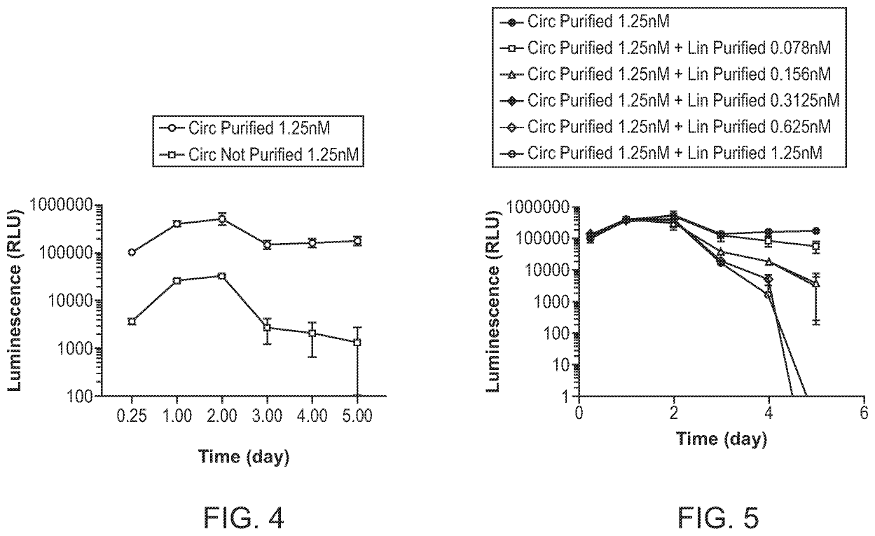

Protein expression from cells transfected with the gel purified circular RNA preparation was detected at higher levels and for longer periods of time than cells transfected with unpurified RNA (FIG. 4). About 100 fold higher luminescence (RLU) was detected with the purified circular RNA preparation as compared to the unpurified RNA over the course of 5 days.

### Example 7: Linear RNA Present in Circular RNA Preparations Affected Expression in a Dose Dependent Manner in Cells

This Example demonstrates that linear RNA in a circular RNA preparation negatively affected expression in a dose dependent manner.

In this Example, the circular RNA and linear RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Gaussia luciferase (gluc) and two spacer elements flanking the IRES-ORF.

Splint ligated circular RNA was generated by treatment of the transcribed linear RNA and a DNA splint with T4 DNA ligase 2 (New England Biolabs, Inc., M0239).

To purify the circular RNAs, ligation mixtures were resolved on 4% denaturing PAGE and RNA bands corresponding to each of the circular RNAs were excised. The linear RNAs were purified using the same 4% denaturing PAGE gel. Excised RNA gel fragments (linear or circular) were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

The impact of varying levels of linear RNA counterparts in preparations with gel purified circular RNA was determined by monitoring cell division in BJ fibroblast cells. Cells in a 96-well plate were suspension (reverse) transfected with either a gel purified circular RNA preparation or an equal amount of gel purified circular RNA preparation but supplemented with varying levels of linear RNA using a lipid-based transfection reagent (ThermoFisher Scientific (LMRNA003). Gaussia Luciferase enzyme activity was monitored at 6 hrs and 1-5 days post-administration as a measure of protein expression using a luciferase enzyme assay (Thermo Scientific Pierce, 16158) following manufacturer's instructions. In brief, 1× coelenterazine substrate was added to cell supernatants from the transfected wells. Plates were read immediately after substrate addition on a luminometer (Promega).

Protein expression from cells transfected with the gel purified circular RNA preparation alone was detected for longer periods of time than from cells transfected with the combined circular and linear RNAs, in a dose dependent manner (FIG. 5). The level of purified circular RNA alone remained stable over the time course of 120 hours, however, the level of RNA preparation with both circular and linear RNAs declined over the time, and the decline rate is proportional to the level of the linear RNAs. Surprisingly, even though the cells were transfected with equal amounts of circular RNA and that some of the samples contained twice as much coding RNA (circular combined with linear), increasing levels of linear RNA inversely affected protein expression levels over time, even while circular RNA amounts were kept constant.

This Example demonstrates that circular RNA with reduced levels of linear RNA affects has improved expression, e.g., improved longevity of expression.

### Example 8: Linear RNA in Circular RNA Preparations Affected Expression Levels and Time (Gel Imaging)

This Example demonstrates that the presence of linear RNA in a circular RNA preparation modifies levels of protein expression and persistence in vivo.

In this Example, the circular RNA was generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Nanoluciferase (Nluc) and two spacer elements flanking the IRES-ORF.

Splint ligated circular RNA was generated by treatment of the transcribed linear RNA and a DNA splint with T4 DNA ligase 2 (New England Biolabs, Inc., M0239).

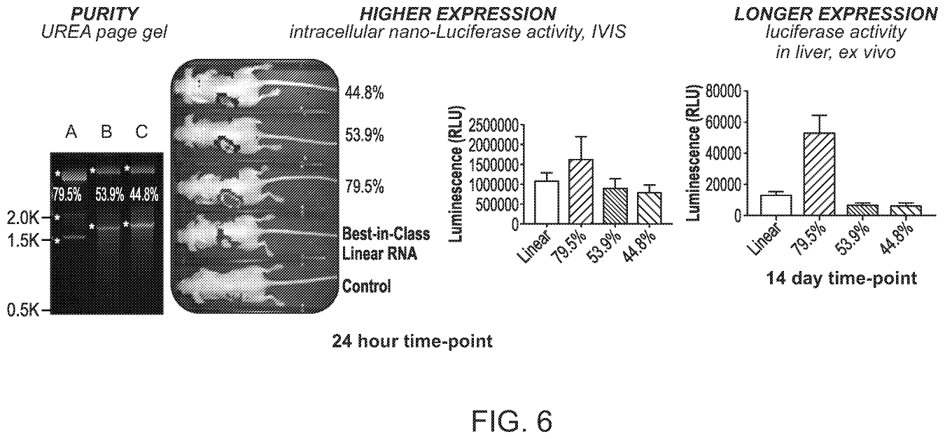

To purify the circular RNAs, ligation mixtures were resolved on 4% denaturing PAGE and RNA bands corresponding to each of the circular RNAs were excised. Excised RNA gel fragments were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol. Eluted circular RNA was analyzed by 6% denaturing PAGE. The gel was stained with SYBR-green and visualized by E-gel Imager. The band intensity on the visualized gel was measured and analyzed by ImageJ (FIG. 6).

RNA bands showing circular and linear RNA in the individual preparations were compared by E-gel imaging. Circular and linear RNA content was quantified by UREA PAGE gel analysis. In short, gels were analyzed for the relative amount of linear and circular RNA species in the individual preparations. Percentage of circular RNA content was calculated as follows: the amount of circular RNA was divided by the total RNA amount (circular+linear RNA). The percentage of circRNA in lane A was 79.5%, in lane B was 53.9%, and in lane C was 44.8%.

Balb/c mice were injected with preparations comprising circular RNA with the Nluc ORF, or linear RNA as a control, via intravenous (IV) tail vein administration. Animals received a single dose of 10 pmol of total RNA formulated in a lipid-based transfection reagent (Mirus) according to manufacturer's instructions.

24 hours after RNA administration, mice were injected with 40 ug furimazine (Promega, N1120; 20 ul substrate, 80 ul PBS/dose) IP and images were acquired after a ten-minute incubation using Bioluminescence Image Acquisition. At 14 days post-dosing animals were were injected with 40 ug of furimazine (Progemega, N1120, 20 ul substrate, 80 ul PBS/dose) intraperitoneal, sacrificed, and then livers were collected. The livers were imaged for 2 minutes immediately after harvest using Bioluminescence Image Acquisition. Bioluminescence Image Acquisition was used to measure the presence nano-luciferase expressed from linear and circular RNA. Images were analyzed using Living Image 4.3.1 (PerkinElmer, MA) software. Whole body fixed-volume ROIs were placed on prone and supine images for each individual animal, and labeled based on animal identification. Total flux (photons/sec) was calculated and exported for all ROIs to facilitate analyses between groups.

A preparation having 79.5% circular RNA showed higher expression in vivo at 24 hrs, compared to linear RNA or preparations with approximately 44.8% or approximately 53.9% circular RNA. Additionally, when luciferase expression from the higher percentage circular RNA preparations was analyzed ex vivo in liver at 14 days post administration, expression was maintained from the approximately 79.5% circular RNA preparation, but not from the approximately 44.8% or approximately 53.9% circular RNA preparations.

Thus, the presence of linear RNA in circular RNA preparations impacts expression and persistence in vivo.

### Example 9: Enrichment of Circular RNA from a Preparation Via Polyadenylation of Linear RNA Using Biotin-Labelled Adinoside and Subsequent Streptavidin-Mediated Capture

This example demonstrates enrichment of circular RNA from a preparation comprising a mixed pool of circular RNA and linear RNA counterpart containing the same nucleotide sequences.

Polyadenylation of RNA using poly(A) polymerase results in the addition of a 3′ polyadenine tail to RNA. This process requires the 3′ end of the RNA to be available for conjugation. Where RNA is circularized, no such free end exists. Poly(A) polymerase can also incorporate modified adenines such as the biotinylated N6-ATP analog. This enables pulldown of the biotinylated, polyadenylated linear RNA using a biotin-streptavidin binding system. Therefore, the biotinylated, polyadenylated linear RNA counterpart, including any fragments thereof such as a monoribonucleotide, can be captured in the pulldown using this biotin-streptavidin binding system.

In this example, circular RNA (1.2 kb in length) was generated from linear RNA produced by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. RppH treated linear RNA was circularized using a splint DNA and T4 RNA ligase 2 (New England Biolabs, M0239).

After ligation, a mix of circular RNA and linear RNA exist in solution with the splint DNA. DNAseI was used to digest the remaining splint using DNase I [Promega, M610A] with the provided reaction buffer (40 mM Tris-HCl, pH 8.0, 10 mM MgSO4, 10 mM CaCl2)). This reaction was incubated for 30 minutes at 37° C. RNA was isolated from reaction mixture via Monarch RNA Cleanup Kit [New England Biolabs, #T2040L].

For the polyadenylation reaction, 2.5 ug of post-ligation, post-DNaseI treated RNA was incubated with Yeast Poly(A) Polymerase [Thermo Fisher Scientific, 74225Z25KU] in the presence of an abundance of biotinylated N6-ATP analog [Jena Bioscience, NU-805-BIO], as well as the provided reaction buffer (100 mM Tris-HCl, pH 7.0, 3.0 mM MnCl2, 0.1 mM EDTA, 1 mM DTT, 500 μg/mL acetylated BSA, 50% glycerol). Reactions were incubated for 30 minutes at 37° C. RNA was isolated from the reaction mixture via Monarch RNA Cleanup Kit [New England Biolabs, #T2030L].

To remove polyadenylated linear RNA, 0.5 ug of RNA from the polyadenylation reaction was equilibrated by a one-to-one dilution with 2× binding/washing buffer (10 mM Tris-HCl, pH 7.5, 1 mM EDTA, 2M NaCl) and incubated with 5 uL of pre-equilibrated MyOne Streptavidin Dynabeads C1 [Thermo Fisher Scientific, 65001] bead slurry. The beads were separated from the supernatant via a two minute exposure to a magnetic scaffold. Supernatant was analyzed by A260 absorbance (Nanodrop) for RNA content and analyzed by 6% Urea PAGE for RNA ligation product enhancement. BioRad ImageLab was used to manually quantitate relative band intensity for all analyzed samples.

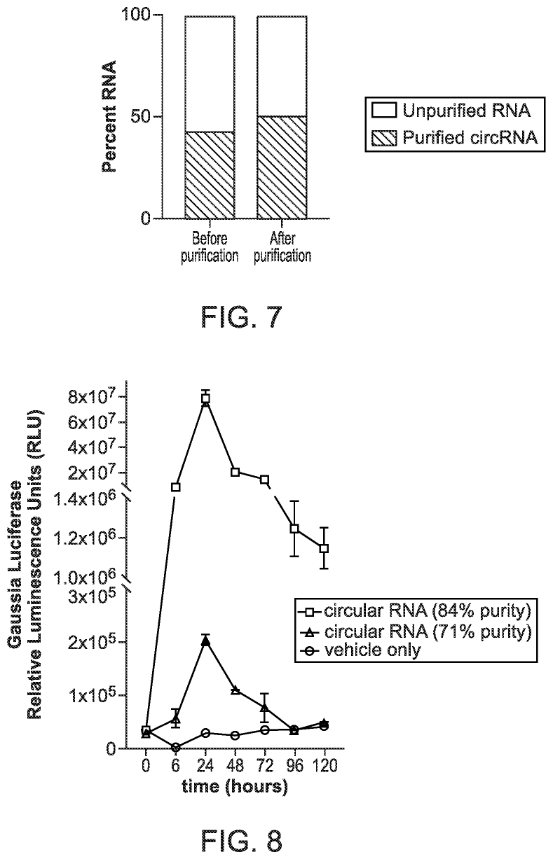

In this example, the ratio of circular RNA to linear RNA was calculated by measuring the band intensities in the 6% Urea PAGE gel. Quantification of the bands are shown in FIG. 7: before purification, 42.6% of the RNA product was circular RNA and 57.4% of the RNA product was linear RNA (unpurified RNA); after purification, 50.4% of the RNA product was circular RNA and 49.6% of the RNA product was linear RNA (purified circRNA). This example demonstrates an 8 percent increase in circular RNA in a mixed pool of circular RNA and linear RNA counterpart containing the same nucleotide sequences using polyadenylation of linear RNA with biotinylated adenine analogs followed by streptavidin-mediated pulldown of the linear RNA.

### Example 10: Reduction of Linear RNA Present in Circular RNA Preparations Increased Expression of the Encoded Protein

This Example demonstrates that reducing linear RNA present in a predominantly circular RNA composition increased expression of the encoded protein in cells.

For this Example, circular RNAs included an IRES, an ORF encoding Gaussia Luciferase (GLuc), and two spacer elements flanking the IRES-ORF.

Two batches of circular RNA were generated. In each case, the circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′phosphohydrolase (RppH) (New England Biolabs, M0356) following the manufacturer's instructions, and purified again with an RNA purification column. RppH treated linear RNA was circularized using a splint DNA 5′-GGCTATTCCCAATAGCCGTT-3′ and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated, and resuspended in RNA storage solution (ThermoFisher Scientific, cat #AM7000).

Linear RNA remained in the final circular RNA product. The purity of circular RNA and percentage of remaining linear RNA in the final product was quantified for each batch by running final products on 6% TBE-urea gels and analyzing bands using ImageJ. Purity of circRNA was assessed by calculating the intensity of circRNA compared to the total RNA intensity and noted as a percentage. Here, batches were of 71% purity and 84% purity.

0.2 pmoles of each RNA batch was used for cell transfections. RNA was combined with Optimem and Messenger Max according to the manufacturer's recommendations. A vehicle only control was similarly prepared but did not contain any RNA. At time=0, each preparation was added to BJ fibroblast cells.

The activity of Gaussia Luciferase was tested using a Gaussia Luciferase Activity assay (Thermo Scientific Pierce). Samples of 20 μL of the cell supernatant were added to a 96 well plate (Corning 3990). Samples were taken at 6, 24, 48, 72, 96 and 120 hours after transfection. In brief, 1× coelenterazine substrate was added to each well. Plates were read immediately after substrate addition and mixing in a luminometer instrument (Promega).

Gaussia Luciferase activity was detected in cells in experiments using circular RNA of 84% purity at 6, 24, 48, 72, 96 and 120 hours post-transfection (FIG. 8) and was higher than the expression afforded by the vehicle only control. Expression derived from circular RNA of 84% purity was significantly higher than the expression derived from circular RNA of 71% purity. At 24 hours post-transfection, GLuc activity was 397-fold greater when circular RNA of 84% purity was transfected compared to when circular RNA of 71% purity was transfected. GLuc activity was detected in cells in experiments using circular RNA of 71% purity at 6, 24, 48 and 72 hours post-transfection (FIG. 8) and was higher than the expression afforded by the vehicle only control.

This Example demonstrated that circular RNA of greater purity (with reduced linear RNA) increases and prolongs expression of the encoded protein.

### Example 11: Linear RNA Present in Circular RNA Preparations Affected Circular RNA Stability in a Dose Dependent Manner in Cells

This Example demonstrates that linear RNA presence in a circular RNA preparation negatively affected circular RNA stability in a dose dependent manner.

In this Example, the circular RNA and linear RNA were generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Circular RNAs were designed to include an IRES with an ORF encoding Gaussia luciferase (GLuc) and two spacer elements flanking the IRES-ORF.

Splint ligated circular RNA was generated by treatment of the transcribed linear RNA and a DNA splint with T4 RNA ligase 2 (New England Biolabs, Inc., M0239).

To purify the circular RNAs, ligation mixtures were resolved on 4% denaturing PAGE and RNA bands corresponding to each of the circular and linear RNAs were excised. Excised RNA gel fragments (linear or circular) were crushed, and RNA was eluted with gel elution buffer (0.5 M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

The impact of varying levels of linear RNA counterparts in preparations of gel purified circular RNA was determined by monitoring circular RNA levels in BJ fibroblast cells. Cells in a 96-well plate were transfected with either a gel purified circular RNA preparation, or an equal amount of gel purified circular RNA preparation supplemented with varying levels of gel-purified linear RNA using a lipid-based transfection reagent (ThermoFisher Scientific (LMRNA003). Circular RNA levels were analyzed by circRNA specific Q-PCR at 6 hrs and 1-5 days post-transfection. In brief, cDNA was generated from cell lysates by random priming using the Power SYBR Green cells to ct kit (ThermoFisher Scientific, cat #4402953) and following manufacturer's instructions. Q-PCR was performed using outward primers design to only amplify the circRNA and not its linear counterpart, and fold-change was calculated using the Pfaffl method using β-Actin as the housekeeping gene.

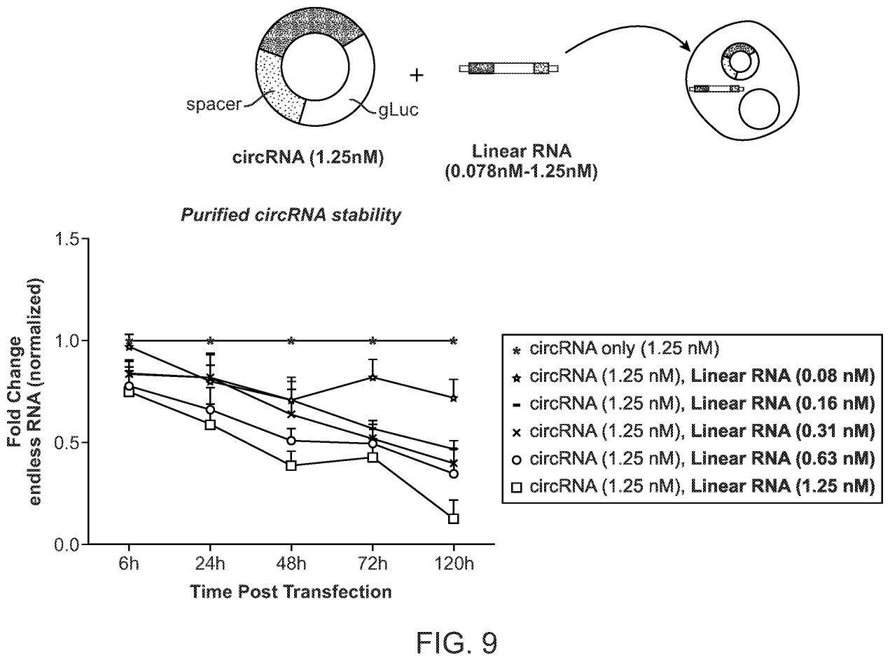

Circular RNA was detected in higher amounts for longer periods of time in cells transfected with the gel purified circular RNA preparation alone, compared to cells transfected with both the combined circular and linear RNAs, in a dose dependent manner (FIG. 9). The level of purified circular RNA alone remained stable over the time course of 120 hours; however, the level of circular RNA detected in cells transfected with a combination of both circular and linear RNAs declined over the time, and the decline rate was proportional to the level of the linear RNAs. Surprisingly, even though the cells were transfected with equal amounts of circular RNA, linear RNA presence inversely affected circular RNA levels over time.

This Example demonstrates that purification of circular RNA from linear RNA affects circular RNA stability.

### Example 12: Linear RNA Present in Circular RNA Preparations Affected Innate Immune Response in a Dose Dependent Manner in Cells

This Example demonstrates that linear RNA present in a circular RNA preparation negatively affected innate immune response in a dose dependent manner.

In this Example, circular RNA and linear RNA were generated as follows. Unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification lsystem. Circular RNAs were designed to include an TRES with an ORF encoding Gaussia luciferase (GLuc) and two spacer elements flanking the IRES-ORF.

Splint ligated circular RNA was generated by treatment of the transcribed linear RNA and a DNA splint with T4 RNA ligase 2 (New England Biolabs, Inc., M0239).

To purify the circular RNAs, ligation mixtures were resolved on 4% denaturing PAGE and RNA bands corresponding to each of the circular and linear RNAs were excised. The linear RNAs were purified using the same 4% denaturing PAGE gel. Excised RNA gel fragments (linear or circular) were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

The impact of varying levels of linear RNA counterparts in preparations of gel purified circular RNA was determined by monitoring circular RNA levels in BJ fibroblast cells. Cells in a 96-well plate were suspension (reverse) transfected with either a gel purified circular RNA preparation, or an equal amount of gel purified circular RNA preparation but supplemented with varying levels of gel purified linear RNA using a lipid-based transfection reagent (ThermoFisher Scientific (LMRNA003). Immune genes levels were analyzed by Q-PCR at 6 hrs and 1-5 days post-transfection. In brief, cDNA was generated from cell lysates by random priming using the Power SYBR Green cells to ct kit (ThermoFisher Scientific, cat #4402953) and following manufacturer's instructions. Q-PCR was performed using immune gene specific primers, and relative RNA levels were calculated using the Pfaffl method and β-Actin as the housekeeping gene.

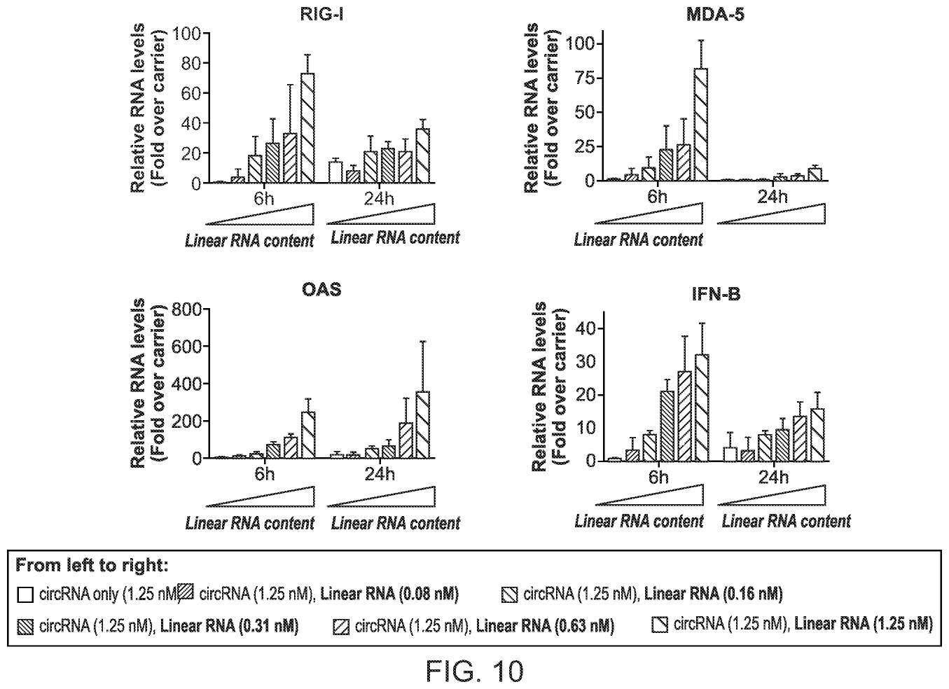

Circular RNA in cells transfected with the gel purified circular RNA preparation alone showed limited increased expression of innate immune genes. Conversely cells transfected with the combined circular and linear RNAs, demonstrated upregulation of innate immune genes in a dose dependent manner (FIG. 10).

### Example 13: Non-Immunogenicity in Cell Culture

This example demonstrates in vivo assessment of immunogenicity of the circular RNA after cell transfection.

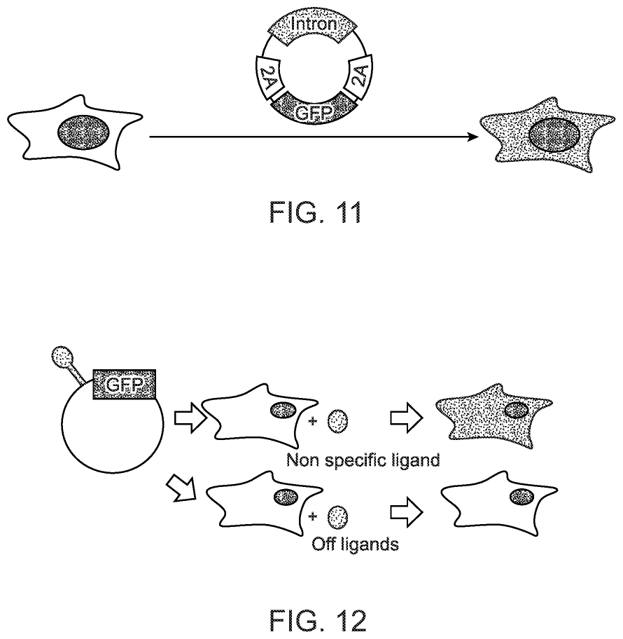

In this Example, circular RNAs designed to include an encryptogen, e.g., a ZKSCAN1 intron and a GFP ORF. In addition, control circular RNA is designed to include a GFP ORF with and without introns, see FIG. 11. The circular RNA is generated either in vitro or in cells as described in Example 1 and 2. HeLa cells are transfected with 500 ng of circular RNAs.

Transfection of the circular RNA include the following conditions: (1) naked circular RNA in cell culture media (Lingor et al 2004); (2) electroporation (Muller et al 2015); (3) cationic lipids (SNALP, Vaxfectin) (Chesnoy and Huang, 2000); (3) cationic polymers (PEI, polybrene, DEAE-dextran) (Turbofect); (4) virus-like particles (L1 from HPV, VP1 from polyomavirus) (Tonges et al 2006); (5) exosomes (Exo-Fect from SBI); (6) nanostructured calcium phosphate (nanoCaP)(Olton et al 2006); (6) peptide transduction domains (TAT, polyR, SP, pVEC, SynB1, etc) (Zhang et al 2009); (7) vesicles (VSV-G, TAMEL) (Liu et al 2017); (8) cell squeezing; (SQZ Biotechnologies) (9) nanoparticles (Neuhaus et al 2016); and/or (10) magnetofection (Mair et al 2009). Transfection methods are performed in cell culture media (DMEM 10% FBS) and cells are subsequently cultured for 24-48 hrs.

After 2-48 hrs post-transfection, media is removed and relative expression of the indicated RNA and transfected RNA is measured by qRT-PCR.

For qRT-PCR analysis, total RNA is isolated from cells using a phenol based RNA isolation solution (TRIzol) and an RNA isolation kit (QIAGEN) following the manufacturer's instructions. qRT-PCR analysis is performed in triplicate using a PCR master mix (Brilliant II SYBR Green qRT-PCR Master Mix) and a PCR cycler (LightCycler 480). mRNA levels for well-known innate immunity regulators such as RIG-I, MDA5, OAS, OASL, and PKR are quantified and normalized to actin, GAPDH, or HPRT values. Relative expression of indicated RNA genes for circular RNA transfection are normalized by level of transfected RNA and compared to the expression level of cells with circular RNA transfection that does not contain encryptogen(s).

In addition to qRT-PCR analysis, western blot analysis and immuno-histochemistry are used, as described above in Example 6, to assess GFP expression efficiency.

It is expected that GFP positive cells containing encryptogen(s) will show an attenuated immunogenicity response.

In addition, (1) primary murine dendritic cells; (2) Human embryonic kidney 293 cells stabile expressing TLR-7, 8 or 9 (InvivoGen); (3) monocyte derived dendritic cells (AllCells) or (4) Raw 264.7 cells are transfected with a DNA plasmid including ZKSCAN1 or td introns that produce a circular RNA encoding GFP as described above. After 6-48 hrs post-transfection, cell culture supernatant is collected and cytokine expression is measured using ELISA. When cell culture supernatant is collected, cells are collected for Northern blot, gene expression array and FACS analysis.

For ELISA, ELISA kits for interferon-β (IFN-β), chemokine (C—C motif) ligand 5 (CCL5), IL-12 (BD Biosciences), IFN-α, TNF-α and IL-8 (Biosource International) are used. ELISAs are performed according to the manufacturers' recommendations. Expression of indicated cytokines for circular RNA transfected cells are compared to the level of control RNA transfected cells. It is expected that cells transfected with circular RNA with an encryptogen will have reduced cytokine expression compared to control transfected cells.

For Northern blot analysis. Samples are processed and analyzed as previously described. Probes are derived from plasmids and are specific for the coding regions of human IFN-alpha 13, IFN-beta (Open Biosystems), TNF-alpha, or GAPDH (ATCC). It is expected that cells transfected with circular RNA with an encryptogen will have reduced cytokine expression compared to control transfected cells.

For the gene expression array, RNA is isolated using a phenol based solution (TRIzol) and/or an RNA isolation kit (RNeasy Qiagen). RNA is amplified and analyzed (e.g. Illumina Human HT12v4 chip in an Illumina BeadStation 500GX). Levels in mock control treated cells are used as the baseline for the calculation of fold increase. It is expected that cells transfected with circular RNA with an encryptogen will have reduced cytokine expression compared to control transfected cells.

For FACS analysis, cells are stained with a directly conjugated antibodies against CD83 (Research Diagnostics Inc), HLA-DR, CD80 or CD86 and analyzed on a flow cytometer. It is expected that cells transfected with circular RNA with an encryptogen will show reduced expression of these markers compared to control transfected cells.

### Example 14: Riboswitches for Selective Expression

This example demonstrates the ability to control protein expression from circular RNA in vivo.

For this Example, circular RNAs are designed to include encryptogen(s) (SEQ ID NO:4), a synthetic riboswitch (SEQ ID NO: 9) regulating the expression of the ORF encoding GFP (SEQ ID NO:2) with stagger elements (2A sequences) (SEQ ID NO:3) flanking the GFP ORF, see FIG. 12. The circular RNA is generated either in vitro or in cells as described in Example 1 and 2 and their corresponding figures described in [0356]-[0365] of International Patent Publication No. WO2019118919A1, are incorporated herein by reference in their entirety.

Theophylline induces activation of the riboswitch, resulting in an off-switch of gene expression (as described by Auslander et al 2010). It is expected that the riboswitch controls GFP expression from the circular RNA. In the presence of theophylline, no GFP expression is expected to be observed.

HeLa cells are transfected with 500 ng of the described circular RNA encoding GFP under the control of the theophylline dependent synthetic riboswitch (SEQ ID NO:9) to assess selective expression. Transfection methods are described in Example 7.

After 24 hr of culture at 37° C. and 5% CO2, cells are treated with and without theophylline with concentrations ranging from 1 nM-3 mM. After 24 hrs of continuous culture, cells are fixed in 4% paraformaldehyde for 15 minutes at room temperature, blocked and permeabilized for 45 minutes with 10% FBS in PBS with 0.2% detergent. Samples are then incubated with primary antibodies against GFP (Invitrogen) and secondary antibodies conjugated with Alexa 488 and DAPI (Invitrogen) in PBS with 10% FBS and 0.1% detergent for 2 hrs at room temperature or overnight at 4° C. Cells are then washed with PBS and subsequently analyzed using a fluorescent microscope for GFP expression.

### Example 15: Non-Immunogenicity In Vivo

This example demonstrates in vivo assessment of immunogenicity of the circular RNA after cell transfection.

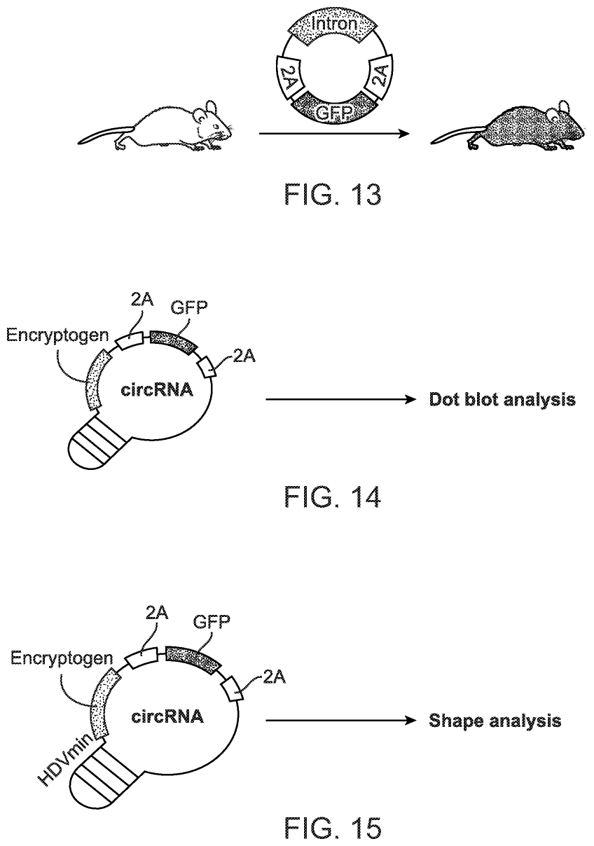

This Example describes quantification and comparison of the immune response after administrations of circular RNA harboring an encryptogen (the intron in this case), see FIG. 13. In an embodiment, any of the circular RNA with an encryptogen, will have a reduced (e.g., reduced compared to administration of control RNA) immunogenic response following one or more administrations of the circular RNA compared to control.

A measure of immunogenicity for circular RNA are the cytokine levels in serum.

In this Example, cytokine serum levels are examined after one or more administrations of circular RNA. Circular RNA from any one of the previous Examples is administered via intradermal (ID), intramuscular (IM), oral (PO), intraperitoneal (IP), or intravenous (IV) into BALB/c mice 6-8 weeks old. Serum is drawn from the different cohorts: mice injected systemically and/or locally with injection(s) of circular RNA harboring an encryptogen and circular RNA without an encryptogen.

Collected serum samples are diluted 1-10× in PBS and analyzed for mouse IFN-α by enzyme-linked immunosorbent assay (PBL Biomedical Labs, Piscataway, N.J.) and TNF-α (R&D, Minneapolis, Minn.).

In addition to cytokine levels in serum, expression of inflammatory markers is another measure of immunogenicity. In this Example, spleen tissue from mice treated with vehicle (no circular RNA), linear RNA, or circular RNA will be harvested 1, 4, and 24 hours post administration. Samples will be analyzed using the following techniques qRT-PCR analysis, Northern blot or FACS analysis.

For qRT-PCR analysis mRNA levels for RIG-I, MDA5, OAS, OASL, TNF-alpha and PKR are quantified as described previously.

For Northern blot analysis. Samples are processed and analyzed for IFN-alpha 13, IFN-beta (Open Biosystems), TNF-alpha, or GAPDH (ATCC) as described above.

For FACS analysis, cells are stained with a directly conjugated antibodies against CD83 (Research Diagnostics Inc), HLA-DR, CD80 or CD86 and analyzed on a flow cytometer.

In an embodiment, circular RNA with an encryptogen will have decreased cytokine levels (as measured by ELISA, Northern blot, FACS and/or qRT-PCR) after one or multiple administrations, as compared control RNA.

### Example 16: Circular RNA Includes at Least One Double-Stranded RNA Segment

This example demonstrates that circular RNA includes at least one double-stranded RNA segment.

In this Example, circular RNA is synthesized through one of the methods described previously, to include a GFP ORF and an IRES, see FIG. 14. Dot blot assays with J2 and K1 monoclonal antibodies will be utilized to measure double stranded RNA structures of at least 40 bp in length. Circular RNA (200 ng) is blotted onto a nylon membrane (super charged Nytran), dried, blocked with 5% non-fat dried milk in TBS-T buffer (50 mM Tris-HCl, 150 mM NaCl, 0.05% Tween-20, pH 7.4), and incubated with dsRNA-specific mAb J2 or K1 (English & Scientific Consulting) for 60 min. Membranes are washed six times with TBS-T then treated with HRP-conjugated donkey anti-mouse Ig (Jackson Immunology), then washed six times and dots are visualized with an enhanced chemiluminescence western blot detection reagent (Amersham).

It is expected that a circular RNA creates an internal quasi-double stranded RNA segment.

### Example 17: Circular RNA Includes a Quasi-Double-Stranded Structure

This example demonstrates that circular RNA includes a quasi-double-stranded structure.

In this Example, circular RNA is synthesized through one of the methods described previously, with and without addition of the expression of HDVmin (Griffin et al 2014). This RNA sequence forms a quasi-helical structure, see FIG. 15, and is used as a positive control (as shown by Griffin et al 2014).

To test if circular RNA structure includes a functional quasi-double-stranded structure we will determine the secondary structure using selective 2′OH acylation analyzed by primer extension (SHAPE). SHAPE assesses local backbone flexibility in RNA at single-nucleotide resolution. The reactivity of base positions to the SHAPE electrophile is related to secondary structure: base-paired positions are weakly reactive, while unpaired positions are more highly reactive.

SHAPE is performed on circular RNA, HDVmin, and linear RNA containing. SHAPE is performed with N-methylisatoic anhydride (NMIA) or benzoyl cyanide (BzCN) essentially as reported by Wilkinson et al 2006 and Griffin 2014 et al, respectively. In brief for SHAPE with BzCN, 1 ul of 800 mM BzCN in dimethyl sulfoxide (DMSO) is added to a 20 ul reaction mixture containing 3 to 6 pmol of RNA in 160 mM Tris, pH 8.0, 1 U/l RNAse inhibitor (e.g. Superaseln RNase inhibitor) and incubated for 1 min at 37° C. Control reaction mixtures include 1 ul DMSO without BzCN. After incubation with BzCN, RNAs is extracted with phenol chloroform, and purified (e.g using a RNA Clean & Concentrator-5 kit) as directed by the manufacturer, and resuspended in 6 ul 10 mM Tris, pH 8.0. A one-dye system is used to detect BzCN adducts. RNAs are annealed with a primer labeled with 6-carboxyfluorescein (6-FAM). Primer extension is performed using a reverse transcriptase (SuperScript III—Invitrogen) according to the manufacturer's recommendations with the following modifications to the incubation conditions: 5 min at 42° C., 30 min at 55° C., 25 min at 65° C., and 15 min at 75° C. Two sequencing ladders are generated using either 0.5 mM ddATP or 0.5 mM ddCTP in the primer extension reaction. Primer extension products are precipitated with ethanol, washed to remove excess salt, and resolved by capillary electrophoresis along with a commercial size standard (e.g. Liz size standard Genewiz Fragment Analysis Service).

Raw electropherograms are analyzed using a primary fragment analysis tool (e.g. PeakScanner Applied Bio-systems). The peaks at each position in the electropherogram are then integrated. For each RNA analyzed, y axis scaling to correct for loading error is performed so that the background for each primer extension reaction is normalized to that of a negative-control reaction performed on RNA that is not treated with BzCN. A signal decay correction is applied to the data for each reaction. The peaks are aligned to a ladder created from two sequencing reactions. At each position, the peak area of the negative control is subtracted from the peak area in BzCN-treated samples; these values are then converted to normalized SHAPE reactivities by dividing the subtracted peak areas by the average of the highest 2% to 10% of the subtracted peak areas.

In addition to SHAPE analysis we will perform NMR (Marchanka et al 2015); Hydroxyl radical probing (Ding et al 2012); or a combination of DMS and CMTC and Kethoxal (Tijerina et al 2007 and Ziehler et al 2001).

It is expected that a circular RNA will have a quasi-double-stranded structure.

### Example 18: Circular RNA Includes a Functional Quasi-Helical Structure

This example demonstrates that circular RNA includes a functional quasi-helical structure.

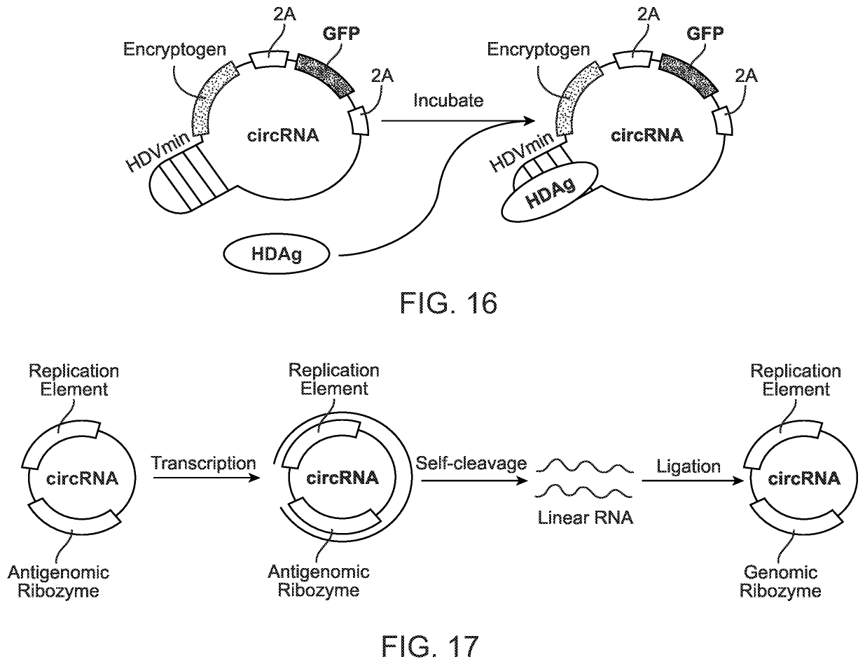

In this Example, circular RNA is synthesized through one of the methods described previously, with the addition of the expression of 395 L (Defenbaugh et al 2009). This RNA sequence forms a quasi-helical structure (as shown above, by RNA secondary structure folding algorithm mfold and Defenbaugh et al 2009), see FIG. 16. This structure is essential for complex formation with hepatitis D antigen (HDAg).

Therefore, to test if circular RNA structure includes a functional quasi-structure we will incubate circular RNA and linear RNA with HDAg-160 or HDAg-195 and analyze binding using EMSA assays. Binding reactions are done in 25 ul including 10 mM Tris-HCl (pH 7.0), 25 mM KCl, 10 mM NaCl, 0.1 g/l bovine serum albumin (New England Biolabs), 5% glycerol, 0.5 mM DTT, 0.2 U/l RNase inhibitor (Applied Biosystems), and 1 mM phenylmethylsulfonyl fluoride solution. circular RNA is incubated with HDAg protein (obtained as described by Defenbaugh et al 2009) at concentrations ranging from 0-110 nM. Reaction mixtures are assembled on ice, incubated at 37° C. for 1 h, and electrophoresed on 6% native polyacrylamide gels in 0.5 Tris-borate-EDTA at 240 V for 2.5 h. Levels of free and bound RNA are determined using nucleic acid staining (e.g., gelred). Binding will be calculated as the intensity of unbound RNA relative to the intensity of the entire lane minus the background.

It is expected that a circular RNA will have a functional quasi-helical structure.

### Example 19: Self-Transcription/Replication

In this Example, circular RNA is synthesized through one of the methods described previously, with the addition of the expression of the HDV replication domain(s) (as described by Beeharry et al 2014), the antigenomic replication competent ribozyme and a nuclear localization signal. These RNA sequences allow for circular RNA to be located in the nucleus where the host RNA polymerase will bind and transcribe the RNA. Then this RNA is self-cleaved using the ribozyme. RNA is then ligated and self-replicated again, see FIG. 17.

Circular RNA (1-5 microgram) will be transfected into HeLa cells using techniques described above. HeLa cells are grown at 37° C. and 5% CO₂ in Dulbecco's modified Eagle's medium (DMEM) with high glucose (Life Technologies), supplemented with penicillin-streptomycin and 10% fetal bovine serum. After transfection HeLa cells are cultured for an additional 4-72 hr, then total RNA from the transfected cells is isolated using a phenol-based RNA isolation reagent (Life Technologies) as per the manufacturer's instructions between 1 hour and 20 days after transfection and total amount of circular RNA encoding the HDV domains will be determined and compared to control circular RNA using qPCR as described herein.

### Example 20: Circular RNA Preservation in Daughter Cells

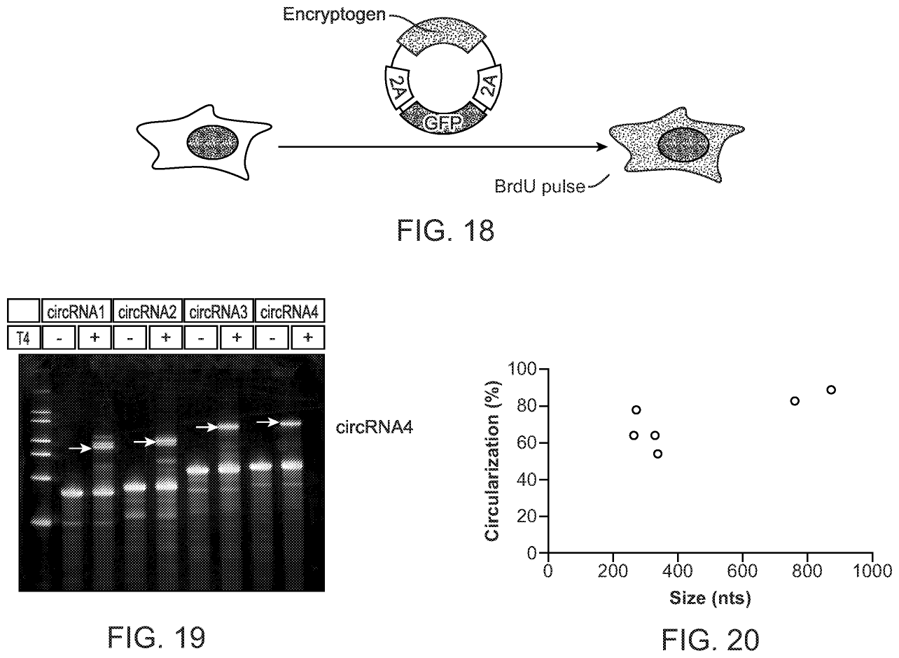

In this Example, circular RNA is synthesized through one of the methods described previously. A circular RNA is designed to include encryptogens (SEQ ID NO:4) and an ORF encoding GFP (SEQ ID NO: 2) with stagger elements (SEQ ID NO: 3) flanking the GFP ORF, see FIG. 18.

Human Fibroblasts (e.g. IMR-90) are grown in Dulbecco's modified Eagle's medium (DMEM; Invitrogen) supplemented with 10% fetal bovine serum (FBS; Invitrogen) at 37° C. under 5% CO2 on tissue culture treated plates. Cells are passaged regularly to maintain exponential growth. Lipid transfection reagent (2 μL; Invitrogen) is added to a mixture of 1 μg circular RNA or linear RNA (described above) and 145 μL reduced serum medium (Opti-MEM I solution) in one well of a 12-well tissue culture treated plate. After incubation at room temperature for 15 min, 1×10\hat{ }5 HeLa cells suspended in DMEM with 10% FBS are added to the circular RNA solution (described above). After incubation for 24 h at 37° C. and 5% CO2, the cells are pulsed with BrdU (e.g. Sigma-Aldrich). BrdU, labeling duration is optimized for each cell type according to their specific population doubling time, e.g. IMR-90 human fibroblasts have a doubling time of 27 hrs and are pulsed for 8-9 hrs as described by Elabd et al 2013.

Cells will be collected at day 1, 2, 3, 4, 5 and 10 after BrdU pulse. A subset of the cells will be isolated q-rt-PCR and another subset for FACS analysis. To measure GFP circular RNA and mRNA levels, qPCR reverse transcription using random hexamers is performed, as described in described in Example 2 and its corresponding figure described in [0360]-[0365] of International Patent Publication No. WO2019118919A1, are incorporated herein by reference in their entirety. Cells will be analyzed with FACS using BrdU and GFP antibodies as described herein.

It is expected that circular RNA will persist in daughter cells and that daughter cells will express GFP protein.

### Example 21: Circular RNA Circularization

This Example demonstrates in vitro production of circular RNA using splint ligation.

A non-naturally occurring circular RNA can be engineered to include one or more desirable properties and may be produced using recombinant DNA technology. As shown in the following Example, splint ligation circularized linear RNA.

CircRNA1 was designed to encode triple FLAG tagged EGF without stop codon (264 nts). It has a Kozak sequence (SEQ ID NO: 11) at the start codon for translation initiation. CirRNA2 has identical sequences with circular RNA1 except it has a termination element (triple stop codons) (273 nts, SEQ ID NO: 12). Circular RNA3 was designed to encode triple FLAG tagged EGF flanked by a stagger element (2A sequence, SEQ ID NO: 13), without a termination element (stop codon) (330 nts). CircRNA4 has identical sequences with circular RNA3 except it has a termination element (triple stop codon) (339 nts).

In this example, the circular RNA was generated as follows. DNA templates for in vitro transcription were amplified from gBlocks gene fragment with corresponding sequences (IDT) with T7 promoter-harboring forward primer and 2-O-methylated nucleotide with a reverse primer. Amplified DNA templates were gel-purified with a DNA gel purification kit (Qiagen). 250-500 ng of purified DNA template was subjected to in vitro transcription. Linear, 5′-mono phosphorylated in vitro transcripts were generated using T7 RNA polymerase from each DNA template having corresponding sequences in the presence of 7.5 mM GMP, 1.5 mM GTP, 7.5 mM UTP, 7.5 mM CTP and 7.5 mM ATP. Around 40 μg of linear RNA was generated in each reaction. After incubation, each reaction was treated with DNase to remove the DNA template. The in vitro transcribed RNA was precipitated with ethanol in the presence of 2.5M ammonium acetate to remove unincorporated monomers.

Transcribed linear RNA was circularized using T4 RNA ligase 2 on a 20 nt splint DNA oligomer (SEQ ID NO: 14) as template. Splint DNA was designed to anneal 10 nt of each 5′ or 3′end of linear RNA. After annealing with the splint DNA (3 μM), 1 μM linear RNA was incubated with 0.5 U/μl T4 RNA ligase 2 at 37 C or 4 hr. Mixture without T4 RNA ligase 2 was used as the negative control.

The circularization of linear RNA was monitored by separating RNA on 6% denaturing PAGE. Slower migrating RNA bands correspond with circular RNA rather than linear RNA on denaturing polyacrylamide gels because of their circular structure. As seen in FIG. 19, the addition of ligase (+lanes) to the RNA mixtures generated new bands to appear above the linear RNA bands that were present in the mixtures that lacked ligase (−lanes). Slower migrating bands appeared in all RNA mixtures indicating successful splint ligation (e.g., circularization) occurred with multiple constructs as compared to negative control.

### Example 22: RNA Circularization Efficiency

This Example demonstrates circularization efficiencies of RNA splint ligation.

A non-naturally occurring circular RNA engineered to include one or more desirable properties may be produced using splint mediated circularization. As shown in the following Example, splint ligation circularized linear RNA with higher efficiency than controls.

CircRNA1, CircRNA2, CircRNA3, and CircRNA4 as described in Example 21 were also used here. CircRNA5 was designed to encode FLAG tagged EGF flanked by a 2A sequence and followed by FLAG tagged nano luciferase (873 nts, SEQ ID NO: 17). CircRNA6 has identical sequence with circular RNA5 except it included a a termination element (triple stop codon) between the EGF and nano luciferase genes, and a termination element (triple stop codon) at the end of the nano luciferase sequence (762 nts, SEQ ID NO: 18).

In this Example, to measure circularization efficiency of RNA, 6 different sizes of linear RNA (264 nts, 273 nts, 330 nts, 339 nts, 873 nts and 762 nts) were generated and circularized as described in Example 1. The circular RNAs were resolved by 6% denaturing PAGE and corresponding RNA bands on the gel for linear or circular RNA were excised for purification. Excised RNA gel bands were crushed and RNA was eluted with 800 μl of 300 mM NaCl overnight. Gel debris was removed by centrifuge filters and RNA was precipitated with ethanol in the presence of 0.3M sodium acetate.

Circularization efficiency was calculated as follows. The amount of eluted circular RNA was divided by the total eluted RNA amount (circular+linear RNA) and the result was depicted as a graph in FIG. 20.

Ligation of linear RNAs using T4 RNAse ligase 2 produced circular RNA at efficiency rates higher than control. Trending data indicated larger constructs circularized at higher rates, for instance, linear RNAs having around 800 nts were shown to have circularization efficiency around 80%, while linear RNAs having around 200-400 nts had circularization efficiency in the range of 50% to 80%.

### Example 23: Circular RNA Lacking Degradation Susceptibility

This Example demonstrates circular RNA susceptibility to degradation by RNase R compared to linear RNA.

Circular RNA is more resistant to exonuclease degradation than linear RNA due to the lack of 5′ and 3′ ends. As shown in the following Example, circular RNA was less susceptible to degradation than its linear RNA counterpart.

CircRNA5 was generated and circularized as described in Example 22 for use in the assay described herein.

To test circularization of CircRNA5, 20 ng/μl of linear or CircRNA5 was incubated with 2 U/μl of RNAse R, a 3′ to 5′ exoribonuclease that digests linear RNAs but does not digest lariat or circular RNA structures, at 37° C. for 30 min. After incubation, the reaction mixture was analyzed by 6% denaturing PAGE.

The linear RNA bands present in the lanes lacking exonuclease were absent in the CircRNA5 lane (see FIG. 21) indicating CircRNA5 showed higher resistance to exonuclease treatment as compared to linear RNA control.

### Example 24: Isolation and Purification of Circular RNA

This Example demonstrates circular RNA purification using UREA gel separation.

CircRNA1, CircRNA2, CircRNA3, CircRNA4, CircRNA5, and CircRNA6, as described in Examples 19 and 20, were isolated as described herein.

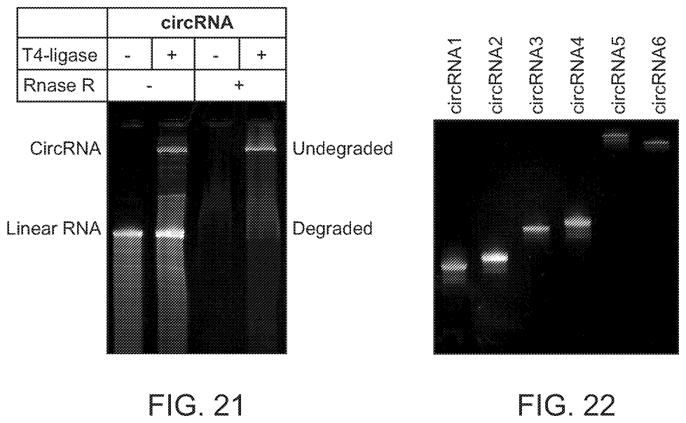

In this Example, linear and circular RNA were generated as described. To purify the circular RNAs, ligation mixtures were resolved on 6% denaturing PAGE and RNA bands corresponding to each of the circular RNAs were excised. Excised RNA gel fragments were crushed and RNA was eluted with 800 μl of 300 mM NaCl overnight. Gel debris was removed by centrifuge filters and RNA was precipitated with ethanol in the presence of 0.3M sodium acetate. Eluted circular RNA was analyzed by 6% denaturing PAGE, see FIG. 22.

Single bands were visualized by PAGE for the circular RNAs having variable sizes.

### Example 25: Detection of Protein Expression

This Example demonstrates in vitro protein expression from a circular RNA.

Protein expression is the process of generating a specific protein from mRNA. This process includes the transcription of DNA into messenger RNA (mRNA), followed by the translation of mRNA into polypeptide chains, which are ultimately folded into functional proteins and may be targeted to specific subcellular or extracellular locations.

As shown in the following Example, a protein was expressed in vitro from a circular RNA sequence.

Circular RNA was designed to encode triple FLAG tagged EGF flanked by a 2A sequence without a termination element (stop codon) (330 nts, SEQ ID NO: 19).

Linear or circular RNA was incubated for 5 hr in rabbit reticulocyte lysate at 30° C. in a volume of 25 μl. The final composition of the reaction mixture contained 70% rabbit reticulocyte lysate, 20 μM amino acids, 0.8 U/μl RNase inhibitor and 1 μg of linear or circular RNA. After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 30° l of 2×SDS sample buffer and incubated at 70° C. for 15 min. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit (see FIG. 23) and western blot band intensity was measured by ImageJ.

Fluorescence was detected, indicating expression product was present. Thus, circular RNA was shown to drive expression of a protein.

### Example 26: IRES-Independent Expression

This Example demonstrates circular RNA driving expression in the absence of an IRES.

An IRES, or internal ribosome entry site, is an RNA element that allows translation initiation in a cap-independent manner. Circular RNA was shown to be drive expression of Flag protein in the absence of an IRES.

Circular RNA was designed to encode triple FLAG tagged EGF flanked by a 2A sequence without a termination element (stop codon) (330 nts, SEQ ID NO: 19).

Linear or circular RNA was incubated for 5 hr in rabbit reticulocyte lysate at 30° C. in a volume of 25 μl. The final composition of the reaction mixture included 70% rabbit reticulocyte lysate, 20 μM amino acids, 0.8 U/μl RNase inhibitor and 1 μg of linear or circular RNA. After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 30° l of 2×SDS sample buffer and incubated at 70° C. for 15 min. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an enhanced chemiluminescence (ECL) kit (see FIG. 23) and western blot band intensity was measured by ImageJ.

Expression product was detected in the circular RNA reaction mixture even in the absence of an IRES.

### Example 27: Cap-Independent Expression

This Example demonstrates circular RNA is able to drive expression in the absence of a cap.

A cap is a specially altered nucleotide on the 5′ end of mRNA. The 5′ cap is useful for stability, as well as the translation initiation, of linear mRNA. Circular RNA drove expression of product in the absence of a cap.

Circular RNA was designed to encode triple FLAG tagged EGF flanked by a 2A sequence without a termination element (stop codon) (330 nts, SEQ ID NO: 19).

Linear or circular RNA was incubated for 5 hr in rabbit reticulocyte lysate at 30° C. in a volume of 25 μl. The final composition of the reaction mixture included 70% rabbit reticulocyte lysate, 20 μM amino acids, 0.8 U/μl RNase inhibitor and 1 μg of linear or circular RNA. After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 30 μl of 2×SDS sample buffer at 70° C. for 15 min. After centrifugation at 1400×g for 5 min, the supernatant was analyzed on 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit (see FIG. 23) and western blot band intensity was measured by ImageJ.

Expression product was detected in the circular RNA reaction mixture even in the absence of a cap.

### Example 28: Expression without a 5′-UTR

This Example demonstrates in vitro protein expression from a circular RNA lacking 5′ untranslated regions.

The 5′ untranslated region (5′ UTR) is the region directly upstream of an initiation codon that aids in downstream protein translation of a RNA transcript.

As shown in the following Example, a 5′-untranslated region in the circular RNA sequence was not necessary for in vitro protein expression.

Circular RNA was designed to encode triple FLAG tagged EGF flanked by a 2A sequence without a termination element (stop codon) (330 nts, SEQ ID NO: 19).

Linear or circular RNA was incubated for 5 hr in rabbit reticulocyte lysate at 30° C. in a volume of 25 μl. The final composition of the reaction mixture included 70% rabbit reticulocyte lysate, 20 μM amino acids, 0.8 U/μl RNase inhibitor and 1 μg of linear or circular RNA. After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 30° l of 2×SDS sample buffer and incubated at 70° C. for 15 min. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit (see FIG. 23) and western blot band intensity was measured by ImageJ.

Expression product was detected in the circular RNA reaction mixture even in the absence of a 5′ UTR.

### Example 29: Expression without a 3′-UTR

This Example demonstrates in vitro protein expression from a circular RNA lacking a 3′-UTR.

The 3′ untranslated region (3′-UTR) is the region directly downstream of a translation termination codon and includes regulatory regions that may post-transcriptionally influence gene expression. The 3′-untranslated region may also play a role in gene expression by influencing the localization, stability, export, and translation efficiency of an mRNA. In addition, the structural characteristics of the 3′-UTR as well as its use of alternative polyadenylation may play a role in gene expression.

As shown in the following Example, a 3′-UTR in the circular RNA sequence was not necessary for in vitro protein expression.

Circular RNA was designed to encode triple FLAG tagged EGF flanked by a 2A sequence without a termination element (stop codon) (330 nts, SEQ ID NO: 19).

Linear or circular RNA was incubated for 5 hr in rabbit reticulocyte lysate at 30° C. in a volume of 25 μl. The final composition of the reaction mixture included 70% rabbit reticulocyte lysate, 20 μM amino acids, 0.8 U/μl RNase inhibitor and 1 μg of linear or circular RNA. After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 30° l of 2×SDS sample buffer and incubated at 70° C. for 15 min. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit (see FIG. 23) and western blot band intensity was measured by ImageJ.

Expression product was detected in the circular RNA reaction mixture even in the absence of a 3′UTR.

### Example 30: Expression without a Termination Element (Stop Codon)

This Example demonstrates generation of a polypeptide product following rolling circle translation from a circular RNA lacking a termination element (stop codon).

Proteins are based on polypeptides, which are comprised of unique sequences of amino acids. Each amino acid is encoded in mRNA by nucleotide triplets called codon. During protein translation, each codon in mRNA corresponds to the addition of an amino acid in a growing polypeptide chain. Termination element or stop codons signal the termination of this process by binding release factors, which cause the ribosomal subunits to disassociate, releasing the amino acid chain.

As shown in the following Example, a circular RNA lacking a termination codon generated a large polypeptide product comprised of repeated polypeptide sequences via rolling circle translation.

Circular RNA was designed to encode triple FLAG tagged EGF without a termination element (stop codon) (264 nts, SEQ ID NO: 20), and included a Kozak sequence at the start codon to favor translation initiation.

Linear or circular RNA was incubated for 5 hr in rabbit reticulocyte lysate at 30° C. in a volume of 25 μl. The final composition of the reaction mixture included 70% rabbit reticulocyte lysate, 20 μM amino acids, 0.8 U/μl RNase inhibitor and 1 μg of linear or circular RNA. After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 30° l of 2×SDS sample buffer and incubated at 70° C. for 15 min. After centrifugation at 1400×g for 5 min, the supernatant was analyzed on a 10-20% gradient polyacrylamide/SDS gel.

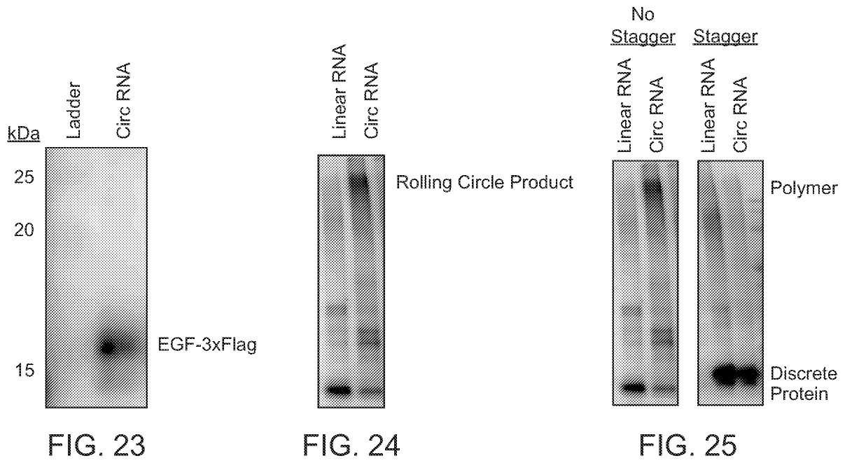

After being electrotransferred to a nitrocellulose membrane using dry transfer method, the blot was incubated with an anti-FLAG antibody and anti-mouse IgG peroxidase. The blot was visualized with an ECL kit (see FIG. 24) and western blot band intensity was measured by ImageJ.

Expression product was detected in the circular RNA reaction mixture even in the absence of a termination element (stop codon).

### Example 31: Expression of Discrete Proteins without a Termination Element (Stop Codon)

This Example demonstrates generation of a discrete protein products translated from a circular RNA lacking a termination element (stop codons).

Stagger elements, such as 2A peptides, may include short amino acid sequences, ˜20 aa, that allow for the production of equimolar levels of multiple genes from a single mRNA. The stagger element may function by making the ribosome skip the synthesis of a peptide bond at the C-terminus of the 2A element, leading to separation between the end of the 2A sequence and the next peptide downstream. The separation occurs between Glycine and Proline residues found on the C-terminus and the upstream cistron has a few additional residues added to the end, while the downstream cistron starts with a Proline.

As shown in the following Example, the circular RNA lacking a termination element (stop codon) generated a large polypeptide polymer (FIG. 16 left panel: no stagger—circular RNA lane) and inclusion of a 2A sequence at the 3′ end of the coding region resulted in production of discrete protein at a size comparable to that generated by the equivalent linear RNA construct (FIG. 16 right panel: stagger—circular RNA lane).

Circular RNA was designed to encode triple FLAG tagged EGF without a termination element (stop codon) (264 nts, SEQ ID NO: 20) and without a stagger element. A second circular RNA was designed to encode triple FLAG tagged EGF flanked by a 2A sequence without a termination element (stop codon) (330 nts, SEQ ID NO: 19).

Linear or circular RNA was incubated for 5 hr in rabbit reticulocyte lysate at 30° C. in a volume of 25 μl. The final composition of the reaction mixture included 70% rabbit reticulocyte lysate, 20 μM amino acids, 0.8 U/μl RNase inhibitor and 1 μg of linear or circular RNA. After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 30° l of 2×SDS sample buffer and incubated at 70° C. for 15 min. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit (FIG. 25) and western blot band intensity was measured by ImageJ.

Discrete expression products were detected indicating circular RNA comprising a stagger element drove expression of the individual proteins even in the absence of a termination element (stop codons).

### Example 32: Rolling Circle Translation

This Example demonstrates elevated in vitro biosynthesis of a protein from circular RNA using a stagger element.

A non-naturally occurring circular RNA was engineered to include a stagger element to compare protein expression with circular RNA lacking a stagger element. As shown in the following Example, a circRNA comprising a stagger element overexpressed protein as compared to an otherwise identical circular RNA lacking such a sequence.

Circular RNA was designed to encode triple FLAG tagged EGF with a termination element (e.g., three stop codons in tandem) (273 nts, SEQ ID NO: 21). A second circular RNA was designed to encode triple FLAG tagged EGF flanked by a 2A sequence without a termination element (stop codon) (330 nts, SEQ ID NO: 19).

Linear or circular RNA was incubated for 5 hr in rabbit reticulocyte lysate at 30° C. in a volume of 25 μl. The final composition of the reaction mixture contained 70% rabbit reticulocyte lysate, 20 μM amino acids, 0.8 U/μl RNase inhibitor and 1 μg of linear or circular RNA. After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 30° l of 2×SDS sample buffer and incubated at 70° C. for 15 min. After centrifugation at 1400×g for 5 min, the supernatant was analyzed on a 10-20% gradient polyacrylamide/SDS gel.

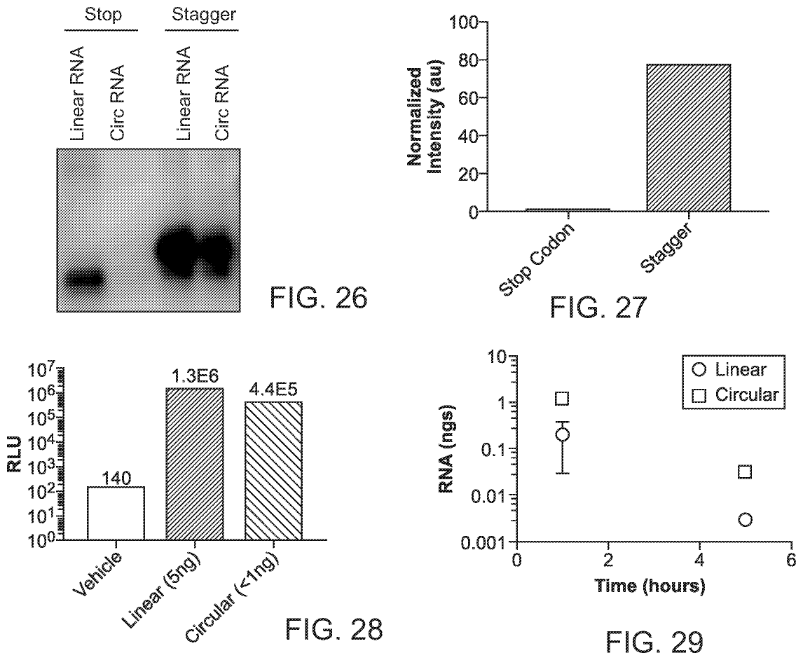

After being electrotransferred to a nitrocellulose membrane using dry transfer method, the blot was incubated with an anti-FLAG antibody and anti-mouse IgG peroxidase. The blot was visualized with an ECL kit (see FIG. 26) and western blot band intensity was measured by ImageJ. FIG. 27 shows a graph summarizing the signal intensity from Western blot analysis of the protein products translated from the two exemplary circular RNAs shown in FIG. 26, demonstrating increased protein expression from a circRNA comprising a stagger element and no stop codon compared to a circRNA comprising a stop codon, during rolling circle translation.

Discrete expression products were detected indicating circular RNA comprising a stagger element drove expression of the individual proteins even in the absence of a termination element (stop codons).

### Example 33: Expression of a Biologically Active Protein In Vitro

This Example demonstrates in vitro biosynthesis of a biologically active protein from circular RNA.

A non-naturally occurring circular RNA was engineered to express a biologically active therapeutic protein. As shown in the following Example, a biologically active protein was expressed from the circular RNA in reticulocyte lysate.

Circular RNA was designed to encode FLAG tagged EGF flanked by a 2A sequence and followed by FLAG tagged nano-luciferase (873 nts, SEQ ID NO:17).

Linear or circular RNA was incubated for 5 hr in rabbit reticulocyte lysate at 30° C. in a volume of 25 μl. The final composition of the reaction mixture contained 70% rabbit reticulocyte lysate, 20 μM amino acids, 0.8 U/μl RNase inhibitor. Luciferase activity in the translation mixture was monitored using a luciferase assay system according to manufacturer's protocol (Promega).

As shown in FIG. 28, much higher fluorescence was detected with both circular RNA and linear RNA than the control vehicle RNA, indicating expression product was present. Thus, circular RNA was shown to express a biologically active protein.

### Example 34: Circular RNA with a Longer Half-Life than Linear RNA Counterpart

This Example demonstrates circular RNA engineered to have a prolonged half-life as compared to a linear RNA.

Circular RNA encoding a therapeutic protein provided recipient cells with the ability to produce greater levels of the encoded protein stemming from a prolonged biological half-life, e.g., as compared to linear RNA. As shown in the following Example, a circular RNA had a greater half-life than its linear RNA counterpart in reticulocyte lysate.

A circular RNA was designed to encode FLAG tagged EGF flanked by a 2A sequence and followed by FLAG tagged nano luciferase (873 nts, SEQ ID NO: 17).

In this Example, a time-course experiment was performed to monitor RNA stability. 100 ng of linear or circular RNA was incubated with rabbit reticulocyte lysate and samples were collected at 1 hr, 5 hr, 18 hr and 30 hr. Total RNA was isolated from lysate using a phenol-based reagent (Invitrogen) and cDNA was generated by reverse transcription. qRT-PCR analysis was performed using a dry-based quantitative PCR reaction mix (BioRad).

As shown in FIG. 29, greater concentrations of the circular RNA were detected at the later timepoint than the linear RNA, and the percent decrease in the concentration of the circular RNA over time was lower than that of the linear RNA. Thus, the circular RNA was more stable or had an increased half-life as compared to its linear counterpart.

### Example 35: Circular RNA Demonstrated a Longer Half-Life than Linear RNA in Cells

This Example demonstrates circular RNA delivered into cells and has an increased half-life in cells compared with linear RNA.

A non-naturally occurring circular RNA was engineered to express a biologically active therapeutic protein. As shown in the following Example, circular RNA was present at higher levels compared to its linear RNA counterpart, demonstrating a longer half-life for circular RNA.

In this Example, circular RNA and linear RNA were designed to encode a Kozak, EGF, flanked by a 2A, a stop or no stop sequence (SEQ ID NOs: 11, 19, 20, 21). To monitor half-life of RNA in cells, 0.1×10⁶ cells were plated onto each well of a 12 well plate. After 1 day, 1 μg of linear or circular RNA was transfected into each well using a lipid-based transfection reagent (Invitrogen). Twenty-four hours after transfection, total RNA was isolated from cells using a phenol-based extraction reagent (Invitrogen). Total RNA (500 ng) was subjected to reverse transcription to generate cDNA. qRT-PCR analysis was performed using a dye-based quantitative PCR mix (BioRad). Primer sequences were as follow: Primers for linear or circular RNA, F: ACGACGGTGTGTGCATGTAT, R: TTCCCACCACTTCAGGTCTC; primers for circular RNA, F: TACGCCTGCAACTGTGTTGT, R: TCGATGATCTTGTCGTCGTC.

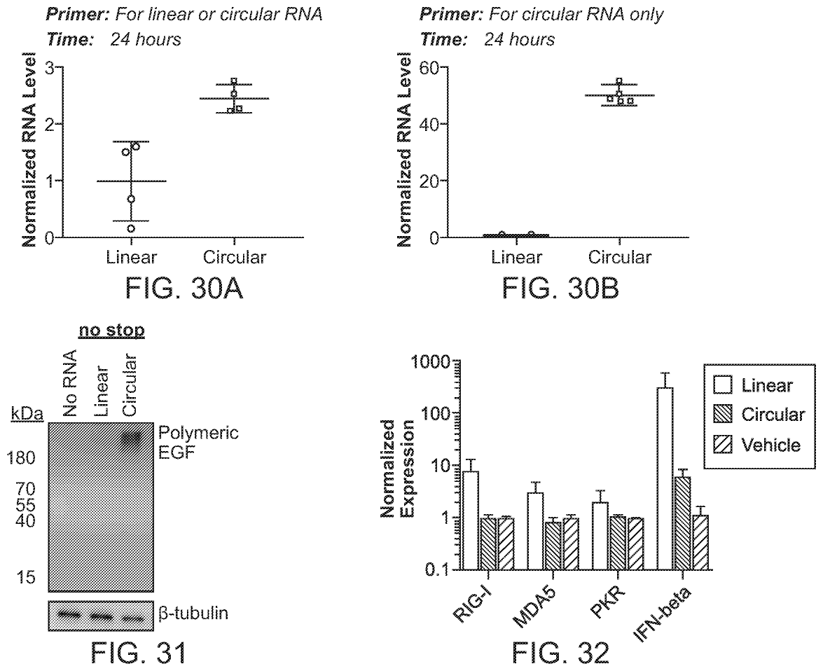

Circular RNA was successfully transfected into 293T cells, as was its linear counterpart. After 24 hours, the circular and linear RNA that remained were measured using qPCR. Circular RNA was shown to have a higher concentration in the cells 24 hours after transfection as compared to the linear RNA, suggesting that circular RNA has a longer half-life in cells than that of linear RNA (FIG. 30A and FIG. 30B).

### Example 36: Synthetic Circular RNA was Translated in Cells, and Synthetic Circular RNA was Translated Via Rolling Circle Translation

This Example demonstrates translation of synthetic circular RNA in cells.

As shown in the following Example, circular RNA and linear RNA were designed to encode a Kozak, 3×FLAG-EGF sequence with no termination element (SEQ ID NO: 11). Circular RNA was translated into polymeric EGF, while linear RNA was not, demonstrating that cells performed rolling circle translation of a synthetic circular RNA.

In this Example, 0.1×10⁶ cells were plated onto each well of a 12 well plate to monitor translation efficiency of linear or circular RNA in cells. After 1 day, 1 μg of linear or circular RNA was transfected into each well using a lipid-based transfection reagent (Invitrogen). Twenty-four hours after transfection, cells were harvested by adding 200 μl of RIPA buffer onto each well. Next, 10 μg of cell lysate proteins were analyzed on 10-20% gradient polyacrylamide/SDS gel. After electrotransfer to a nitrocellulose membrane using dry transfer method, the blot was incubated with an anti-FLAG antibody and anti-mouse IgG peroxidase. As a loading control, anti-beta tubulin antibody was used. The blot was visualized with an enhanced chemiluminescent (ECL) kit. Western blot band intensity was measured by ImageJ.

Circular RNA was successfully transfected into 293T cells, as was its linear counterpart. However, FIG. 31 shows that 24 hours after transfection, EGF protein was detected in the circular RNA transfected cells, while none was detected in the linear RNA transfected cells. Thus, circular RNA was translated in cells via rolling circle translation as compared to linear RNA.

### Example 37: Synthetic Circular RNA Demonstrated Reduced Immunogenic Gene Expression in Cells

This Example demonstrates circular RNA engineered to have a reduced immune response as compared to a linear RNA.

Circular RNA that encoded a therapeutic protein provided a reduced induction of immune-related genes (RIG-I, MDA5, PKR and IFN-beta) in recipient cells, as compared to linear RNA. RIG-I can recognize short 5′ triphosphate uncapped double stranded or single stranded RNA, while MDA5 can recognize longer dsRNAs. RIG-I and MDA5 can both be involved in activating MAVS and triggering antiviral responses. PKR can be activated by dsRNA and induced by interferons, such as IFN-beta. As shown in the following Example, circular RNA was shown to have a reduced activation of an immune related genes in 293T cells than an analogous linear RNA, as assessed by expression of RIG-I, MDA5, PKR and IFN-beta by q-PCR.

The circular RNA and linear RNA were designed to encode either (1) a Kozak, 3×FLAG-EGF sequence with no termination element (SEQ ID NO:11); (2) a Kozak, 3×FLAG-EGF, flanked by a termination element (stop codon) (SEQ ID NO:21); (3) a Kozak, 3×FLAG-EGF, flanked by a 2A sequence (SEQ ID NO:19); or (4) a Kozak, 3×FLAG-EGF sequence flanked by a 2A sequence followed by a termination element (stop codon) (SEQ ID NO:20).

In this Example, the level of innate immune response genes were monitored in cells by plating 0.1×10⁶ cells into each well of a 12 well plate. After 1 day, 1 μg of linear or circular RNA was transfected into each well using a lipid-based transfection reagent (Invitrogen). Twenty-four hours after transfection, total RNA was isolated from cells using a phenol-based extraction reagent (Invitrogen). Total RNA (500 ng) was subjected to reverse transcription to generate cDNA. qRT-PCR analysis was performed using a dye-based quantitative PCR mix (BioRad).

Primer sequences used:
Primers for GAPDH,
F: AGGGCTGCTTTTAACTCTGGT,
R: CCCCACTTGATTTTGGAGGGA;
RIG-I,
F: TGTGGGCAATGTCATCAAAA,
R: GAAGCACTTGCTACCTCTTGC;
MDA5,
F: GGCACCATGGGAAGTGATT,
R: ATTTGGTAAGGCCTGAGCTG;
PKR,
F: TCGCTGGTATCACTCGTCTG,
R: GATTCTGAAGACCGCCAGAG;
IFN-beta,
F: CTCTCCTGTTGTGCTTCTCC,
R: GTCAAAGTTCATCCTGTCCTTG.

As shown in FIG. 32, qRT-PCR levels of immune related genes from 293T cells transfected with circular RNA showed reduction of RIG-I, MDA5, PKR and IFN-beta as compared to linear RNA transfected cells. Thus, induction of immunogenic related genes in recipient cells was reduced in circular RNA transfected cells, as compared to linear RNA transfected cells.

### Example 38: Increased Expression from Synthetic Circular RNA Via Rolling Circle Translation in Cells

This Example demonstrates increased expression from rolling circle translation of synthetic circular RNA in cells.

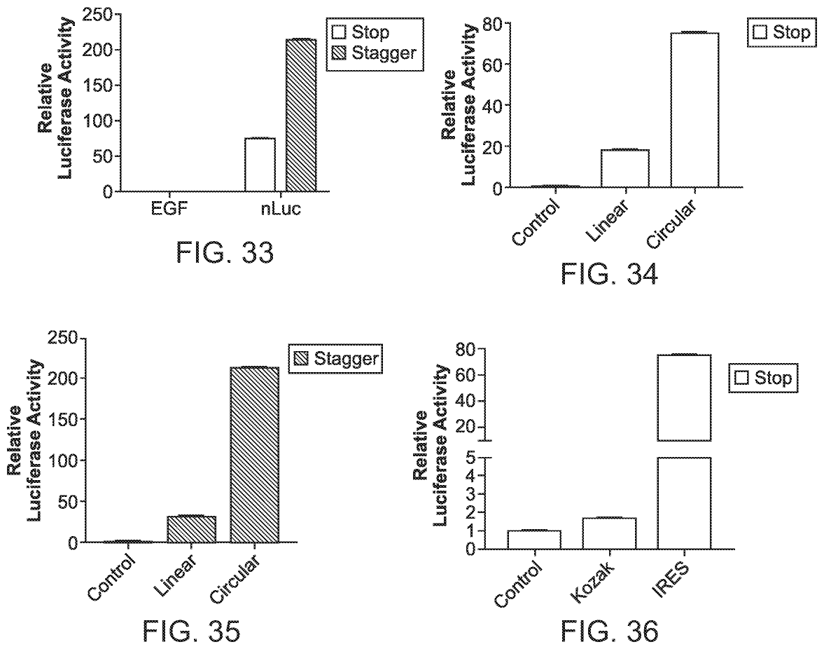

Circular RNAs were designed to include an IRES with a nanoluciferase gene or an EGF negative control gene without a termination element (stop codon). Cells were transfected with EGF negative control (SEQ ID NO:22); nLUC stop (SEQ ID NO:23): EMCV IRES, stagger sequence (2A sequence), 3× FLAG tagged nLUC sequences, stagger sequence (2A sequence), and a stop codon; or nLUC stagger (SEQ ID NO:24): EMCV IRES, stagger sequence (2A sequence), 3× FLAG tagged nLUC sequences, and stagger sequence (2A sequence). As shown in the FIG. 33, both circular RNAs produced translation product having functional luciferase activity.

In this Example, translation of circular RNA was monitored in cells. Specifically, 0.1×10⁶ cells were plated onto each well of a 12 well plate. After 1 day, 300 ng of circular RNA was transfected into each well using a lipid-based transfection reagent (Invitrogen). After 24 hrs, cells were harvested by adding 100 μl of RIPA buffer. Nanoluciferase activity in lysates was measured using a luciferase assay system according to its manufacturer's protocol (Promega).

As shown in FIG. 33, both circular RNAs expressed protein in cells. However, circular RNA with a stagger element, e.g., 2A sequence, that lacks a termination element (stop codon), produced higher levels of protein product having functional luciferase activity than circular RNA with a termination element (stop codon).

### Example 39: Synthetic Circular RNA Translated in Cells

This Example demonstrates synthetic circular RNA translation in cells. Additionally, this Example shows that circular RNA produced more expression product than its linear counterpart.

Circular RNA was successfully transfected into 293T cells, as was its linear counterpart. Cells were transfected with circular RNA encoding EGF as a negative control (SEQ ID NO:22): EMCV IRES, stagger sequence (2A sequence), 3× FLAG tagged EGF sequences, stagger sequence (2A sequence); linear or circular nLUC (SEQ ID NO:23): EMCV IRES, stagger sequence (2A sequence), 3× FLAG tagged nLuc sequences, a stagger sequence (2A sequence), and stop codon. As shown in FIG. 34, circular RNA was translated into nanoluciferase in cells.

Linear or circular RNA translation was monitored in cells. Specifically, 0.1×10⁶ cells were plated onto each well of a 12 well plate. After 1 day, 300 ng of linear or circular RNA was transfected into each well using a lipid-based transfection reagent (Invitrogen). After 24 hrs, cells were harvested by adding 100 μl of RIPA buffer. Nanoluciferase activity in lysates was measured using a luciferase assay system according to its manufacturer's protocol (Promega).

As shown in FIG. 34, circular RNA translation product was detected in cells. In particular, circular RNA had higher levels of luciferase activity or increased protein produced as compared to its linear RNA counterpart.

### Example 40: Rolling Circle Translation from Synthetic Circular RNA Produced Functional Protein Product in Cells

This Example demonstrates rolling circle translation of functional protein product from synthetic circular RNA lacking a termination element (stop codon), e.g., having a stagger element lacking a termination element (stop codon), in cells. Additionally, this Example shows that circular RNA with a stagger element expressed more functional protein product than its linear counterpart.

Circular RNA was successfully transfected into 293T cells, as was its linear counterpart. Cells were transfected with circular RNA EGF negative control (SEQ ID NO:22); linear and circular nLUC (SEQ ID NO:24): EMCV IRES, stagger sequence (2A sequence), 3× FLAG tagged nLuc sequences, a stagger sequence (2A sequence). As shown in FIG. 25, circular RNA was translated into nanoluciferase in cells.

Linear or circular RNA translation was monitored in cells. Specifically, 0.1×10⁶ cells were plated onto each well of a 12 well plate. After 1 day, 300 ng of linear or circular RNA was transfected into each well using a lipid-based transfection reagent (Invitrogen). After 24 hrs, cells were harvested by adding 100 μl of RIPA buffer. Nanoluciferase activity in lysates was measured using a luciferase assay system according to its manufacturer's protocol (Promega).

As shown in FIG. 35, circular RNA translation product was detected in cells. In particular, circular RNA without a termination element (stop codon) produced higher levels of protein product having functional luciferase activity than its linear RNA counterpart.

### Example 41: Synthetic Circular RNA Translated Via IRES Initiation in Cells

This Example demonstrates synthetic circular RNA translation initiation with an IRES in cells.

Circular RNAs were designed to include a Kozak sequence or IRES with a nanoluciferase gene or an EGF negative control gene. Cells were transfected with EGF negative control (SEQ ID NO:22), nLUC Kozak (SEQ ID NO:25): Kozak sequence, 1× FLAG tagged EGF sequence, a stagger sequence (T2A sequence), 1× FLAG tagged nLUC, stagger sequence (P2A sequence), and a stop codon; or nLUC IRES (SEQ ID NO:23): EMCV IRES, stagger sequence (2A sequence), 3× FLAG tagged nLUC sequences, stagger sequence (2A sequence) and a stop codon. As shown in the FIG. 36, the circular RNA with an IRES demonstrated higher levels of luciferase activity, corresponding to higher protein levels, as compared to circular RNA with a Kozak sequence.

In this Example, translation of circular RNA was monitored in cells. Specifically, 0.1×10⁶ cells were plated onto each well of a 12 well plate. After 1 day, 300 ng of circular RNA was transfected into each well using a lipid-based transfection reagent (Invitrogen). After 24 hrs, cells were harvested by adding 100 μl of RIPA buffer. Nanoluciferase activity in lysates was measured using a luciferase assay system according to its manufacturer's protocol (Promega).

As shown in FIG. 36, circular RNA initiated protein expression with an IRES and produced higher levels of protein product having functional luciferase activity than circular RNA with Kozak initiated protein expression.

### Example 42: Rolling Circle Translation of Synthetic Circular RNA in Cells

This Example demonstrates greater protein production via rolling circle translation of synthetic circular RNA in cells that initiated protein production with an IRES.

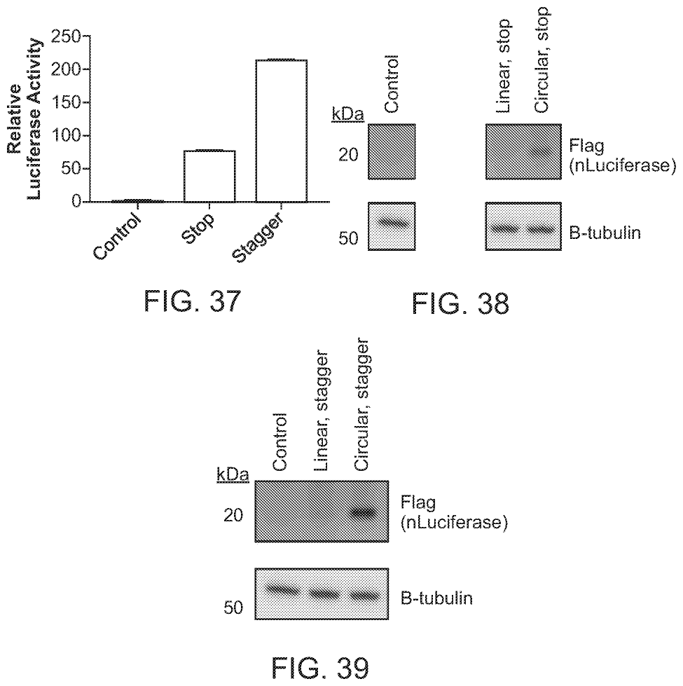

Circular RNAs were designed to include an a Kozak sequence or IRES with a nanoluciferase gene or an EGF negative control with or without a termination element (stop codon). Cells were transfected with EGF negative control (SEQ ID NO:22); nLUC IRES stop (SEQ ID NO:23): EMCV IRES, stagger sequence (2A sequence), 3× FLAG tagged nLUC sequences, stagger sequence (2A sequence) and a stop codon; or nLUC IRES stagger (SEQ ID NO:24): EMCV IRES, stagger sequence (2A sequence), 3× FLAG tagged nLUC sequences, and stagger sequence (2A sequence). As shown in the FIG. 37, both circular RNAs produced expression product demonstrated rolling circle translation and the circular RNA without a termination element an IRES (e.g., without a Kozak sequence) initiated and produced higher levels of protein product with functional luciferase activity than circular RNA with a termination element out an IRES (e.g., with a Kozak sequence), demonstrating rolling circle translation.

In this Example, translation of circular RNA was monitored in cells. Specifically, 0.1×10⁶ cells were plated onto each well of a 12 well plate. After 1 day, 300 ng of circular RNA was transfected into each well using a lipid-based transfection reagent (Invitrogen). After 24 hrs, cells were harvested by adding 100 μl of RIPA buffer. Nanoluciferase activity in lysates was measured using a luciferase assay system according to its manufacturer's protocol (Promega).

As shown in FIG. 37, circular RNA was translated into protein in cells via a rolling circle method given from both circular RNAs. However, the rolling circle translation of the circular RNA initiated greater protein production with an IRES and produced more protein product having functional luciferase activity as compared to a circular RNA with a termination element Kozak translation initiation.

### Example 43: Increased Protein Expressed from Circular RNA

This Example demonstrates demonstrates synthetic circular RNA translation in cells. Additionally, this Example shows that circular RNA produced more expression product of the correct molecular weight than its linear counterpart.

Linear and circular RNAs were designed to include a nanoluciferase gene with a termination element (stop codon). Cells were transfected with vehicle: transfection reagent only; linear nLUC (SEQ ID NO:23): EMCV IRES, stagger element (2A sequence), 3× FLAG tagged nLuc sequences, a stagger element (2A sequence), and termination element (stop codon); or circular nLUC (SEQ ID NO:23): EMCV IRES, stagger element (2A sequence), 3× FLAG tagged nLuc sequences, a stagger element (2A sequence), and a termination element (stop codon). As shown in the FIG. 28, circular RNA produced greater levels of protein having the correct molecular weight as compared to linear RNA.

After 24 hrs, cells were harvested by adding 100 μl of RIPA buffer. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit and western blot band intensity was measured by ImageJ.

As shown in FIG. 38, circular RNA was translated into protein in cells. In particular, circular RNA produced higher levels of protein having the correct molecular weight as compared to its linear RNA counterpart.

### Example 44: Rolling Circle Translation of Synthetic Circular RNA Produced Discrete Protein Products in Cells

This Example demonstrates discrete protein products were translated via rolling circle translation from synthetic circular RNA lacking a termination element (stop codon), e.g., having a stagger element in lieu of a termination element (stop codon), in cells. Additionally, this Example shows that circular RNA with a stagger element expressed more protein product having the correct molecular weight than its linear counterpart.

Circular RNAs were designed to include a nanoluciferase gene with a stagger element in place of a termination element (stop codon). Cells were transfected with vehicle: transfection reagent only; linear nLUC (SEQ ID NO:24): EMCV IRES, stagger element (2A sequence), 3× FLAG tagged nLuc sequences, and a stagger element (2A sequence); or circular nLUC (SEQ ID NO:24): EMCV IRES, stagger element (2A sequence), 3× FLAG tagged nLuc sequences, and a stagger element (2A sequence). As shown in the FIG. 39, circular RNA produced greater levels of protein having the correct molecular weight as compared to linear RNA.

After 24 hrs, cells were harvested by adding 100 μl of RIPA buffer. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit and western blot band intensity was measured by ImageJ.

As shown in FIG. 39, circular RNA translation product was detected in cells. In particular, circular RNA without a termination element (stop codon) produced higher levels of discrete protein product having the correct molecular weight than its linear RNA counterpart.

### Example 45: Preparation of Circular RNA with a Quasi-Double Stranded, Helical Structure

This Example demonstrates circular RNA possessed both quasi-double stranded and helical structure.

A non-naturally occurring circular RNA was engineered to adopt a quasi-double stranded, helical structure. A similar structure was shown to be involved in condensation of a naturally occurring circular RNA that possessed a uniquely long in vivo half-life (Griffin et al 2014, J Virol. 2014 July; 88(13):7402-11. doi: 10.1128/JVI.00443-14, Guedj et al, Hepatology. 2014 December; 60(6):1902-10. doi: 10.1002/hep.27357).

In this Example, circular RNA was designed to encode a EMCV IRES, Nluc tagged with 3× FLAG as ORF and stagger sequence (EMCV 2A 3× FLAG Nluc 2A no stop). To evaluate RNA secondary structure, thermodynamic RNA structure prediction tool (RNAfold) was used (Vienna RNA). Additionally, RNA tertiary structure was analyzed using an RNA modeling algorithm.

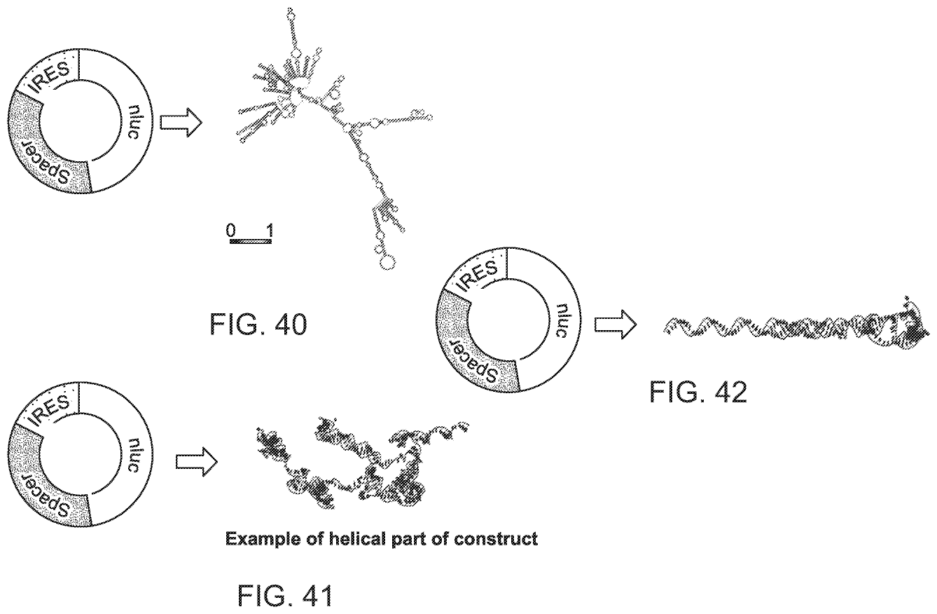

As shown in FIG. 40 and FIG. 41, circular RNA is modeled to have adopted a quasi-double stranded, helical structure.

### Example 46: Preparation of Circular RNA with a Quasi-Helical Structure Linked with a Repetitive Sequence

This Example demonstrates circular RNA can be designed to possess a quasi-helical structure linked with a repetitive sequence.

A non-naturally occurring circular RNA was engineered to adopt a quasi-helical structure linked with a repetitive sequence. A similar structure was shown to be involved in condensation of a naturally occurring circular RNA that possessed a uniquely long in vivo half-life (Griffin et al 2014, Guedj et al 2014).

In this Example, circular RNA was designed to encode a EMCV IRES, Nluc and spacer including a repetitive sequence (SEQ ID NO: 26). To evaluate RNA tertiary structure, an RNA modeling algorithm was used.

As shown in FIG. 42, circular RNA is modeled to have adopted a quasi-helical structure.

### Example 47: Circularized RNA is Circular and not Concatemeric

This Example demonstrates circular RNA degradation by RNase H produced nucleic acid degradation products consistent with a circular and not a concatemeric RNA.

RNA, when incubated with a ligase, can either not react or form an intra- or intermolecular bond, generating a circular (no free ends) or a concatemeric RNA, respectively. Treatment of each type of RNA with a complementary DNA primer and RNAse H, a nonspecific endonuclease that recognizes DNA/RNA duplexes, is expected to produce a unique number of degradation products of specific sizes depending on the starting RNA material.

As shown in the following Example, a ligated RNA was shown to be circular and not concatemeric based on the number and size of RNAs produced by RNase H degradation.

Circular RNA and linear RNA containing EMCV T2A 3× FLAG-Nluc P2A were generated.

To test circularization status of the RNA (1299 nts), 0.05 pmol/μl of linear or circular RNA was incubated with 0.25 U/μl of RNase H, an endoribonuclease that digests DNA/RNA duplexes, and 0.3 pmole/μl oligomer against 1037-1046 nts of RNA (CACCGCTCAGGACAATCCTT, SEQ ID NO: 55) at 37° C. for 20 min. After incubation, the reaction mixture was analyzed by 6% denaturing PAGE.

For the linear RNA used described above, it is expected that after binding of the DNA primer and subsequent cleavage by RNase H two cleavage products are obtained of 1041 nt and 258 nt. A concatemer is expected to produce three cleavage products of 258, 1041 and 1299 nt. A circular is expected to produce a single 1299 nt cleavage product.

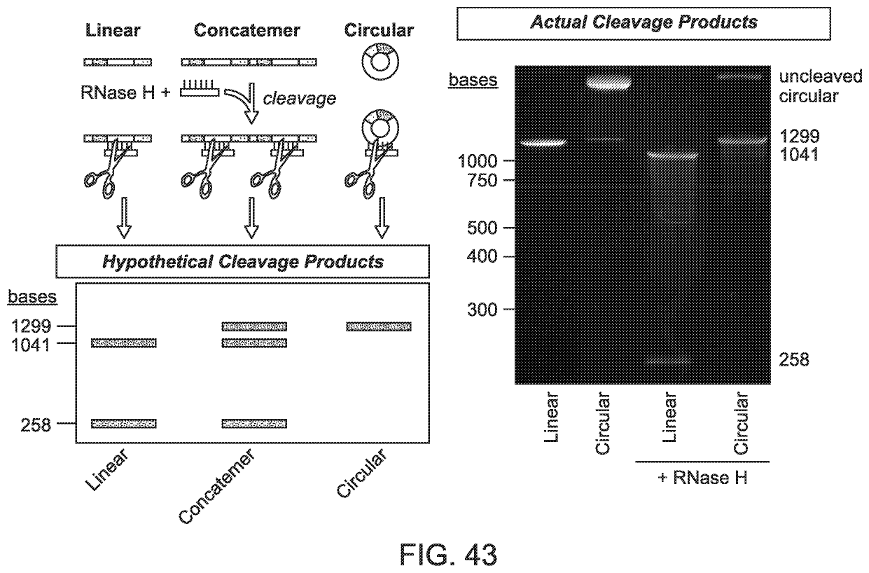

The number of bands in the linear RNA lane incubated with RNase endonuclease produced two bands of 1041 nt and 258 nt as expected, whereas a single band of 1299 nt was produced in the circular RNA lane (see FIG. 43), indicating that the circular RNA was in fact circular and not concatemeric.

### Example 48: Preparation of Large circRNAs

This Example demonstrates the generation of circular polyribonucleotide from in the range of about 20 bases to about 6.2 Kb.

A non-naturally occurring circular RNA engineered to include one or more desirable properties was produced in a range of sizes depending on the desired function. As shown in the following Example, linear RNA of up to 6200 nt was circularized.

The plasmid pCDNA3.1/CAT (6.2 kb) was used here. Primers were designed to anneal to pCDNA3.1/CAT at regular intervals to generate DNA oligonucleotides corresponding to 500 nts, 1000 nts, 2000 nts, 4000 nts, 5000 nts and 6200 nts. In vitro transcription of the indicated DNA oligonucleotides was performed to generate linear RNA of the corresponding sizes. Circular RNAs were generated from these RNA oligonucleotides using splint DNA.

To measure circularization efficiency of RNA, 6 different sizes of linear RNA (500 nts, 1000 nts, 2000 nts, 4000 nts, 5000 nts and 6200 nts) were generated. They were circularized using a DNA splint and T4 DNA ligase 2. As a control, one reaction was performed without T4 RNA ligase. Half of the circularized sample was treated with RNAse R to remove linear RNA.

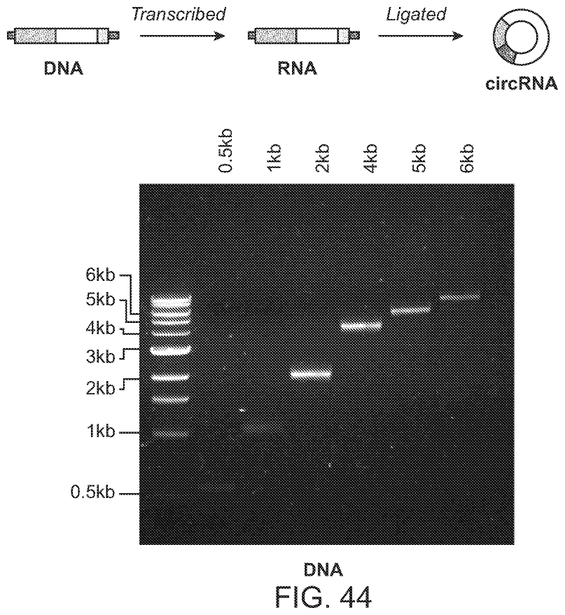

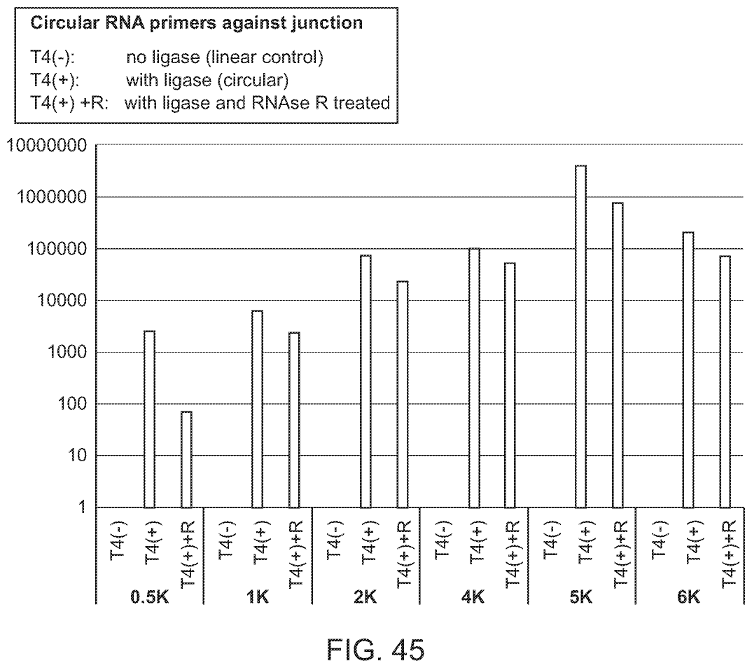

To monitor circularization efficiency, each sample was analyzed using qPCR. As shown in FIG. 44, circular RNA was generated from a wide variety of DNA of different lengths. As shown in FIG. 45, circularization of RNA was confirmed using RNAse R treatment and qPCR analysis against circular junctions. This Example demonstrates circular RNA production for a variety of lengths.

### Example 49: Circular RNA Engineered with a Protein Binding Site

This Example demonstrates generation of a circular RNA with a protein binding site.

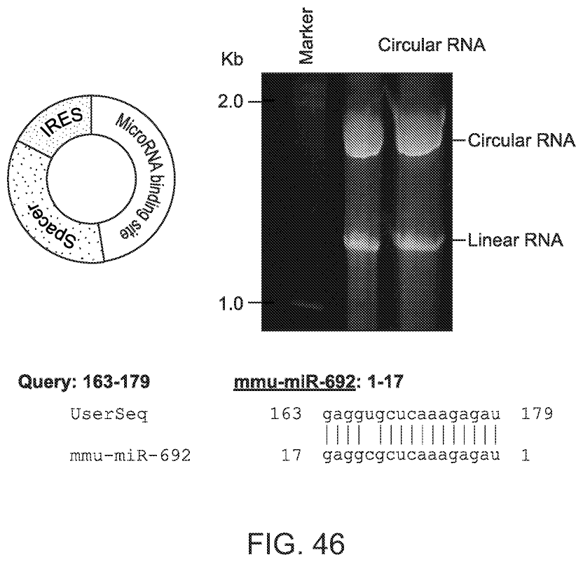

In this Example, one circular RNA is designed to include CVB3 IRES (SEQ ID NO:56), and an ORF encoding Gaussia luciferase (Gluc) (SEQ ID NO:57) followed by at least one protein binding site. For a specific example, a HuR binding sequence (SEQ ID NO:52) from Sindbis virus 3′UTR is used to test the effect of protein binding to circular RNA immunogenicity. HuR binding sequence comprises two elements, URE (U-rich element; SEQ ID NO: 50) and CSE (Conserved sequence element; SEQ ID NO: 51). Circular RNA without HuR binding sequence or with URE is used as a control. Part of the Anabaena autocatalytic intron and exon sequences are located prior to the CVB3 IRES (SEQ ID NO:56). The circular RNAs are generated in vitro as described. As shown in FIG. 46, circular RNA was generated to contain an HuR binding site.

To monitor the effect of RNA binding protein on circular RNA immunogenicity, cells are plated into each well of a 96 well plate. After 1 day, 500 ng of circular RNA is transfected into each well using a lipid-based transfection reagent (Invitrogen). Translation efficiency/RNA stability/immunogenicity are monitored daily, up to 72 hrs. Media is harvested to monitor Gluc activity. Cell lysate for measuring RNA level is prepared with a kit that allows measurements of relative gene expression by real-time RT-PCR (Invitrogen).

Translation efficiency is monitored by measuring Gluc activity with Gaussia luciferase flash assay kit according to the manufacturer's instruction (Pierce).

For qRT-PCR analysis, cDNA is generated with cell lysate preparation kit according to manufacturer's instruction (Invitrogen). qRT-PCR analysis is performed in triplicate using a PCR master mix (Brilliant II SYBR Green qRT-PCR Master Mix) and a PCR cycler (LightCycler 480). Circular RNA stability is measured by primers against Nluc. mRNA levels for well-known innate immunity regulators such as RIG-I, MDA5, OAS, OASL, and PKR are quantified and normalized to actin values.

### Example 50: Preparation of Circular RNA with Regulatory Nucleic Acid Sites

This Example demonstrates in vitro production of circular RNA with a regulatory RNA binding site.

Different cell types possess unique nucleic acid regulatory machinery to target specific RNA sequences. Encoding these specific sequences in a circular RNA could confer unique properties in different cell types. As shown in the following Example, circular RNA was engineered to encode a microRNA binding site.

In this Example, circular RNA included a sequence encoding a WT EMCV IRES, a mir692 microRNA binding site (GAGGUGCUCAAAGAGAU), and two spacer elements flanking the IRES-ORF.

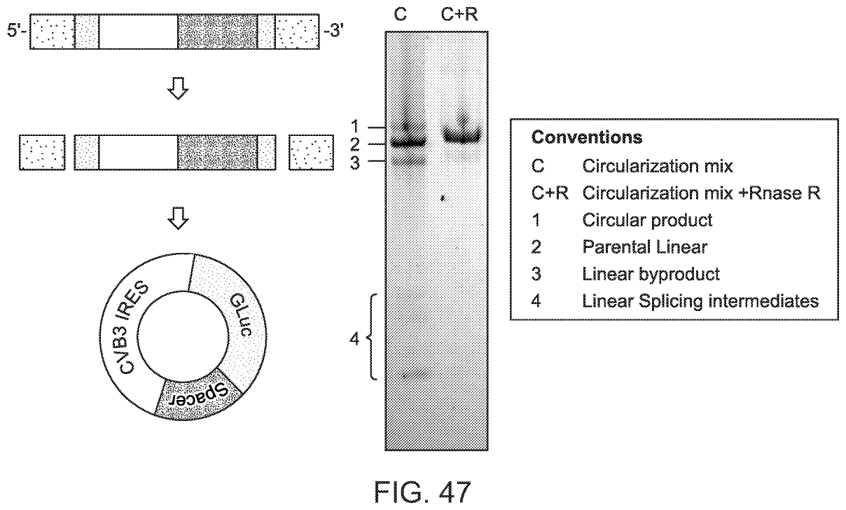

The circular RNA was generated in vitro. Unmodified linear RNA was in vitro transcribed from a DNA template including all the motifs listed above, in addition to the T7 RNA polymerase promoter to drive transcription. Transcribed RNA was purified with an RNA cleanup kit (New England Biolabs, T2050), treated with RNA 5′-phosphohydrolase (RppH) (New England Biolabs, M0356) following the manufacturer's instructions, and purified again with an RNA purification column. RppH treated RNA was circularized using a splint DNA (GGCTATTCCCAATAGCCGTT) and T4 RNA ligase 2 (New England Biolabs, M0239). Circular RNA was Urea-PAGE purified (FIG. 47), eluted in a buffer (0.5M Sodium Acetate, 0.1% SDS, 1 mM EDTA), ethanol precipitated and resuspended in RNase free water.

As shown in FIG. 47, circular RNA was generated with a miRNA binding site.

### Example 51: Self-Splicing of Circular RNA

This example demonstrates the ability to produce a circular RNA by self-splicing.

For this Example, circular RNAs included a CVB3 IRES, an ORF encoding Gaussia Luciferase (GLuc), and two spacer elements flanking the IRES-ORF.

The circular RNA was generated in vitro. Unmodified linear RNA was in vitro transcribed from a DNA template including all the motifs listed above. In vitro transcription reactions included 1 μg of template DNA T7 RNA polymerase promoter, 10×T7 reaction buffer, 7.5 mM ATP, 7.5 mM CTP, 7.5 mM GTP, 7.5 mM UTP, 10 mM DTT, 40 U RNase Inhibitor, and T7 enzyme. Transcription was carried out at 37° C. for 4 h. Transcribed RNA was DNase treated with 1 U of DNase I at 37° C. for 15 min. To favor circularization by self splicing, additional GTP was added to a final concentration of 2 mM, incubated at 55° C. for 15 min. RNA was then column purified and visualized by UREA-PAGE.

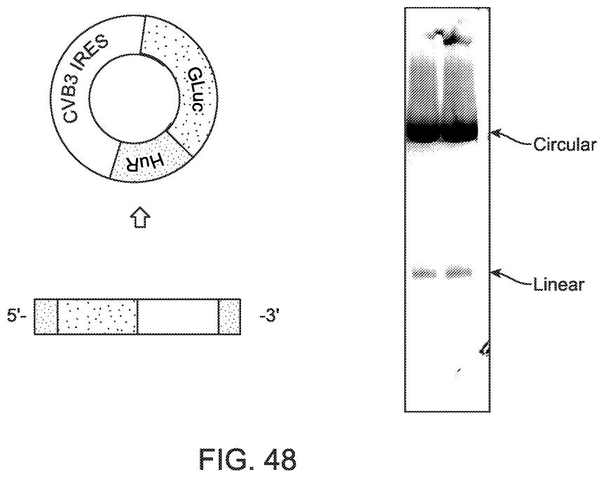

FIG. 48 shows circular RNA generated by self-splicing.

### Example 52: Circular RNA with a Splicing Element Comprising an Encryptogen

For this Example, a circular RNAs includes a CVB3 IRES, an ORF encoding Gaussia Luciferase (GLuc), and two spacer elements flanking the IRES-ORF, these two spacer elements comprise a splicing element that are part of the Anabaena autocatalytic intron and exon sequences (SEQ ID NO:59).

The circular RNA is generated in vitro.

In this Example, the level of innate immune response genes is monitored in cells by plating cells into each well of a 12 well plate. After 1 day, 1 μg of linear or circular RNA is transfected into each well using a lipid-based transfection reagent (Invitrogen). Twenty-four hours after transfection, total RNA is isolated from cells using a phenol-based extraction reagent (Invitrogen). Total RNA (500 ng) is subjected to reverse transcription to generate cDNA. qRT-PCR analysis is performed using a dye-based quantitative PCR mix (BioRad).

qRT-PCR levels of immune related genes from BJ cells transfected with circular RNA comprising a splicing element are evaluated for a reduction of RIG-I, MDA5, PKR and IFN-beta as compared to linear RNA transfected cells.

### Example 53: Persistence of Circular RNA During Cell Division

This Example demonstrates the persistence of circular polyribonucleotide during cell division. A non-naturally occurring circular RNA engineered to include one or more desirable properties may persist in cells through cell division without being degraded. As shown in the following Example, circular RNA expressing Gaussia luciferase (GLuc) was monitored over 72 h days in HeLa cells.

In this Example, a 1307 nt circular RNA included a CVB3 IRES, an ORF encoding Gaussia Luciferase (GLuc), and two spacer elements flanking the IRES-ORF.

Persistence of circular RNA over cell division was monitored in HeLa cells. 5000 cells/well in a 96-well plate were suspension transfected with circular RNA. Bright cell imaging was performed in a Avos imager (ThermoFisher) and cell counts were performed using luminescent cell viability assay (Promega) at 0 h, 24 h, 48 h, 72 h, and 96 h. Gaussia Luciferase enzyme activity was monitored daily as measure of protein expression and gLuc expression was monitored daily in supernatant removed from the wells every 24 h by using the Gaussia Luciferase activity assay (Thermo Scientific Pierce). 50 μl of 1×Gluc substrate was added to 5 μl of plasma to carry out the Gluc luciferase activity assay. Plates were read right after mixing on a luminometer instrument (Promega).

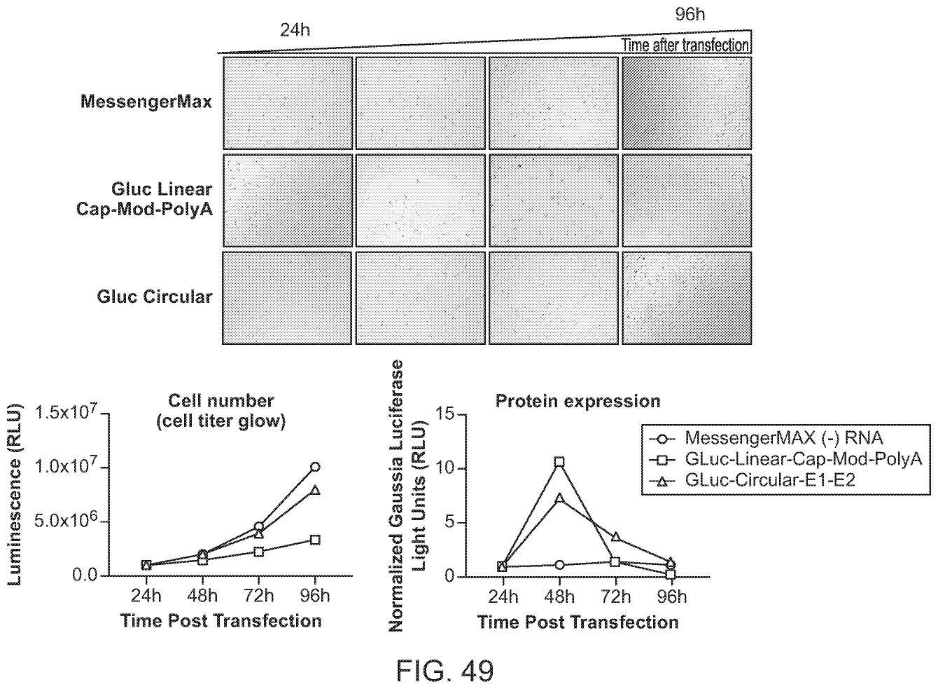

Expression of protein from circular RNA was detected at higher levels (about 10 RLU at 48 hr post transfection) than linear RNA (about 7 RLU at 48 hr post-transfection) in dividing cells (FIG. 49). Cells with circular RNA had higher cell division rates as compared to linear RNA at all timepoints measured. This Example demonstrates increased detection of circular RNA during cell division than its linear RNA counterpart.

### Example 54: Rolling Circle Translation Produced a Plurality of Expression Sequences

This Example demonstrates the ability of circular RNA to express multiple proteins from a single construct. Additionally, this Example demonstrates rolling circle translation of circular RNA encoding multiple ORFs. This Example further demonstrates expression of two proteins from a single construct.

One circular RNA (mtEMCV T2A 3× FLAG-GFP F2A 3× FLAG-Nluc P2A IS spacer) was designed for rolling circle translation to include EMCV IRES (SEQ ID NO:58), and an ORF encoding GFP with 3× FLAG tag and an ORF encoding Nanoluciferase (Nluc) with 3× FLAG tag. Stagger elements (2A) were flanking the GFP and Nluc ORFs. Another circular RNA was designed similarly, but included a triple stop codon inbetween the Nluc ORF and the spacer. Part of the Anabaena autocatalytic intron and exon sequences were included prior to the EMCV IRES. The circular RNAs were generated either in vitro as described.

The expression of proteins from circular RNA was monitored either in vitro or in cells. For in vitro analysis, the circular RNAs were incubated for 3 h in rabbit reticulocyte lysate (Promega, Fitchburg, Wis., USA) at 30° C. The final composition of the reaction mixture included 70% rabbit reticulocyte lysate, 20 μM complete amino acids, and 0.8 U/μL RNase inhibitor (Toyobo, Osaka, Japan).

After incubation, hemoglobin protein was removed by adding acetic acid (0.32 μl) and water (300 μl) to the reaction mixture (16 μl) and centrifuging at 20,817×g for 10 min at 15° C. The supernatant was removed and the pellet was dissolved in 2×SDS sample buffer and incubated at 70° C. for 15 min. After centrifugation at 1400×g for 5 min, the supernatant was analyzed on a 10-20% gradient polyacrylamide/SDS gel.

For analysis in cells, cells were plated into each well of a 12 well plate to monitor translation efficiency of circular RNA in cells. After 1 day, 500 ng of circular RNA was transfected into each well using a lipid-based transfection reagent (Invitrogen). 48 hours after transfection, cells were harvested by adding 200 μl of RIPA buffer onto each well. Next, 10 μg of cell lysate proteins were analyzed on 10-20% gradient polyacrylamide/SDS gel.

After electrotransfer of samples from reticulocyte lysate and cells to a nitrocellulose membrane using dry transfer method, the blot was incubated with an anti-FLAG antibody and anti-mouse IgG peroxidase. As a loading control, anti-beta tubulin antibody was used. The blot was visualized with an enhanced chemiluminescent (ECL) kit. Western blot band intensity was measured by ImageJ.

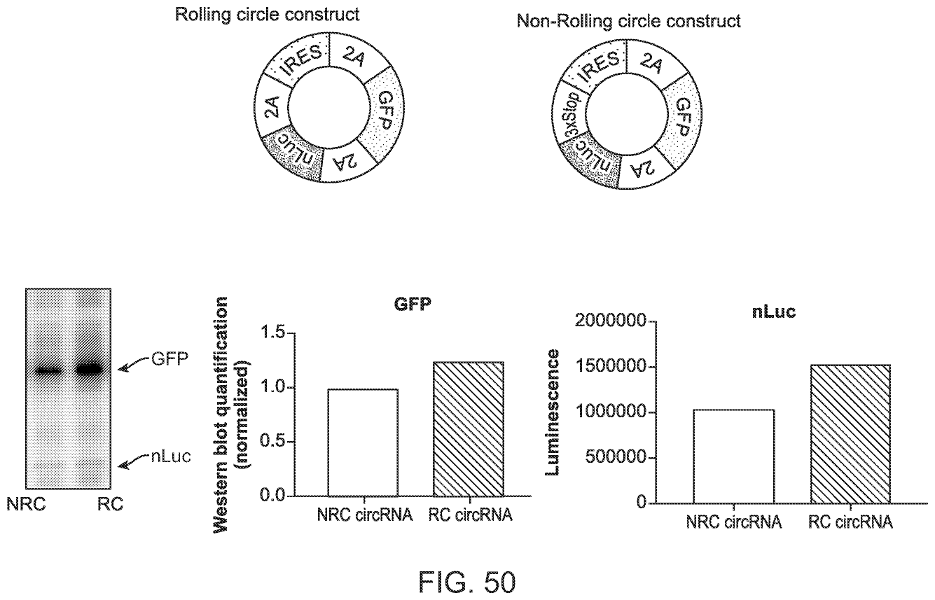

As shown in FIG. 50, the circular RNA encoding GFP and nLuc produced 2 protein products. Translation from the circular RNA without the triple stop generated more of both protein products than circular RNA with the triple stop codon. Finally, both circular RNA with and without the triple stop expressed proteins at 1/3.24 and 1/3.37 ratios, respectively.

### Example 55: Circular RNA Shows Reduced Toxicity Compared to Linear RNA

This Example demonstrates that circular RNA is less toxic than linear RNA.

For this Example, the circular RNA includes an EMCV IRES, an ORF encoding NanoLuc with a 3× FLAG tag and flanked on either side by stagger elements (2A) and a termination element (Stop codon). The circular RNA was generated in vitro and purified as described herein. The linear RNA used in this Example was cap-modified-poly A tailed RNA or cap-unmodified-poly A tailed RNA encoding nLuc with globin UTRs.

To monitor toxicity of RNA in cells, BJ human fibroblast cells were plated onto each well of a 96 well plate. 50 ng of either circular or cap-modified-polyA tailed linear RNA were transfected after zero, forty-eight, and seventy-two hours, using a lipid-based transfection reagent (ThermoFisher) following the manufacturer's recommendations. Bright cell imaging was performed in a Avos imager (ThermoFisher) at 96 h. Total cells per condition were analyzed using ImageJ.

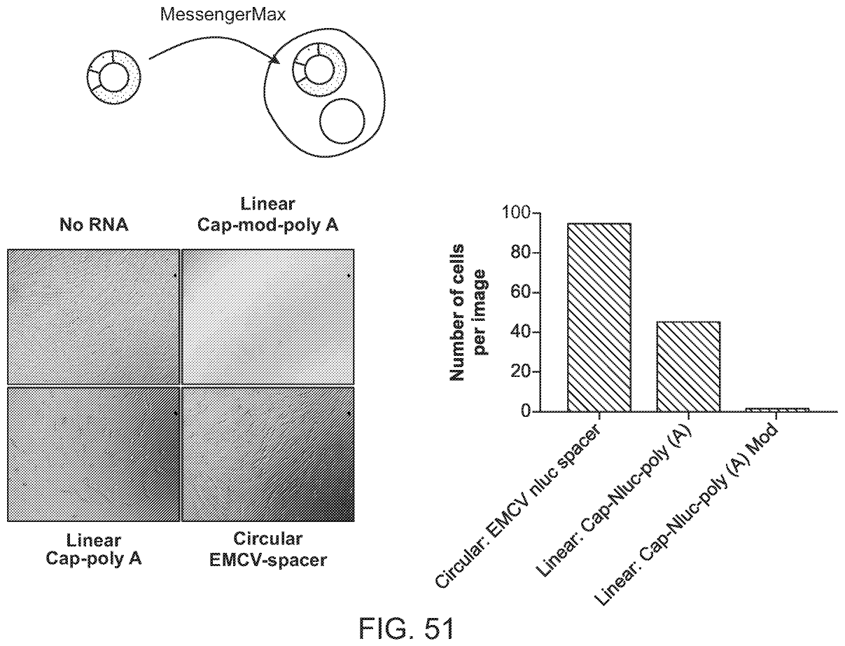

As shown in FIG. 51, there were about 90-100 cells per image in the culture transfected with circular RNA, while only about 40 cells per image in the culture transfected with linear Cap-Nluc-poly (A) RNA, indicating that transfection of circular RNA demonstrated reduced toxicity compared to linear RNA.

### Example 56: Expression Under Stress Conditions

This Example demonstrates that circular RNA expressed better under stress conditions than linear RNA.

For this Example, the circular RNAs includes an EMCV TRES, an ORF encoding NanoLuc with a 3× FLAG tag, and flanked by stagger elements. The circular RNA was generated in vitro and purified as described. The linear RNA used in this Example was capped-poly A tailed RNA encoding nLuc with globin UTRs.

To monitor expression of Gaussia Luciferase from cells, BJ human fibroblast cells were plated into each well of a 96 well plate. 50 ng of either circular or cap-polyA tailed linear RNA was transfected after zero, forty-eight, and seventy-two hours, using a lipid-based transfection reagent following the manufacturer's recommendations. Gaussia Luciferase enzyme activity was monitored daily as measure of protein expression and gLuc expression was monitored daily in supernatant removed from the wells every 24 h by using the Gaussia Luciferase activity assay (Thermo Scientific Pierce). 50 μl of 1×Gluc substrate was added to 5 μl of plasma to carry out the Gluc luciferase activity assay. Plates were read right after mixing on a luminometer instrument (Promega).

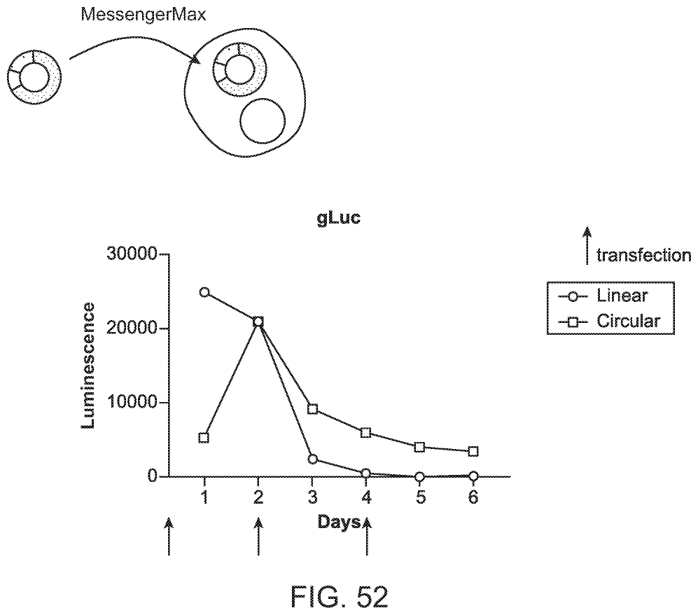

As shown in FIG. 52, circular RNA was translated at a higher level (about 1000 RLU at 3 days post-transfection and above 2000 RLU at 6 days post-transfection) as compared to linear RNA (about 2000 RLU at 3 days post-transfection and decreasing to undetectable 4 days after transfection) under stress condition.

### Example 57: Riboswitches for Selective Expression

This Example demonstrates the ability to control protein expression from circular RNA.

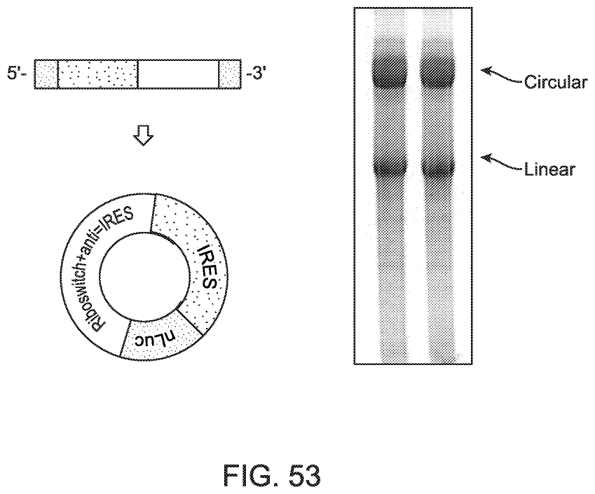

For this Example, circular RNAs were designed to include a synthetic riboswitch (SEQ ID NO: 60) regulating the expression of the ORF encoding NanoLuc, see FIG. 43. The circular RNA was generated in vitro. Unmodified linear RNA was in vitro transcribed from a DNA template including all the motifs listed above, in addition the T7 RNA polymerase promoter to drive transcription. Transcribed RNA was purified with an RNA cleanup kit (New England Biolabs, T2050), treated with RNA 5′-phosphohydrolase (RppH) (New England Biolabs, M0356) following the manufacturer's instructions, and purified again with an RNA purification column. RppH treated RNA was circularized using a splint DNA (CCGTTGTGGTCTCCCAGATAAACAGTATTTTGTCC) and T4 RNA ligase 2 (New England Biolabs, M0239). Circular RNA was Urea-PAGE purified (FIG. 53).

Theophylline or Tetracycline induce the activation of its specific riboswitch, resulting in an off-switch of gene expression (as described by Auslander et al Mol Biosyst. 2010 May; 6(5):807-14 and Ogawa et al, RNA. 2011 March; 17(3):478-88. doi: 10.1261/rna.2433111. Epub 2011 Jan. 11). It is expected that the riboswitch controls GFP or NLuc expression from the circular RNA. Thus, no GFP or NLuc expression is expected after the addition of theophylline or tetracycline.

The efficiency of the riboswitch is tested in a cell-free translation system and in HeLa cells. Cell-free translation is carried out by using a cell-free translation kit (Promega, L4140) following manufacturer's recommendations and measuring luminescence with a luminometer instrument (Promega) for the NLuc ORF and a cell imaging multi-mode reader (BioTek) for the GFP ORF.

For cellular assays, HeLa cells/well are transfected with 1 nM of the described circular RNA encoding GFP or NLuc under the control of either the theophylline or the tetracycline dependent synthetic riboswitch (first PCR forward primer for theoN5, ATACCAGCCGAAAGGCCCTTGGCAGAGAGGTCTGAAAAGACCTCTGCTGACTATGT GATCTTATTAAAATTAGG, second PCR forward primer for theoN5, GAAATTAATACGACTCACTATAGGGAGACCACAACGGTTTCCCTCCTCTATACCAGC CGAAAGGCCCTTGGCAG; first PCR forward primer for tc-N5, ACATACCAGATTTCGATCTGGAGAGGTGAAGAATACGACCACCTAGAGGTCTGAAA AGACCTCTGCTGACTATGTGATC, second PCR forward primer for tc-N5, GAAATTAATACGACTCACTATAGGGAGACCACAACGGTTTCCCTCCTCTAAAACATA CCAGATTTCGATC) to assess selective expression. Lipid-based transfection reagent is used according to the manufacturer's recommendations.

After 24 hr of culture at 37° C. and 5% CO2, cells are treated with and without theophylline or tetracycline, depending on the riboswitch encoded in the circular RNA, with concentrations ranging from 1 nM-3 mM. After 24 hrs of continuous culture, fluorescence or luminescence is evaluated. For GFP, live cells are imaged in a fluorescence neutral DMEM media with 5% FBS and penicillin/streptomycin and a stain for the nuclei. For NLuc, luminescence is evaluated using a luciferase system, following the manufacturer's instructions using a luminometer instrument (Promega).

DNA template for NLuc (Blue: Plautia stali
intestine virus IRES, Orange: NLuc ORF)
GACACGCGGCCTTCCAAGCAGTTAGGGAAACCGACTTCTTTGAAGAAGAA
AGCTGACTATGTGATCTTATTAAAATTAGGTTAAATTTCGAGGTTAAAAA
TAGTTTTAATATTGCTATAGTCTTAGAGGTCTTGTATATTTATACTTACC
ACACAAGATGGACCGGAGCAGCCCTCCAATATCTAGTGTACCCTCGTGCT
CGCTCAAACATTAAGTGGTGTTGTGCGAAAAGAATCTCACTTCAAGAAAA
AGAAACTAGTATGGTCTTCACACTCGAAGATTTCGTTGGGGACTGGCGAC
AGACAGCCGGCTACAACCTGGACCAAGTCCTTGAACAGGGAGGTGTGTCC
AGTTTGTTTCAGAATCTCGGGGTGTCCGTAACTCCGATCCAAAGGATTGT
CCTGAGCGGTGAAAATGGGCTGAAGATCGACATCCATGTCATCATCCCGT
ATGAAGGTCTGAGCGGCGACCAAATGGGCCAGATCGAAAAAATTTTTAAG
GTGGTGTACCCTGTGGATGATCATCACTTTAAGGTGATCCTGCACTATGG
CACACTGGTAATCGACGGGGTTACGCCGAACATGATCGACTATTTCGGAC
GGCCGTATGAAGGCATCGCCGTGTTCGACGGCAAAAAGATCACTGTAACA
GGGACCCTGTGGAACGGCAACAAAATTATCGACGAGCGCCTGATCAACCC
CGACGGCTCCCTGCTGTTCCGAGTAACCATCAACGGAGTGACCGGCTGGC
GGCTGTGCGAACGCATTCTGGCGTAACTCGAGCTCGGTACCTGTCCGCGG
TCGCGACGTACGCGGGCGGCCGCCATAAATTGGATCCATATATAGGGCCC
GGGTTATAATTACCTCAGGTCGACGTCCCATGGTTTTGTATAGAATTTAC
GGCTAGCGCCGGATGCGACGCCGGTCGCGTCTTATCCGGCCTTCCTATAT
CAGGCGGTGTTTAAGACGCCGCCGCTTCGCCCAAATCCTTATGCCGGTTC
GACGACTGGACAAAATACTGTTTATCT
DNA template for eGFP (Blue: Plautia stali
intestine virus IRES, Orange: eGFP ORF)
GACACGCGGCCTTCCAAGCAGTTAGGGAAACCGACTTCTTTGAAGAAGAA
AGCTGACTATGTGATCTTATTAAAATTAGGTTAAATTTCGAGGTTAAAAA
TAGTTTTAATATTGCTATAGTCTTAGAGGTCTTGTATATTTATACTTACC
ACACAAGATGGACCGGAGCAGCCCTCCAATATCTAGTGTACCCTCGTGCT
CGCTCAAACATTAAGTGGTGTTGTGCGAAAAGAATCTCACTTCAAGAAAA
AGAAACTAGTATGGTGAGCAAGGGCGAGGAGCTGTTCACCGGGGTGGTGC
CCATCCTGGTCGAGCTGGACGGCGACGTAAACGGCCACAAGTTCAGCGTG
TCCGGCGAGGGCGAGGGCGATGCCACCTACGGCAAGCTGACCCTGAAGTT
CATCTGCACCACCGGCAAGCTGCCCGTGCCCTGGCCCACCCTCGTGACCA
CCCTGACCTACGGCGTGCAGTGCTTCAGCCGCTACCCCGACCACATGAAG
CAGCACGACTTCTTCAAGTCCGCCATGCCCGAAGGCTACGTCCAGGAGCG
CACCATCTTCTTCAAGGACGACGGCAACTACAAGACCCGCGCCGAGGTGA
AGTTCGAGGGCGACACCCTGGTGAACCGCATCGAGCTGAAGGGCATCGAC
TTCAAGGAGGACGGCAACATCCTGGGGCACAAGCTGGAGTACAACTACAA
CAGCCACAACGTCTATATCATGGCCGACAAGCAGAAGAACGGCATCAAGG
TGAACTTCAAGATCCGCCACAACATCGAGGACGGCAGCGTGCAGCTCGCC
GACCACTACCAGCAGAACACCCCCATCGGCGACGGCCCCGTGCTGCTGCC
CGACAACCACTACCTGAGCACCCAGTCCGCCCTGAGCAAAGACCCCAACG
AGAAGCGCGATCACATGGTCCTGCTGGAGTTCGTGACCGCCGCCGGGATC
ACTCTCGGCATGGACGAGCTGTACAAGTAACTCGAGCTCGGTACCTGTCC
GCGGTCGCGACGTACGCGGGCGGCCGCCATAAATTGGATCCATATATAGG
GCCCGGGTTATAATTACCTCAGGTCGACGTCCCATGGTTTTGTATAGAAT
TTACGGCTAGCGCCGGATGCGACGCCGGTCGCGTCTTATCCGGCCTTCCT
ATATCAGGCGGTGTTTAAGACGCCGCCGCTTCGCCCAAATCCTTATGCCG
GTTCGACGACTGGACAAAATACTGTTTATCT
Primer Sequences
Forward primer for 2 (underlined: T7 promoter)
GAAATTAATACGACTCACTATAGGGAGACCACAACGGTTTCCCTGACTAT
GTGATC
Forward primer in the 1st PCR for theoN5 (orange:
aptamer; red: aIRES; purple: aaIRES)
ATACCAGCCGAAAGGCCCTTGGCAGAGAGGTCTGAAAAGACCTCTGCTGA
CTATGTGATCTTATTAAAATTAGG
TheoN5 2nd PCR
GAAATTAATACGACTCACTATAGGGAGACCACAACGGTTTCCCTCCTCTA
TACCAGCCGAAAGGCCCTTGGCAG
Forward primer in the 1st PCR for tc-N5
ACATACCAGATTTCGATCTGGAGAGGTGAAGAATACGACCACCTAGAGGT
CTGAAAAGACCTCTGCTGACTATGTGATC
Forward primer in the 2nd PCR for tc-N5
GAAATTAATACGACTCACTATAGGGAGACCACAACGGTTTCCCTCCTCTA
AAACATACCAGATTTCGATC
Reverse primer in all PCRs
AGATAAACAGTATTTTGTCCAGTCGTCGAAC
Junction
GGACAAAATACTGTTTATCTGGGAGACCACAACGG
Splint
5′-CCG TTG TGG TCT CCC AGA TAA ACA GTA TTT TGT
CC-3′

### Example 58: Circular RNA with Modified Nucleotides was Generated, Translated, and Reduced Immune Effect of Circular RNA

This Example demonstrates the generation of modified circular polyribonucleotide that produced protein product. In addition, this Example demonstrates circular RNA engineered with nucleotide modifications had reduced immune effect as compared to a linear RNA.

A non-naturally occurring circular RNA engineered to include one or more desirable properties and with complete or partial incorporation of modified nucleotides was produced. As shown in the following Example, full length modified linear RNA or a hybrid of modified and unmodified linear RNA was circularized and expression of nLuc was assessed. In addition, modified circular RNA was shown to have reduced activation of immune related genes (q-PCR of MDA5, OAS and IFN-beta expression) in BJ cells, as compared to a non-modified circular RNA.

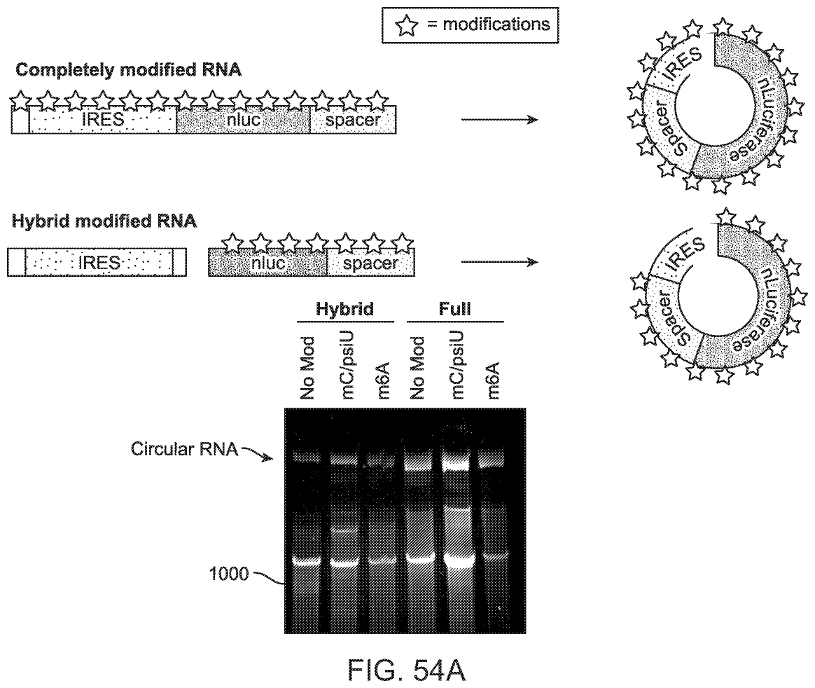

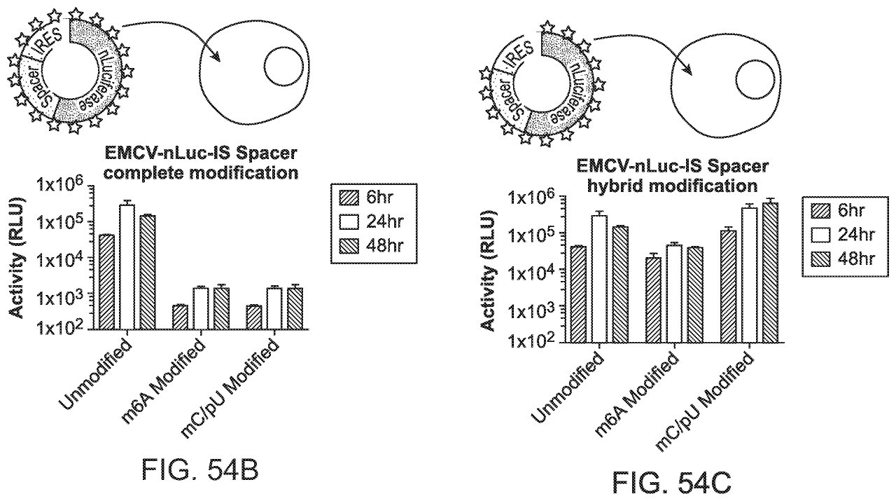

Circular RNA with a WT EMCV Nluc stop spacer was generated. For complete modification substitution, the modified nucleotides, pseudouridine and methylcytosine or m6A, were added in place of the standard unmodified nucleotides, uridine and cytosine or adenosine, respectively, during the in vitro transcription reaction. For the hybrid construct, the WT EMCV IRES was synthesized separately from the nLuc ORF. The WT EMCV IRES was synthesized using either modified or non-modified nucleotides. In contrast, the nLuc ORF sequence was synthesized using the modified nucleotides, pseudouridine and methylcytosine or m6A, in place of the standard unmodified nucleotides, uridine and cytosine or adenosine, respectively, during the in vitro transcription reaction. Following synthesis of the modified or unmodified IRES and the modified ORF, these two oligonucleotides were ligated together using T4 DNA ligase. As shown in FIG. 54A modified circular RNA was generated.

To measure expression efficiency of nLuc from the fully modified or hybrid modified constructs, 0.1 pmol of linear and circular RNA was transfected into BJ fibroblasts for 6 h. nLuc expression was measured at 6 h, 24 h, 48 h and 72 h post-transfection.

The level of innate immune response genes was monitored in cells from total RNA isolated from the cells using a phenol-based extraction reagent (Invitrogen). Total RNA (500 ng) was subjected to reverse transcription to generate cDNA. qRT-PCR analysis was performed using a dye-based quantitative PCR mix (BioRad).

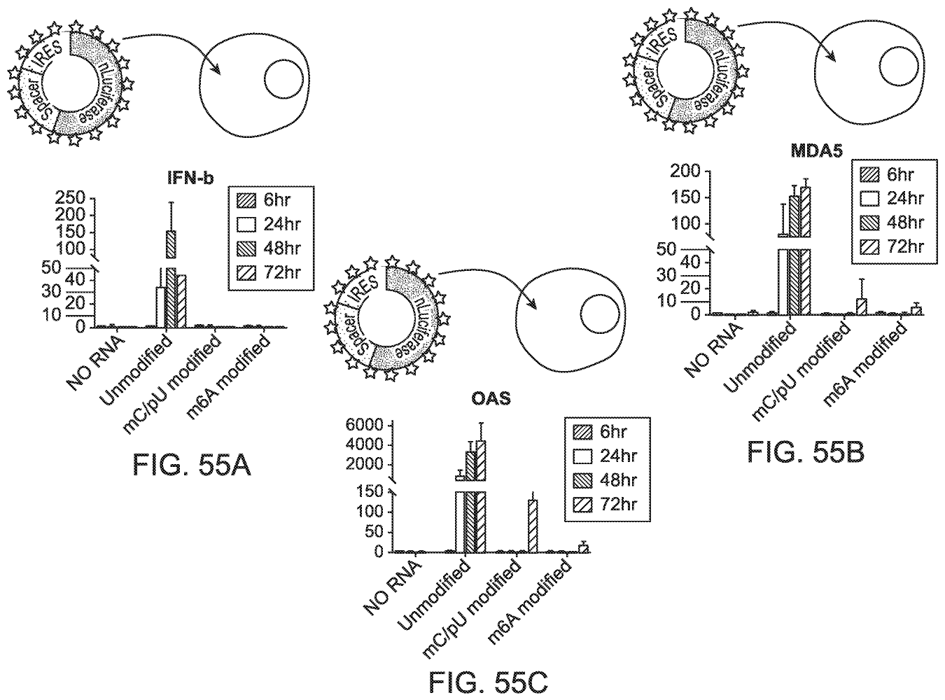

As shown in Figures FIG. 54B and FIG. 54C, modified circular RNA was translated as measured by luciferase activity. As shown in FIG. 55A, FIG. 55B, and FIG. 55C, qRT-PCR levels of immune related genes from BJ cells transfected with circular RNA showed reduction of MDA5, OAS and IFN-beta expression as compared to unmodified circular RNA transfected cells. Thus, induction of immunogenic related genes in recipient cells was reduced in cells transfected with modified circular RNA, as compared to unmodified circular RNA transfected cells.

### Example 59: Circular RNA Administered In Vivo Displayed a Longer Half-Life/Increased Stability Compared to Linear RNA

This Example demonstrates the ability to deliver circular RNA and the increased stability of circular RNA compared to linear RNA in vivo.

For this Example, circular RNAs were designed to include an EMCV IRES with an ORF encoding Nanoluciferase (Nluc) and stagger sequence (EMCV 2A 3× FLAG Nluc 2A no stop and EMCV 2A 3× FLAG Nluc 2A stop). The circular RNA was generated in vitro.

Balb/c mice were injected with circular RNA with Nluc ORF, or linear RNA as a control, via intravenous (IV) tail vein administration. Animals received a single dose of 5 μg of RNA formulated in a lipid-based transfection reagent (Mirus) according to manufacturer's instructions.

Mice were sacrificed, and livers were collected at 3, 4, and 7 days post-dosing (n=2 mice/time point). The livers were collected and stored in an RNA stabilization reagent (Invitrogen). The tissue was homogenized in RIPA buffer with micro tube homogenizer (Fisher scientific) and RNA was extracted using a phenol-based RNA extraction reagent for cDNA synthesis. qPCR was used to measure the presence of both linear and circular RNA in the liver.

RNA detection in tissues was performed by qPCR. To detect linear and circular RNA primers that amplify the Nluc ORF were used. (F: AGATTTCGTTGGGGACTGGC, R: CACCGCTCAGGACAATCCTT). To detect only circular RNA, primers that amplified the 5′-3′ junction allowed for detection of circular but not linear RNA constructs (F: CTGGAGACGTGGAGGAGAAC, R: CCAAAAGACGGCAATATGGT).

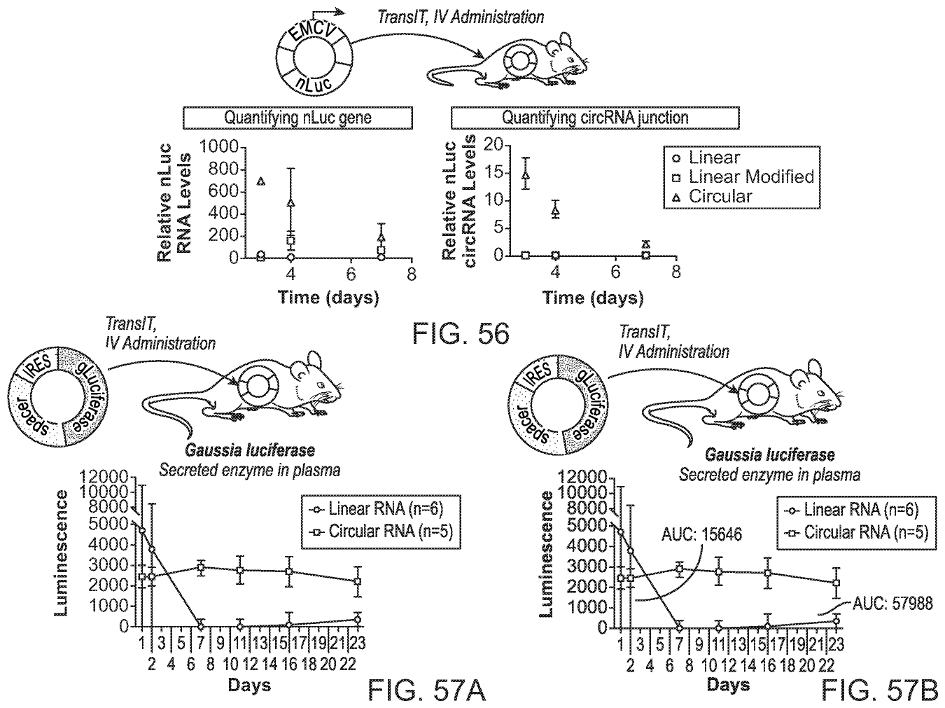

Circular RNA was detected at higher levels than linear RNA in livers of mice at 3, 4- and 7-days post-injection (FIG. 56). Therefore, circular RNA was administered and detectable in vivo for at least 7 days post administration.

### Example 60: In Vivo Expression, Half-Life, and Non-Immunogenicity of Circular RNA

This Example demonstrates the ability to drive expression from circular RNA in vivo. It demonstrates increased half-life of circular RNA compared to linear RNA. Finally, it demonstrates that circular RNA had a reduced immune effect in vivo

For this Example, circular RNAs included a CVB3 IRES, an ORF encoding Gaussia Luciferase (GLuc), and two spacer elements flanking the IRES-ORF.

The circular RNA was generated in vitro. Unmodified linear RNA was in vitro transcribed from a DNA template including all the motifs listed above, as well as a T7 RNA polymerase promoter to drive transcription. Transcribed RNA was purified with an RNA cleanup kit (New England Biolabs, T2050), treated with RNA 5′-phosphohydrolase (RppH) (New England Biolabs, M0356) following the manufacturer's instructions, and purified again with an RNA purification column. RppH treated RNA was circularized using a splint DNA (GTCAACGGATTTTCCCAAGTCCGTAGCGTCTC) and T4 RNA ligase 2 (New England Biolabs, M0239). Circular RNA was Urea-PAGE purified, eluted in a buffer (0.5M Sodium Acetate, 0.1% SDS, 1 mM EDTA), ethanol precipitated and resuspended in RNase free water.

Mice received a single tail vein injection dose of 2.5 μg of circular RNA with the Gaussia Luciferase ORF, or linear RNA as a control, both formulated in a lipid-based transfection reagent (Mirus) as a carrier.

Blood samples (50 μl) were collected from the tail-vein of each mouse into EDTA tubes, at 1, 2, 7, 11, 16, and 23 days post-dosing. Plasma was isolated by centrifugation for 25 min at 1300 g at 4° C. and the activity of Gaussia Luciferase, a secreted enzyme, was tested using a Gaussia Luciferase activity assay (Thermo Scientific Pierce). 50 μl of 1×Gluc substrate was added to 5 μl of plasma to carry out the Gluc luciferase activity assay. Plates were read right after mixing in a luminometer instrument (Promega).

Gaussia Luciferase activity was detected in plasma at 1, 2, 7, 11, 16, and 23 days post-dosing of circular RNA (FIG. 57A and FIG. 57B).

In contrast, Gaussia Luciferase activity was only detected in plasma at 1, and 2 days post-dosing of modified linear RNA. Enzyme activity from linear RNA derived protein was not detected above background levels at day 6 or beyond (FIG. 57A and FIG. 57B).

At day 16, livers were dissected from three animals and total RNA was isolated from cells using a phenol-based extraction reagent (Invitrogen). Total RNA (500 ng) was subjected to reverse transcription to generate cDNA. qRT-PCR analysis was performed using a dye-based quantitative PCR mix (BioRad).

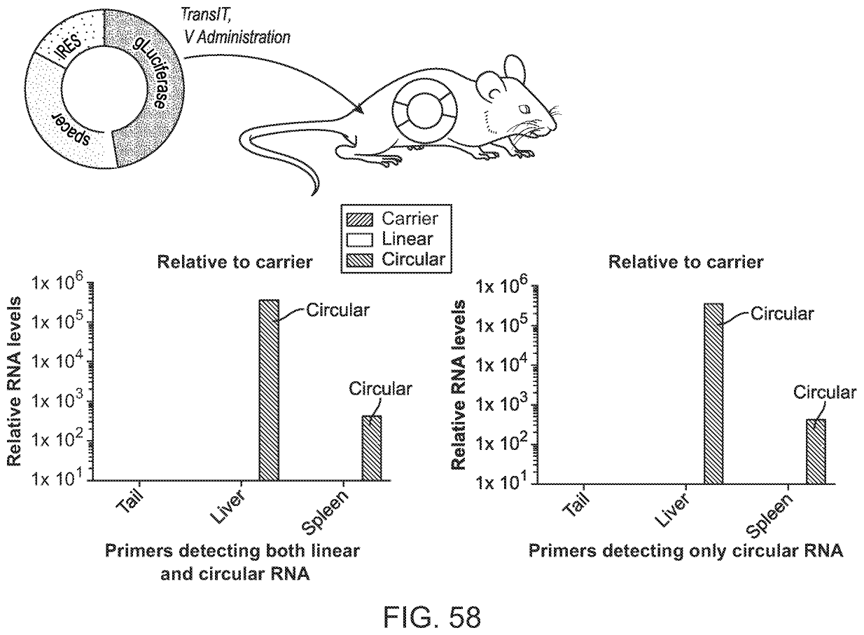

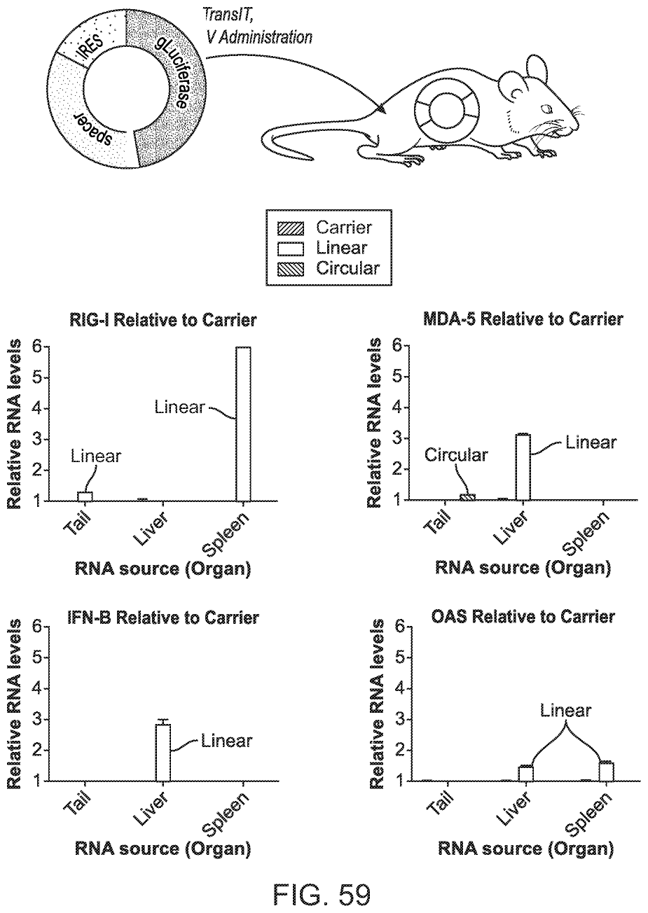

As shown in FIG. 58, qRT-PCR levels of circular RNA but not linear RNA were detected in both liver and spleen at day 16. As shown in FIG. 59, immune related genes from livers transfected with linear RNA showed increased expression of RIG-I, MDA5, IFN-B and OAS, while livers transfected with circular RNA did not show increased expression RIG-I, MDA5, PKR and IFN-beta of these markers as compared to carrier transfected animals at day 16. Thus, induction of immunogenic related genes in recipient cells was not present in circular RNA from transfected livers.

This Example demonstrated that circular RNA expressed protein in vivo for prolonged periods of time, with levels of protein activity in the plasma at multiple days post injection. Given the half-life of Gaussian Luciferase in mouse plasma is about 20 mins (see Tannous, Nat Protoc., 2009, 4(4):582-591), the similar levels of activity indicate continual expression from circular RNA. Further, circular RNA displayed a longer expression profile than its modified linear RNA counterpart without inducing immune related genes.

Sequence listing
(Start Codon)
SEQ ID NO: 1
AUG
(GFP)
EGFP:
SEQ ID NO: 2
atggtgagcaagggcgaggagctgttcaccggggtggtgcccatcctggtcgagctggacggcgacgtaaacggccacaagttcagcgt
gtccggcgagggcgagggcgatgccacctacggcaagctgaccctgaagttcatctgcaccaccggcaagctgcccgtgccctggccca
ccctcgtgaccaccctgacctacggcgtgcagtgcttcagccgctaccccgaccacatgaagcagcacgacttcttcaagtccgccatg
cccgaaggctacgtccaggagcgcaccatcttcttcaaggacgacggcaactacaagacccgcgccgaggtgaagttcgagggcgacac
cctggtgaaccgcatcgagctgaagggcatcgacttcaaggaggacggcaacatcctggggcacaagctggagtacaactacaacagcc
acaacgtctatatcatggccgacaagcagaagaacggcatcaaggtgaacttcaagatccgccacaacatcgaggacggcagcgtgcag
ctcgccgaccactaccagcagaacacccccatcggcgacggccccgtgctgctgcccgacaaccactacctgagcacccagtccgccct
gagcaaagaccccaacgagaagcgcgatcacatggtcctgctggagttcgtgaccgccgccgggatcactctcggcatggacgagctgt
acaag
(stagger element)
SEQ ID NO: 3
P2A:
gctactaacttcagcctgctgaagcaggctggcgacgtggaggagaaccctggacct
T2A:
gagggcaggggaagtctactaacatgcggggacgtggaggaaaatcccggccca
E2A:
cagtgtactaattatgctctcttgaaattggctggagatgttgagagcaacccaggtccc
Others: F2A, BmCPV2A, BmIFV2A
ZKSCAN introns
SEQ ID NO: 4
GTAAAAAGAGGTGAAACCTATTATGTGTGAGCAGGGCACAGACGTTGAAACTGGAG
CCAGGAGAAGTATTGGCAGGCTTTAGGTTATTAGGTGGTTACTCTGTCTTAAAAATG
TTCTGGCTTTCTTCCTGCATCCACTGGCATACTCATGGTCTGTTTTTAAATATTTTAAT
TCCCATTTACAAAGTGATTTACCCACAAGCCCAACCTGTCTGTCTTCAG
Or
GTAAGAAGCAAGGTTTCATTTAGGGGAAGGGAAATGATTCAGGACGAGAGTCTTTG
TGCTGCTGAGTGCCTGTGATGAAGAAGCATGTTAGTcctgggcaacgtagcgagaccccatctctacaa
aaaatagaaaaattagccaggtatagtggcgcacacctgtgattccagctacgcaggaggctgaggtgggaggattgcttgagcccagg
aggttgaggctgcagtgagctgtaatcatgccactactccaacctgggcaacacagcaaggaccctgtctcaaaaGCTACTTACA
GAAAAGAATTAggctcggcacggtagctcacacctgtaatcccagcactttgggaggctgaggcgggcagatcacttgaggtc
aggagtttgagaccagcctggccaacatggtgaaaccttgtctctactaaaaatatgaaaattagccaggcatggtggcacattcctgt
aatcccagctactcgggaggctgaggcaggagaatcacttgaacccaggaggtggaggttgcagtaagccgagatcgtaccactgtgct
ctagccttggtgacagagcgagactgtcttaaaaaaaaaaaaaaaaaaaaaagaattaattaaaaatttaaaaaaaaatgaaaaaaaGC
TGCATGCTTGTTTTTTGTTTTTAGTTATTCTACATTGTTGTCATTATTACCAAATATTGGGGA
AAATACAACTTACAGACCAATCTCAGGAGTTAAATGTTACTACGAAGGCAAATGAA
CTATGCGTAATGAACCTGGTAGGCATTA
(IRES)
IRES (EMCV):
SEQ ID NO: 5
Acgttactggccgaagccgcttggaataaggccggtgtgcgtttgtctatatgttattttccaccatattgccgtcttttggcaatgtg
agggcccggaaacctggccctgtcttcttgacgagcattcctaggggtctttcccctctcgccaaaggaatgcaaggtctgttgaatgt
cgtgaaggaagcagttcctctggaagatcttgaagacaaacaacgtctgtagcgaccctttgcaggcagcggaaccccccacctggcga
caggtgcctctgcggccaaaagccacgtgtataagatacacctgcaaaggcggcacaaccccagtgccacgttgtgagttggatagttg
tggaaagagtcaaatggctctcctcaagcgtattcaacaaggggctgaaggatgcccagaaggtaccccattgtatgggatctgatctg
gggcctcggtgcacatgctttacatgtgtttagtcgaggttaaaaaacgtctaggccccccgaaccacggggacgtggttttcctttga
aaaacacgatgataata
(addgene p3.1 laccase)
pcDNA3.1(+) Laccase2 MCS Exon Vector sequence 6926 bps
SEQ ID NO: 6
GACGGATCGGGAGATCTCCCGATCCCCTATGGTGCACTCTCAGTACAATCTGCTCTG
ATGCCGCATAGTTAAGCCAGTATCTGCTCCCTGCTTGTGTGTTGGAGGTCGCTGAGT
AGTGCGCGAGCAAAATTTAAGCTACAACAAGGCAAGGCTTGACCGACAATTGCATG
AAGAATCTGCTTAGGGTTAGGCGTTTTGCGCTGCTTCGCGATGTACGGGCCAGATAT
ACGCGTTGACATTGATTATTGACTAGTTATTAATAGTAATCAATTACGGGGTCATTA
GTTCATAGCCCATATATGGAGTTCCGCGTTACATAACTTACGGTAAATGGCCCGCCT
GGCTGACCGCCCAACGACCCCCGCCCATTGACGTCAATAATGACGTATGTTCCCATA
GTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTT
CCTACTTGGCAGTACATCTACGTATTAGTCATCGCTATTACCATGGTGATGCGGTTTT
GGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAA
AATGTCGTAACAACTCCGCCCCATTGACGCAAATGGGCGGTAGGCGTGTACGGTGG
GAGGTCTATATAAGCAGAGCTCTCTGGCTAACTAGAGAACCCACTGCTTACTGGCTT
ATCGAAATTAATACGACTCACTATAGGGAGACCCAAGCTGGCTAGCGTTTAAACTTA
AGCTTGGTACCGAGCTCGGATCCACTAGTCCAGTGTGGTGGAATTCCATTGAGAAAT
GACTGAGTTCCGGTGCTCTCAAGTCATTGATCTTTGTCGACTTTTATTTGGTCTCTGT
AATAACGACTTCAAAAACATTAAATTCTGTTGCGAAGCCAGTAAGCTACAAAAAGA
AAaaacaagagagaatgctatagtcgtatagtatagtttcccgactatctgatacccattacttatctagggggaatgcgaacccaaaa
ttttatcagttttctcggatatcgatagatattggggaataaatttaaataaataaattttgggcgggtttagggcgtggcaaaaagtt
ttttggcaaatcgctagaaatttacaagacttataaaattatgaaaaaatacaacaaaattttaaacacgtgggcgtgacagttttggG
cggttttagggcgttagagtaggcgaggacagggttacatcgactaggattgatcctgatcaagaatatatatactttataccgcttcc
ttctacatgttacctatttttcaacgaatctagtatacctttttactgtacgatttatgggtataaTAATAAGCTAAATCGAGACTAAG
ttttattgttatatatattttttttattttatGCAGAAATTAATTAAACCGGTCCTGCAGGTGATCAGGCGCGCCGGTTACCGG
CCGGCCCCGCGGAGCGTAAGTATTCAAAATTCCAAAATTTTTTACTAGAAATATTCG
ATTTTTTAATAGGCAGTTTCTATACTATTGTATACTATTGtagattcgttgaaaagtatgtaacaggaag
aataaagcatttccgaccatgtaaagtatatatattataataaggatcaatagccgagtcgatctcgccatgtccgtctgtcttattGt
tttattaccgccgagacatcaggaactataaaagctagaaggatgagttttagcatacagattctagagacaaggacgcagagcaagtt
tgttgatccatgctgccacgctttaactttctcaaattgcccaaaactgccatgcccacatttttgaactattttcgaaattttttcat
aattgtattactcgtgtaaatttccatcaatttgccaaaaaactttttgtcacgcgttaacgccctaaagccgccaatttggtcacgcc
cacactattgaGcaattatcaaattttttctcattttattccccaatatctatcgatatccccgattatgaaattattaaatttcgcgt
tcgcattcacactagctgagtaacgagtatctgatagttggggaaatcgactTATTTTTTATATACAATGAAAATGAATTTAATCATAT
GAATATCGATTATAGCTTTTTATTTAATATGAATATTTATTTGGGCTTAAGGTGTAACCTcctcgacataagactcacat
ggcgcaggcacattgaagacaaaaatactcaTTGTCGGGTCTCGCACCCTCCAGCAGCACCTAAAATT
ATGTCTTCAATTATTGCCAACATTGGAGACACAATTAGTCTGTGGCACCTCAGGCGG
CCGCTCGAGTCTAGAGGGCCCGTTTAAACCCGCTGATCAGCCTCGACTGTGCCTTCT
AGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCTTCTGAGGCGGA
AAGAACCAGCTGGGGCTCTAGGGGGTATCCCCACGCGCCCTGTAGCGGCGCATTAA
GCGCGGCGGGTGTGGTGGTTACGCGCAGCGTGACCGCTACACTTGCCAGCGCCCTA
GCGCCCGCTCCTTTCGCTTTCTTCCCTTCCTTTCTCGCCACGTTCGCCGGCTTTCCCCG
TCAAGCTCTAAATCGGGGGCTCCCTTTAGGGTTCCGATTTAGTGCTTTACGGCACCT
CGACCCCAAAAAACTTGATTAGGGTGATGGTTCACGTAGTGGGCCATCGCCCTGATA
GACGGTTTTTCGCCCTTTGACGTTGGAGTCCACGTTCTTTAATAGTGGACTCTTGTTC
CAAACTGGAACAACACTCAACCCTATCTCGGTCTATTCTTTTGATTTATAAGGGATTT
TGCCGATTTCGGCCTATTGGTTAAAAAATGAGCTGATTTAACAAAAATTTAACGCGA
ATTAATTCTGTGGAATGTGTGTCAGTTAGGGTGTGGAAAGTCCCCAGGCTCCCCAGC
AGGCAGAAGTATGCAAAGCATGCATCTCAATTAGTCAGCAACCAGGTGTGGAAAGT
CCCCAGGCTCCCCAGCAGGCAGAAGTATGCAAAGCATGCATCTCAATTAGTCAGCA
ACCATAGTCCCGCCCCTAACTCCGCCCATCCCGCCCCTAACTCCGCCCAGTTCCGCC
CATTCTCCGCCCCATGGCTGACTAATTTTTTTTATTTATGCAGAGGCCGAGGCCGCCT
CTGCCTCTGAGCTATTCCAGAAGTAGTGAGGAGGCTTTTTTGGAGGCCTAGGCTTTT
GCAAAAAGCTCCCGGGAGCTTGTATATCCATTTTCGGATCTGATCAAGAGACAGGAT
GAGGATCGTTTCGCATGATTGAACAAGATGGATTGCACGCAGGTTCTCCGGCCGCTT
GGGTGGAGAGGCTATTCGGCTATGACTGGGCACAACAGACAATCGGCTGCTCTGAT
GCCGCCGTGTTCCGGCTGTCAGCGCAGGGGCGCCCGGTTCTTTTTGTCAAGACCGAC
CTGTCCGGTGCCCTGAATGAACTGCAGGACGAGGCAGCGCGGCTATCGTGGCTGGC
CACGACGGGCGTTCCTTGCGCAGCTGTGCTCGACGTTGTCACTGAAGCGGGAAGGG
ACTGGCTGCTATTGGGCGAAGTGCCGGGGCAGGATCTCCTGTCATCTCACCTTGCTC
CTGCCGAGAAAGTATCCATCATGGCTGATGCAATGCGGCGGCTGCATACGCTTGATC
CGGCTACCTGCCCATTCGACCACCAAGCGAAACATCGCATCGAGCGAGCACGTACT
CGGATGGAAGCCGGTCTTGTCGATCAGGATGATCTGGACGAAGAGCATCAGGGGCT
CGCGCCAGCCGAACTGTTCGCCAGGCTCAAGGCGCGCATGCCCGACGGCGAGGATC
TCGTCGTGACCCATGGCGATGCCTGCTTGCCGAATATCATGGTGGAAAATGGCCGCT
TTTCTGGATTCATCGACTGTGGCCGGCTGGGTGTGGCGGACCGCTATCAGGACATAG
CGTTGGCTACCCGTGATATTGCTGAAGAGCTTGGCGGCGAATGGGCTGACCGCTTCC
TCGTGCTTTACGGTATCGCCGCTCCCGATTCGCAGCGCATCGCCTTCTATCGCCTTCT
TGACGAGTTCTTCTGAGCGGGACTCTGGGGTTCGAAATGACCGACCAAGCGACGCC
CAACCTGCCATCACGAGATTTCGATTCCACCGCCGCCTTCTATGAAAGGTTGGGCTT
CGGAATCGTTTTCCGGGACGCCGGCTGGATGATCCTCCAGCGCGGGGATCTCATGCT
GGAGTTCTTCGCCCACCCCAACTTGTTTATTGCAGCTTATAATGGTTACAAATAAAG
CAATAGCATCACAAATTTCACAAATAAAGCATTTTTTTCACTGCATTCTAGTTGTGGT
TTGTCCAAACTCATCAATGTATCTTATCATGTCTGTATACCGTCGACCTCTAGCTAGA
GCTTGGCGTAATCATGGTCATAGCTGTTTCCTGTGTGAAATTGTTATCCGCTCACAAT
TCCACACAACATACGAGCCGGAAGCATAAAGTGTAAAGCCTGGGGTGCCTAATGAG
TGAGCTAACTCACATTAATTGCGTTGCGCTCACTGCCCGCTTTCCAGTCGGGAAACC
TGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGG
GATAACGCAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACGAGCATCACA
AAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTG
TAGGTATCTCAGTTCGGTGTAGGTCGTTCGCTCCAAGCTGGGCTGTGTGCACGAACC
CCCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGAG
CGAGGTATGTAGGCGGTGCTACAGAGTTCTTGAAGTGGTGGCCTAACTACGGCTAC
ACTAGAAGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTTTTTTTGTT
TGCAAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAA
ATCAATCTAAAGTATATATGAGTAAACTTGGTCTGACAGTTACCAATGCTTAATCAG
TGAGGCACCTATCTCAGCGATCTGTCTATTTCGTTCATCCATAGTTGCCTGACTCCCC
GTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGC
CGGAAGGGCCGAGCGCAGAAGTGGTCCTGCAACTTTATCCGCCTCCATCCAGTCTAT
TAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAA
GCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGAT
GCTTTTCTGTGACTGGTGAGTACTCAACCAAGTCATTCTGAGAATAGTGTATGCGGC
GACCGAGTTGCTCTTGCCCGGCGTCAATACGGGATAATACCGCGCCACATAGCAGA
ACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCA
GCATCTTTTACTTTCACCAGCGTTTCTGGGTGAGCAAAAACAGGAAGGCAAAATGCC
GCAAAAAAGGGAATAAGGGCGACACGGAAATGTTGAATACTCATACTCTTCCTTTTT
CAATATTATTGAAGCATTTATCAGGGTTATTGTCTCATGAGCGGATACATATTTGAA
TGTATTTAGAAAAATAAACAAATAGGGGTTCCGCGCACATTTCCCCGAAAAGTGCC
ACCTGACGTC
(RFP)
mCherry:
SEQ ID NO: 8
atggtgagcaagggcgaggaggataacatggccatcatcaaggagttcatgcgcttcaaggtgcacatggagggctccgtgaacggcca
cgagttcgagatcgagggcgagggcgagggccgccectacgagggcacccagaccgccaagctgaaggtgaccaagggtggccccctgc
ccttcgcctgggacatcctgtcccctcagttcatgtacggctccaaggcctacgtgaagcaccccgccgacatccccgactacttgaag
ctgtccttccccgagggcttcaagtgggagcgcgtgatgaacttcgaggacggcggcgtggtgaccgtgacccaggactcctccctgca
ggacggcgagttcatctacaaggtgaagctgcgcggcaccaacttcccctccgacggccccgtaatgcagaagaagaccatgggctggg
aggcctcctccgagcggatgtaccccgaggacggcgccctgaagggcgagatcaagcagaggctgaagctgaaggacggcggccacta
cgacgctgaggtcaagaccacctacaaggccaagaagcccgtgcagctgcccggcgcctacaacgtcaacatcaagttggacatcacct
cccacaacgaggactacaccatcgtggaacagtacgaacgcgccgagggccgccactccaccggcggcatggacgagctgtacaag
(riboswitch)
Aptazyme (Theophylline Dependent see Auslander 2010 Mol Biosys):
SEQ ID NO: 9
cugagaugcagguacauccagcugaugagucccaaauaggacgaaagccauaccagccgaaaggcccuuggcaggguuccugg
auuccacugcuauccac
(luciferase)
nLuc:
SEQ ID NO: 10
ATGGTCTTCACACTCGAAGATTTCGTTGGGGACTGGCGACAGACAGCCGGCTACAA
CCTGGACCAAGTCCTTGAACAGGGAGGTGTGTCCAGTTTGTTTCAGAATCTCGGGGT
GTCCGTAACTCCGATCCAAAGGATTGTCCTGAGCGGTGAAAATGGGCTGAAGATCG
ACATCCATGTCATCATCCCGTATGAAGGTCTGAGCGGCGACCAAATGGGCCAGATC
GAAAAAATTTTTAAGGTGGTGTACCCTGTGGATGATCATCACTTTAAGGTGATCCTG
CACTATGGCACACTGGTAATCGACGGGGTTACGCCGAACATGATCGACTATTTCGGA
CGGCCGTATGAAGGCATCGCCGTGTTCGACGGCAAAAAGATCACTGTAACAGGGAC
CCTGTGGAACGGCAACAAAATTATCGACGAGCGCCTGATCAACCCCGACGGCTCCC
TGCTGTTCCGAGTAACCATCAACGGAGTGACCGGCTGGCGGCTGTGCGAACGCATTC
TGGCGTAA
Kozak 3XFLAG-EGF nostop (264 bps)
SEQ ID NO: 11
GGGAGCCACCATGGACTACAAGGACGACGACGACAAGATCATCGACTATAAAGAC
GACGACGATAAAGGTGGCGACTATAAGGACGACGACGACAAAGCCATTAATAGTG
ACTCTGAGTGTCCCCTGTCCCACGACGGGTACTGCCTCCACGACGGTGTGTGCATGT
ATATTGAAGCATTGGACAAGTACGCCTGCAACTGTGTTGTTGGCTACATCGGGGAGC
GCTGTCAGTACCGAGACCTGAAGTGGTGGGAACTGCGCCT
5-13: Kozak sequence
14-262: 3XFLAG-EGF
Kozak 3XFLAG-EGF stop (273 bps)
SEQ ID NO: 12
GGGAGCCACCATGGACTACAAGGACGACGACGACAAGATCATCGACTATAAAGAC
GACGACGATAAAGGTGGCGACTATAAGGACGACGACGACAAAGCCATTAATAGTG
ACTCTGAGTGTCCCCTGTCCCACGACGGGTACTGCCTCCACGACGGTGTGTGCATGT
ATATTGAAGCATTGGACAAGTACGCCTGCAACTGTGTTGTTGGCTACATCGGGGAGC
GCTGTCAGTACCGAGACCTGAAGTGGTGGGAACTGCGCTGATAGTAACT
5-13: Kozak sequence
14-262: 3XFLAG-EGF
263-271: Triple stop codon
Kozak 3XFLAG-EGF P2A nostop (330 bps)
SEQ ID NO: 13
GGGAGCCACCATGGACTACAAGGACGACGACGACAAGATCATCGACTATAAAGAC
GACGACGATAAAGGTGGCGACTATAAGGACGACGACGACAAAGCCATTAATAGTG
ACTCTGAGTGTCCCCTGTCCCACGACGGGTACTGCCTCCACGACGGTGTGTGCATGT
ATATTGAAGCATTGGACAAGTACGCCTGCAACTGTGTTGTTGGCTACATCGGGGAGC
GCTGTCAGTACCGAGACCTGAAGTGGTGGGAACTGCGCGGAAGCGGAGCTACTAAC
TTCAGCCTGCTGAAGCAGGCTGGAGACGTGGAGGAGAACCCTGGACCTCT
5-13: Kozak sequence
14-262: 3XFLAG-EGF
263-328: P2A
Splint for construct Kozak 3XFLAG-EGF nostop (264 bps)
SEQ ID NO: 14
GGTGGCTCCCAGGCGCAGTT
Splint for construct Kozak 3XFLAG-EGF stop (273 bps)
SEQ ID NO: 15
GGTGGCTCCCAGTTACTATC
Splint for construct Kozak 3XFLAG-EGF P2A nostop (330 bps)
SEQ ID NO: 16
GGTGGCTCCCAGAGGTCCAG
Kozak 1XFLAG-EGF T2A 1XFLAG-Nluc P2A nostop (873 bps)
SEQ ID NO: 17
GGGAGCCACCATGGACTACAAGGACGACGACGACAAGATCATCAATAGTGACTCTG
AGTGTCCCCTGTCCCACGACGGGTACTGCCTCCACGACGGTGTGTGCATGTATATTG
AAGCATTGGACAAGTACGCCTGCAACTGTGTTGTTGGCTACATCGGGGAGCGCTGTC
AGTACCGAGACCTGAAGTGGTGGGAACTGCGCGGCTCCGGCGAGGGCAGGGGAAG
TCTTCTAACATGCGGGGACGTGGAGGAAAATCCCGGCCCAGACTATAAGGACGACG
ACGACAAAATCATCGTCTTCACACTCGAAGATTTCGTTGGGGACTGGCGACAGACA
GCCGGCTACAACCTGGACCAAGTCCTTGAACAGGGAGGTGTGTCCAGTTTGTTTCAG
AATCTCGGGGTGTCCGTAACTCCGATCCAAAGGATTGTCCTGAGCGGTGAAAATGG
GCTGAAGATCGACATCCATGTCATCATCCCGTATGAAGGTCTGAGCGGCGACCAAA
TGGGCCAGATCGAAAAAATTTTTAAGGTGGTGTACCCTGTGGATGATCATCACTTTA
AGGTGATCCTGCACTATGGCACACTGGTAATCGACGGGGTTACGCCGAACATGATC
GACTATTTCGGACGGCCGTATGAAGGCATCGCCGTGTTCGACGGCAAAAAGATCAC
TGTAACAGGGACCCTGTGGAACGGCAACAAAATTATCGACGAGCGCCTGATCAACC
CCGACGGCTCCCTGCTGTTCCGAGTAACCATCAACGGAGTGACCGGCTGGCGGCTGT
GCGAACGCATTCTGGCGGGAAGCGGAGCTACTAACTTCAGCCTGCTGAAGCAGGCT
GGAGACGTGGAGGAGAACCCTGGACCTCT
5-13: Kozak sequence
14-202: 1XFLAG-EGF
203-265: T2A
266-805: 1XFLAG-Nluc
806-871: P2A
Kozak 1XFLAG-EGF stop 1XFLAG-Nluc stop (762 bps)
SEQ ID NO: 18
GGGAGCCACCATGGACTACAAGGACGACGACGACAAGATCATCAATAGTGACTCTG
AGTGTCCCCTGTCCCACGACGGGTACTGCCTCCACGACGGTGTGTGCATGTATATTG
AAGCATTGGACAAGTACGCCTGCAACTGTGTTGTTGGCTACATCGGGGAGCGCTGTC
AGTACCGAGACCTGAAGTGGTGGGAACTGCGCTGATAGTAAGACTATAAGGACGAC
GACGACAAAATCATCGTCTTCACACTCGAAGATTTCGTTGGGGACTGGCGACAGAC
AGCCGGCTACAACCTGGACCAAGTCCTTGAACAGGGAGGTGTGTCCAGTTTGTTTCA
GAATCTCGGGGTGTCCGTAACTCCGATCCAAAGGATTGTCCTGAGCGGTGAAAATG
GGCTGAAGATCGACATCCATGTCATCATCCCGTATGAAGGTCTGAGCGGCGACCAA
ATGGGCCAGATCGAAAAAATTTTTAAGGTGGTGTACCCTGTGGATGATCATCACTTT
AAGGTGATCCTGCACTATGGCACACTGGTAATCGACGGGGTTACGCCGAACATGAT
CGACTATTTCGGACGGCCGTATGAAGGCATCGCCGTGTTCGACGGCAAAAAGATCA
CTGTAACAGGGACCCTGTGGAACGGCAACAAAATTATCGACGAGCGCCTGATCAAC
CCCGACGGCTCCCTGCTGTTCCGAGTAACCATCAACGGAGTGACCGGCTGGCGGCTG
TGCGAACGCATTCTGGCGTGATAGTAACT
5-13: Kozak sequence
14-202: 1XFLAG-EGF
203-211: Triple stop codon
212-751: 1XFLAG-Nluc
752-760: Triple stop codon
Kozak 3XFLAG-EGF P2A nostop (330 bps)
SEQ ID NO: 19
GGGAGCCACCATGGACTACAAGGACGACGACGACAAGATCATCGACTATAAAGAC
GACGACGATAAAGGTGGCGACTATAAGGACGACGACGACAAAGCCATTAATAGTG
ACTCTGAGTGTCCCCTGTCCCACGACGGGTACTGCCTCCACGACGGTGTGTGCATGT
ATATTGAAGCATTGGACAAGTACGCCTGCAACTGTGTTGTTGGCTACATCGGGGAGC
GCTGTCAGTACCGAGACCTGAAGTGGTGGGAACTGCGCGGAAGCGGAGCTACTAAC
TTCAGCCTGCTGAAGCAGGCTGGAGACGTGGAGGAGAACCCTGGACCTCT
5-13: Kozak sequence
14-262: 3XFLAG-EGF
263-328: P2A
Kozak 3XFLAG-EGF nostop (264 bps)
SEQ ID NO: 20
GGGAGCCACCATGGACTACAAGGACGACGACGACAAGATCATCGACTATAAAGAC
GACGACGATAAAGGTGGCGACTATAAGGACGACGACGACAAAGCCATTAATAGTG
ACTCTGAGTGTCCCCTGTCCCACGACGGGTACTGCCTCCACGACGGTGTGTGCATGT
ATATTGAAGCATTGGACAAGTACGCCTGCAACTGTGTTGTTGGCTACATCGGGGAGC
GCTGTCAGTACCGAGACCTGAAGTGGTGGGAACTGCGCCT
5-13: Kozak sequence
14-262: 3XFLAG-EGF
Kozak 3XFLAG-EGF stop (273 bps)
SEQ ID NO: 21
GGGAGCCACCATGGACTACAAGGACGACGACGACAAGATCATCGACTATAAAGAC
GACGACGATAAAGGTGGCGACTATAAGGACGACGACGACAAAGCCATTAATAGTG
ACTCTGAGTGTCCCCTGTCCCACGACGGGTACTGCCTCCACGACGGTGTGTGCATGT
ATATTGAAGCATTGGACAAGTACGCCTGCAACTGTGTTGTTGGCTACATCGGGGAGC
GCTGTCAGTACCGAGACCTGAAGTGGTGGGAACTGCGCTGATAGTAACT
5-13: Kozak sequence
14-262: 3XFLAG-EGF
263-271: Triple stop codon
EMCV IRES T2A 3XFLAG-EGF P2A nostop (954 bps)
SEQ ID NO: 22
GGGACCTAACGTTACTGGCCGAAGCCGCTTGGAACAAGGCCGGTGTGCGTTTGTCTA
TATGTTATTTTCCACCATATTGCCGTCTTTTGGCAATGTGAGGGCCCGGAAACCTGG
CCCTGTCTTCTTGACGAGCATTCCTAGGGGTCTTTCCCCTCTCGCCAAAGGAATGCA
AGGTCTGTTGAATGTCGTGAAGGAAGCAGTTCCTCTGGAAGCTTCTTCAAGACAAAC
AACGTCTGTAGCGACCCTTTGCAGGCAGCGGAACCCCCCACCTGGCGACAGGTGCC
TCTGCGGCCAAAAGCCACGTGTATACGATACACCTGCAAAGGCGGCACAACCCCAG
TGCCACGTTGTGAGTTGGATAGTTGTGGAAAGAGTCAAATGGCTCTCCTCAAGCGTA
TTCAACAAGGGGCTGAAGGATGCCCAGAAGGTACCCCATTGTATGGGATCTGATCT
GGGGCCTCGGTGCACATGCTTTACATGTGTTCAGTCGAGGTTAAAAAACGTCCAGGC
CCCCCGAACCACGGGGACGTGGTTTTCCTTTGAAAAACACGATGATAATATGGCCAC
AACCATGGGCTCCGGCGAGGGCAGGGGAAGTCTTCTAACATGCGGGGACGTGGAGG
AAAATCCCGGCCCAGACTACAAGGACGACGACGACAAGATCATCGACTATAAAGAC
GACGACGATAAAGGTGGCGACTATAAGGACGACGACGACAAAGCCATTAATAGTG
ACTCTGAGTGTCCCCTGTCCCACGACGGGTACTGCCTCCACGACGGTGTGTGCATGT
ATATTGAAGCATTGGACAAGTACGCCTGCAACTGTGTTGTTGGCTACATCGGGGAGC
GCTGTCAGTACCGAGACCTGAAGTGGTGGGAACTGCGCGGAAGCGGAGCTACTAAC
TTCAGCCTGCTGAAGCAGGCTGGAGACGTGGAGGAGAACCCTGGACCTCT
5-574: EMCV IRES
575-637: T2A
638-886: 3XFALG-EGF
887-952: P2A
EMCV T2A 3XFLAG Nluc P2A stop (1314 nts)
SEQ ID NO: 23
GGGACCTAACGTTACTGGCCGAAGCCGCTTGGAACAAGGCCGGTGTGCGTTTGTCTA
TATGTTATTTTCCACCATATTGCCGTCTTTTGGCAATGTGAGGGCCCGGAAACCTGG
CCCTGTCTTCTTGACGAGCATTCCTAGGGGTCTTTCCCCTCTCGCCAAAGGAATGCA
AGGTCTGTTGAATGTCGTGAAGGAAGCAGTTCCTCTGGAAGCTTCTTCAAGACAAAC
AACGTCTGTAGCGACCCTTTGCAGGCAGCGGAACCCCCCACCTGGCGACAGGTGCC
TCTGCGGCCAAAAGCCACGTGTATACGATACACCTGCAAAGGCGGCACAACCCCAG
TGCCACGTTGTGAGTTGGATAGTTGTGGAAAGAGTCAAATGGCTCTCCTCAAGCGTA
TTCAACAAGGGGCTGAAGGATGCCCAGAAGGTACCCCATTGTATGGGATCTGATCT
GGGGCCTCGGTGCACATGCTTTACATGTGTTCAGTCGAGGTTAAAAAACGTCCAGGC
CCCCCGAACCACGGGGACGTGGTTTTCCTTTGAAAAACACGATGATAATATGGCCAC
AACCATGGGCTCCGGCGAGGGCAGGGGAAGTCTTCTAACATGCGGGGACGTGGAGG
AAAATCCCGGCCCAGACTACAAGGACGACGACGACAAGATCATCGACTATAAAGAC
GACGACGATAAAGGTGGCGACTATAAGGACGACGACGACAAAGCCATTGTCTTCAC
ACTCGAAGATTTCGTTGGGGACTGGCGACAGACAGCCGGCTACAACCTGGACCAAG
TCCTTGAACAGGGAGGTGTGTCCAGTTTGTTTCAGAATCTCGGGGTGTCCGTAACTC
CGATCCAAAGGATTGTCCTGAGCGGTGAAAATGGGCTGAAGATCGACATCCATGTC
ATCATCCCGTATGAAGGTCTGAGCGGCGACCAAATGGGCCAGATCGAAAAAATTTT
TAAGGTGGTGTACCCTGTGGATGATCATCACTTTAAGGTGATCCTGCACTATGGCAC
ACTGGTAATCGACGGGGTTACGCCGAACATGATCGACTATTTCGGACGGCCGTATG
AAGGCATCGCCGTGTTCGACGGCAAAAAGATCACTGTAACAGGGACCCTGTGGAAC
GGCAACAAAATTATCGACGAGCGCCTGATCAACCCCGACGGCTCCCTGCTGTTCCGA
GTAACCATCAACGGAGTGACCGGCTGGCGGCTGTGCGAACGCATTCTGGCGGGAAG
CGGAGCTACTAACTTCAGCCTGCTGAAGCAGGCTGGAGACGTGGAGGAGAACCCTG
GACCTTGATAGTAACT
5-574: EMCV IRES
575-637: T2A
638-1237: 3XFLAG Nluc
1238-1303: P2A
1304-1312: Triple stop codon
EMCV T2A 3XFLAG Nluc P2A nostop (1305 nts)
SEQ ID NO: 24
GGGACCTAACGTTACTGGCCGAAGCCGCTTGGAACAAGGCCGGTGTGCGTTTGTCTA
TATGTTATTTTCCACCATATTGCCGTCTTTTGGCAATGTGAGGGCCCGGAAACCTGG
CCCTGTCTTCTTGACGAGCATTCCTAGGGGTCTTTCCCCTCTCGCCAAAGGAATGCA
AGGTCTGTTGAATGTCGTGAAGGAAGCAGTTCCTCTGGAAGCTTCTTCAAGACAAAC
AACGTCTGTAGCGACCCTTTGCAGGCAGCGGAACCCCCCACCTGGCGACAGGTGCC
TCTGCGGCCAAAAGCCACGTGTATACGATACACCTGCAAAGGCGGCACAACCCCAG
TGCCACGTTGTGAGTTGGATAGTTGTGGAAAGAGTCAAATGGCTCTCCTCAAGCGTA
TTCAACAAGGGGCTGAAGGATGCCCAGAAGGTACCCCATTGTATGGGATCTGATCT
GGGGCCTCGGTGCACATGCTTTACATGTGTTCAGTCGAGGTTAAAAAACGTCCAGGC
CCCCCGAACCACGGGGACGTGGTTTTCCTTTGAAAAACACGATGATAATATGGCCAC
AACCATGGGCTCCGGCGAGGGCAGGGGAAGTCTTCTAACATGCGGGGACGTGGAGG
AAAATCCCGGCCCAGACTACAAGGACGACGACGACAAGATCATCGACTATAAAGAC
GACGACGATAAAGGTGGCGACTATAAGGACGACGACGACAAAGCCATTGTCTTCAC
ACTCGAAGATTTCGTTGGGGACTGGCGACAGACAGCCGGCTACAACCTGGACCAAG
TCCTTGAACAGGGAGGTGTGTCCAGTTTGTTTCAGAATCTCGGGGTGTCCGTAACTC
CGATCCAAAGGATTGTCCTGAGCGGTGAAAATGGGCTGAAGATCGACATCCATGTC
ATCATCCCGTATGAAGGTCTGAGCGGCGACCAAATGGGCCAGATCGAAAAAATTTT
TAAGGTGGTGTACCCTGTGGATGATCATCACTTTAAGGTGATCCTGCACTATGGCAC
ACTGGTAATCGACGGGGTTACGCCGAACATGATCGACTATTTCGGACGGCCGTATG
AAGGCATCGCCGTGTTCGACGGCAAAAAGATCACTGTAACAGGGACCCTGTGGAAC
GGCAACAAAATTATCGACGAGCGCCTGATCAACCCCGACGGCTCCCTGCTGTTCCGA
GTAACCATCAACGGAGTGACCGGCTGGCGGCTGTGCGAACGCATTCTGGCGGGAAG
CGGAGCTACTAACTTCAGCCTGCTGAAGCAGGCTGGAGACGTGGAGGAGAACCCTG
GACCTCT
5-574: EMCV IRES
575-637: T2A
638-1237: 3XFLAG Nluc
1238-1303: P2A
Kozak 1XFLAG-EGF T2A 1XFLAG-NLuc P2A stop (882 bps)
SEQ ID NO: 25
GGGAGCCACCATGGACTACAAGGACGACGACGACAAGATCATCAATAGTGACTCTG
AGTGTCCCCTGTCCCACGACGGGTACTGCCTCCACGACGGTGTGTGCATGTATATTG
AAGCATTGGACAAGTACGCCTGCAACTGTGTTGTTGGCTACATCGGGGAGCGCTGTC
AGTACCGAGACCTGAAGTGGTGGGAACTGCGCGGCTCCGGCGAGGGCAGGGGAAG
TCTTCTAACATGCGGGGACGTGGAGGAAAATCCCGGCCCAGACTATAAGGACGACG
ACGACAAAATCATCGTCTTCACACTCGAAGATTTCGTTGGGGACTGGCGACAGACA
GCCGGCTACAACCTGGACCAAGTCCTTGAACAGGGAGGTGTGTCCAGTTTGTTTCAG
AATCTCGGGGTGTCCGTAACTCCGATCCAAAGGATTGTCCTGAGCGGTGAAAATGG
GCTGAAGATCGACATCCATGTCATCATCCCGTATGAAGGTCTGAGCGGCGACCAAA
TGGGCCAGATCGAAAAAATTTTTAAGGTGGTGTACCCTGTGGATGATCATCACTTTA
AGGTGATCCTGCACTATGGCACACTGGTAATCGACGGGGTTACGCCGAACATGATC
GACTATTTCGGACGGCCGTATGAAGGCATCGCCGTGTTCGACGGCAAAAAGATCAC
TGTAACAGGGACCCTGTGGAACGGCAACAAAATTATCGACGAGCGCCTGATCAACC
CCGACGGCTCCCTGCTGTTCCGAGTAACCATCAACGGAGTGACCGGCTGGCGGCTGT
GCGAACGCATTCTGGCGGGAAGCGGAGCTACTAACTTCAGCCTGCTGAAGCAGGCT
GGAGACGTGGAGGAGAACCCTGGACCTTGATAGTAACT
5-13: Kozak sequence
14-202: 1XFLAG-EGF
266-805: 1XFLAG-NLuc
806-871: P2A
872-880: Triple stop codon
Exemplary Repetitive Spacer Sequence
SEQ ID NO: 26
AAAAAACAAAAAACAAAACGGCTATTATGCGTTACCGGCGAGACGCTACGGACTTG
GGAAAATCCGTTGACCTTAAACGGTCGTGTGGGTTCAAGTCCCTCCACCCCCACGCC
GGAAACGCAATAGCCGAAAAACAAAAAACAAAAAAAACAAAAAAAAAACCAAAA
AAACAAAACACA
Forward primer used in Example 55 to amplify template from pCDNA3.1/CAT
SEQ ID NO: 27
CGCGGATCCTAATACGACTCACTATAGGGAGACCCAAGCTGGC
Reverse primer used in Example 55 to amplify 0.5 kb template from pCDNA3.1/CAT
SEQ ID NO: 28
AATAGCCGTTTTGTTTTTTGGATTACCAGTGTGCCATAGTGCAGGATCACATCGTCGT
GGTATTCACTCCAGAGCGATG
Reverse primer used in Example 55 to amplify 1 kb template from pCDNA3.1/CAT
SEQ ID NO: 29
AATAGCCGTTTTGTTTTTTGGATTACCAGTGTGCCATAGTGCAGGATCACACGGGGG
AGGGGCAAACAACAGATGG
Reverse primer used in Example 55 to amplify 2 kb template from pCDNA3.1/CAT
SEQ ID NO: 30
AATAGCCGTTTTGTTTTTTGGATTACCAGTGTGCCATAGTGCAGGATCACGCTTTTTG
CAAAAGCCTAGGCCTCCAAAAAAGCC
Reverse primer used in Example 55 to amplify 4 kb template from pCDNA3.1/CAT
SEQ ID NO: 31
AATAGCCGTTTTGTTTTTTGGATTACCAGTGTGCCATAGTGCAGGATCACTAGCACC
GCCTACATACCTCGCTCTGC
Reverse primer used in Example 55 to amplify 5 kb template from pCDNA3.1/CAT
SEQ ID NO: 32
AATAGCCGTTTTGTTTTTTGGATTACCAGTGTGCCATAGTGCAGGATCACCTATGTG
GCGCGGTATTATCCCGTATTGAC
Reverse primer used in Example 55 to amplify 6.2 kb template from pCDNA3.1/CAT
SEQ ID NO: 33
AATAGCCGTTTTGTTTTTTGGATTACCAGTGTGCCATAGTGCAGGATCACATTTCGAT
AAGCCAGTAAGCAGTGGGTTCTCTAG
Forward qPCR primer used in Example 55 to detect linear transcript from
pCDNA3.1/CAT
SEQ ID NO: 34
ATTCTTGCCCGCCTGATGAA
Reverse qPCR primer used in Example 55 to detect linear transcript from pCDNA3.1/CAT
SEQ ID NO: 35
TTGCTCATGGAAAACGGTGT
Forward qPCR primer used in Example 55 to detect circular transcript from
pCDNA3.1/CAT
SEQ ID NO: 36
TGATCCTGCACTATGGCACA
Reverse qPCR primer used in Example 55 to detect circular transcript from
pCDNA3.1/CAT
SEQ ID NO: 37
CTGGACTAGTGGATCCGAGC
Forward primer sequence used in Example 56 to detect ACTIN
SEQ ID NO: 38
GACGAGGCCCAGAGCAAGAGAGG
Reverse primer sequence used in Example 56 to detect ACTIN
SEQ ID NO: 39
GGTGTTGAAGGTCTCAAACATG
Forward primer sequence used in Example 56 to detect RIG-I
SEQ ID NO: 40
TGTGGGCAATGTCATCAAAA
Reverse primer sequence used in Example 56 to detect RIG-I
SEQ ID NO: 42
GAAGCACTTGCTACCTCTTGC
Forward primer sequence used in Example 56 to detect MDA5
SEQ ID NO: 42
GGCACCATGGGAAGTGATT
Reverse primer sequence used in Example 56 to detect MDA5
SEQ ID NO: 43
ATTTGGTAAGGCCTGAGCTG
Forward primer sequence used in Example 56 to detect PKR
SEQ ID NO: 44
TCGCTGGTATCACTCGTCTG
Reverse primer sequence used in Example 56 to detect PKR
SEQ ID NO: 45
GATTCTGAAGACCGCCAGAG
Forward primer sequence used in Example 56 to detect IFN-beta
SEQ ID NO: 46
CTCTCCTGTTGTGCTTCTCC
Reverse primer sequence used in Example 56 to detect IFN-beta
SEQ ID NO: 47
GTCAAAGTTCATCCTGTCCTTG
EMCV T2A 3XFLAG-GFP F2A 3XFALG-Nluc P2A IS
SEQ ID NO: 48
GGGAATAGCCGAAAAACAAAAAACAAAAAAAACAAAAAAAAAACCAAAAAAACA
AAACACAACGTTACTGGCCGAAGCCGCTTGGAACAAGGCCGGTGTGCGTTTGTCTAT
ATGTTATTTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGGAATGCAA
GGTCTGTTGAATGTCGTGAAGGAAGCAGTTCCTCTGGAAGCTTCTTGTAGACAAACA
ACGTCTGTAGCGACCCTTTGCAGGCAGCGGAACCCCCCACCTGGCGACAGGTGCCTC
TGCGGCCAAAAGCCACGTGTATACGATACACCTGCAAAGGCGGCACAACCCCAGTG
CCACGTTGTGAGTTGGATAGTTGTGGAAAGAGTCAAATGGCTCTCCTCAAGCGTATT
CAACAAGGGGCTGAAGGATGCCCAGAAGGTACCCCATTGTATGGGATCTGATCTGG
GGCCTCGGTGCACATGCTTTACATGTGTTCAGTCGAGGTTAAAAAACGTCCAGGCCC
CCCGAACCACGGGGACGTGGTTTTCCTTTGAAAAACACGATGATAATATGGCCACA
ACCATGGGCTCCGGCGAGGGCAGGGGAAGTCTTCTAACATGCGGGGACGTGGAGGA
AAATCCCGGCCCAGACTACAAGGACGACGACGACAAGATCATCGACTATAAAGACG
ACGACGATAAAGGTGGCGACTATAAGGACGACGACGACAAAGCCATTGTGAGCAA
GGGCGAGGAGCTGTTCACCGGGGTGGTGCCCATCCTGGTCGAGCTGGACGGCGACG
TAAACGGCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATG
AAGCAGCACGACTTCTTCAAGTCCGCCATGCCCGAAGGCTACGTCCAGGAGCGCAC
CATCTTCTTCAAGGACGACGGCAACTACAAGACCCGCGCCGAGGTGAAGTTCGAGG
GCGACACCCTGGTGAACCGCATCGAGCTGAAGGGCATCGACTTCAAGGAGGACGGC
AACATCCTGGGGCACAAGCTGGAGTACAACTACAACAGCCACAACGTCTATATCAT
GGCCGACAAGCAGAAGAACGGCATCAAGGTGAACTTCAAGATCCGCCACAACATCG
AGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAA
AGACCCCAACGAGAAGCGCGATCACATGGTCCTGCTGGAGTTCGTGACCGCCGCCG
GGATCACTCTCGGCATGGACGAGCTGTACAAGGGAAGCGGAGTGAAACAGACTTTG
AATTTTGACCTTCTCAAGTTGGCGGGAGACGTGGAGTCCAACCCTGGACCTGACTAC
AAGGACGACGACGACAAGATCATCGACTATAAAGACGACGACGATAAAGGTGGCG
ACTATAAGGACGACGACGACAAAGCCATTATCATCGTCTTCACACTCGAAGATTTCG
TTGGGGACTGGCGACAGACAGCCGGCTACAACCTGGACCAAGTCCTTGAACAGGGA
GGTGTGTCCAGTTTGTTTCAGAATCTCGGGGTGTCCGTAACTCCGATCCAAAGGATT
GTCCTGAGCGGTGAAAATGGGCTGAAGATCGACATCCATGTCATCATCCCGTATGA
AGGTCTGAGCGGCGACCAAATGGGCCAGATCGAAAAAATTTTTAAGGTGGTGTACC
CTGTGGATGATCATCACTTTAAGGTGATCCTGCACTATGGCACACTGGTAATCGACG
GGGTTACGCCGAACATGATCGACTATTTCGGACGGCCGTATGAAGGCATCGCCGTGT
TCGACGGCAAAAAGATCACTGTAACAGGGACCCTGTGGAACGGCAACAAAATTATC
GACGAGCGCCTGATCAACCCCGACGGCTCCCTGCTGTTCCGAGTAACCATCAACGG
AGTGACCGGCTGGCGGCTGTGCGAACGCATTCTGGCGGGAAGCGGAGCTACTAACT
TCAGCCTGCTGAAGCAGGCTGGAGACGTGGAGGAGAACCCTGGACCTTAAAAAAAA
CAAAAAACAAAACGGCTATT
EMCV T2A 3XFLAG-GFP F2A 3XFALG-Nluc P2A IS
SEQ ID NO: 49
GGGAATAGCCGAAAAACAAAAAACAAAAAAAACAAAAAAAAAACCAAAAAAACA
AAACACAACGTTACTGGCCGAAGCCGCTTGGAACAAGGCCGGTGTGCGTTTGTCTAT
ATGTTATTTTCCACCATATTGCCGTCTTTTGGCAATGTGAGGGCCCGGAAACCTGGC
CCTGTCTTCTTGACGAGCATTCCTAGGGGTCTTTCCCCTCTCGCCAAAGGAATGCAA
GGTCTGTTGAATGTCGTGAAGGAAGCAGTTCCTCTGGAAGCTTCTTGTAGACAAACA
ACGTCTGTAGCGACCCTTTGCAGGCAGCGGAACCCCCCACCTGGCGACAGGTGCCTC
TGCGGCCAAAAGCCACGTGTATACGATACACCTGCAAAGGCGGCACAACCCCAGTG
CCACGTTGTGAGTTGGATAGTTGTGGAAAGAGTCAAATGGCTCTCCTCAAGCGTATT
CAACAAGGGGCTGAAGGATGCCCAGAAGGTACCCCATTGTATGGGATCTGATCTGG
GGCCTCGGTGCACATGCTTTACATGTGTTCAGTCGAGGTTAAAAAACGTCCAGGCCC
CCCGAACCACGGGGACGTGGTTTTCCTTTGAAAAACACGATGATAATATGGCCACA
ACCATGGGCTCCGGCGAGGGCAGGGGAAGTCTTCTAACATGCGGGGACGTGGAGGA
AAATCCCGGCCCAGACTACAAGGACGACGACGACAAGATCATCGACTATAAAGACG
ACGACGATAAAGGTGGCGACTATAAGGACGACGACGACAAAGCCATTGTGAGCAA
GGGCGAGGAGCTGTTCACCGGGGTGGTGCCCATCCTGGTCGAGCTGGACGGCGACG
TAAACGGCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATG
AAGCAGCACGACTTCTTCAAGTCCGCCATGCCCGAAGGCTACGTCCAGGAGCGCAC
CATCTTCTTCAAGGACGACGGCAACTACAAGACCCGCGCCGAGGTGAAGTTCGAGG
GCGACACCCTGGTGAACCGCATCGAGCTGAAGGGCATCGACTTCAAGGAGGACGGC
AACATCCTGGGGCACAAGCTGGAGTACAACTACAACAGCCACAACGTCTATATCAT
GGCCGACAAGCAGAAGAACGGCATCAAGGTGAACTTCAAGATCCGCCACAACATCG
AGGACGGCAGCGTGCAGCTCGCCGACCACTACCAGCAGAACACCCCCATCGGCGAC
GGCCCCGTGCTGCTGCCCGACAACCACTACCTGAGCACCCAGTCCGCCCTGAGCAA
AGACCCCAACGAGAAGCGCGATCACATGGTCCTGCTGGAGTTCGTGACCGCCGCCG
GGATCACTCTCGGCATGGACGAGCTGTACAAGGGAAGCGGAGTGAAACAGACTTTG
AATTTTGACCTTCTCAAGTTGGCGGGAGACGTGGAGTCCAACCCTGGACCTTGATAG
TAAGACTACAAGGACGACGACGACAAGATCATCGACTATAAAGACGACGACGATA
AAGGTGGCGACTATAAGGACGACGACGACAAAGCCATTATCATCGTCTTCACACTC
GAAGATTTCGTTGGGGACTGGCGACAGACAGCCGGCTACAACCTGGACCAAGTCCT
TGAACAGGGAGGTGTGTCCAGTTTGTTTCAGAATCTCGGGGTGTCCGTAACTCCGAT
CCAAAGGATTGTCCTGAGCGGTGAAAATGGGCTGAAGATCGACATCCATGTCATCA
TCCCGTATGAAGGTCTGAGCGGCGACCAAATGGGCCAGATCGAAAAAATTTTTAAG
GTGGTGTACCCTGTGGATGATCATCACTTTAAGGTGATCCTGCACTATGGCACACTG
GTAATCGACGGGGTTACGCCGAACATGATCGACTATTTCGGACGGCCGTATGAAGG
CATCGCCGTGTTCGACGGCAAAAAGATCACTGTAACAGGGACCCTGTGGAACGGCA
ACAAAATTATCGACGAGCGCCTGATCAACCCCGACGGCTCCCTGCTGTTCCGAGTAA
CCATCAACGGAGTGACCGGCTGGCGGCTGTGCGAACGCATTCTGGCGGGAAGCGGA
GCTACTAACTTCAGCCTGCTGAAGCAGGCTGGAGACGTGGAGGAGAACCCTGGACC
TTAAAAAAAACAAAAAACAAAACGGCTATT
URE
SEQ ID NO: 50
UCAUAAUCAAUUUAUUAUUUUCUUUUAUUUUAUUCACAUAAUUUUGUUUUU
CSE
SEQ ID NO: 51
AUUUUGUUUUUAACAUUUC
URE/CSE
SEQ ID NO: 52
UCAUAAUCAAUUUAUUAUUUUCUUUUAUUUUAUUCACAUAAUUUUGUUUUUAUU
UUGUUUUUAACAUUUC
CVB3-GLuc-STOP-URE
SEQ ID NO: 53
AAAAUCCGUUGACCUUAAACGGUCGUGUGGGUUCAAGUCCCUCCACCCCCACGCC
GGAAACGCAAUAGCCGAAAAACAAAAAACAAAAAAAACAAAAAAAAAACCAAAA
AAACAAAACACAUUAAAACAGCCUGUGGGUUGAUCCCACCCACAGGCCCAUUGGG
CGCUAGCACUCUGGUAUCACGGUACCUUUGUGCGCCUGUUUUAUACCCCCUCCCC
CAACUGUAACUUAGAAGUAACACACACCGAUCAACAGUCAGCGUGGCACACCAGC
CACGUUUUGAUCAAGCACUUCUGUUACCCCGGACUGAGUAUCAAUAGACUGCUCA
CGCGGUUGAAGGAGAAAGCGUUCGUUAUCCGGCCAACUACUUCGAAAAACCUAG
UAACACCGUGGAAGUUGCAGAGUGUUUCGCUCAGCACUACCCCAGUGUAGAUCAG
GUCGAUGAGUCACCGCAUUCCCCACGGGCGACCGUGGCGGUGGCUGCGUUGGCGG
CCUGCCCAUGGGGAAACCCAUGGGACGCUCUAAUACAGACAUGGUGCGAAGAGUC
UAUUGAGCUAGUUGGUAGUCCUCCGGCCCCUGAAUGCGGCUAAUCCUAACUGCGG
AGCACACACCCUCAAGCCAGAGGGCAGUGUGUCGUAACGGGCAACUCUGCAGCGG
AACCGACUACUUUGGGUGUCCGUGUUUCAUUUUAUUCCUAUACUGGCUGCUUAU
GGUGACAAUUGAGAGAUCGUUACCAUAUAGCUAUUGGAUUGGCCAUCCGGUGAC
UAAUAGAGCUAUUAUAUAUCCCUUUGUUGGGUUUAUACCACUUAGCUUGAAAGA
GGUUAAAACAUUACAAUUCAUUGUUAAGUUGAAUACAGCAAAAUGGGAGUCAAA
GUUCUGUUUGCCCUGAUCUGCAUCGCUGUGGCCGAGGCCAAGCCCACCGAGAACA
ACGAAGACUUCAACAUCGUGGCCGUGGCCAGCAACUUCGCGACCACGGAUCUCGA
UGCUGACCGCGGGAAGUUGCCCGGCAAGAAGCUGCCGCUGGAGGUGCUCAAAGAG
AUGGAAGCCAAUGCCCGGAAAGCUGGCUGCACCAGGGGCUGUCUGAUCUGCCUGU
CCCACAUCAAGUGCACGCCCAAGAUGAAGAAGUUCAUCCCAGGACGCUGCCACAC
CUACGAAGGCGACAAAGAGUCCGCACAGGGCGGCAUAGGCGAGGCGAUCGUCGAC
AUUCCUGAGAUUCCUGGGUUCAAGGACUUGGAGCCCAUGGAGCAGUUCAUCGCAC
AGGUCGAUCUGUGUGUGGACUGCACAACUGGCUGCCUCAAAGGGCUUGCCAACGU
GCAGUGUUCUGACCUGCUCAAGAAGUGGCUGCCGCAACGCUGUGCGACCUUUGCC
AGCAAGAUCCAGGGCCAGGUGGACAAGAUCAAGGGGGCCGGUGGUGACUAAUCA
UAAUCAAUUUAUUAUUUUCUUUUAUUUUAUUCACAUAAUUUUGUUUUUAUUUUG
UUUUUAACAUUUCAAAAAACAAAAAACAAAACGGCUAUUAUGCGUUACCGGCGA
GACGCUACGGACUU
CVB3-GLuc-STOP-URE/CSE
SEQ ID NO: 54
AAAAUCCGUUGACCUUAAACGGUCGUGUGGGUUCAAGUCCCUCCACCCCCACGCC
GGAAACGCAAUAGCCGAAAAACAAAAAACAAAAAAAACAAAAAAAAAACCAAAA
AAACAAAACACAUUAAAACAGCCUGUGGGUUGAUCCCACCCACAGGCCCAUUGGG
CGCUAGCACUCUGGUAUCACGGUACCUUUGUGCGCCUGUUUUAUACCCCCUCCCC
CAACUGUAACUUAGAAGUAACACACACCGAUCAACAGUCAGCGUGGCACACCAGC
CACGUUUUGAUCAAGCACUUCUGUUACCCCGGACUGAGUAUCAAUAGACUGCUCA
CGCGGUUGAAGGAGAAAGCGUUCGUUAUCCGGCCAACUACUUCGAAAAACCUAG
UAACACCGUGGAAGUUGCAGAGUGUUUCGCUCAGCACUACCCCAGUGUAGAUCAG
GUCGAUGAGUCACCGCAUUCCCCACGGGCGACCGUGGCGGUGGCUGCGUUGGCGG
CCUGCCCAUGGGGAAACCCAUGGGACGCUCUAAUACAGACAUGGUGCGAAGAGUC
UAUUGAGCUAGUUGGUAGUCCUCCGGCCCCUGAAUGCGGCUAAUCCUAACUGCGG
AGCACACACCCUCAAGCCAGAGGGCAGUGUGUCGUAACGGGCAACUCUGCAGCGG
AACCGACUACUUUGGGUGUCCGUGUUUCAUUUUAUUCCUAUACUGGCUGCUUAU
GGUGACAAUUGAGAGAUCGUUACCAUAUAGCUAUUGGAUUGGCCAUCCGGUGAC
UAAUAGAGCUAUUAUAUAUCCCUUUGUUGGGUUUAUACCACUUAGCUUGAAAGA
GGUUAAAACAUUACAAUUCAUUGUUAAGUUGAAUACAGCAAAAUGGGAGUCAAA
GUUCUGUUUGCCCUGAUCUGCAUCGCUGUGGCCGAGGCCAAGCCCACCGAGAACA
ACGAAGACUUCAACAUCGUGGCCGUGGCCAGCAACUUCGCGACCACGGAUCUCGA
UGCUGACCGCGGGAAGUUGCCCGGCAAGAAGCUGCCGCUGGAGGUGCUCAAAGAG
AUGGAAGCCAAUGCCCGGAAAGCUGGCUGCACCAGGGGCUGUCUGAUCUGCCUGU
CCCACAUCAAGUGCACGCCCAAGAUGAAGAAGUUCAUCCCAGGACGCUGCCACAC
CUACGAAGGCGACAAAGAGUCCGCACAGGGCGGCAUAGGCGAGGCGAUCGUCGAC
AUUCCUGAGAUUCCUGGGUUCAAGGACUUGGAGCCCAUGGAGCAGUUCAUCGCAC
AGGUCGAUCUGUGUGUGGACUGCACAACUGGCUGCCUCAAAGGGCUUGCCAACGU
GCAGUGUUCUGACCUGCUCAAGAAGUGGCUGCCGCAACGCUGUGCGACCUUUGCC
AGCAAGAUCCAGGGCCAGGUGGACAAGAUCAAGGGGGCCGGUGGUGACUAAUCA
UAAUCAAUUUAUUAUUUUCUUUUAUUUUAUUCACAUAAUUUUGUUUUUAUUUUG
UUUUUAACAUUUCAAAAAACAAAAAACAAAACGGCUAUUAUGCGUUACCGGCGA
GACGCUACGGACUU
Complementary primer used for example 54
SEQ ID NO: 55
CACCGCTCAGGACAATCCTT
CVB3 IRES
SEQ ID NO: 56
TTAAAACAGCCTGTGGGTTGATCCCACCCACAGGCCCATTGGGCGCTAGCACTCTGG
TATCACGGTACCTTTGTGCGCCTGTTTTATACCCCCTCCCCCAACTGTAACTTAGAAG
TAACACACACCGATCAACAGTCAGCGTGGCACACCAGCCACGTTTTGATCAAGCAC
TTCTGTTACCCCGGACTGAGTATCAATAGACTGCTCACGCGGTTGAAGGAGAAAGC
GTTCGTTATCCGGCCAACTACTTCGAAAAACCTAGTAACACCGTGGAAGTTGCAGAG
TGTTTCGCTCAGCACTACCCCAGTGTAGATCAGGTCGATGAGTCACCGCATTCCCCA
CGGGCGACCGTGGCGGTGGCTGCGTTGGCGGCCTGCCCATGGGGAAACCCATGGGA
CGCTCTAATACAGACATGGTGCGAAGAGTCTATTGAGCTAGTTGGTAGTCCTCCGGC
CCCTGAATGCGGCTAATCCTAACTGCGGAGCACACACCCTCAAGCCAGAGGGCAGT
GTGTCGTAACGGGCAACTCTGCAGCGGAACCGACTACTTTGGGTGTCCGTGTTTCAT
TTTATTCCTATACTGGCTGCTTATGGTGACAATTGAGAGATCGTTACCATATAGCTAT
TGGATTGGCCATCCGGTGACTAATAGAGCTATTATATATCCCTTTGTTGGGTTTATAC
CACTTAGCTTGAAAGAGGTTAAAACATTACAATTCATTGTTAAGTTGAATACAGCAA
A
Gluc
SEQ ID NO: 57
ATGGGAGTCAAAGTTCTGTTTGCCCTGATCTGCATCGCTGTGGCCGAGGCCAAGCCC
ACCGAGAACAACGAAGACTTCAACATCGTGGCCGTGGCCAGCAACTTCGCGACCAC
GGATCTCGATGCTGACCGCGGGAAGTTGCCCGGCAAGAAGCTGCCGCTGGAGGTGC
TCAAAGAGATGGAAGCCAATGCCCGGAAAGCTGGCTGCACCAGGGGCTGTCTGATC
TGCCTGTCCCACATCAAGTGCACGCCCAAGATGAAGAAGTTCATCCCAGGACGCTG
CCACACCTACGAAGGCGACAAAGAGTCCGCACAGGGCGGCATAGGCGAGGCGATC
GTCGACATTCCTGAGATTCCTGGGTTCAAGGACTTGGAGCCCATGGAGCAGTTCATC
GCACAGGTCGATCTGTGTGTGGACTGCACAACTGGCTGCCTCAAAGGGCTTGCCAAC
GTGCAGTGTTCTGACCTGCTCAAGAAGTGGCTGCCGCAACGCTGTGCGACCTTTGCC
AGCAAGATCCAGGGCCAGGTGGACAAGATCAAGGGGGCCGGTGGTGACTAA
EMCV IRES with stop mutations
SEQ ID NO: 58
ACGTTACTGGCCGAAGCCGCTTGGAACAAGGCCGGTGTGCGTTTGTCTATATGTTAT
TTTCCACCATATTGCCGTCTTTTGGCAATGTGAGGGCCCGGAAACCTGGCCCTGTCTT
CTTGACGAGCATTCCTAGGGGTCTTTCCCCTCTCGCCAAAGGAATGCAAGGTCTGTT
GAATGTCGTGAAGGAAGCAGTTCCTCTGGAAGCTTCTTCAAGACAAACAACGTCTGT
AGCGACCCTTTGCAGGCAGCGGAACCCCCCACCTGGCGACAGGTGCCTCTGCGGCC
AAAAGCCACGTGTATACGATACACCTGCAAAGGCGGCACAACCCCAGTGCCACGTT
GTGAGTTGGATAGTTGTGGAAAGAGTCAAATGGCTCTCCTCAAGCGTATTCAACAA
GGGGCTGAAGGATGCCCAGAAGGTACCCCATTGTATGGGATCTGATCTGGGGCCTC
GGTGCACATGCTTTACATGTGTTCAGTCGAGGTTAAAAAACGTCCAGGCCCCCCGAA
CCACGGGGACGTGGTTTTCCTTTGAAAAACACGATGATAATATGGCCACAACCATG
SPACER 1
SEQ ID NO: 59
AAAAUCCGUUGACCUUAAACGGUCGUGUGGGUUCAAGUCCCUCCACCCCCACGCC
GGAAACGCAAUAGCCGAAAAACAAAAAACAAAAAAAACAAAAAAAAAACCAAAA
AAACAAAACACA
SPACER 2
AAAAAACAAAAAACAAAACGGCUAUUAUGCGUUACCGGCGAGACGCUACGGACU
U
SEQ ID: 60
ATACCAGCCGAAAGGCCCTTGGCAGAGAGGTCTGAAAAGACCTCTGCTGACTATGT
GATCTTATTAAAATTAGGTTAAATTTCGAGGTTAAAAATAGTTTTAATATTGCTATA
GTCTTAGAGGTCTTGTATATTTATACTTACCACACAAGATGGACCGGAGCAGCCCTC
CAATATCTAGTGTACCCTCGTGCTCGCTCAAACATTAAGTGGTGTTGTGCGAAAAGA
ATCTCACTTCAAGAAAAAGAAACTAGT
Gaussia Luciferase DNA template used in Example 5
ATGGGAGTCAAAGTTCTGTTTGCCCTGATCTGCATCGCTGTGGCCGAGGCCAAGCCC
ACCGAGAACAACGAAGACTTCAACATCGTGGCCGTGGCCAGCAACTTCGCGACCAC
GGATCTCGATGCTGACCGCGGGAAGTTGCCCGGCAAGAAGCTGCCGCTGGAGGTGC
TCAAAGAGATGGAAGCCAATGCCCGGAAAGCTGGCTGCACCAGGGGCTGTCTGATC
TGCCTGTCCCACATCAAGTGCACGCCCAAGATGAAGAAGTTCATCCCAGGACGCTG
CCACACCTACGAAGGCGACAAAGAGTCCGCACAGGGCGGCATAGGCGAGGCGATC
GTCGACATTCCTGAGATTCCTGGGTTCAAGGACTTGGAGCCCATGGAGCAGTTCATC
GCACAGGTCGATCTGTGTGTGGACTGCACAACTGGCTGCCTCAAAGGGCTTGCCAAC
GTGCAGTGTTCTGACCTGCTCAAGAAGTGGCTGCCGCAACGCTGTGCGACCTTTGCC
AGCAAGATCCAGGGCCAGGTGGACAAGATCAAGGGGGCCGGTGGTGACTAA
EMCV IRES used in Example 5
ACGTTACTGGCCGAAGCCGCTTGGAATAAGGCCGGTGTGCGTTTGTCTATATGTTAT
TTTCCACCATATTGCCGTCTTTTGGCAATGTGAGGGCCCGGAAACCTGGCCCTGTCTT
CTTGACGAGCATTCCTAGGGGTCTTTCCCCTCTCGCCAAAGGAATGCAAGGTCTGTT
GAATGTCGTGAAGGAAGCAGTTCCTCTGGAAGCTTCTTGAAGACAAACAACGTCTG
TAGCGACCCTTTGCAGGCAGCGGAACCCCCCACCTGGCGACAGGTGCCTCTGCGGC
CAAAAGCCACGTGTATAAGATACACCTGCAAAGGCGGCACAACCCCAGTGCCACGT
TGTGAGTTGGATAGTTGTGGAAAGAGTCAAATGGCTCTCCTCAAGCGTATTCAACAA
GGGGCTGAAGGATGCCCAGAAGGTACCCCATTGTATGGGATCTGATCTGGGGCCTC
GGTGCACATGCTTTACATGTGTTTAGTCGAGGTTAAAAAACGTCTAGGCCCCCCGAA
CCACGGGGACGTGGTTTTCCTTTGAAAAACACGATGATAATA
Gaussia Luciferase ORF used in Example 4
AUGGGAGUCAAAGUUCUGUUUGCCCUGAUCUGCAUCGCUGUGGCCGAGGCCAAGC
CCACCGAGAACAACGAAGACUUCAACAUCGUGGCCGUGGCCAGCAACUUCGCGAC
CACGGAUCUCGAUGCUGACCGCGGGAAGUUGCCCGGCAAGAAGCUGCCGCUGGAG
GUGCUCAAAGAGAUGGAAGCCAAUGCCCGGAAAGCUGGCUGCACCAGGGGCUGUC
UGAUCUGCCUGUCCCACAUCAAGUGCACGCCCAAGAUGAAGAAGUUCAUCCCAGG
ACGCUGCCACACCUACGAAGGCGACAAAGAGUCCGCACAGGGCGGCAUAGGCGAG
GCGAUCGUCGACAUUCCUGAGAUUCCUGGGUUCAAGGACUUGGAGCCCAUGGAGC
AGUUCAUCGCACAGGUCGAUCUGUGUGUGGACUGCACAACUGGCUGCCUCAAAGG
GCUUGCCAACGUGCAGUGUUCUGACCUGCUCAAGAAGUGGCUGCCGCAACGCUGU
GCGACCUUUGCCAGCAAGAUCCAGGGCCAGGUGGACAAGAUCAAGGGGGCCGGUG
GUGACUAA
EMCV IRES used in Example 4
ACGUUACUGGCCGAAGCCGCUUGGAAUAAGGCCGGUGUGCGUUUGUCUAUAUGU
UAUUUUCCACCAUAUUGCCGUCUUUUGGCAAUGUGAGGGCCCGGAAACCUGGCCC
UGUCUUCUUGACGAGCAUUCCUAGGGGUCUUUCCCCUCUCGCCAAAGGAAUGCAA
GGUCUGUUGAAUGUCGUGAAGGAAGCAGUUCCUCUGGAAGCUUCUUGAAGACAA
ACAACGUCUGUAGCGACCCUUUGCAGGCAGCGGAACCCCCCACCUGGCGACAGGU
GCCUCUGCGGCCAAAAGCCACGUGUAUAAGAUACACCUGCAAAGGCGGCACAACC
CCAGUGCCACGUUGUGAGUUGGAUAGUUGUGGAAAGAGUCAAAUGGCUCUCCUC
AAGCGUAUUCAACAAGGGGCUGAAGGAUGCCCAGAAGGUACCCCAUUGUAUGGG
AUCUGAUCUGGGGCCUCGGUGCACAUGCUUUACAUGUGUUUAGUCGAGGUUAAA
AAACGUCUAGGCCCCCCGAACCACGGGGACGUGGUUUUCCUUUGAAAAACACGAU
GAUAAUA

1. A method of making a pharmaceutical composition, comprising:
a) providing a plurality of linear polyribonucleotide molecules;
b) circularizing the linear polyribonucleotide molecules to provide a preparation of circular polyribonucleotide molecules;
c) processing the preparation to substantially remove linear polyribonucleotide molecules remaining in the preparation;
d) optionally evaluating the amount of linear polyribonucleotide molecules in the preparation remaining after the processing step; and
e) further processing the preparation to produce the pharmaceutical composition for pharmaceutical use.

2. The method of claim 1, wherein further processing of step d) comprises one or more of:
f) processing the preparation to substantially remove deoxyribonucleotide molecules;
g) evaluating the amount of deoxyribonucleotide molecules in the preparation;
h) formulating the preparation with a pharmaceutical excipient;
i) concentrating the preparation; and
j) documenting the amount of deoxyribonucleotide molecules in the preparation in a print or digital media.

3. The method of claim 1 or claim 2, wherein further processing of step d) comprises one or more of:
f) processing the preparation to substantially remove protein contamination;
g) evaluating the amount of protein contamination in the preparation;
h) formulating the preparation with a pharmaceutical excipient; and
i) concentrating the preparation.

4. The method of any one of claims 1-3, wherein further processing of step d) comprises one or more of:
f) processing the preparation to substantially remove endotoxin;
g) evaluating the amount of endotoxin in the preparation;
h) formulating the preparation with a pharmaceutical excipient; and
i) concentrating the preparation.

5. The method of any of claims 1-4, wherein the circularizing step is performed by splint ligation.

6. The method of any one of claims 1-5, wherein the linear polyribonucleotide molecules comprises a linear polyribonucleotide molecule counterpart of the circular polyribonucleotide molecules, a linear polyribonucleotide molecule non-counterpart of the circular polyribonucleotide molecules, or a combination thereof.

7. The method of any one of claims 1-6, wherein the protein contamination comprises an enzyme.

8. The method of any one of claims 1-7, wherein the pharmaceutical composition comprises no more than 20% (w/w) linear polyribonucleotide molecules of the total ribonucleotide molecules in the preparation.

9. The method of any one of claims 1-8, wherein the pharmaceutical composition comprises no more than 10% (w/w) linear polyribonucleotide molecules of the total ribonucleotide molecules in the preparation.

10. A pharmaceutical preparation of circular polyribonucleotide molecules, the pharmaceutical preparation comprising circular polyribonucleotide molecules and no more than 5% (w/w) nicked polyribonucleotide molecules of the total ribonucleotide molecules in the pharmaceutical preparation.

11. The pharmaceutical preparation of claim 10, comprising no more than 2% (w/w) nicked polyribonucleotide molecules of the total ribonucleotide molecules in the pharmaceutical preparation.

12. The pharmaceutical preparation of claim 10 or claim 11, wherein the pharmaceutical preparation:
(i) comprises less than 10 EU/kg or lacks endotoxin as measured by a Limulus amebocyte lysate test; and/or
(ii) comprises a bioburden of less than 100 CFU/100 ml or less than 10 CFU/100 ml before sterilization; and/or
(iii) is a sterile pharmaceutical preparation, e.g., supports growth of fewer than 100 viable microorganisms as tested under aseptic conditions; and/or
(iv) meets the standard of USP <71>; and/or
(v) meets the standard of USP <85>; and/or
(vi) is an intermediate pharmaceutical preparation of a final drug product; and/or
(vii) is a final drug product for administration to a subject; and/or
(viii) comprises a concentration of at least 0.1 ng/mL of the circular polyribonucleotide molecules; and/or
(ix) comprises no more than about 9% (w/w), 8% (w/w), 7% (w/w), 6% (w/w), 5% (w/w), 4% (w/w), 3% (w/w), 2% (w/w), 1% (w/w), or 0.5% (w/w) nicked polyribonucleotide molecules; and/or
(x) is substantially free of a process-related impurity selected from a cell protein, a cell deoxyribonucleic acid, an enzyme, a reagent component, a gel component, or a chromatographic material; and/or
(xi) has a reduced level of one or more markers of an immune or inflammatory response after purification compared to prior to purification, e.g., wherein the one or more markers of an immune or inflammatory response is: (a) a cytokine or an immunogenic related gene; and/or (b) expression of a gene selected from the group consisting of RIG-I, MDA5, PKR, IFN-beta, OAS, and OASL.

13. The pharmaceutical preparation of any one of claims 10-12, wherein the circular polyribonucleotide molecules comprise one or more expression sequences and a stagger element at a 3′ end of at least one expression sequence.

14. The pharmaceutical preparation of any one claims 10-13, wherein at least 80% (w/w) of total ribonucleotide molecules in the pharmaceutical preparation are circular polyribonucleotide molecules.

15. The pharmaceutical preparation of any one of claims 10-14, wherein the pharmaceutical preparation comprises no more than 20% (w/w) linear polyribonucleotide molecules of the total ribonucleotide molecules in the preparation.

16. A method of making a pharmaceutical drug substance, comprising:
a) providing a plurality of linear polyribonucleotide molecules;
b) circularizing the plurality of linear polyribonucleotide molecules to provide a preparation of circular polyribonucleotide molecules;
c) evaluating the amount of linear polyribonucleotide molecules remaining in the preparation; and
d) processing the preparation of circular polyribonucleotide molecules as a pharmaceutical drug substance if the preparation meets a reference criterion for an amount of linear polyribonucleotide molecules present in the preparation.

17. A method of making a pharmaceutical drug product, comprising:
a) providing a plurality of linear polyribonucleotide molecules;
b) circularizing the plurality of linear polyribonucleotide molecules to provide a preparation of circular polyribonucleotide molecules;
c) measuring the amount of linear polyribonucleotide molecules in the preparation;
d) formulating the preparation of circular polyribonucleotide molecules as a pharmaceutical drug product if the preparation meets a reference criterion for an amount of linear polyribonucleotide molecules present in the preparation; and
e) labelling and shipping the pharmaceutical drug product if it meets a reference criterion for the amount of linear polyribonucleotide molecules present in the pharmaceutical drug product.

18. The method of claim 16 or claim 17, wherein the circularizing step is performed by splint ligation.

19. The method of any one of claims 16-18, wherein the formulating the preparation of circular polyribonucleotide molecules comprises combining the preparation of circular polyribonucleotide molecules with a pharmaceutical excipient.

20. The method of any one of claims 16-19, wherein the reference criterion for the amount of linear polyribonucleotide molecules present in the preparation is:
(i) a pharmaceutical release specification; or
(ii) the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules; or
(iii) the presence of no more than a specified amount of (e.g., an undetectable level or level below a detection limit when measured) of linear polyribonucleotide molecules when measured by microscopy, by spectrophotometry, by fluorometry, by denaturing urea polyacrylamide gel electrophoresis imaging, by UV-Vis spectrophotometry, by RNA electrophoresis, or by RNAse H analysis;
and optionally, wherein the pharmaceutical preparation further meets a reference criterion for the sequence of the circular polyribonucleotide molecules, e.g., a sequence having at least 80% (e.g., 85%, 90%, 95%, 97%, 99%, 100%) sequence identity to a reference circular polyribonucleotide sequence.

21. The method of any one of claims 16-20, wherein the pharmaceutical drug product or pharmaceutical drug substance comprises:
(i) a concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 5 mg/mL, 10 mg/mL, 100 mg/mL, 200 mg/mL, or 500 mg/mL circular polyribonucleotide molecules; or
(ii) at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to total ribonucleotide molecules in the pharmaceutical preparation.

22. The method of claim 21, wherein the at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to total ribonucleotide molecules in the pharmaceutical preparation is measured by microscopy, by spectrophotometry, by fluorometry, by denaturing urea polyacrylamide gel electrophoresis imaging, by UV-Vis spectrophotometry, by RNA electrophoresis, by RNAse H analysis, by UV spectroscopic or fluorescence detectors, by light scattering techniques, by surface plasmon resonance (SPR) with or without the use of methods of separation including HPLC, by chip or gel based electrophoresis with or without using either pre or post separation derivatization methodologies, by using methods of detection that use silver or dye stains or radioactive decay for detection of linear polyribonucleotide molecules, or by methods that utilize microscopy, visual methods or a spectrophotometer.

23. The method of any one of claims 16-22, wherein the pharmaceutical drug product or pharmaceutical drug substance:
(i) comprises less than 10 EU/kg or lacks endotoxin as measured by the Limulus amebocyte lysate test; and/or
(ii) comprises a bioburden of less than 100 CFU/100 ml or less than 10 CFU/100 ml before sterilization; and/or
(iii) is a sterile drug product or sterile drug substance, and optionally, supports growth of fewer than 100 viable microorganisms as tested under aseptic conditions; and/or
(iv) meets the standard of USP <71>; and/or
(v) meets the standard of USP <85>; and/or
(vi) comprises an A260/A280 absorbance ratio of from about 1.6 to 2.3 as measured by a spectrophotometer.

24. The method of any one of claims 16-23, wherein the circular polyribonucleotide molecules comprise one or more expression sequences and a stagger element at a 3′ end of at least one expression sequence.

25. The method of any one of claims 16-24, wherein the preparation further meets a reference criterion:
(i) for the amount of deoxyribonucleotide molecules present in the preparation, e.g., is the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml, 1000 μg/mL, 5000 μg/mL, 10,000 μg/mL, or 100,000 μg/mL of deoxyribonucleotide molecules; and/or
(ii) for the amount of protein contamination present in the preparation, e.g., is the presence of a protein contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of the protein contamination per milligram (mg) of the circular polyribonucleotide molecules.

26. A pharmaceutical preparation of circular polyribonucleotide molecules, wherein at least 91% (w/w), 92% (w/w), 93% (w/w), 94% (w/w), 95% (w/w), 96% (w/w), 97% (w/w), 98% (w/w) or 99% (w/w) of total ribonucleotide molecules in the pharmaceutical preparation are circular polyribonucleotide molecules.

27. A pharmaceutical preparation of circular polyribonucleotide molecules, the pharmaceutical preparation comprising no more than 0.5% (w/w), 1% (w/w), 2% (w/w) or 5% (w/w) linear polyribonucleotide molecules of the total ribonucleotide molecules in the pharmaceutical preparation.

28. The pharmaceutical preparation of claim 26 or claim 27, wherein the pharmaceutical preparation:
(i) comprises less than 10 EU/kg or lacks endotoxin as measured by a Limulus amebocyte lysate test; and/or
(ii) comprises a bioburden of less than 100 CFU/100 ml or less than 10 CFU/100 ml before sterilization; and/or
(iii) is a sterile pharmaceutical preparation, e.g., supports growth of fewer than 100 viable microorganisms as tested under aseptic conditions; and/or
(iv) meets the standard of USP <71>; and/or
(v) meets the standard of USP <85>; and/or
(vi) is an intermediate pharmaceutical preparation of a final drug product; and/or
(vii) is a final drug product for administration to a subject; and/or
(viii) comprises a concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1 mg/mL, 2 mg/mL, 3 mg/mL, 5 mg/mL, 10 mg/mL, 100 mg/mL, 200 mg/mL, or 500 mg/mL circular polyribonucleotide molecules; and/or
(ix) comprises no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL of deoxyribonucleotide molecules; and/or
(x) comprises a protein contamination (e.g. an enzyme) of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of the protein contamination per milligram (mg) of the circular polyribonucleotide molecules; and/or
(xi) comprises an A260/A280 absorbance ratio of from about 1.6 to 2.3 as measured by a spectrophotometer;
(xii) is substantially free of a process-related impurity selected from a cell protein, a cell deoxyribonucleic acid, an enzyme, a reagent component, a gel component, or a chromatographic material; and/or
(xiii) has a reduced level of one or more markers of an immune or inflammatory response after purification compared to prior to purification.

29. The pharmaceutical preparation of any one of claims 26-28, wherein the circular polyribonucleotide molecules:
(i) comprise a quasi-helical structure; and/or
(ii) comprise a quasi-double stranded secondary structure; and/or
(iii) comprise one or more expression sequences and a stagger element at a 3′ end of at least one expression sequence.

30. A method of delivering a circular polyribonucleotide to a subject or to a cell or tissue of a subject, comprising administering the pharmaceutical preparation of any one of claims 10-15 and 26-29, the pharmaceutical composition of any one of claims 1-9, the pharmaceutical drug substance of any one of claims 16 and 18-25, or the pharmaceutical drug product of any one of claims 17-25 to the cell or tissue of the subject, wherein the circular polyribonucleotide or a product translated from the circular polyribonucleotide is detected in the cell, tissue, or subject at least 3 days after the administering step.

31. The method of claim 30, further comprising:
(i) evaluating the presence the circular polyribonucleotide or a product translated from the circular polyribonucleotide in the cell, tissue or subject before the administering step; and/or
(ii) evaluating the presence the circular polyribonucleotide or a product translated from the circular polyribonucleotide in the cell, tissue or subject after the administering step.

32. A parenteral nucleic acid delivery system comprising (i) the pharmaceutical preparation of any one of claims 10-15 and 26-29, the pharmaceutical composition of any one of claims 1-9, the pharmaceutical drug substance of any one of claims 16 and 18-25, or the pharmaceutical drug product of any one of claims 17-25, and (ii) a parenterally acceptable diluent.

33. A method of delivering a circular polyribonucleotide to a subject comprising parenterally administering to a subject in need thereof the pharmaceutical preparation of any one of claims 10-15 and 26-29, the pharmaceutical composition of any one of claims 1-9, the pharmaceutical drug substance of any one of claims 16 and 18-25, or the pharmaceutical drug product of any one of claims 17-25 to a subject in need thereof.

34. A method of delivering a circular polyribonucleotide to a cell or tissue of a subject, comprising administering parenterally to the cell or tissue the pharmaceutical preparation of any one of claims 10-15 and 26-29, the pharmaceutical composition of any one of claims 1-9, the pharmaceutical drug substance of any one of claims 16 and 18-25, or the pharmaceutical drug product of any one of claims 17-25.

35. The method of claim 33 or 34, wherein the the pharmaceutical preparation of any one of claims 10-15 and 26-29 the pharmaceutical composition of any one of claims 1-9, the pharmaceutical drug substance of any one of claims 16 and 18-25, or the pharmaceutical drug product of any one of claims 17-25 comprises a carrier.

36. The parenteral nucleic acid delivery system of claim 32, or the method of claims 34-35, wherein the the pharmaceutical preparation of any one of claims 10-15 and 26-29, the pharmaceutical composition of any one of claims 1-9, the pharmaceutical drug substance of any one of claims 16 and 18-25, or the pharmaceutical drug product of any one of claims 17-25 comprises a diluent and is free of any carrier.

37. The method of any one of claims 33-36, wherein parenteral administration is intravenously, intramuscularly, ophthalmically or topically.

38. A method of making a pharmaceutical drug substance, comprising:
a) providing a plurality of linear polyribonucleotide molecules;
b) circularizing the plurality of linear polyribonucleotide molecules to provide a preparation of circular polyribonucleotide molecules;
c) evaluating the amount of linear and/or nicked polyribonucleotide molecules remaining in the preparation; and
d) processing the preparation of circular polyribonucleotide molecules as a pharmaceutical drug substance if the preparation meets a reference criterion for an amount of linear and/or nicked polyribonucleotide molecules present in the preparation.

39. A method of making a pharmaceutical drug product, comprising:
a) providing a plurality of linear polyribonucleotide molecules;
b) circularizing the plurality of linear polyribonucleotide molecules to provide a preparation of circular polyribonucleotide molecules;
c) measuring the amount of linear and/or nicked polyribonucleotide molecules in the preparation;
d) formulating the preparation of circular polyribonucleotide molecules as a pharmaceutical drug product if the preparation meets a reference criterion for an amount of linear and/or nicked polyribonucleotide molecules present in the preparation; and
e) labelling and shipping the pharmaceutical drug product if it meets a reference criterion for the amount of linear polyribonucleotide molecules present in the pharmaceutical drug product.

40. The method of claim 38 or 39, wherein the reference criterion for the amount of linear and/or nicked polyribonucleotide molecules present in the preparation is selected from:
(a) no more than 20%, 15%, 10%, 5%, 2%, 1%, or 0.5% (w/w) linear polyribonucleotide molecules relative to the total ribonucleotide molecules in the preparation;
(b) no more than 5%, 4%, 3%, 2%, 1%, 0.5%, or 0.1% (w/w) nicked polyribonucleotide molecules relative to the total ribonucleotide molecules in the preparation; or
(c) no more than 20%, 15%, 10%, 5%, 2%, 1%, or 0.5% (w/w) combined linear and nicked polyribonucleotide molecules relative to the total ribonucleotide molecules in the preparation.
